(19) United States
(12) Patent Application Publication
(10) Pub. No.: US 2010/0041089 A1
(43) Pub. Date: Feb. 18, 2010

(54) IMPROVED METHOD FOR DETECTING CHEMICAL SUBSTANCES

(75) Inventor: Chiaki Katou, Kusatsu-shi (JP)

Correspondence Address: BIRCH STEWART KOLASCH & BIRCH, PO BOX 747, FALLS CHURCH, VA, 22040-0747, US

(73) Assignee: Daikin Industries, Lrd., Osaka-shi (JP)

(21) Appl. No.: 12/302,416

(22) PCT Filed: May 29, 2007
(86) PCT No.: PCT/JP2007/060866

§ 371 Date: Oct. 23, 2009

(30) Foreign Application Priority Data
May 29, 2006 (JP) 2006-149044

(51) Int. Cl. C12Q 1/02; C12N 1/19; C12M 1/34

(52) U.S. Cl. 435/29; 435/254.2; 435/287.1

(57) ABSTRACT

The present invention provides an assay method for conveniently and easily detecting existence or a quantity of a chemical substance in the environment without relying on expensive measurement devices used in fluorometry. More specifically, the present invention is characterized in that a transformed cell which is transformed by operably linking a polynucleotide encoding oxidoreductase at a downstream of a promoter gene for monitoring whose promoter activity varies with responding to a chemical substance is used.

### FIELD OF THE INVENTION

This invention relates to a biology-based method for detecting chemical substances by using an oxygen electrode. It also relates to a transformed cell and a device as used for the method.

### BACKGROUND ART

Environmental chemical fate search has been conducted every year for 24 years from 1974 through 1998 by Environment Agency, and revealed that about 40% of 775 chemical substances that have been searched so far are emitted into the environment. Chemical substances that are industrially produced at the present in Japan are estimated about 50,000, and the production scale and the kinds of chemical substances are increasing year by year. It is known that chemical substances that are accidentally produced by water treatment with chlorine and incineration pollute the environment. Although such facts allow us to predict that there are a large number of chemical substances that have been accumulated in the environment, it is extremely difficult to search and examine individually the all chemical substances.

Conventional bioassays (approaches to evaluate the harmful effects on biological materials on the basis of their responses) wherein inhibited growth and particular biological responses in individuals and cells of fishes, daphnia and shellfish are used as indicators make it possible to determine the presence or absence of the toxicity of chemical substances in the environment, but neither possible to evaluate the characters nor origins of the toxicity. The evaluation methods based on the activity of nitrite-forming bacteria or nitrate-forming bacteria (Japanese Patent Publication (kokai) No. 123705/1994, Japanese Patent Publication (kokai) No. 2000-206087) and the activity of iron bacteria have been proposed, and devices such as Acute toxicants monitor (Fuji Electric Corporate Research and Development, Ltd. Japan) are marketed. In foreign countries, the devices for evaluation based on emission intensity of luminous bacteria are commercially available (MICROTOX, azur, Co., USA; LUMIS, drlange, Co. Germany). However, those devices still involve conventional bioassays, and never provide any detailed information of toxic chemical substances.

In Japan, the risk control of chemical substances is reconsidered every time chemical substance pollution is newly found, and official regulations and self-imposed regulations are combined to organize the system for risk control. However, any system has not been yet organized that could quickly respond to the present complicated and diversified conditions including accidental productions and environmental emission of toxic chemical substance as typified by trihalomethane and dioxin. Animal experiments as used in the method for evaluation of toxic substance of “Law Concerning Examination and Manufacture, etc. of Chemical Substances” are expensive and time-consuming, and are not accepted across the world. Although, as such, the control system has been continuously discussed, it has not been successfully accomplished because there is no way to dissolve the problem.

Thus, it is desired to evaluate effects of chemical substances on human bodies and the ecosystem.

As a method for evaluating effects of chemical substances on human bodies and the ecosystem, a reporter gene assay is known. This reporter gene assay is a technique for measuring an activity of a specific gene of target to investigate gene functions such as mainly a transcription activity, and it includes a promoter assay and the like. This promoter assay is a technique for indirectly measuring an expression of a gene by operably linking a polynucleotide encoding a marker protein to a base sequence for a promoter of the gene [Barelle C J, Manson C L, MacCallum D M, Odds F C, Gow Na, Brown A J.: GFP as a quantitative reporter of gene regulation in Candida albicans. Yeast 2004 March; 21 (4):333-40].

In conventional bioassay techniques, existence of a chemical substance is assayed by using cell responses of microorganisms against the chemical substance, such as life and death, a proliferative activity, a respiration rate of a cell, and a change in an expression of a specific gene, as an index. In a promoter assay technique, existence of a chemical substance is assayed by using cell responses of a marker protein linked to a promoter such as a change in a promoter activity as an index.

Examples of the marker proteins include GFP (Green Fluorescence Protein) [Heim, R., Cubitt, A. B. and Tsien, R. Y. (1995) Nature 373, 663-664; Heim, R., Prasher D C. and Tsien, R. Y. (1994) Proc. Natl. Acad. Sci., 91, 12501-12504; Warg, S and Hazerigg, T. (1994) Nature 639, 400-403; Youvan, D.C. and Michel-Beyerle, M. E. (1996)Nature Biotechnology 14 1219-1220; Chalfie, M., Tu, Y., Euskirchen, G., Ward, W. W. and Prasher, D. C. (1994) Science 263, 802-805], β-galactosidase [Canestro C, Albalat R, Escriva H, Gonzalez-Duarte R. Endogenous beta-galactosidase activity in amphioxus: a useful histochemical marker for the digestive system. Dev Genes Evol 2001 March 211 (3):154-6], luciferase [Arch Toxicol 2002 June; 76 (5-6):257-61, Estrogenic activity of UV filters determined by an in vitro reporter gene assay and an in vivo transgenic zebrafish assay. Schreurs R, Lanser P, Seinen W, Van Der Burg B.], and acetyl transferase [J Recept Signal Transduct Res 2001 February; 21 (1):71-84, A simplified method for large scale quantification of ranscriptional activity and its use in studies of steroids and steroid receptors. Zhang S, Lu J, Iyama K, Lo S C, Danielsen M.].

The change in a promoter activity may be monitored, for example, by fluorometry for fluorescent proteins such as GFP; and by spectrometry based on an enzyme-substrate reaction for luciferase.

WO03/018792 discloses a method for easily identifying chemical substances present in the circumstances by fluorometry. According to this identifying method, yeast is transformed with a vector comprising a polynucleotide in which a base sequence encoding a fluorescent marker protein such as GFP is operably linked to a base sequence comprising a yeast gene promoter which is specifically activated by a harmful substance. Existence of the harmful substance may be detected when after the transformed yeast is contacted to a sample solution, an expression amount of the fluorescent protein increases by detecting it by fluorometry.

### DISCLOSURE OF THE INVENTION

However, a fluorescent measurement relies on an introduction of expensive fluorescence measurement devices, which leads to cost increase for a chemical substance detection system. Further, since a location for measurement is limited to a specific site such as a laboratory, it is not considered as a convenient system.

Therefore, it is desired to provide a system for easily detecting existence or quantity of chemical substances in an environment without relying on fluorometry.

The inventors focused on oxygen consumption by an enzyme-substrate reaction between luciferase, which is an example of a marker protein, and its substrate luciferine and have found that an expression amount of the marker protein could be detected by measuring oxygen consumption during the enzyme-substrate reaction rather than a fluorescence intensity.

For example, an enzyme-substrate reaction between firefly luciferase and D-firefly luciferine is shown below.

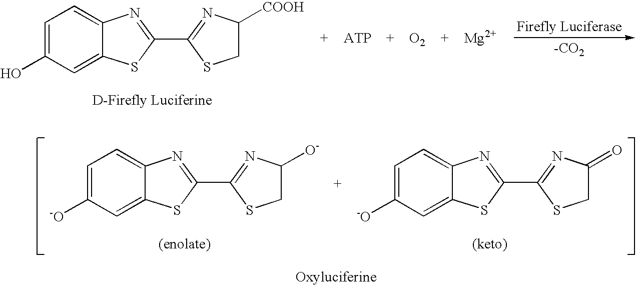

Such enzymes which consume oxygen as an electron receptor in an enzymatic reaction to oxidize a substrate (referred to as “redox enzymes” in this text) include, besides luciferase, for example, oxidase such as Cytochrome C oxidase, Pyridoxamine phosphate oxidase, glutathione oxidase, glucose oxidase, proline oxidase, amino acid oxidase, ascorbate oxidase, acyl-CoA oxidase, galactose oxidase, xanthine oxidase, cholesterol oxidase, oxalate oxidase, sarcosine oxidase; and oxygenase such as kynurenine 3-monooxygenase, squalene oxygenase, monooxygenase, tryptophan 2,3-dioxygenase.

For example, an enzyme-substrate reaction between glucose oxidase and glucose is shown below.

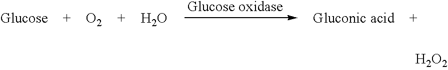

Therefore, the present inventors found that when a specific chemical substance activates a promoter of a yeast gene, existence or a quantity of the chemical substance in a sample may be detected by producing a transformed cell in which a polynucleotide encoding oxidoreductase as a marker protein is operably linked or a transformed cell which is transformed with a vector comprising a polynucleotide in which a polynucleotide encoding oxidoreductase as a marker protein is operably linked at a downstream of a base sequence selected from a group consisting of a base sequence comprising a promoter responding to the chemical substance and a base sequence comprising a promoter from a gene that is homologous to the genes and is derived from other species; by adding any of the transformed cells and a substrate to oxidoreductase to a sample containing the chemical substance; and by measuring a dissolved oxygen consumption to determine an amount of the expressed oxidoreductase.

Conventionally, methods for detecting dissolved oxygen by using oxygen electrodes are well known.

For example, Japanese Patent Publication (kokai) No. 2001-252066 discloses a method for measuring the number of bacteria in a sample solution by preparing a sample solution with dispersing a food wherein the number of bacteria is measured into a solution and detecting dissolved oxygen in the sample solution.

In addition, Japanese Patent Publication (kokai) No. 2001-258592 discloses a method for measuring drug sensitivity of microorganisms to be measured accurately in a short period by detecting a dissolved oxygen rate in a solution with oxygen electrodes. According to this method, a reaction between the microorganisms to be measured and the drug may be detected in an extremely easy and accurate manner because current values measured from oxygen electrode are stored separately in time-series, and moving-average values are calculated from the measured current values stored separately in time-series and time-differentiate values are calculated from a pair of moving-average values separately calculated with a least square approximation with respect to a first solution containing the microorganisms to be mesured and the drug and a second solution free from the drug.

The method for detecting a chemical substance according to the present invention comprises: (a) using a transformed yeast which has been transformed with a polynucleotide encoding an enzyme oxidizing a substrate by using oxygen as an electron acceptor in an enzymatic reaction or a vector comprising a polynucleotide in which a polynucleotide encoding oxidoreductase as a marker protein is operably linked, at a downstream of a base sequence selected from a group consisting of a base sequence comprising a promoter responding to the chemical substance among promoters of yeast genes and a base sequence comprising a promoter from a gene that is homologous to the genes and is derived from other species; and (b) detecting existence or a quantity of chemical substances in the environment regarding a solution comprising at least a sample to be evaluated, a transformed yeast and a substrate to a marker protein oxydoreductase, by storing in time-series measured current values from the oxygen electrodes and by calculating consumption of dissolved oxygen based on a change in the measured current values which have been stored.

That is, the present method relies on the finding that upon measurement of an oxygen amount consumed in an enzyme-substrate reaction between oxidoreductase such as luciferase, oxidase and oxygenase produced from a transformed cell according to the present invention and substrates to these enzymes, an existence or a quantity of the produced oxidoreductase may be detected.

Since an increase in a quantity of the produced oxidoreductase is caused by activation of a gene promoter responding to a chemical substance due to the existence of the chemical substance, an increase in oxygen consumption indicates existence or a quantity of the chemical substance.

In the present invention, since a transformed cell is used to detect existence or a quantity of a chemical substance, it is required to take into account an oxygen consumption rate due to basal metabolism such as an aspiration by the transformed cells in measurement.

In the present invention, an oxygen consumption rate due to an enzyme-substrate reaction may be detected by calculating an oxygen consumption rate due to basal metabolism previously and by subtracting the rate from the measured rate when an oxygen consumption rate due to an enzyme-substrate reaction is overwhelmingly greater than that due to basal metabolism.

In the present invention, only an enzyme-substrate reaction may be observed by disrupting the transformed cells to eliminate effects of the oxygen consumption due to basal metabolism.

Further, an oxygen consumption rate due to an enzyme-substrate reaction may be detected by using oxidoreductase permeable through a cell membrane as a marker protein and by recovering the transformed cells from the solution without disrupting the transformed cells so as to avoid effects from contents from the disrupted cells.

### BRIEF EXPLANATION OF THE DRAWINGS

FIG. 1 depicts a block diagram showing an embodiment of a device for measuring chemical substances according to the present invention.

FIG. 2 depicts a schematic illustration explaining the principle of oxygen electrodes of the device for measuring chemical substances according to the present invention.

FIG. 3 depicts a flowchart showing an embodiment of the method for measuring chemical substances according to the present invention.

FIG. 4 depicts a graphic indicating a temporal change in current values in a measurement of dissolved oxygen according to the present invention.

### BEST MODE FOR CARRYING OUT THE INVENTION

As host cells for use in transformation according to the present invention, human cells are obviously preferred, but cells from other mammals such as mice and the like may be used. In addition, from a view point of a toxicity evaluation for environment, cells from fish, nematoda and the like which have been used in bioassay techniques may be used. Further, microorganism cells are preferably used because of easiness in culturing them.

Since the method of the present invention relies on genes from yeast cells, and a growth of yeast is not affected by variable factors in circumstance specimens such as a salt concentration and the like, yeast cell is preferred.

Method for transforming cells are well known in the art [See, e.g., Kaiser C, Michaelis S, Mitchell A: Lithium acetate yeast transformation, Methods in Yeast Genetics, A Cold Spring Harbor Laboratory Course Manual 1994 edition (Cold Spring Harbor Laboratory Press) pp. 133-134, 1994].

In addition, the object of the present invention can be also achieved by replacing a coding region of the yeast gene with a polynucleotide sequence encoding a marker protein without use of a vector. The above polynucleotide construct may also be directly introduced into a cell, and such a method is well known.

Accordingly, in the present invention, transformed yeast is made by introducing a base sequence in which a polynucleotide encoding a marker protein is operably linked at a downstream of a nucleotide sequence selected from nucleotide sequences comprising a promoter from yeast genes which are selected from a group consisting of the following, and base sequences comprising a promoter from a gene that is homologous to the genes and is derived from other species.
YBR072W, YCR102C, YCR107W, YDL218W, YDL243C, YDR453C, YDR533C, YFL014W, YFL056C, YFL057C, YGR110W, YJR155W, YKL071W, YKR076W, YLL060C, YLR460C, YMR090W, YNL331C, YNL332W, YNL335W, YOL150C, YOL165C, YPL171C, YPR167C, YBL048W, YBL064C, YBL107C, YBR008C, YBR173C, YBR256C, YBR296C, YDL021W, YFL022C, YFL024C, YFL061W, YGL121C, YGL158W, YGR043C, YHR029C, YHR112C, YHR139C, YHR179W, YHR209W, YIR030C, YJR010W, YJR048W, YKL001C, YKL107W, YKR075C, YKR097W, YLL056C, YLR297W, YLR303W, YML087C, YMR096W, YNL274C, YOL151W, YOR226C, YOR338W, YOR391C, YPL280W, YDR406W, YJL153C, YLR346C, YOR049C, YOR153W, YPL088W, YAL034C, YDL124W, YDL174C, YDR476C, YGL156W, YGR035C, YGR157W, YGR213C, YGR281W, YGR284C, YHL047C, YHR043C, YHR044C, YHR054C, YJR073C, YKL165C, YLR008C, YMR315W, YNL211C, YOL031C, YOL101C, YOR303W, YAL005C, YAR031W, YBL005W-A, YBL022C, YBL041W, YBL049W, YBL075C, YBL078C, YBR062C, YBR169C, YBR294W, YCL020W, YCL035C, YCL043C, YCL050C, YCL057W, YCR012W, YCR013C, YCR060W, YDL007W, YDL027C, YDL097C, YDL110C, YDL126C, YDL169C, YDR070C, YDR155C, YDR158W, YDR204W, YDR210W, YDR214W, YDR258C, YDR313C, YDR368W, YDR435C, YER012W, YER037W, YER091C, YER103W, YFL044C, YFR003C, YFR010W, YFR020W, YFR024C, YFR044C, YFR053C, YGL006W, YGL048C, YGL062W, YGL141W, YGL157W, YGL163C, YGL180W, YGL184C, YGR010W, YGR028W, YGR032W, YGR048W, YGR124W, YGR135W, YGR142W, YGR161C, YGR192C, YGR197C, YGR201C, YGR212W, YGR231C, YGR244C, YGR254W, YGR268C, YHL030W, YHR016C, YHR018C, YHR055C, YHR087W, YHR166C, YIL160C, YIR017C, YJL034W, YJL048C, YJL052W, YJL144W, YJL163C, YJR009C, YJR069C, YJR074W, YJR130C, YJR149W, YKL065C, YKL073W, YKL103C, YKL142W, YKL210W, YKL218C, YKR011C, YKR018C, YKR046C, YKR049C, YLL024C, YLL026W, YLR027C, YLR080W, YLR107W, YLR121C, YLR132C, YLR133W, YLR155C, YLR158C, YLR161W, YLR195C, YLR217W, YLR328W, YLR336C, YLR345W, YLR370C, YLR423C, YML004C, YML092C, YML128C, YML130C, YML131W, YMR040W, YMR118C, YMR214W, YMR251W, YMR297W, YMR322C, YNL036W, YNL055C, YNL071W, YNL094W, YNL134C, YNL155W, YNL160W, YNL239W, YNL241C, YOL005C, YOR020C, YOR027W, YOR037W, YOR059C, YOR120W, YOR134W, YOR152C, YOR173W, YOR289W, YOR362C, YPL240C, YPR030W, YAL008W, YAL023C, YAL060W, YAL062W, YAR009C, YBL101C, YBR006W, YBR046C, YBR052C, YBR053C, YBR056W, YBR099C, YBR137W, YBR139W, YBR149W, YBR170C, YBR177C, YBR203W, YBR207W, YBR212W, YBR239C, YBR284W, YBR293W, YCL018W, YCL033C, YCL040W, YCL049C, YCR062W, YCR067C, YCR082W, YDL010W, YDL020C, YDL024C, YDL054C, YDL095W, YDL100C, YDL115C, YDL144C, YDL198C, YDL223C, YDL245C, YDL246C, YDR001C, YDR032C, YDR058C, YDR072C, YDR127W, YDR168W, YDR169C, YDR188W, YDR231C, YDR261C, YDR264C, YDR272W, YDR293C, YDR304C, YDR330W, YDR403W, YDR411C, YDR427W, YDR436W, YDR497C, YDR511W, YDR516C, YDR519W, YDR545W, YEL012W, YEL030W, YER004W, YER009W, YER021W, YER035W, YER053C, YER079W, YER094C, YER096W, YER125W, YER158C, YER163C, YER175C, YER177W, YER178W, YER185W, YFL006W, YFL010C, YFL016C, YFL029C, YFL030W, YFL031W, YFL032W, YFL038C, YFL041W, YFR004W, YFR047C, YFR050C, YFR052W, YGL011C, YGL013C, YGL037C, YGL047W, YGL053W, YGL091C, YGL094C, YGL127C, YGL150C, YGL199C, YGL207W, YGL248W, YGR008C, YGR037C, YGR055W, YGR101W, YGR130C, YGR154C, YGR194C, YGR221C, YGR232W, YGR248W, YGR253C, YGR256W, YHL008C, YHR027C, YHR053C, YHR057C, YHR111W, YHR138C, YHR161C, YHR164C, YHR169W, YHR174W, YHR176W, YHR199C, YIL010W, YIL034C, YIL041W, YIL045W, YIL087C, YIL107C, YIL142W, YIL155C, YIR034C, YIR036C, YIR037W, YIR038C, YIR039C, YJL001W, YJL031C, YJL035C, YJL053W, YJL057C, YJL066C, YJL068C, YJL082W, YJL099W, YJL102W, YJL151C, YJL152W, YJL161W, YJL164C, YJL171C, YJL172W, YJL210W, YJL219W, YJR008W, YJR045C, YJR046W, YJR106W, YJR117W, YJR137C, YKL007W, YKL026C, YKL035W, YKL091C, YKL104C, YKL117W, YKL145W, YKL146W, YKL151C, YKL152C, YKL153W, YKL193C, YKL195W, YKL213C, YKL215C, YLL028W, YLL039C, YLL058W, YLR054C, YLR103C, YLR120C, YLR136C, YLR149C, YLR152C, YLR178C, YLR259C, YLR299W, YLR324W, YLR327C, YLR348C, YLR350W, YLR356W, YLR362W, YLR387C, YLR429W, YML054C, YML070W, YML100W, YML117W, YML125C, YMR004W, YMR008C, YMR009W, YMR020W, YMR067C, YMR089C, YMR097C, YMR102C, YMR105C, YMR107W, YMR152W, YMR180C, YMR184W, YMR191W, YMR219W, YMR271C, YMR275C, YMR295C, YMR314W, YMR316W, YNL006W, YNL007C, YNL012W, YNL044W, YNL045W, YNL074C, YNL092W, YNL093W, YNL104C, YNL115C, YNL156C, YNL231C, YNL234W, YNL237W, YNL281W, YNL305C, YNL333W, YNR010W, YNR019W, YNR033W, YNR059W, YNR068C, YNR069C, YOL032W, YOL036W, YOL047C, YOL071W, YOL082W, YOL083W, YOL117W, YOL119C, YOL126C, YOL131W, YOL153C, YOL162W, YOL163W, YOL164W, YOR019W, YOR035C, YOR036W, YOR099W, YOR117W, YOR124C, YOR130C, YOR132W, YOR157C, YOR185C, YOR197W, YOR259C, YOR261C, YOR273C, YOR288C, YOR332W, YOR336W, YOR347C, YPL017C, YPL087W, YPL106C, YPL109C, YPL149W, YPL154C, YPL196W, YPL206C, YPL222W, YPR023C, YPR024W, YPR026W, YPR067W, YPR103W, YPR108W, YPR151C, YAL012W, YBR029C, YBR222C, YCL009C, YCL027W, YCL064C, YCR098C, YDL222C, YDR055W, YDR077W, YDR502C, YEL001C, YEL042W, YER026C, YER106W, YGR136W, YGR138C, YHR137W, YHR142W, YIL023C, YIL153W, YJL073W, YJR004C, YJR054W, YKL039W, YKL086W, YKL163W, YKR091W, YLR109W, YLR194C, YLR250W, YMR095C, YMR189W, YNL106C, YNL169C, YNL322C, YOR181W, YOR198C, YOR208W, YOR247W, YPL089C, YAL038W, YAL053W, YBR023C, YBR214W, YBR295W, YCR048W, YDL072C, YDL204W, YDR085C, YDR098C, YDR259C, YDR380W, YDR388W, YDR391C, YDR432W, YDR481C, YDR510W, YER069W, YGL022W, YGL126W, YGL209W, YGL255W, YGR189C, YGR282C, YHL035C, YHR030C, YIL022W, YIL024C, YIL117C, YIL123W, YIL140W, YIL154C, YJL088W, YJL108C, YJL149W, YJL159W, YJL186W, YJR148W, YKL096W, YLR180W, YLR273C, YLR300W, YLR307W, YLR378C, YLR391W, YMR094W, YMR104C, YMR276W, YMR296C, YNL190W, YNL208W, YNL300W, YNR064C, YOL013C, YOL058W, YOR248W, YOR355W, YPL052W, YPL163C, YPR079W, YAR028W, YBR146W, YBR183W, YCL038C, YCR071C, YDL008W, YDR019C, YDR031W, YDR115W, YDR486C, YER038C, YER130C, YFL054C, YGL136C, YGR146C, YGR207C, YHL040C, YIL167W, YJL020C, YKR039W, YLR031W, YLR205C, YMR072W, YMR140W, YMR173W, YMR195W, YMR226C, YNL037C, YNR002C, YOL143C, YOR136W, YOR215C, YOR382W, YOR383C, YPL054W, YPL271W, YPR127W, YAL044C, YAL054C, YAR010C, YAR027W, YAR071W, YBL001C, YBL043W, YBL057C, YBR014C, YBR024W, YBR035C, YBR068C, YBR111C, YBR116C, YBR147W, YBR168W, YBR246W, YBR273C, YCR004C, YCR021C, YCR037C, YCR088W, YDL022W, YDL128W, YDL238C, YDR003W, YDR009W, YDR033W, YDR084C, YDR104C, YDR270W, YDR315C, YDR340W, YDR357C, YDR358W, YDR396W, YDR405W, YDR410C, YDR434W, YDR482C, YDR487C, YDR520C, YDR534C, YDR539W, YEL011W, YEL065W, YEL066W, YER039C, YER044C, YER067W, YER080W, YER107C, YFL020C, YFL028C, YFL043C, YFR015C, YGL001C, YGL008C, YGL068W, YGL073W, YGL104C, YGL113W, YGL154C, YGL167C, YGL229C, YGL242C, YGL249W, YGL253W, YGR052W, YGR060W, YGR065C, YGR106C, YGR111W, YGR220C, YGR257C, YHL023C, YHL048W, YHR004C, YHR037W, YHR071W, YHR092C, YHR190W, YIL007C, YIL033C, YIL070C, YIL088C, YIL111W, YIR002C, YIR016W, YIR035C, YIR043C, YJL012C, YJL083W, YJL089W, YJL116C, YJL131C, YJL132W, YJL196C, YJR061W, YJR086W, YJR142W, YJR161C, YKL008C, YKL013C, YKL041W, YKL067W, YKL138C, YKL139W, YKL150W, YKL175W, YKR006C, YKR014C, YKR070W, YLL023C, YLR023C, YLR093C, YLR118C, YLR142W, YLR225C, YLR241W, YLR251W, YLR252W, YLR270W, YML030W, YML110C, YMR021C, YMR027W, YMR148W, YMR181C, YMR262W, YMR272C, YMR298W, YNL011C, YNL130C, YNL214W, YNL259C, YOL129W, YOR042W, YOR052C, YOR137C, YOR149C, YOR165W, YOR270C, YOR285W, YOR367W, YPL018W, YPL156C, YPL186C, YPL203W, YPL216W, YPL255W, YPR006C, YPR073C, YPR098C, YBR050C, YBR145W, YBR299W, YDR518W, YEL020C, YFL062W, YGL039W, YGL134W, YJL217W, YJR159W, YLR126C, YNL249C, YNL284C, YNL336W, YOL157C, YOR344C, YOR381W, YPL265W, YPR124W, YBR074W, YBR109C, YBR126C, YBR201W, YCR005C, YDL248W, YDR041W, YDR105C, YDR268W, YDR452W, YEL075C, YER046W, YER050C, YER136W, YER159C, YGL250W, YGR019W, YGR042W, YGR053C, YGR066C, YGR247W, YGR255C, YGR295C, YHL044W, YHR145C, YIL058W, YIL065C, YIL083C, YIL098C, YIL172C, YJL030W, YJL185C, YJL213W, YJR029W, YJR099W, YJR122W, YJR125C, YKL190W, YKR020W, YLL025W, YLL051C, YLR043C, YLR090W, YLR100W, YLR108C, YLR290C, YML068W, YMR051C, YMR139W, YMR178W, YMR193W, YNL015W, YNL079C, YNL122C, YNL223W, YNL285W, YNL293W, YNR007C, YNR035C, YNR061C, YOL016C, YOL104C, YOR220W, YOR221C, YOR374W, YPL123C, YPR077C, YPR107C, YPR147C, YBR093C, YBR196C, YEL041W, YEL047C, YER023W, YER119C, YFL055W, YGR209C, YIL124W, YKL187C, YLL055W, YMR318C, YOL152W, YAL007C, YBR067C, YBR115C, YBR285W, YBR292C, YDL043C, YDL123W, YDL131W, YDL168W, YDL212W, YDR056C, YDR132C, YDR154C, YDR183W, YDR216W, YDR253C, YDR295C, YDR494W, YDR513W, YEL072W, YER045C, YER061C, YER181C, YFL052W, YFL058W, YFR030W, YGL089C, YGL096W, YGL114W, YGL193C, YGL202W, YGL204C, YGL259W, YGR006W, YGR070W, YGR088W, YHL034C, YHL036W, YHR048W, YHR104W, YHR163W, YIL060W, YIL136W, YIR024C, YJL036W, YJL045W, YJL060W, YJL101C, YJL155C, YJR085C, YJR109C, YJR156C, YKL070W, YKL161C, YKL221W, YKR071C, YLL009C, YLL050C, YLR092W, YLR145W, YLR156W, YLR163C, YLR220W, YLR280C, YLR311C, YLR390W, YML116W, YMR034C, YMR038C, YMR081C, YMR250W, YNL240C, YNL260C, YNL277W, YNR074C, YOL044W, YOL084W, YOL147C, YOL159C, YOR184W, YOR228C, YOR255W, YPL223C, YPR160W, YDL182W, YBR047W, YBR054W, YBR291C, YDR069C, YER124C, YER131W, YGR044C, YIL094C, YKR007W, YMR240C, YNR050C, YOR007C, YAL015C, YBL065W, YBR105C, YBR182C, YBR186W, YBR244W, YBR272C, YCL069W, YDL025C, YDL059C, YDL085W, YDL113C, YDL244W, YDR018C, YDR054C, YDR202C, YDR223W, YDR350C, YDR353W, YDR374C, YDR512C, YEL052W, YEL070W, YER098W, YFR017C, YGL046W, YGL067W, YGL098W, YGL117W, YGL146C, YGL240W, YGR011W, YGR067C, YGR133W, YGR153W, YGR223C, YHR116W, YHR124W, YIL097W, YIL168W, YJL103C, YJL221C, YJR036C, YJR095W, YKL085W, YKL133C, YKL162C, YKL188C, YKL217W, YKR061W, YKR105C, YLL062C, YLR174W, YLR216C, YLR247C, YLR260W, YLR267W, YLR389C, YML007W, YMR041C, YMR177W, YMR253C, YNL009W, YNL117W, YNL128W, YNL183C, YNR073C, YOL133W, YOL158C, YOR133W, YOR225W, YOR227W, YPL161C, YPL166W, YPL202C, YPL224C, YPR015C, YPR086W, YPR201W, YAL061W, YAL067C, YAR007C, YBL033C, YBL056W, YBL086C, YBR026C, YBR073W, YBR101C, YBR117C, YBR123C, YBR213W, YBR269C, YBR280C, YCR036W, YDL132W, YDL149W, YDL200C, YDL234C, YDL242W, YDR099W, YDR177W, YDR256C, YDR392W, YDR394W, YDR531W, YEL071W, YER014W, YER042W, YER090W, YER184C, YFL059W, YFR042W, YFR046C, YFR049W, YGL026C, YGL058W, YGL185C, YGL227W, YGL252C, YGL254W, YGR089W, YGR112W, YGR134W, YGR276C, YHL019C, YHR012W, YHR017W, YHR028C, YHR106W, YHR109W, YHR156C, YIL036W, YIL046W, YIL143C, YIL152W, YIL159W, YIL164C, YJL071W, YJL094C, YJL154C, YJR056C, YJR072C, YJR110W, YJR139C, YKL025C, YKL034W, YKL064W, YKL171W, YKL196C, YKR012C, YKR068C, YKR069W, YLL001W, YLL057C, YLL061W, YLR064W, YLR070C, YLR099C, YLR144C, YLR157C, YLR160C, YLR164W, YLR364W, YLR421C, YML032C, YML042W, YML112W, YML118W, YMR114C, YMR115W, YMR258C, YNL181W, YNL191W, YNL212W, YNL213C, YNL250W, YNL265C, YNL312W, YNR032W, YOL038W, YOL049W, YOL064C, YOR088W, YOR155C, YOR257W, YOR265W, YOR377W, YOR386W, YPL031C, YPL113C, YPL124W, YPL151C, YPL249C, YPL260W, YPL274W, YPR048W, YPR061C, YPR093C, YPR125W, YPR158W, YPR168W, YPR169W, YPR174C, YPR180W, YPR193C, YPR200C, YAL014C, YAL017W, YAL049C, YBL019W, YBL058W, YBR001C, YBR013C, YBR018C, YBR037C, YBR045C, YBR051W, YBR063C, YBR128C, YBR129C, YBR204C, YBR241C, YBR255W, YBR281C, YCL044C, YCL055W, YCR014C, YCR019W, YCR024C, YCR105W, YDL065C, YDL089W, YDL143W, YDL173W, YDL193W, YDL197C, YDL206W, YDL230W, YDL233W, YDR040C, YDR071C, YDR078C, YDR109C, YDR140W, YDR194C, YDR212W, YDR221W, YDR257C, YDR271C, YDR287W, YDR294C, YDR316W, YDR329C, YDR338C, YDR369C, YDR421W, YDR425W, YDR485C, YDR488C, YDR504C, YDR505C, YDR506C, YDR515W, YEL005C, YEL037C, YEL044W, YER017C, YER048C, YER052C, YER078C, YER089C, YER092W, YER100W, YER162C, YER182W, YFL021W, YFL042C, YFR045W, YFR051C, YPR056C, YGL040C, YGL041C, YGL045W, YGL057C, YGL093W, YGL105W, YGL125W, YGL166W, YGL181W, YGL183C, YGL215W, YGL216W, YGL221C, YGL223C, YGR007W, YGR029W, YGR155W, YGR156W, YGR186W, YGR198W, YGR210C, YGR211W, YGR237C, YGR250C, YGR258C, YGR266W, YGR270W, YGR274C, YGR277C, YHL021C, YHL037C, YHL038C, YHR082C, YHR083W, YHR134W, YHR160C, YHR171W, YHR180W, YHR205W, YIL062C, YIL072W, YIL075C, YIL099W, YIL108W, YIL165C, YIL170W, YIR009W, YIR018W, YIR031C, YIR032C, YJL032W, YJL049W, YJL128C, YJL165C, YJR044C, YJR052W, YJR090C, YJR091C, YJR103W, YJR104C, YJR153W, YKL059C, YKL079W, YKL090W, YKL094W, YKL192C, YKL209C, YKR052C, YKR102W, YKR106W, YLL054C, YLR025W, YLR097C, YLR200W, YLR226W, YLR248W, YLR266C, YLR392C, YLR427W, YML013W, YML029W, YML041C, YML051W, YML078W, YML079W, YML088W, YML099C, YMR056C, YMR068W, YMR091C, YMR110C, YMR160W, YMR186W, YMR255W, YNL005C, YNL026W, YNL039W, YNL063W, YNL064C, YNL077W, YNL083W, YNL147W, YNL176C, YNL194C, YNL253W, YNL257C, YNL261W, YNL264C, YNL276C, YNR006W, YNR034W, YNR047W, YNR051C, YNR071C, YOL065C, YOL067C, YOR005C, YOR008C, YOR022C, YOR023C, YOR058C, YOR069W, YOR087W, YOR138C, YOR229W, YOR256C, YOR267C, YPL005W, YPL020C, YPL022W, YPL105C, YPL147W, YPL150W, YPL152W, YPL164C, YPL168W, YPL180W, YPL188W, YPL194W, YPR025C, YPR047W, YPR049C, YPR066W, YPR081C, YPR134W, YPR140W, YPR148C, YPR155C, YPR172W, YPR185W, YAL018C, YAR064W, YBR012C, YBR076W, YBR287W, YDR043C, YDR250C, YDR373W, YFR014C, YGL191W, YGR180C, YHR136C, YJL026W, YJL037W, YLR038C, YNL058C, YOR031W, YGR087C, YIL166C, YHR008C, YIL129C, YGL256W, YJR030C, YMR077C, YBR264C, YPL177C, YKR040C, YGL056C, YDR128W, YGR139W, YBL101W-A, YOR253W, YOL026C, YDR278C, YHR095W, YCL042W, YNL200C, YPL221W, YLR415C, YMR058W, YPR037C, YER072W, YML028W, YOR325W, YAL039C, YMR112C, YJR107W, YGL088W, YJR058C, YNL142W, YDR090C, YMR071C, YBL093C, YGR293C, YML055W, YDL017W, YDL210W, YGL055W, YCL025C, YDR080W, YDL181W, YNR030W, YJL017W, YIL127C, YDR281C, YDR366C, YFR026C, YJL212C, YPL215W, YEL019C, YBR132C, YHL018W, YNL196C, YPL038W, YAR047C, YPL262W, YHL006C, YPL225W, YBR124W, YOR148C, YKR053C, YBL044W, YER029C, YLR360W, YCL056C, YCR007C, YGR239C, YNL256W, YPR146C, YLR377C, YKL097C, YBR066C, YLR338W, YDL229W, YBR253W, YJR027W, YKL198C, YBL030C, YBR031W, YBR118W, YBR162C, YBR221C, YCR024C-A, YCR106W, YDL046W, YDR012W, YDR133C, YDR134C, YDR276C, YDR342C, YDR343C, YEL027W, YEL034W, YGR038W, YGR243W, YGR279C, YHR094C, YHR105W, YHR175W, YHR181W, YIL056W, YIL162W, YJL059W, YJL097W, YJL158C, YJR105W, YKL051W, YKL056C, YKL097W-A, YKL100C, YKL141W, YKR066C, YLR134W, YLR258W, YLR339C, YML058W, YMR083W, YMR203W, YNL209W, YNL307C, YOL030W, YOR178C, YPL028W, YPR028W, YPR113W, YPR149W, YPR150W, YPR183W, YAL016W, YBL099W, YBL100C, YBR011C, YBR096W, YBR100W, YBR127C, YBR283C, YBR286W, YCL008C, YCL058C, YCR030C, YCR034W, YCR069W, YDL015C, YDL023C, YDL061C, YDL086W, YDR038C, YDR039C, YDR050C, YDR151C, YDR178W, YDR233C, YDR284C, YDR298C, YDR345C, YDR359C, YDR382W, YDR385W, YDR400W, YDR407C, YDR538W, YEL024W, YEL033W, YEL063C, YER057C, YER081W, YER120W, YFL011W, YGL012W, YGL206C, YGR022C, YGR026W, YGR082W, YGR107W, YGR172C, YGR191W, YGR204W, YGR260W, YHL005C, YHL046C, YHR025W, YHR026W, YHR123W, YHR126C, YHR143W, YIL011W, YIL015W, YIL018W, YIL157C, YIR041W, YJL016W, YJL121C, YJL133W, YJL138C, YJL191W, YJR018W, YJR047C, YJR077C, YJR119C, YJR121W, YJR123W, YJR143C, YJR145C, YKL060C, YKL147C, YKL148C, YKL157W, YKL164C, YKL169C, YKR033C, YLL041C, YLL064C, YLR041W, YLR044C, YLR056W, YLR058C, YLR081W, YLR089C, YLR110C, YLR177W, YLR264W, YLR284C, YLR304C, YLR340W, YLR354C, YLR372W, YLR388W, YML022W, YMR007W, YMR011W, YMR015C, YMR092C, YMR101C, YMR156C, YMR205C, YMR215W, YMR261C, YMR323W, YNL069C, YNL135C, YNL195C, YNR076W, YOL039W, YOL073C, YOL086C, YOL120C, YOL156W, YOL161C, YOR002W, YOR009W, YOR010C, YOR085W, YOR108W, YOR128C, YOR129C, YOR142W, YOR161C, YOR 176W, YOR230W, YOR298W, YPL004C, YPL036W, YPL048W, YPL057C, YPL059W, YPL061W, YPL135W, YPL179W, YPL218W, YPL220W, YPL246C, YPL272C, YPR063C, YPR080W, YPR181C, YBR290W, YCR010C, YCR091W, YDL107W, YDL129W, YDR066C, YDR529C, YFL026W, YGL018C, YGL059W, YNL144C, YOR003W, YAL037W, YAR023C, YBR003W, YBR020W, YBR044C, YBR091C, YBR185C, YBR282W, YCR015C, YCR038C, YCR043C, YDL119C, YDL146W, YDL220C, YDR057W, YDR123C, YDR125C, YDR222W, YDR225W, YDR277C, YDR286C, YDR347W, YDR408C, YDR438W, YDR479C, YDR483W, YEL039C, YEL057C, YEL073C, YER066W, YER076C, YER084W, YER121W, YER189W, YFL017C, YFL046W, YFR006W, YFR008W, YGL115W, YGL208W, YGL214W, YGL218W, YGR021W, YGR023W, YGR024C, YGR064W, YGR076C, YGR096W, YGR108W, YGR174C, YGR182C, YGR236C, YGR288W, YHL042W, YHR195W, YHR210C, YIL006W, YIL012W, YIL028W, YIL050W, YIL057C, YIL089W, YIL102C, YIL113W, YIL122W, YJL100W, YJL169W, YJL199C, YJR039W, YJR050W, YJR101W, YKL003C, YKL016C, YKL061W, YKL093W, YKL121W, YKL160W, YKL170W, YKL194C, YKR034W, YKR067W, YLR006C, YLR016C, YLR030W, YLR036C, YLR112W, YLR125W, YLR128W, YLR204W, YLR211C, YLR233C, YLR257W, YLR288C, YLR326W, YLR334C, YLR395C, YLR408C, YLR414C, YLR444C, YML050W, YML107C, YML120C, YMR031C, YMR053C, YMR073C, YMR162C, YMR204C, YMR206W, YMR284W, YNL010W, YNL025C, YNL127W, YNL139C, YNL217W, YOL116W, YOL118C, YOR053W, YOR100C, YOR103C, YOR122C, YOR150W, YOR187W, YOR251C, YOR312C, YOR327C, YOR348C, YOR352W, YOR388C, YOR394W, YPL001W, YPL033C, YPL066W, YPL148C, YPL230W, YPL275W, YPL276W, YPR005C, YPR014C, YPR192W, YPR194C, YBR005W, YER025W, YFL027C, YGL080W, YGL205W, YHL028W, YHR185C, YIL076W, YJL166W, YLR046C, YMR035W, YMR238W, YMR252C, YNL192W, YNL202W, YOL108C, YOR385W, YPR165W, YAR033W, YBL038W, YBR009C, YBR010W, YBR151W, YCL067C, YCR096C, YDL137W, YDL192W, YDR073W, YDR086C, YDR224C, YDR377W, YDR378C, YER015W, YGL187C, YHR162W, YJL167W, YJL216C, YKR009C, YLR165C, YMR197C, YNL157W, YOL002C, YOL109W, YOR180C, YPL010W, YPL233W, YBR036C, YDR297W, YGR149W, YGR224W, YNL043C, YPL067C, YPL170W, YCR046C, YDR387C, YFL050C, YGL051W, YHR132C, YIL112W, YJL141C, YKR098C, YLR052W, YLR206W, YML129C, YNL203C, YNR014W, YOL043C, YOL096C, YPR184W, YAL028W, YAL055W, YAR062W, YBL095W, YBL102W, YBR122C, YBR157C, YBR161W, YBR251W, YBR298C, YCR039C, YCR083W, YDL018C, YDL067C, YDL078C, YDL091C, YDL215C, YDL216C, YDR022C, YDR067C, YDR079W, YDR181C, YDR186C, YDR196C, YDR262W, YDR306C, YDR319C, YER188W, YGL004C, YGL035C, YGR036C, YGR062C, YGR120C, YGR131W, YGR141W, YGR167W, YGR287C, YHL024W, YHR080C, YHR097C, YIL077C, YJL046W, YJL070C, YJL096W, YJL113W, YJL146W, YJL180C, YJR019C, YJR049C, YKR058W, YLL005C, YLR078C, YLR151C, YLR271W, YLR295C, YLR351C, YLR375W, YMR023C, YMR025W, YMR135C, YMR210W, YMR267W, YMR278W, YMR293C, YNL073W, YNR037C, YNR040W, YNR072W, YOR028C, YOR316C, YOR328W, YOR363C, YPL039W, YPL040C, YPL099C, YPL107W, YPL134C, YPL138C, YPL140C.

The base sequences and the amino acid sequences of the yeast genes are disclosed in public databases such as MIPS in Germany: Munich Information Center for Protein Sequence, and SGD in USA: Saccharomyces Genome Database, and are known via the internet. The sequences of promoters are also disclosed in a public database of SCPD: The Promoter Database of Saccharomyces cerevisiae.

In addition to the promoters from yeast genes as described above, promoters from a gene that is homologous to the yeast genes and is derived from other species may be used in the invention. In this text, “a gene that is homologous to the yeast genes” means a gene that comprises a base sequence having a 50% or more, preferably 80% or more homology to the base sequences of yeast genes, wherein the base sequence encodes a protein having the same functions as those of the proteins encoded by the yeast genes.

A polynucleotide encoding a marker protein is operably linked at a downstream of a base sequence of a promoter from the gene as described above so as to provide a polynucleotide construct. Methods for operably linking a polynucleotide encoding a protein to a promoter are well known in the art. See, for example, R. W. Old, S. B. Primrose Principles of Gene Manipulation 5th Ed., BAIFUKAN CO., LTD, pp 138-165, pp 234-263, 2000.

Examples of the marker proteins include luciferase such as firefly luciferase, Renilla reniformis luciferase, click beetle luciferase; oxidase such as Cytochrome C oxidase, Pyridoxamine phosphate oxidase, glutathione oxidase, glucose oxidase, proline oxidase, amino acid oxidase, ascorbate oxidase, acyl-CoA oxidase, galactose oxidase, xanthine oxidase, cholesterol oxidase, oxalate oxidase, sarcosine oxidase; and oxygenase such as kynurenine 3-monooxygenase, squalene oxygenase, monooxygenase, tryptophan 2,3-dioxygenase.

As an example of base sequences encoding luciferase, a sequence of luciferase from pGL3-Basic Vector (Promega) is shown as SEQ ID No. 1.

Among yeast gene whose genomic sequences are well known, oxidase-related genes include Q0045, Q0250, Q0275, YBL064c, YBR035c, YBR244w, YDL067c, YDR044w, YDR079w, YDR231c, YDR453c, YDR506c, YEL045c, YER014w, YER021w, YER058w, YER141w, YER145c, YER153c, YER154w, YFL041w, YGL187c, YGL191w, YGL205w, YGR029w, YGR060w, YGR062c, YGR112w, YGR234w, YHR051w, YHR116w, YIL111w, YIR037w, YJL003w, YJR034w, YKL026c, YKR066c, YLL009c, YLL018c-a, YLR038c, YLR142w, YLR205c, YLR395c, YML086c, YML129c, YMR020w, YMR058w, YMR256c, YNL052w, YOR350c, YPL132w, and YPR037c.

Among yeast gene whose genomic sequences are well known, oxygenase-related genes include YBL098w, YDR402c, YGL055w, YGR175c, YGR255c, YHR007c, YHR176w, YJR025c, YJR069c, YJR078w, YJR149w, YLL057c, YLR205c, and YMR015c.

The base sequences of the yeast genes are disclosed in public databases such as MIPS in Germany: Munich Information Center for Protein Sequence, and SGD in USA: Saccharomyces Genome Database, and are known via the internet. The sequences of promoters are also disclosed in a public database of SCPD: The Promoter Database of Saccharomyces cerevisiae.

In the present invention, a gene that is homologous to the oxidase-related genes and oxygenase-related genes and is derived from other species may be used.

Toxic substances to be detected according to the present invention include, but are not limited to, Na₂As, CdCl₂, HgCl₂, PbCl₂, 4-nitroquinoline-N-oxide, 2,4,5-trichlorophenol, γ-hexachlorocyclohexane, manganese ethylenebis (dithiocarbamate), 2,4,5,6-tetrachloro-1,3-benzenedicarbonitrile, tetramethylthiuram disulfide, zinc N,N′-ethylenebis (dithiocarbamate), 8-methyl-N-vanillyl-6-nonenamide, gingerol, acrolein, dimethyl sulfoxide, Roundup (resistered trademark, Herbicide) (N-(phosphomethyl)glycinate ammonium 41.0%, surfactant 59.0%), sodium dodecylbenzosulfonate, sodium lauryl sulfate, 2,4-dichlorophenoxyacetic acid, potassium cyanide, benzo(a)pyrene, formaldehyde, bisphenol-A, 2,5-dichlorophenol, methylmercury chloride, p-nonylphenol, pentachlorophenol, nickel (II) chloride, potassium bichromate, triphenyltin chloride, phenol, S-4-chlorobenzyl-N,N-diethylcarbamate, hexachlorophene, triclosan, and copper sulfate.

When the method as described above is conducted using two or more kinds of transformed microorganisms, that is, two or more kinds of microorganisms transformed with a vector comprising a polynucleotide of a promoter from different yeast genes operably linked to a polynucleotide encoding a marker protein, then toxic substances can be further examined. For example, as shown in the working examples hereinafter, YLL057C can be used as a yeast gene promoter to detect 2,4-dichlorophenoxyacetic acid, arsenious acid or a salt thereof cadmium salt, and cyanide or a salt thereof, and YLR303W can be used as a yeast gene promoter to detect 2,4-dichlorophenoxyacetic acid, arsenious acid or a salt thereof cadmium salt, cyanide or a salt thereof, benzo(a)pyrene, formaldehyde, manganese ethylenebis(dithiocarbamate), and a mercuric salt. Accordingly, when for example the expression of a marker protein is induced by using YLR303W as yeast gene, and not induced by YLL057C, then toxic substances are identified as benzo(a)pyrene, mercuric salt, manganese ethylenebis(dithiocarbamate) or formaldehyde. When the expression of a marker protein is induced by using both yeast genes, then toxic substances are identified as 2,4-dichlorophenoxyacetic acid, arsenious acid or a salt thereof, cadmium salt, or cyanide or a salt thereof.

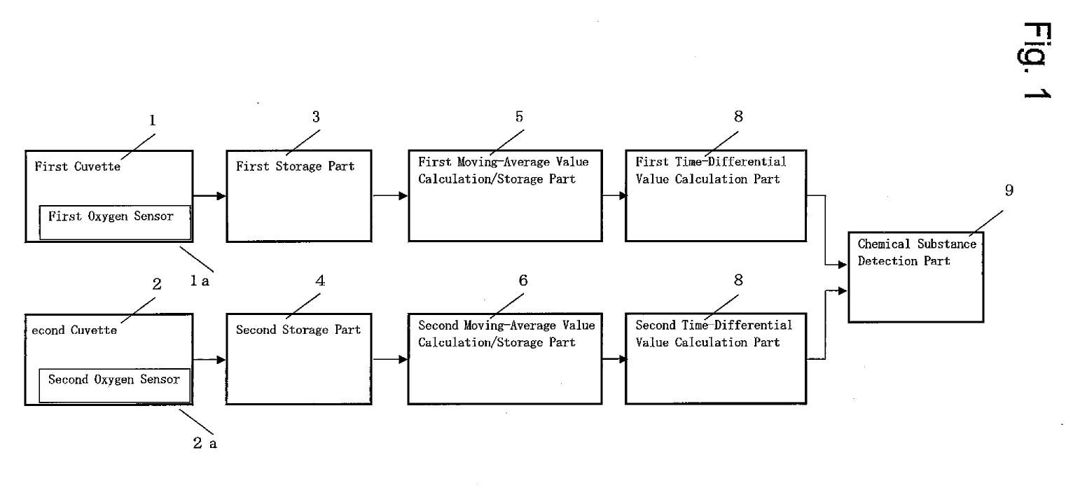

FIG. 1 depicts a block diagram showing an embodiment of a device for measuring chemical substances according to the present invention.

The device comprises a first cuvette 1 which contains a first solution comprising a sample to be measured regarding existence or a quantity of a chemical substance, a transformed cell and a substrate to oxidoreductase which is encoded by a polynucleotide introduced into the transformed cell; a second cuvette 2 which contains a second solution comprising the transformed cell and free from the sample; a first oxygen sensor 1a which is attached to the first cuvette 1 to detect dissolved oxygen rate in the first solution and to output a measured current value; a second oxygen sensor 2a which is attached to the first cuvette 2 to detect dissolved oxygen rate in the second solution and to output a measured current value; a first storage part 3 which stores in time-series the measured current value output from the first oxygen sensor 1a; a second storage part 4 which stores in time-series the measured current value output from the second oxygen sensor 2a; a first moving-average value calculation/storage part 5 which calculates a moving average value from the measured current value stored in time-series in the first storage part 3 and stores it in time-series; a second moving-average value calculation/storage part 6 which calculates a moving average value from the measured current value stored in time-series in the second storage part 4 and stores it in time-series; a first time-differential value calculation part 7 which calculates a first time-differential value from the moving average value stored in time-series in the first moving-average value calculation/storage part 5; a second time-differential value calculation part 8 which calculates a second time-differential value from the moving average value stored in time-series in the second moving-average value calculation/storage part 6; and a chemical substance detection part 9 which detects existence or a quantity of the chemical substance based on the first time-differential value and the second time-differential value.

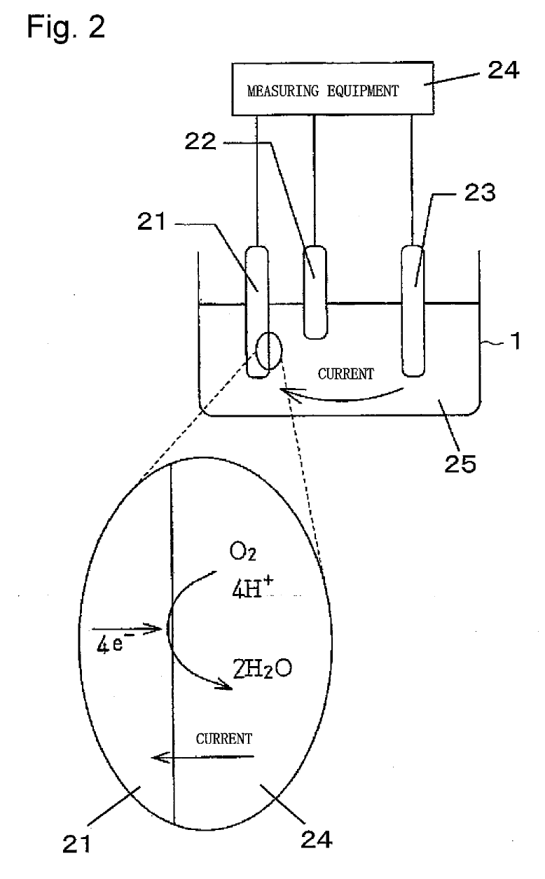

FIG. 2 depicts a schematic illustration explaining the principle of oxygen electrodes of the device for measuring chemical substances according to the present invention.

As shown in FIG. 2, the device comprises a working site (a working electrode) 21, a reference site (a reference electrode) 22 and a counter site (a counter electrode) 23, and a cuvette 1 which is provided with an oxygen electrode connected to a measuring equipment 24 contains a solution 25.

In the solution, a reaction of O₂+4H⁺→2H₂O occurs at the working site 21 to transport 4e⁻ resulting in an electric current flow. Since this reaction depends on dissolved oxygen concentrations, the flowing current rate also depends on the dissolved oxygen concentrations.

Therefore, when current values for the first solution and the second solution are separately measured and the current value for the first solution is smaller than the current value for the second solution, that is, when the dissolved oxygen rate is small, it is considered that the enzyme-substrate reaction is enhanced. The enhancement of the enzyme-substrate reaction indicates that the promoter responding to the chemical substance is activated with the chemical substance so that an increased concentration of the enzyme was expressed.

Accordingly, upon measurement of a dissolved oxygen rate, existence or a quantity of a chemical substance in a sample to be evaluated may be detected.

The present invention also provides a method for detecting a chemical substance which comprises:

detecting a dissolved oxygen rate with an oxygen electrode in a solution comprising a sample to be evaluated, a transformed cell which is transformed with a polynucleotide encoding an enzyme oxidizing a substrate by using oxygen as an electron acceptor in an enzymatic reaction or a vector comprising a polynucleotide in which a polynucleotide encoding an enzyme oxidizing a substrate by using oxygen as an electron acceptor in an enzymatic reaction is operably linked, and a substrate to the enzyme oxidizing a substrate by using oxygen as an electron acceptor in an enzymatic reaction;

collecting output signals from the oxygen electrode for a first time period at an interval of a second time period; and

calculating a change in the dissolved oxygen rate based on the change in the output signals to detect existence or a quantity of the chemical substance in the sample to be evaluated.

In this text, the first time period means a time period from an onset of a measurement to a time when a current value becomes lower than a predetermined current value or to a time when a current value reaches to a plateau, or to a time at which a predetermined time period passes.

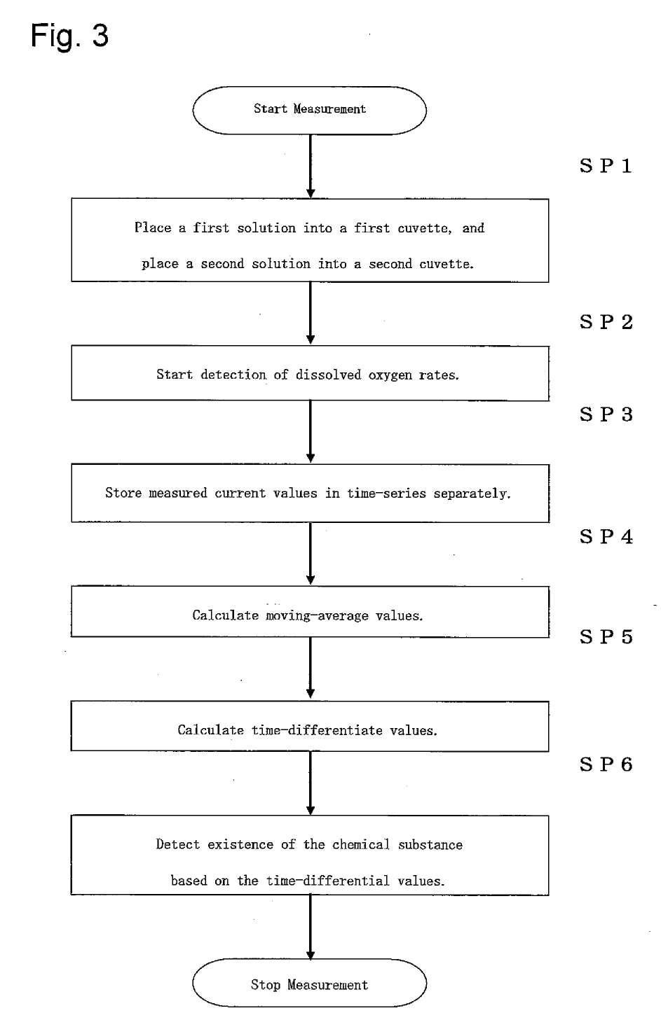

Steps for the chemical substance measurement according to the present invention will be explained based on the flowchart depicted in FIG. 3. At a step SP1, a sample to be evaluated and a transformed cell are added to a solution contained in the first cuvette 1 and the transformed cell is added to solution contained in the second cuvette 2 without adding the sample to be evaluated. At a step SP2, detections of dissolved oxygen rates with a first oxygen sensor 1a and a second oxygen sensor 2a is started. At a step SP3, measured current values output from the first oxygen sensor 1a and the second oxygen sensor 2a are separately stored in time series. At a step SP4, moving-average values are calculated from the measured current values separately stored in time series. At a step SP5, time-differentiated values are calculated from a pair of the separately calculated moving-average values with a least square approximation. At a step SP6, existence of the chemical substance is detected based on the separately calculated time-differentiated values and a series of processes are stopped

As above, an embodiment which involves simultaneously measuring dissolved oxygen rates for the first solution and the second solution and detecting existence or a quantity of a chemical substance based on moving-average values was explained. However, the dissolved oxygen rates in the first solution and the second solution may be measured stepwise. In addition, a measurement method which uses the moving-average values is preferable because accuracy for measurement is high, but it may be also preferable to measure a time period for thoroughly consuming the dissolved oxygen in a solution contained in a cuvette.

The following examples are presented for purpose of further illustration of the invention, and such examples are not intended to be limiting the invention in any respect.

### Examples

### Example 1

The following experiment was conducted to examine which yeast gene is useful for the detection of a toxic substance.

Yeast (Saccharomyces cerevisiae S288C (a SUC2mal mel gap2 CUP1)) were cultured at 25° C. on YPD medium (yeast extract 1%, polypeptone 2%, glucose 2%). One of toxic chemical substances was added to the cell at logarithmic growth phase, and the cell was further cultured for two hours. Cell was cultured without any chemical substance in the same condition, and was used as a control. Concentrations of the chemical substances were defined to inhibit the growth of the yeast but not lead to death.

| Chemical Substances | | Concentrations | |
|---|---|---|---|
| (1) | Na₂As | 0.3 | mM |
| (2) | CdCl₂ | 0.3 | mM |
| (3) | HgCl₂ | 0.7 | mM |
| (4) | PbCl₂ | 2 | mM |
| (5) | 4-Nitroquinoline-N-oxide | 0.2 | μM |
| (6) | 2,4,5-Trichlorophenol | 16 | μM |
| (7) | γ-Hexachlorocyclohexane | 1.3 | mM |
| (8) | Manganese ethylenebis(dithiocarbamate) | 2 | ppm |
| (9) | 2,4,5,6-Tetrachloro-1,3-benzenedicarbonitrile | 10 | μM |
| | (TPN) | | |
| (10) | Tetramethylthiuram disulfide | 75 | μM |
| (11) | Zinc N,N′-ethylenebis(dithiocarbamate) | 2 | ppm |
| (12) | 8-Methyl-N-vanillyl-6-nonenamide | 0.82 | mM |
| (13) | Gingerol | 1.36 | mM |
| (14) | Acrolein | 0.20 | mM |
| (15) | Dimethyl sulfoxide | 1.41 | M |

| | | |
|---|---|---|
| (16) | Roundup (registered trademark, herbicide)¹⁾ | 1500-fold dilution |
| (17) | Sodium dodecylbenzosulfonate | 0.02% |
| (18) | Sodium lauryl sulfate | 0.01% |

¹⁾N-(Phosphomethyl) glycinate ammonium 41.0%, surfactant 59.0%

After the completion of cultivation, the culture was centrifuged to collect the cells. To the cells, sodium acetate buffer (50 mM sodium acetate, 10 mM EDTA, 1% SDS) was added, and the mixture was shaken at 65° C. for five minutes, followed by returning to room temperature, and obtaining the supernatant, which procedure was repeated two times. To the supernatant, ½ amount of a solution of phenol/chloroform (1:1) was added, and the mixture was centrifuged to give a supernatant, an equal amount of chloroform was added, and the mixture was centrifuged to give a supernatant. To the supernatant, an equal amount of isopropanol containing 0.3 M sodium acetate was added, and the mixture was allowed to stand at room temperature for 30 minutes, after which the mixture was centrifuged to give sediment of the whole RNAs. Seventy % ethanol was added to the sediment, and the mixture was again centrifuged to give sediment, which was then dried and dissolved in water. mRNA was isolated from the whole RNAs as followings. In view of the fact that a poly A chain is attached to the 3′ terminus of mRNA, a polynucleotide having a poly T structure which was immobilized on the surface of latex beads was used to trap the mRNA, and then the mRNA was washed and eluted with spine column (Oligotex-dT30<Super>mRNA Purification Kit, Takara). Reverse transcription of the mRNA was conducted with a reverse transcriptase (Super Script II Reverse Transcriptase; catalogue No. 18064-014, GibcoBRL) using fluorescence-labeled nucleotides to give cDNAs that were introduced with Cy3-dUTP or Cy5-dUTP during the reverse transcription.

The labeled cDNAs were dissolved in TE buffer (10 mM Tris-HCl/1 mM EDTA, pH 8.0), and the solution was dropped on the DNA chip containing the whole genes of yeast (DNA Chip Research Inc. Japan) so that the cDNAs were hybridized on the DNA chip at 65° C. for over 12 hours. The fluoresces intensity of the DNA chip was read with a scanner, and the ratio relative to the fluorescence intensity resulting from the absence of chemical substance was estimated as the following, which is shown in Tables 1 to 9.

In the Tables, “Intensity” indicated in the rightmost column is a value of the level of expressed mRNA of each gene in control cells as divided by the average level of the expressions of the whole genes. A gene of which mRNA level is lower in control, and is higher in cases of addition of chemical substance, is especially useful in the detection of toxic substances.

TABLE 1
Unknown protein yeast genes

| | The level of expressed mRNA in the presence of chemical substance/ | | | | | | | | | | | | | | | | | | |
|---|---|---|---|---|---|---|---|---|---|---|---|---|---|---|---|---|---|---|---|
| | The level of expressed mRNA in the absence of chemical substance | | | | | | | | | | | | | | | | | | |
| yeast genes | (1) | (2) | (3) | (4) | (5) | (6) | (7) | (8) | (9) | (10) | (11) | (12) | (13) | (14) | (15) | (16) | (17) | (18) | Intensity |
| YCR102C | 3.4 | 3.2 | 2.4 | 1.1 | 0.7 | 0.4 | 1.1 | 4.9 | 9.8 | 61.5 | 13.5 | 0.5 | 1.1 | 4.1 | 0.9 | 1.2 | 1.1 | 1.2 | 0.53 |
| YDL218W | 3.5 | 1.2 | 2.6 | 1.4 | 0.7 | 0.9 | 1.1 | 1.5 | 72.5 | 12.6 | 5.7 | 0.9 | 1.1 | 4.8 | 1.1 | 3.5 | 1.1 | 0.9 | 0.42 |
| YDR533C | 3.4 | 4.9 | 3.5 | 1.8 | 1.3 | 2.2 | 1.7 | 5.4 | 7.3 | 12.7 | 7.7 | 1.4 | 3.0 | 2.9 | 2.5 | 6.7 | 2.3 | 2.7 | 1.82 |
| YGR110W | 1.6 | 3.1 | 2.2 | 5.9 | 0.9 | 0.9 | 1.7 | 1.5 | 6.4 | 4.8 | 0.5 | 1.4 | 3.6 | 2.8 | 1.2 | 17.1 | 0.9 | 2.1 | 0.19 |
| YKL071W | 8.3 | 3.8 | 5.4 | 2.8 | 1.1 | 18.6 | 3.7 | 24.7 | 162.2 | 109.2 | 40.3 | 1.0 | 8.4 | 21.9 | 1.7 | 2.2 | 1.0 | 0.9 | 0.31 |
| YLR460C | 2.1 | 2.1 | 2.0 | 1.1 | 1.3 | 0.6 | 1.1 | 8.1 | 13.9 | 31.6 | 14.3 | 0.4 | 0.7 | 3.5 | 0.7 | 1.8 | 1.0 | 1.1 | 0.48 |
| YMR090W | 6.9 | 9.5 | 2.3 | 3.5 | 0.7 | 3.8 | 2.7 | 3.9 | 16.7 | 6.1 | 8.8 | 2.2 | 6.9 | 6.5 | 1.9 | 5.1 | 1.8 | 2.8 | 1.30 |
| YOL150C | 4.1 | 3.7 | 9.9 | 0.9 | 1.0 | 5.2 | 2.4 | 3.7 | 10.8 | 8.3 | 9.4 | 2.9 | 8.9 | 5.7 | 1.8 | 3.1 | 2.7 | 2.2 | 0.47 |
| YBL048W | 2.0 | 7.9 | 2.3 | 2.8 | 1.3 | 2.0 | 1.9 | 1.2 | 7.2 | 4.4 | 2.0 | 1.2 | 2.7 | 2.0 | 4.1 | 5.7 | 1.3 | 1.0 | 0.29 |
| YBL107C | 1.4 | 1.1 | 0.4 | 1.5 | 1.2 | 1.1 | 2.1 | 1.9 | 1.0 | 3.1 | 2.9 | 1.4 | 2.6 | 2.5 | 1.4 | 1.5 | 0.9 | 1.4 | 1.03 |
| YFL024C | 1.0 | 1.1 | 1.1 | 0.7 | 1.1 | 2.0 | 3.3 | 0.9 | 0.5 | 0.5 | 0.6 | 0.8 | 0.9 | 3.3 | 0.9 | 1.1 | 0.9 | 0.8 | 0.37 |
| YGL121C | 2.6 | 5.0 | 1.4 | 4.2 | 0.9 | 9.8 | 4.7 | 3.0 | 7.0 | 15.0 | 6.1 | 1.6 | 7.8 | 5.8 | 1.6 | 8.5 | 2.7 | 3.5 | 0.60 |
| YHR029C | 2.8 | 1.8 | 1.8 | 1.8 | 1.0 | 2.7 | 2.0 | 2.7 | 13.6 | 8.6 | 3.8 | 1.5 | 4.7 | 3.5 | 1.0 | 1.0 | 2.1 | 2.1 | 0.87 |
| YHR209W | 2.8 | 1.1 | 0.8 | 4.5 | 1.8 | 3.2 | 5.0 | 2.0 | 2.9 | 6.4 | 3.3 | 1.0 | 2.0 | 2.2 | 5.0 | 3.5 | 2.6 | 2.6 | 0.56 |
| YKL107W | 2.4 | 2.5 | 0.5 | 0.8 | 1.1 | 2.0 | 1.1 | 0.9 | 28.2 | 8.3 | 2.0 | 1.0 | 1.9 | 3.4 | 1.7 | 3.5 | 1.6 | 0.9 | 0.17 |
| YKR075C | 1.5 | 0.7 | 2.7 | 4.5 | 1.2 | 1.5 | 1.4 | 2.0 | 0.2 | 0.6 | 1.3 | 2.3 | 3.8 | 2.7 | 0.4 | 4.3 | 2.4 | 1.9 | 1.15 |
| YLL056C | 2.1 | 2.3 | 3.0 | 2.0 | 1.1 | 0.6 | 1.0 | 1.3 | 58.8 | 8.8 | 1.7 | 1.3 | 6.8 | 11.0 | 1.5 | 4.8 | 5.3 | 2.5 | 0.32 |
| YLR297W | 2.4 | 2.1 | 3.5 | 1.7 | 1.3 | 0.8 | 1.1 | 1.3 | 2.1 | 5.5 | 3.5 | 0.8 | 0.8 | 2.3 | 0.7 | 1.8 | 0.7 | 1.0 | 0.81 |
| YOR338W | 1.8 | 1.8 | 0.5 | 1.7 | 1.6 | 2.6 | 1.3 | 2.2 | 0.6 | 3.5 | 0.8 | 1.2 | 1.4 | 2.5 | 1.8 | 1.0 | 1.1 | 0.5 | 0.52 |
| YOR391C | 2.1 | 2.0 | 0.9 | 1.2 | 1.3 | 1.6 | 2.0 | 2.8 | 18.8 | 18.1 | 3.2 | 1.1 | 2.3 | 2.4 | 5.8 | 2.2 | 1.0 | 0.9 | 0.22 |
| YPL280W | 1.7 | 1.9 | 1.9 | 2.0 | 1.4 | 1.9 | 1.8 | 2.3 | 49.8 | 14.0 | 1.9 | 0.9 | 2.4 | 2.4 | 4.3 | 1.5 | 1.2 | 0.9 | 0.22 |
| YLR346C | 4.4 | 2.0 | 2.1 | 4.5 | 1.4 | 5.5 | 5.6 | 1.3 | 8.6 | 6.6 | 0.5 | 4.2 | 6.2 | 2.2 | 1.0 | 2.4 | 10.6 | 7.8 | 0.53 |
| YOR049C | 2.5 | 0.9 | 12.8 | 1.4 | 1.3 | 8.4 | 4.2 | 0.9 | 1.1 | 1.9 | 1.1 | 6.7 | 7.1 | 1.5 | 1.3 | 4.3 | 2.6 | 2.9 | 0.22 |
| YAL034C | 1.1 | 1.8 | 2.8 | 1.1 | 1.0 | 1.5 | 1.8 | 1.0 | 3.0 | 3.3 | 1.5 | 2.8 | 4.9 | 1.2 | 1.4 | 1.0 | 1.2 | 1.3 | 0.52 |
| YDR476C | 0.7 | 1.8 | 6.0 | 1.2 | 0.5 | 3.1 | 1.5 | 1.3 | 1.7 | 3.4 | 1.6 | 2.1 | 2.8 | 1.1 | 2.2 | 2.0 | 2.4 | 1.5 | 0.58 |
| YGR035C | 1.6 | 0.6 | 1.2 | 0.6 | 1.3 | 3.3 | 1.1 | 0.9 | 1.1 | 0.5 | 0.4 | 3.3 | 2.1 | 0.8 | 0.3 | 1.9 | 2.5 | 4.3 | 1.04 |
| YGR284C | 1.0 | 2.0 | 4.0 | 1.4 | 1.2 | 1.4 | 2.2 | 1.1 | 4.3 | 1.8 | 1.7 | 2.1 | 3.6 | 1.2 | 3.9 | 1.9 | 1.3 | 2.1 | 2.57 |
| YHR054C | 1.7 | 0.9 | 2.2 | 1.0 | 1.0 | 2.9 | 1.6 | 1.8 | 2.6 | 7.7 | 0.9 | 3.6 | 5.8 | 1.0 | 0.8 | 2.2 | 5.1 | 3.4 | 0.80 |
| YLR008C | 0.9 | 0.5 | 0.7 | 0.9 | 1.3 | 2.7 | 2.0 | 1.0 | 0.4 | 0.7 | 0.8 | 3.5 | 6.2 | 1.6 | 0.7 | 1.7 | 5.4 | 4.9 | 2.00 |
| YMR315W | | 4.3 | 1.8 | 1.7 | | 4.2 | 2.9 | 2.0 | 2.4 | 2.6 | 3.2 | 2.0 | 2.5 | 1.3 | 2.5 | 1.6 | 2.1 | 2.8 | 1.76 |
| YNL211C | 1.2 | 1.0 | 0.8 | 1.0 | 1.0 | 7.4 | 3.1 | 1.1 | 0.8 | 1.1 | 1.1 | 3.2 | 9.9 | 1.1 | 0.8 | 9.3 | 4.2 | 6.8 | 0.52 |
| YOL031C | 0.9 | 1.9 | 1.0 | 1.7 | 1.2 | 1.0 | 2.5 | 2.1 | 2.3 | 1.4 | 1.3 | 2.1 | 2.4 | 1.1 | 4.4 | 1.5 | 1.2 | 1.9 | 1.20 |
| YOL101C | 0.9 | 0.8 | 0.4 | 1.7 | 1.6 | 0.5 | 0.7 | 0.9 | 0.1 | 0.3 | 0.9 | 2.4 | 0.2 | 0.8 | 0.6 | 3.5 | 0.6 | 0.6 | 1.37 |
| YAR031W | 1.5 | 1.1 | 1.1 | 1.7 | 1.1 | 2.9 | 1.6 | 1.2 | 2.7 | 1.6 | 1.4 | 1.2 | 3.3 | 1.4 | 0.9 | 3.5 | 1.6 | 2.5 | 1.00 |
| YBL049W | 2.9 | 11.8 | 2.1 | 2.9 | 1.1 | | 1.7 | 1.9 | 5.2 | 5.2 | 2.2 | 0.7 | 3.9 | 1.5 | 7.2 | 6.4 | 1.1 | 1.1 | 0.47 |
| YBR062C | 1.3 | 1.7 | 0.4 | 1.2 | 1.4 | 1.9 | 1.5 | 1.9 | 5.4 | 2.2 | 2.3 | 1.2 | 2.8 | 2.6 | 0.7 | 2.2 | 1.4 | 1.6 | 0.93 |
| YCR013C | 1.3 | 1.4 | 5.0 | 1.1 | 1.1 | 1.8 | 1.6 | 0.7 | 0.8 | 1.3 | 2.4 | 1.4 | 3.9 | 1.5 | 1.6 | 1.6 | 1.1 | 1.3 | 2.80 |
| YDL027C | 1.2 | 2.0 | 1.7 | 1.7 | 1.2 | 2.1 | 2.9 | 1.5 | 2.0 | 2.3 | 1.7 | 1.7 | 3.2 | 1.1 | 1.4 | 1.8 | 1.4 | 2.0 | 0.69 |
| YDL110C | 1.5 | 1.4 | 1.1 | 2.1 | 0.8 | 1.7 | 2.5 | 1.8 | 3.0 | 2.3 | 1.9 | 1.5 | 2.7 | 2.7 | 1.3 | 3.6 | 2.0 | 4.4 | 1.27 |
| YDL169C | 1.7 | 1.7 | 1.1 | 1.3 | 1.1 | 2.6 | 2.1 | 1.3 | 7.3 | 4.7 | 1.2 | 1.2 | 3.7 | 2.7 | 2.2 | 3.8 | 1.5 | 1.6 | 0.42 |
| YDR070C | 2.2 | 6.4 | 3.6 | 3.5 | 0.9 | 6.2 | 1.4 | 1.9 | 13.9 | 6.0 | 4.1 | 4.3 | 6.6 | 2.0 | 2.1 | 15.1 | 1.2 | 3.1 | 0.44 |
| YDR210W | 1.1 | 1.0 | 0.9 | 1.0 | 2.0 | 1.2 | 1.5 | 1.4 | 0.5 | 1.0 | 1.0 | 1.0 | 2.3 | 1.1 | 1.0 | 1.5 | 1.9 | 1.9 | 1.82 |
| YDR214W | 1.3 | 4.0 | 4.5 | 1.3 | 1.1 | 1.7 | 1.5 | 1.5 | 5.5 | 2.4 | 1.9 | 1.1 | 5.3 | 1.2 | 1.1 | 0.7 | 1.2 | 1.0 | 1.07 |
| YDR435C | 1.2 | 1.1 | 1.2 | 1.4 | 1.2 | 2.5 | 2.1 | 1.2 | 0.8 | 2.1 | 1.3 | 1.1 | 2.5 | 1.3 | 0.6 | 2.0 | 1.3 | 2.0 | 1.30 |
| YER037W | 2.0 | 2.1 | 0.8 | 1.1 | 1.3 | 3.2 | 2.6 | 1.0 | 1.0 | 5.5 | 1.5 | 1.2 | 3.0 | 1.7 | 0.8 | 1.8 | 1.7 | 2.6 | 1.17 |
| YFL044C | 1.0 | 1.5 | 0.8 | 2.3 | 1.6 | 1.3 | 1.6 | 1.4 | 5.2 | 3.7 | 2.2 | 1.2 | 4.7 | 1.2 | 1.0 | 1.8 | 1.3 | 1.4 | 0.50 |
| YFR003C | 1.2 | 1.5 | 0.8 | 1.6 | 1.0 | 2.2 | 1.4 | 1.4 | 8.3 | 3.1 | 1.4 | 0.9 | 2.5 | 2.1 | 1.0 | 1.4 | 1.3 | 1.8 | 1.01 |
| YFR020W | 1.0 | 0.9 | 1.2 | 1.0 | 0.9 | 1.4 | 1.1 | 0.9 | 3.7 | 3.5 | 1.4 | 0.9 | 2.3 | 1.8 | 2.6 | 1.0 | 1.2 | 0.9 | 0.61 |
| YFR044C | 0.9 | 1.3 | 3.8 | 1.1 | 0.9 | 2.1 | 1.2 | 0.8 | 2.1 | 2.2 | 1.9 | 1.2 | 3.1 | 0.8 | 1.5 | 1.2 | 1.0 | 1.0 | 1.99 |
| YGR010W | 1.2 | 1.6 | 0.9 | 1.2 | 0.8 | 1.2 | 1.7 | 1.6 | 13.6 | 8.9 | 1.5 | 0.9 | 5.2 | 1.9 | 1.3 | 1.4 | 0.8 | 1.3 | 0.55 |
| YGR142W | 2.7 | 6.2 | 15.3 | 1.3 | 1.3 | 0.9 | 1.4 | 1.5 | 36.8 | 12.2 | 2.3 | 2.0 | 8.9 | 0.9 | 1.3 | 0.3 | 1.9 | 1.5 | 1.28 |
| YGR161C | 2.0 | 2.2 | 5.4 | 3.8 | 1.0 | 1.4 | 1.4 | 1.3 | 2.0 | 3.8 | 0.8 | 1.7 | 2.8 | 0.9 | 1.7 | 1.1 | 1.5 | 1.8 | 0.65 |
| YGR212W | 1.5 | 1.7 | 1.5 | 1.3 | 1.2 | | | 1.0 | 1.1 | 3.8 | 0.9 | 1.5 | 10.0 | 1.1 | 1.1 | 2.1 | 3.9 | 2.2 | 0.28 |
| YGR268C | 1.1 | 2.3 | 2.0 | 2.5 | 1.1 | 1.1 | 1.0 | 1.1 | 4.1 | 1.6 | 0.8 | 1.0 | 2.8 | 1.0 | 1.5 | 1.2 | 1.5 | 1.4 | 0.48 |
| YHR016C | 0.8 | 1.5 | 1.7 | 1.5 | | 1.8 | 3.7 | 0.8 | 2.8 | 2.0 | 2.2 | 1.3 | 3.3 | 0.9 | 0.6 | 2.1 | 1.2 | 1.4 | 0.51 |
| YHR087W | 1.2 | 2.7 | 8.6 | 2.8 | 0.7 | 4.2 | 3.4 | 1.2 | 4.1 | 3.9 | 3.7 | 1.4 | 4.0 | 1.5 | 1.9 | 3.8 | 1.2 | 2.9 | 1.34 |
| YJL048C | 1.7 | 2.5 | 2.0 | 2.9 | 0.9 | 1.5 | 0.7 | 1.5 | 1.2 | 3.8 | 3.2 | 1.1 | 2.4 | 1.7 | 0.6 | 2.3 | 1.6 | 2.5 | 0.82 |
| YJL144W | 1.9 | 1.6 | 2.2 | 1.6 | 1.2 | 1.7 | 1.7 | 1.3 | 14.6 | 8.9 | 3.0 | 0.8 | 3.9 | 1.6 | 1.0 | 2.5 | 2.2 | 2.4 | 0.60 |
| YJL163C | 1.2 | 1.9 | 1.7 | 1.5 | 0.9 | 2.8 | 2.3 | 0.7 | 14.5 | 2.5 | 3.1 | 2.0 | 4.5 | 1.0 | 2.0 | 2.8 | 1.5 | 1.7 | 0.28 |
| YJR074W | 1.0 | 1.4 | 2.0 | 2.0 | 0.7 | 1.2 | 1.8 | 1.3 | 3.7 | 3.1 | 1.2 | 2.0 | 2.8 | 1.7 | 3.4 | 1.5 | 1.8 | 1.9 | 0.68 |
| YKL065C | 1.9 | 2.0 | 1.3 | 1.7 | 1.0 | 2.3 | 3.8 | 1.8 | 3.1 | 3.9 | 1.3 | 1.3 | 3.0 | 2.1 | 1.7 | 2.8 | 2.8 | 4.6 | 2.29 |
| YKR011C | 1.1 | 1.6 | 0.9 | 1.6 | 1.2 | 2.3 | 2.7 | 2.0 | 4.7 | 4.4 | 1.5 | 1.1 | 3.5 | 1.4 | 1.8 | 1.3 | 1.3 | 1.9 | 0.66 |
| YKR018C | 0.7 | 1.1 | 1.7 | 0.7 | 0.9 | 1.7 | 1.0 | 0.7 | 3.9 | 1.7 | 1.4 | 1.0 | 3.0 | 1.1 | 1.0 | 1.8 | 1.0 | 1.2 | 0.98 |
| YKR046C | 1.9 | 7.8 | 2.8 | 1.8 | 0.6 | 1.9 | 1.3 | 0.8 | 3.9 | 2.0 | 2.6 | 1.3 | 2.8 | 1.9 | 2.9 | 3.5 | 1.2 | 1.9 | 0.73 |
| YKR049C | 1.6 | 4.2 | 1.0 | 3.2 | 0.9 | 3.0 | 2.4 | 1.3 | 2.2 | 3.2 | 2.2 | 1.2 | 4.1 | 2.1 | 0.9 | 4.2 | 1.6 | 3.3 | 1.17 |
| YLR132C | 1.0 | 1.1 | 0.3 | 1.1 | 1.2 | 1.1 | 1.1 | 0.7 | 4.5 | 2.5 | 0.5 | 1.0 | 2.7 | 1.3 | 2.3 | 1.6 | 1.2 | 1.6 | 0.36 |
| YLR161W | 0.9 | 0.8 | 0.8 | 0.7 | 0.9 | 1.2 | 1.3 | 0.8 | 1.0 | 1.5 | 1.5 | 0.8 | 2.1 | 1.0 | 0.6 | 1.1 | 1.1 | 0.9 | 0.33 |
| YLR217W | 0.9 | 2.0 | 2.1 | 0.7 | 1.4 | 1.4 | 1.5 | 1.0 | 3.2 | 8.2 | | 0.9 | 3.8 | 0.9 | 0.9 | 0.8 | 1.0 | 1.2 | 0.35 |
| YLR328W | 1.3 | 1.2 | 1.3 | 0.9 | 0.9 | 4.8 | 5.4 | 1.1 | 0.9 | 1.2 | 1.4 | 1.2 | 4.3 | 0.5 | 0.8 | 1.4 | 1.1 | 0.8 | 1.04 |
| YML128C | 1.5 | 3.3 | 4.1 | 4.1 | 0.5 | 5.1 | 2.8 | 1.1 | 5.1 | 3.1 | 2.9 | 1.4 | 4.9 | 1.4 | 2.6 | 5.6 | 2.0 | 5.5 | 1.66 |
| YMR040W | 3.1 | 2.9 | 0.9 | 2.8 | 1.7 | 2.8 | 5.1 | 1.7 | 1.4 | 1.4 | 1.5 | 2.1 | 5.2 | 1.8 | 2.0 | 2.7 | 3.1 | 3.7 | 0.62 |
| YMR251W | 1.3 | 1.3 | 1.8 | 0.6 | 2.2 | 1.6 | 3.1 | 1.7 | 3.1 | 11.6 | 2.8 | 1.2 | 2.5 | 1.5 | 0.4 | 1.3 | 1.5 | 0.9 | 0.20 |
| YMR322C | 2.0 | 4.3 | 1.5 | 2.9 | 1.4 | 1.8 | 1.7 | 2.1 | 21.3 | 10.3 | 3.2 | 1.1 | 2.1 | 2.7 | 3.6 | 1.8 | 1.4 | 0.8 | 0.18 |
| YNL094W | 0.9 | 1.4 | 1.1 | 1.0 | 1.4 | 2.9 | 1.2 | 2.0 | 3.5 | 2.6 | 1.7 | 1.2 | 3.4 | 1.0 | 1.5 | 1.5 | 1.1 | 1.3 | 0.66 |
| YNL134C | 2.3 | 6.0 | 4.5 | 0.9 | 1.7 | 1.4 | 1.5 | 3.2 | 6.1 | 5.9 | 5.0 | 1.1 | 3.1 | 1.4 | 0.8 | 2.1 | 1.3 | 2.0 | 2.51 |
| YNL155W | 1.1 | 1.2 | 1.1 | 0.9 | 1.4 | 3.0 | 1.3 | 1.7 | 5.8 | 2.7 | 2.2 | 1.1 | 4.4 | 2.1 | 2.0 | 2.0 | 1.1 | 1.6 | 1.89 |
| YOR059C | 1.2 | 1.2 | 0.4 | 0.9 | 1.4 | 1.1 | 2.2 | 1.3 | 7.6 | 4.6 | 1.0 | 1.0 | 3.3 | 2.2 | 3.5 | 1.8 | 1.3 | 1.7 | 0.71 |
| YOR152C | 4.5 | 2.5 | 3.4 | 2.9 | 0.8 | 4.5 | 3.2 | 1.2 | 10.0 | 5.0 | 2.7 | 2.9 | 7.4 | 1.7 | 3.6 | 2.1 | 4.0 | 2.2 | 0.42 |
| YOR173W | 0.7 | 2.3 | 6.3 | 2.0 | 0.9 | 3.5 | 4.9 | 1.4 | 5.2 | 3.7 | 2.3 | 1.9 | 3.8 | 2.8 | 0.7 | 5.7 | 1.7 | 3.8 | 0.54 |
| YOR289W | 1.6 | 1.5 | 1.0 | 2.0 | 1.2 | 4.2 | 6.1 | 2.4 | 2.4 | 1.7 | 2.6 | 1.5 | 4.3 | 2.0 | 1.6 | 3.0 | 2.3 | 3.9 | 0.74 |
| YPR030W | 1.7 | 1.4 | 1.4 | 1.0 | 1.2 | 1.3 | 1.2 | 0.9 | 2.5 | 2.4 | 1.2 | 1.4 | 3.2 | 1.5 | 1.5 | 3.1 | 1.4 | 2.1 | 0.30 |
| YAL008W | 1.4 | 2.4 | 2.0 | 2.4 | 0.7 | 1.6 | 1.9 | 1.3 | 1.8 | 1.5 | 2.2 | 1.3 | 2.2 | 2.6 | 0.9 | 4.2 | 1.4 | 1.9 | 1.04 |
| YBR053C | 1.0 | 1.6 | 2.4 | 2.4 | 1.6 | 2.7 | 4.1 | 1.2 | 2.9 | 2.1 | 2.3 | 1.1 | 2.4 | 1.0 | 0.7 | 2.8 | 1.9 | 2.4 | 1.25 |
| YBR099C | 1.0 | 1.4 | 7.2 | 1.8 | 1.0 | 0.5 | 0.9 | 1.5 | 3.4 | 12.9 | 0.4 | 1.0 | 2.1 | 1.6 | 0.8 | 0.5 | 0.8 | 0.9 | 0.47 |
| YBR137W | 1.1 | 2.2 | 1.4 | 3.5 | 0.9 | 2.4 | 2.7 | 2.0 | 2.2 | 1.8 | 2.4 | 1.4 | 2.1 | 1.8 | 0.6 | 2.8 | 1.4 | 2.6 | 1.40 |
| YBR203W | 1.4 | 1.4 | 1.6 | 2.4 | 0.9 | 0.6 | 0.9 | 1.3 | 1.1 | 1.8 | 1.3 | 1.6 | 2.4 | 1.3 | 1.0 | 18.0 | 1.4 | 2.2 | 0.38 |
| YCL049C | 1.9 | 1.3 | 1.4 | 1.2 | 1.6 | 2.3 | 2.1 | 1.0 | 2.0 | 1.9 | 1.6 | 1.4 | 2.4 | 1.8 | 1.4 | 2.1 | 1.4 | 1.4 | 0.86 |
| YCR082W | 1.4 | 1.2 | 0.7 | 1.2 | 1.3 | 1.3 | 1.5 | 1.5 | 4.3 | 2.3 | 2.0 | 1.0 | 2.0 | 2.4 | 1.6 | 2.2 | 1.2 | 1.7 | 1.62 |
| YDL054C | 1.3 | 2.2 | 2.7 | 1.1 | 1.2 | 1.2 | 0.9 | 0.7 | 3.5 | 2.1 | 2.1 | 1.1 | 2.7 | 1.2 | 1.0 | 1.0 | 1.0 | 1.0 | 0.92 |
| YDL115C | 1.4 | 1.0 | 0.5 | 1.8 | 0.8 | 1.5 | 1.8 | 1.6 | 6.6 | 3.6 | 1.8 | 1.1 | 1.9 | 2.3 | 1.2 | 1.6 | 1.3 | 1.8 | 0.83 |
| YDL144C | 0.8 | 0.9 | 1.2 | 1.5 | 1.0 | 1.8 | 1.0 | 0.9 | 3.6 | 1.2 | 1.9 | 1.0 | 2.1 | 1.2 | 1.0 | 1.1 | 0.9 | 0.9 | 0.52 |
| YDL223C | 0.7 | 1.1 | 5.0 | 2.5 | 0.9 | 0.8 | 1.0 | 0.3 | 3.8 | 1.5 | 5.2 | 1.0 | 3.1 | 1.3 | 3.1 | 7.2 | 0.9 | 1.0 | 0.32 |
| YDR032C | 1.7 | 2.2 | 3.5 | 2.5 | 1.4 | 2.7 | 2.8 | 2.4 | 3.1 | 2.9 | 3.3 | 1.8 | 2.9 | 2.8 | 1.3 | 3.8 | 1.9 | 3.5 | 3.50 |
| YDR330W | 0.8 | 1.5 | 1.2 | 0.8 | 1.2 | 2.3 | 1.3 | 1.1 | 3.9 | 2.6 | 0.9 | 1.0 | 2.2 | 1.1 | 1.6 | 1.4 | 1.5 | 1.3 | 0.53 |
| YDR411C | 0.9 | 2.8 | 1.8 | 1.2 | 1.6 | 0.8 | 2.0 | 0.9 | 0.7 | 1.0 | 1.3 | 1.5 | 1.9 | 0.8 | 4.1 | 1.5 | 1.5 | 1.3 | 0.56 |
| YDR511W | 1.3 | 1.7 | 0.9 | 2.3 | 1.0 | 2.0 | 1.5 | 1.7 | 2.5 | 2.2 | 1.2 | 0.8 | 3.1 | 2.2 | 0.8 | 2.2 | 1.3 | 1.5 | 0.66 |
| YDR545W | 0.6 | 1.2 | 0.6 | 0.7 | 1.4 | 1.0 | 1.2 | 0.6 | 1.8 | 0.7 | 0.7 | 0.9 | 2.0 | 0.7 | 0.9 | 0.9 | 0.9 | 0.9 | 1.06 |
| YER004W | 1.0 | 1.5 | 1.9 | 1.4 | 1.5 | 1.2 | 1.4 | 1.1 | 4.5 | 2.9 | 2.2 | 1.7 | 4.6 | 1.8 | 1.4 | 2.3 | 1.4 | 1.2 | 1.48 |
| YER035W | 1.4 | 1.6 | 1.6 | 1.9 | 1.5 | 0.9 | 1.9 | 1.2 | 1.2 | 1.4 | 1.4 | 1.1 | 1.9 | 1.0 | 0.9 | 2.1 | 2.2 | 2.1 | 0.85 |
| YER079W | 1.1 | 1.6 | 1.9 | 2.0 | 0.8 | 1.5 | 1.9 | 1.4 | 5.3 | 3.4 | 2.1 | 0.9 | 2.1 | 1.6 | 0.5 | 1.4 | 1.0 | 1.6 | 0.69 |
| YER158C | 1.2 | 0.9 | 0.9 | 3.1 | 0.8 | 0.7 | 1.2 | 0.9 | 2.1 | 1.6 | 2.3 | 1.0 | 3.1 | 0.9 | 0.6 | 1.6 | 1.6 | 1.2 | 0.61 |
| YER163C | 1.1 | 2.4 | 1.2 | 1.6 | 0.8 | 2.0 | 1.5 | 1.4 | 5.4 | 3.4 | 1.6 | 1.0 | 2.9 | 1.9 | 1.6 | 2.4 | 1.2 | 1.2 | 0.62 |
| YER175C | 0.9 | 1.2 | 1.3 | 2.2 | 0.9 | 0.8 | 2.3 | 1.1 | 1.9 | 11.5 | 1.1 | 0.9 | 4.3 | 1.1 | 1.3 | 2.8 | 0.9 | 1.2 | 0.53 |
| YFL006W | 0.8 | 2.8 | 1.6 | 1.0 | 1.4 | 1.2 | 1.0 | 1.0 | 3.2 | 1.7 | 1.2 | 1.2 | 3.1 | 1.7 | 2.4 | 1.1 | 1.0 | 0.8 | 0.82 |
| YFL010C | 1.0 | 4.8 | 1.3 | 1.0 | 1.3 | 1.9 | 1.5 | 1.0 | 3.1 | 1.4 | 1.4 | 1.5 | 3.3 | 1.4 | 1.7 | 1.4 | 1.9 | 1.6 | 0.77 |
| YFL032W | 0.4 | 1.5 | 1.6 | 0.8 | 0.9 | 0.6 | 1.1 | 0.4 | 0.5 | 0.6 | 1.1 | 0.7 | 2.4 | 0.6 | 2.0 | 0.7 | 1.7 | 0.8 | 1.62 |
| YGL037C | 1.5 | 2.7 | 6.5 | 2.1 | 0.9 | 1.3 | 2.9 | 1.2 | 2.1 | 1.6 | 3.9 | 0.8 | 2.3 | 1.0 | 0.8 | 1.5 | 1.3 | 2.6 | 4.58 |
| YGL047W | 1.0 | 1.9 | 1.6 | 2.3 | 1.3 | 1.5 | 1.7 | 1.1 | 4.5 | 5.7 | 1.9 | 1.1 | 2.6 | 2.3 | 1.7 | 2.9 | 1.1 | 1.2 | 0.97 |
| YGL053W | 1.5 | 1.5 | 1.2 | 2.5 | 2.0 | 2.7 | 3.7 | 1.2 | 3.3 | 2.4 | 1.3 | 1.3 | 3.3 | 1.1 | 1.1 | 3.9 | 2.2 | 3.7 | 1.26 |
| YGL199C | 1.1 | 1.7 | 1.7 | 1.0 | 0.7 | 0.7 | 1.3 | 0.7 | | 0.1 | | 0.9 | 2.0 | 0.8 | 1.6 | 1.6 | 1.2 | 1.0 | 0.36 |
| YGR101W | 1.0 | 1.3 | 1.2 | 1.1 | 1.2 | 0.9 | 1.5 | 0.8 | 4.0 | 1.4 | 2.0 | 1.0 | 2.4 | 0.9 | 1.1 | 2.0 | 1.0 | 1.1 | 1.04 |
| YGR130C | 0.9 | 0.7 | 0.6 | 1.6 | 1.4 | 0.9 | 1.3 | 0.9 | 2.0 | 1.5 | 0.8 | 0.7 | 1.9 | 0.9 | 1.2 | 2.2 | 1.3 | 1.6 | 0.78 |
| YGR154C | 1.8 | 2.9 | 2.2 | 0.5 | 1.2 | 1.4 | 2.0 | 1.7 | 25.2 | 9.1 | 1.5 | 0.9 | 2.5 | 2.2 | 0.7 | 2.0 | 1.0 | 1.2 | 0.36 |
| YGR221C | 0.9 | 2.2 | 0.9 | 1.4 | 0.9 | 3.0 | 1.2 | 1.1 | | 0.7 | 0.5 | 1.6 | 2.3 | 1.4 | 1.5 | 2.7 | 1.3 | 1.3 | 0.29 |
| YHR138C | 2.8 | 1.8 | 2.2 | 2.8 | 1.5 | 3.8 | 3.0 | 1.4 | 5.3 | 4.6 | 1.6 | 1.8 | 4.0 | 3.4 | 2.2 | 3.3 | 5.0 | 7.1 | 1.89 |
| YHR199C | 1.9 | 2.0 | 3.2 | 2.0 | 1.2 | 1.1 | 1.1 | 1.0 | 8.6 | 6.0 | 1.9 | 1.1 | 2.1 | 2.0 | 2.8 | 3.3 | 1.1 | 0.9 | 0.63 |
| YIL041W | 1.0 | 1.2 | 2.1 | 1.3 | 0.5 | 1.0 | 0.8 | 0.9 | 1.8 | 1.4 | 1.4 | 1.0 | 2.3 | 1.3 | 1.3 | 0.7 | 0.8 | 1.1 | 1.33 |
| YIL087C | 1.3 | 1.9 | 3.4 | 1.6 | 1.4 | 4.1 | 1.8 | 1.1 | 2.4 | 1.8 | 2.8 | 1.1 | 2.4 | 1.3 | 0.4 | 5.6 | 1.7 | 1.6 | 0.68 |
| YJL057C | 1.5 | 1.2 | 1.6 | 1.7 | 1.5 | 1.4 | 2.7 | 1.5 | 7.3 | 6.1 | 2.0 | 1.0 | 2.5 | 1.6 | 1.4 | 1.9 | 1.4 | 1.5 | 0.51 |
| YJL066C | 0.9 | 1.2 | 1.2 | 2.5 | 0.8 | 1.4 | 1.6 | 0.8 | 2.0 | 2.1 | 0.8 | 1.0 | 2.2 | 1.0 | 1.3 | 2.8 | 1.4 | 1.5 | 0.43 |
| YJL082W | 1.4 | 0.8 | 2.4 | 1.1 | 1.7 | 0.2 | 0.4 | 0.9 | 1.4 | 2.9 | 1.0 | 1.3 | 2.1 | 0.5 | 3.2 | 0.2 | 0.6 | 0.3 | 2.07 |
| YJL151C | 1.0 | 2.4 | 3.2 | 1.2 | 1.3 | 2.2 | 1.4 | 0.8 | 0.5 | 1.6 | 1.0 | 1.3 | 2.4 | 1.3 | 1.0 | 2.5 | 3.5 | 2.4 | 1.69 |
| YJL152W | 0.9 | 1.5 | 1.8 | 1.3 | 1.6 | 1.5 | 1.2 | 0.7 | 0.3 | 1.0 | 1.0 | 1.0 | 1.8 | 1.4 | 0.8 | 1.6 | 2.5 | 1.7 | 0.80 |
| YJL161W | 1.7 | 2.2 | 1.5 | 4.6 | 1.1 | 8.0 | 3.6 | 1.4 | 3.1 | 2.1 | 3.7 | 1.0 | 2.5 | 2.2 | 0.6 | 4.4 | 1.9 | 2.1 | 0.44 |
| YJL171C | 2.3 | 0.7 | 2.8 | 1.4 | 1.0 | 2.7 | 1.4 | 0.9 | 1.0 | 1.7 | 2.2 | 1.0 | 2.9 | 1.0 | 1.8 | 1.0 | 3.6 | 2.9 | 1.94 |
| YJR008W | 1.6 | 1.5 | 1.2 | 1.7 | 1.4 | 2.2 | 2.9 | 1.1 | 1.8 | 1.9 | 1.4 | 1.3 | 2.0 | 1.2 | 0.9 | 3.3 | 1.5 | 2.7 | 0.87 |
| YKL151C | 1.2 | 3.1 | 1.7 | 2.5 | 1.0 | 2.0 | 5.6 | 1.0 | 3.3 | 3.5 | 2.3 | 1.0 | 2.0 | 1.3 | 1.4 | 3.7 | 1.4 | 1.8 | 1.02 |
| YKL153W | 1.1 | 1.4 | 2.0 | 0.7 | 1.0 | 2.9 | 1.1 | 0.7 | 0.8 | 1.4 | 2.1 | 1.1 | 2.4 | 1.1 | 1.5 | 1.1 | 0.9 | 1.3 | 1.60 |
| YKL195W | 0.8 | 0.5 | 0.4 | 1.1 | 1.9 | 2.5 | 2.1 | 1.2 | 3.6 | 2.1 | 1.0 | 1.1 | 2.4 | 1.6 | 0.9 | 1.7 | 1.4 | 1.6 | 1.58 |
| YLR054C | 2.0 | 1.5 | 0.4 | 2.4 | 1.3 | 1.2 | 1.1 | 1.1 | 2.7 | 1.1 | 0.7 | 1.1 | 3.2 | 1.1 | 0.7 | 0.9 | 1.6 | 1.1 | 0.14 |
| YLR149C | 1.4 | 1.9 | 3.4 | 2.3 | 0.8 | 1.4 | 1.6 | 1.1 | 6.2 | 5.2 | 2.8 | 2.0 | 3.0 | 1.2 | 1.3 | 2.8 | 1.3 | 3.0 | 0.33 |
| YLR152C | 0.7 | 1.3 | 1.1 | 0.9 | 1.1 | 2.1 | 1.4 | 1.2 | 0.6 | 1.1 | 3.6 | 1.0 | 2.8 | 1.2 | 0.8 | 2.8 | 1.5 | 1.8 | 0.46 |
| YLR324W | 0.7 | 1.2 | 0.9 | 1.0 | 1.3 | 1.2 | 1.0 | 1.0 | 3.1 | 1.4 | 0.9 | 0.8 | 2.3 | 1.4 | 1.6 | 1.7 | 1.2 | 1.2 | 0.49 |
| YLR350W | 1.2 | 1.9 | 3.2 | 2.3 | 0.8 | 1.6 | 1.2 | 1.0 | 0.6 | 1.4 | 0.9 | 1.8 | 4.5 | 2.8 | 1.6 | 2.9 | 1.6 | 1.1 | 1.52 |
| YLR387C | 0.9 | 1.6 | 0.8 | 0.6 | 1.3 | 1.9 | 1.1 | 1.4 | 7.8 | 3.0 | 0.9 | 1.1 | 3.6 | 1.7 | 1.9 | 1.0 | 1.0 | 1.4 | 1.20 |
| YML117W | 0.9 | 2.2 | 0.9 | 1.6 | 0.8 | 1.0 | 0.7 | 0.7 | 1.0 | 1.4 | 0.8 | 0.8 | 2.0 | 0.8 | 2.8 | 0.7 | 1.1 | 1.3 | 0.70 |
| YMR009W | 2.3 | 1.9 | 0.5 | 1.9 | 1.9 | 1.2 | 1.9 | 1.4 | 3.4 | 2.2 | 2.1 | 0.6 | 2.3 | 1.9 | 0.6 | 2.3 | 2.7 | 2.5 | 0.75 |
| YMR067C | 0.9 | 0.9 | 1.1 | 0.4 | 1.1 | 1.4 | 1.2 | 1.1 | 4.0 | 3.5 | 1.1 | 1.1 | 2.2 | 1.5 | 1.4 | 1.0 | 1.0 | 1.0 | 0.49 |
| YMR097C | 1.5 | 1.0 | 0.7 | 1.6 | 0.7 | 1.2 | 1.5 | 1.5 | 2.1 | 2.1 | 1.5 | 1.2 | 2.1 | 2.1 | 1.7 | 1.7 | 1.4 | 1.8 | 0.59 |
| YMR102C | 1.0 | 2.9 | 1.1 | 0.8 | 1.2 | 6.3 | 1.8 | 1.0 | 1.7 | 2.6 | 1.5 | 2.4 | 4.4 | 0.7 | 0.7 | 1.1 | 2.4 | 2.8 | 0.94 |
| YMR107W | 5.0 | 3.9 | 1.7 | 18.4 | 1.1 | 1.0 | 1.2 | 1.3 | 6.0 | 17.1 | 3.0 | 0.8 | 1.8 | 8.1 | 1.9 | 12.0 | 1.3 | 8.1 | 0.49 |
| YMR180C | 1.2 | 0.9 | 1.0 | 1.3 | 1.0 | 2.4 | 1.7 | 1.8 | 1.8 | 2.2 | 0.7 | 1.2 | 3.1 | 1.7 | 1.0 | 1.8 | 1.4 | 1.7 | 0.57 |
| YMR184W | 1.3 | 5.4 | 1.0 | 1.1 | 0.8 | 0.9 | 1.6 | 1.9 | 2.1 | 0.9 | 1.5 | 1.7 | 2.3 | 1.5 | 1.1 | 1.2 | 1.4 | 2.0 | 0.68 |
| YMR191W | 1.3 | 2.4 | 1.9 | 1.6 | 1.5 | 1.0 | 2.0 | 1.1 | 2.2 | 3.5 | 2.3 | 1.8 | 3.4 | 1.5 | 2.7 | 1.7 | 1.2 | 2.1 | 0.99 |
| YMR295C | 1.7 | 1.0 | 1.2 | 1.6 | 1.4 | 1.9 | 1.3 | 1.0 | 1.0 | 0.8 | 1.2 | 1.1 | 2.3 | 1.3 | 0.9 | 1.9 | 2.1 | 3.4 | 3.43 |
| YMR316W | 3.2 | 2.9 | 1.1 | 1.9 | 1.1 | 0.7 | 2.0 | 1.6 | 2.1 | 3.0 | 1.5 | 1.9 | 2.9 | 1.3 | 0.5 | 1.3 | 6.9 | 6.4 | 1.25 |
| YNL044W | 1.3 | 0.9 | 2.3 | 1.3 | 1.2 | 0.9 | 0.9 | 1.8 | 0.8 | 0.8 | 1.1 | 1.0 | 3.1 | 1.2 | 2.4 | 1.0 | 3.1 | 3.4 | 2.56 |
| YNL074C | 0.9 | 1.3 | 1.8 | 1.2 | 1.3 | 1.7 | 1.1 | 0.6 | 1.5 | 2.0 | 1.3 | 0.9 | 2.3 | 0.6 | 1.0 | 0.7 | 1.1 | 1.3 | 0.68 |
| YNL092W | 1.3 | 1.4 | 2.4 | 1.8 | 0.9 | 1.2 | 1.6 | 0.9 | 2.3 | 1.5 | 0.7 | 1.1 | 2.3 | 1.2 | 2.7 | 1.3 | 1.7 | 1.6 | 0.16 |
| YNL115C | 0.7 | 1.8 | 1.6 | 1.4 | 0.8 | 9.1 | 2.4 | 0.8 | 2.6 | 1.9 | 1.1 | 0.8 | 2.5 | 1.0 | 1.6 | 3.1 | 1.6 | 2.6 | 0.80 |
| YNL156C | 1.1 | 1.2 | 0.9 | 1.3 | 1.6 | 1.3 | 1.9 | 1.2 | 2.4 | 1.9 | 1.4 | 0.9 | 2.0 | 1.4 | 1.8 | 2.1 | 1.8 | 1.9 | 1.85 |
| YNL234W | 1.1 | 1.1 | 0.6 | 1.8 | 1.7 | 1.3 | 1.2 | 1.3 | 2.6 | 3.3 | 1.3 | 1.1 | 2.5 | 1.5 | 1.4 | 2.6 | 1.0 | 1.0 | 0.49 |
| YNL281W | 1.0 | 1.3 | 2.0 | 1.4 | 1.7 | 0.9 | 1.2 | 1.2 | 2.8 | 1.6 | 1.6 | 0.7 | 2.0 | 1.2 | 1.2 | 0.5 | 0.9 | 0.9 | 1.79 |
| YNL305C | 0.9 | 2.2 | 2.7 | 1.4 | 1.3 | 1.1 | 1.7 | 0.7 | 2.9 | 2.1 | 1.8 | 1.1 | 2.6 | 1.3 | 1.6 | 2.5 | 1.7 | 1.6 | 1.35 |
| YNR068C | 1.2 | 1.5 | 2.3 | 2.6 | 1.7 | 0.4 | 1.8 | 1.1 | 6.7 | 14.4 | 1.1 | 1.3 | 3.7 | 1.8 | 1.0 | 1.4 | 1.0 | 1.0 | 0.24 |
| YOL032W | 1.3 | 0.8 | 2.0 | 1.3 | 1.4 | 2.1 | 1.4 | 1.5 | 4.5 | 3.0 | 1.4 | 1.0 | 2.3 | 1.3 | 0.9 | 2.4 | 1.7 | 1.8 | 0.77 |
| YOL036W | 0.8 | 0.9 | 1.3 | 0.8 | 1.2 | 1.2 | 1.0 | 0.8 | 3.1 | 2.8 | 2.2 | 1.0 | 2.5 | 1.0 | 2.0 | 1.0 | 1.1 | 0.9 | 0.48 |
| YOL047C | 1.4 | 1.1 | 1.1 | 2.5 | 1.4 | 1.5 | 1.3 | 1.1 | 0.6 | 2.3 | 0.9 | 0.8 | 2.0 | 1.2 | 12.3 | 1.3 | 1.5 | 1.0 | 0.19 |
| YOL071W | 1.3 | 2.0 | 1.7 | 2.0 | 0.9 | 1.7 | 2.0 | 1.6 | 4.4 | 2.3 | 4.3 | 1.0 | 2.5 | 2.5 | 0.7 | 4.6 | 1.3 | 2.0 | 1.08 |
| YOL082W | 1.0 | 1.6 | 1.6 | 2.1 | 1.5 | 1.8 | 2.5 | 1.1 | 3.4 | 2.0 | 1.6 | 1.0 | 2.4 | 1.4 | 1.0 | 2.4 | 1.6 | 2.5 | 0.60 |
| YOL083W | 1.1 | 2.3 | 1.2 | 2.8 | 1.4 | 4.5 | 3.1 | 1.3 | 2.7 | 2.3 | 1.3 | 1.2 | 2.3 | 1.4 | 1.0 | 3.8 | 1.4 | 3.2 | 0.41 |
| YOL117W | 1.0 | 1.1 | 0.5 | 1.4 | 1.7 | 2.0 | 1.6 | 1.1 | 1.3 | 4.1 | 1.0 | 0.9 | 2.2 | 1.7 | 1.1 | 2.2 | 1.3 | 1.4 | 0.21 |
| YOL131W | 1.0 | 1.5 | 0.6 | 1.0 | 1.5 | 1.1 | 1.3 | 1.1 | 8.4 | 8.1 | 1.4 | 0.9 | 3.1 | 2.3 | 1.4 | 0.7 | 1.1 | 0.9 | 0.15 |
| YOL162W | 2.0 | 6.9 | 1.7 | 1.4 | 0.9 | 1.2 | 2.0 | 1.6 | 12.0 | 6.8 | 4.3 | 1.4 | 3.8 | 1.4 | 0.6 | 0.9 | 1.2 | 0.9 | 0.26 |
| YOR019W | 2.9 | 1.2 | 0.8 | 4.7 | 1.4 | 1.3 | 2.8 | 1.1 | 3.5 | 7.1 | 1.7 | 1.0 | 2.2 | 1.4 | 0.7 | 2.6 | 1.7 | 2.8 | 0.40 |
| YOR197W | 0.8 | 1.0 | 1.1 | 1.4 | 0.6 | 1.3 | 0.9 | 0.9 | 0.8 | 0.7 | 1.0 | 1.0 | 2.1 | 0.7 | 1.7 | 1.0 | 1.5 | 1.5 | 1.30 |
| YPL087W | 1.0 | 5.1 | 3.0 | 7.3 | 0.6 | 4.3 | 4.2 | 1.0 | 1.0 | 2.2 | 2.5 | 1.8 | 4.4 | 1.2 | 1.3 | 1.8 | 2.1 | 2.7 | 1.49 |
| YPL196W | 1.3 | 1.6 | 1.3 | 1.7 | 1.5 | 2.0 | 2.4 | 1.2 | 10.9 | 2.8 | 1.7 | 0.9 | 2.1 | 1.6 | 0.7 | 4.3 | 1.4 | 2.3 | 0.92 |
| YPL206C | 1.0 | 2.9 | 1.4 | 1.9 | 0.7 | 1.8 | 1.2 | 1.0 | 1.7 | 1.1 | 1.5 | 0.8 | 2.5 | 1.1 | 0.8 | 1.2 | 1.4 | 1.9 | 1.22 |
| YPL222W | 0.9 | 3.5 | 0.8 | 1.3 | 1.2 | 2.2 | 1.1 | 0.7 | 7.4 | 3.4 | 1.2 | 1.3 | 3.0 | 1.1 | 0.9 | 2.2 | 1.1 | 0.9 | 0.26 |
| YPR023C | 1.2 | 1.4 | 1.2 | 0.8 | 1.1 | 1.4 | 1.2 | 0.9 | 4.4 | 2.8 | 1.7 | 0.9 | 2.0 | 0.9 | 0.8 | 1.4 | 1.0 | 1.1 | 0.81 |
| YPR151C | 2.3 | 2.0 | 1.5 | 4.1 | 1.3 | 1.6 | 4.4 | 1.4 | 3.0 | 9.5 | 1.4 | 0.9 | 2.0 | 1.4 | 0.7 | 2.8 | 1.3 | 1.4 | 0.16 |
| YDL222C | 0.9 | 1.1 | 1.5 | 3.9 | 1.0 | 1.4 | 1.0 | 0.7 | 1.9 | 1.0 | 2.6 | 1.0 | 2.2 | 1.3 | 7.1 | 6.5 | 0.9 | 1.3 | 0.50 |
| YEL001C | 1.0 | 1.0 | 1.4 | 0.7 | 0.9 | 1.3 | 1.1 | 1.2 | 2.0 | 1.2 | 1.2 | 1.1 | 0.9 | 1.3 | 3.0 | 1.2 | 0.7 | 1.3 | 2.81 |
| YER106W | 1.3 | 0.7 | 1.3 | 1.2 | 1.6 | 1.1 | 1.0 | 1.2 | 0.4 | 1.8 | 1.0 | 0.9 | 1.0 | 1.5 | 6.8 | 0.9 | 0.8 | 0.8 | 0.25 |
| YIL023C | 1.1 | 1.2 | 2.1 | 1.8 | 1.3 | 0.9 | 1.2 | 0.7 | 3.5 | 1.6 | 1.1 | 0.9 | 1.1 | 0.7 | 5.6 | 1.1 | 1.2 | 1.0 | 0.93 |
| YJR054W | 0.8 | 0.9 | 0.3 | 0.9 | 1.0 | 0.7 | 0.9 | 1.6 | | 0.6 | 0.7 | 0.9 | 0.4 | 1.2 | 3.1 | 0.6 | 0.9 | 0.9 | 0.52 |
| YKL086W | 1.0 | 1.1 | 2.4 | 2.6 | 0.8 | 1.0 | 0.7 | 1.1 | 8.2 | 20.5 | 0.7 | 0.6 | 1.3 | 2.9 | 5.0 | 0.6 | 0.9 | 1.0 | 0.28 |
| YKR091W | 1.5 | 2.3 | 0.9 | 3.5 | 1.6 | 1.0 | 1.3 | 1.3 | 1.0 | 1.6 | 1.1 | 0.9 | 1.0 | 1.9 | 6.3 | 1.6 | 1.6 | 1.3 | 0.45 |
| YLR194C | 2.1 | 2.2 | 1.3 | 3.6 | 1.0 | 0.9 | 1.0 | 1.0 | 0.9 | 1.1 | 1.4 | 1.6 | 4.0 | 1.2 | 3.6 | 0.9 | 1.0 | 0.9 | 0.60 |
| YMR095C | 1.1 | 2.0 | 1.6 | 1.0 | 0.9 | 0.7 | 1.2 | 1.2 | | 1.4 | 2.1 | 0.8 | 3.4 | 2.2 | 41.7 | 1.0 | 1.0 | 1.0 | 0.23 |
| YAL053W | 0.9 | 0.9 | 1.3 | 2.1 | 0.8 | 1.1 | 0.7 | 0.8 | 1.4 | 0.9 | 2.3 | 0.6 | 1.1 | 0.7 | 2.9 | 1.1 | 1.2 | 1.6 | 1.60 |
| YDL072C | 1.3 | 1.1 | 3.5 | 2.1 | 1.4 | 1.5 | 1.8 | 0.8 | 1.1 | 1.5 | 1.4 | 1.2 | 1.6 | 1.8 | 2.6 | 2.6 | 1.6 | 2.8 | 3.25 |
| YDL204W | 1.4 | 1.9 | 4.8 | 3.4 | 1.0 | 5.6 | 3.2 | 1.1 | 2.3 | 2.0 | 3.2 | 0.9 | 3.2 | 2.4 | 3.6 | 8.6 | 1.8 | 2.7 | 0.73 |
| YDR391C | 1.2 | 2.0 | 1.3 | 1.6 | 1.2 | 2.8 | 2.0 | 1.4 | 1.5 | 2.7 | 1.9 | 1.4 | 1.8 | 2.3 | 3.5 | 3.0 | 1.8 | 2.4 | 1.05 |
| YIL024C | 1.4 | 1.3 | 1.1 | 2.1 | 1.3 | 1.0 | 1.3 | 1.1 | 2.2 | 1.8 | 0.6 | 0.7 | 1.0 | 1.5 | 3.1 | 1.0 | 1.1 | 1.0 | 0.35 |
| YIL117C | 2.5 | 1.2 | 1.0 | 3.3 | 0.8 | 1.2 | 1.5 | 1.8 | 2.3 | 3.5 | 1.8 | 1.4 | 1.3 | 2.6 | 7.5 | 1.0 | 2.6 | 2.6 | 1.22 |
| YJL108C | 1.2 | 1.2 | 1.0 | 2.2 | 1.5 | 0.6 | 0.6 | 1.0 | 0.3 | 0.7 | 1.0 | 0.4 | 0.5 | 1.0 | 4.0 | 0.6 | 1.0 | 0.9 | 0.40 |
| YJL149W | 1.2 | 1.8 | 1.5 | 6.8 | 0.8 | 0.7 | 1.3 | 1.0 | 1.5 | 3.8 | 1.5 | 1.1 | 1.9 | 1.1 | 9.0 | 1.4 | 0.8 | 1.2 | 0.28 |
| YJL186W | 0.6 | 0.9 | 1.2 | 0.8 | 0.7 | 0.7 | 0.6 | 0.6 | 0.2 | 0.5 | 0.6 | 0.6 | 0.4 | 0.8 | 2.5 | 1.0 | 0.7 | 0.8 | 1.07 |
| YNL190W | 1.0 | 1.4 | 2.6 | 0.9 | 1.0 | 0.5 | 0.8 | 0.8 | 1.1 | 1.1 | 1.0 | 1.0 | 1.3 | 0.6 | 2.6 | 1.3 | 0.7 | 0.8 | 4.72 |
| YNL208W | 1.5 | 3.2 | 2.9 | 2.1 | 1.2 | 1.4 | 0.9 | 1.5 | 2.3 | 3.1 | 2.5 | 0.8 | 1.7 | 1.0 | 2.6 | 0.9 | 1.0 | 1.0 | 1.55 |
| YNL300W | 0.7 | 0.3 | 2.9 | 1.5 | 0.9 | 0.4 | 0.4 | 0.9 | | 0.2 | 0.9 | 0.8 | 0.7 | 0.8 | 3.3 | 1.0 | 1.0 | 0.8 | 0.39 |
| YNR064C | 0.9 | 0.6 | 0.9 | 1.8 | 1.5 | 2.1 | 0.8 | 0.9 | | 2.1 | 0.7 | 0.7 | 1.6 | 0.8 | 6.1 | 1.4 | 1.1 | 1.9 | 2.17 |
| YOR248W | 2.4 | 0.7 | 2.9 | 1.3 | 0.7 | 0.2 | 0.3 | 0.7 | 0.1 | 0.4 | 2.3 | 0.5 | 0.4 | 0.5 | 8.8 | 0.3 | 1.0 | 0.8 | 2.47 |
| YPL052W | 1.8 | 1.7 | 0.4 | 1.0 | 1.7 | 1.9 | 1.4 | 1.7 | 2.0 | 4.8 | 1.2 | 1.1 | 1.6 | 3.2 | 3.3 | 1.1 | 1.3 | 1.5 | 0.88 |
| YPR079W | 1.0 | 1.8 | 1.3 | 1.5 | 0.9 | 1.0 | 2.3 | 1.1 | 1.7 | 2.8 | 0.8 | 1.0 | 1.3 | 1.3 | 2.9 | 1.9 | 1.4 | 1.5 | 0.42 |
| YAR028W | 1.2 | 1.0 | 0.7 | 1.2 | 0.9 | 3.3 | 1.8 | 1.3 | 1.0 | 0.9 | 0.9 | 0.8 | 1.4 | 1.2 | 0.9 | 1.5 | 1.9 | 2.1 | 1.40 |
| YDR031W | 1.5 | 1.1 | 1.7 | 1.5 | 1.2 | 3.6 | 1.9 | 1.4 | 1.1 | 2.7 | 1.2 | 0.9 | 1.3 | 2.5 | 0.8 | 2.4 | 1.3 | 2.5 | 1.23 |
| YDR486C | 1.1 | 1.5 | 1.2 | 0.9 | 1.2 | 2.7 | 1.8 | 1.8 | 2.5 | 5.8 | 1.4 | 1.3 | 1.7 | 2.4 | 1.1 | 2.6 | 1.1 | 1.7 | 1.18 |
| YER038C | 1.2 | 1.4 | 0.6 | 1.2 | 1.4 | 2.8 | 1.5 | 1.1 | 0.7 | 2.5 | 0.6 | 0.9 | 2.2 | 1.3 | 0.8 | 1.6 | 1.2 | 1.4 | 0.51 |
| YGL136C | 1.1 | 1.0 | 1.2 | 1.1 | 1.0 | 3.1 | 0.7 | 1.1 | 1.3 | 1.0 | 1.4 | 1.1 | 1.1 | 2.3 | 0.9 | 1.4 | 1.3 | 1.1 | 0.64 |
| YGR146C | 1.6 | 2.0 | 2.0 | 2.0 | 0.9 | 4.1 | 2.7 | 1.4 | 0.8 | 3.8 | 0.9 | 1.4 | 1.7 | 1.3 | 1.4 | 1.8 | 1.3 | 0.9 | 0.75 |
| YJL020C | 0.7 | 1.2 | 0.8 | 0.8 | 1.0 | 2.1 | 1.3 | 0.6 | 1.3 | 1.0 | 0.6 | 0.7 | 1.6 | 1.2 | 1.4 | 0.8 | 2.0 | 1.3 | 0.95 |
| YLR031W | 1.3 | 1.0 | 0.7 | 1.3 | 1.3 | 3.0 | 1.5 | 0.9 | 0.9 | 1.4 | 0.7 | 1.0 | 1.6 | 1.5 | 0.7 | 1.6 | 1.2 | 1.2 | 0.30 |
| YLR205C | 1.2 | 5.1 | 0.7 | 1.3 | 1.3 | 4.4 | 9.0 | 1.7 | 1.5 | 5.7 | 0.4 | 1.3 | 1.4 | 2.4 | 2.5 | 2.3 | 2.0 | 1.4 | 0.29 |
| YMR140W | 1.0 | 0.9 | 1.2 | 0.7 | 1.1 | 17.1 | 4.3 | 0.8 | 1.8 | 2.3 | 0.7 | 0.9 | 1.6 | 1.2 | 1.4 | 2.8 | 1.4 | 2.0 | 0.54 |
| YMR195W | 1.7 | 3.1 | 1.2 | 2.2 | 1.1 | 2.6 | 1.1 | 1.0 | 0.2 | 0.7 | 1.1 | 0.9 | 0.7 | 1.3 | 0.6 | 2.5 | 1.6 | 2.2 | 1.28 |
| YOR215C | 1.5 | 1.3 | 0.7 | 1.8 | 1.8 | 2.6 | 2.6 | 1.7 | 2.1 | 1.3 | 1.6 | 1.1 | 1.3 | 1.7 | 0.8 | 3.3 | 1.3 | 2.9 | 2.08 |
| YOR382W | 0.7 | 7.6 | 3.3 | 0.5 | 1.2 | 13.1 | 28.4 | 2.0 | 1.1 | 12.8 | 2.5 | 2.8 | 1.7 | 0.4 | 2.1 | 4.3 | 1.9 | 1.9 | 0.79 |
| YPL054W | 4.2 | 5.3 | 4.9 | 3.1 | 1.1 | 2.3 | 1.8 | 1.3 | 2.5 | 9.2 | 1.9 | 1.3 | 1.5 | 1.4 | 1.0 | 1.3 | 1.5 | 1.4 | 0.25 |
| YPR127W | 1.1 | 1.4 | 1.0 | 1.6 | 1.1 | 3.2 | 1.7 | 1.2 | 1.9 | 2.4 | 1.6 | 0.9 | 1.3 | 1.4 | 0.4 | 2.4 | 1.3 | 1.2 | 0.65 |
| YAR027W | 1.8 | 1.0 | 1.3 | 1.4 | 1.2 | 2.3 | 2.2 | 1.3 | 2.3 | 1.7 | 1.7 | 1.2 | 2.6 | 1.3 | 1.2 | 2.8 | 2.0 | 4.7 | 2.15 |
| YBL057C | 1.1 | 0.9 | 0.6 | 1.0 | 1.3 | 2.2 | 1.1 | 1.4 | 1.6 | 1.2 | 0.9 | 1.0 | 1.0 | 1.6 | 1.0 | 1.1 | 1.2 | 1.2 | 1.88 |
| YBR116C | 0.9 | 4.8 | 2.4 | 2.3 | 1.1 | 2.8 | 1.5 | 1.1 | 9.0 | 8.7 | 3.1 | 1.1 | 2.0 | 2.7 | 1.7 | 9.9 | 0.9 | 1.0 | 0.45 |
| YBR147W | 2.2 | 1.1 | 1.5 | 3.2 | 1.2 | 2.2 | 1.2 | 1.9 | 0.9 | 3.6 | 4.0 | 1.3 | 1.2 | 1.1 | 2.1 | 6.0 | 1.3 | 1.9 | 0.63 |
| YBR168W | 0.9 | 0.8 | 1.0 | 1.3 | 1.6 | 3.5 | 1.6 | 1.0 | 1.1 | 1.7 | 0.8 | 0.9 | 1.9 | 1.8 | 0.9 | 1.0 | 1.1 | 1.5 | 0.56 |
| YBR246W | 1.0 | 0.8 | 1.7 | 0.6 | 1.1 | 3.1 | 1.1 | 0.9 | 0.5 | 0.9 | 0.8 | 0.9 | 1.1 | 1.3 | 0.9 | 1.8 | 1.4 | 1.7 | 0.82 |
| YBR273C | 0.8 | 0.6 | 0.6 | 1.3 | 1.9 | 2.2 | 1.4 | 1.4 | 2.2 | 2.0 | 0.6 | 0.8 | 2.1 | 0.9 | 0.8 | 0.9 | 1.4 | 1.4 | 1.33 |
| YDR003W | 2.0 | 2.4 | 1.6 | 1.5 | 1.8 | 1.8 | 1.8 | 1.1 | 6.6 | 3.6 | 1.8 | 0.8 | 1.5 | 1.2 | 1.0 | 1.8 | 1.4 | 2.0 | 0.95 |
| YDR340W | 1.2 | 1.4 | 0.7 | 0.8 | 1.1 | 1.9 | 2.3 | 1.3 | 1.4 | 2.4 | 1.4 | 0.9 | 1.3 | 2.0 | 1.4 | 1.0 | 1.1 | 1.6 | 1.90 |
| YDR357C | 1.3 | 1.5 | 0.3 | 1.3 | 2.0 | 1.9 | 2.2 | 1.7 | 1.0 | 0.9 | 1.1 | 0.6 | 1.0 | 1.4 | 0.5 | 2.2 | 1.3 | 1.7 | 0.73 |
| YDR396W | 0.7 | 1.3 | 0.6 | 0.7 | 1.4 | 2.0 | 1.5 | 1.4 | 0.3 | 1.2 | 1.0 | 1.2 | 0.6 | 1.4 | 0.9 | 1.3 | 1.1 | 1.3 | 0.63 |
| YDR434W | 0.8 | 0.8 | 1.5 | 1.2 | 1.0 | 2.2 | 1.4 | 0.9 | 0.8 | 1.2 | 0.8 | 0.9 | 1.2 | 0.9 | 1.3 | 0.8 | 1.2 | 1.5 | 1.53 |
| YDR482C | 1.1 | 0.9 | 0.7 | 1.1 | 1.5 | 1.7 | 1.2 | 1.0 | 0.9 | 0.8 | 0.8 | 0.8 | 1.1 | 1.0 | 0.5 | 1.7 | 1.6 | 1.2 | 0.85 |
| YDR520C | 0.9 | 2.1 | 0.9 | 0.6 | 1.3 | 2.3 | 1.2 | 1.0 | 0.8 | 0.7 | 0.6 | 1.2 | 1.4 | 1.0 | 1.3 | 0.9 | 1.1 | 1.0 | 0.46 |
| YDR534C | 0.8 | 4.8 | 1.8 | 0.9 | 1.0 | 2.1 | 9.7 | 1.0 | 0.6 | 7.5 | 1.7 | 1.5 | 1.8 | 0.7 | 4.2 | 1.0 | 0.6 | 1.0 | 0.34 |
| YDR539W | 0.7 | 2.0 | 1.1 | 1.0 | 1.8 | 2.1 | 2.3 | 1.3 | 0.7 | 1.2 | 1.3 | 1.2 | 1.3 | 1.1 | 1.5 | 1.6 | 1.2 | 1.4 | 0.61 |
| YER044C | 1.2 | 1.0 | 2.1 | 1.3 | 1.8 | 3.0 | 1.8 | 0.9 | 0.3 | 0.7 | 1.7 | 0.7 | 0.9 | 1.4 | 0.7 | 3.0 | 1.3 | 2.1 | 1.62 |
| YER067W | 4.0 | 1.9 | 5.2 | 6.0 | 1.1 | 2.1 | 4.9 | 2.1 | 1.8 | 3.8 | 2.2 | 1.1 | 1.2 | 1.7 | 0.5 | 3.5 | 1.5 | 3.0 | 1.57 |
| YER080W | 0.8 | 0.8 | 1.5 | 0.5 | 1.1 | 5.3 | 3.0 | 1.4 | 4.0 | 2.7 | 1.2 | 0.9 | 1.8 | 1.1 | 0.8 | 1.9 | 1.2 | 1.3 | 0.55 |
| YGL113W | 0.7 | 0.9 | 0.9 | 0.4 | 1.5 | 2.1 | 0.9 | 0.9 | 0.7 | 0.8 | 0.7 | 0.8 | 1.2 | 0.9 | 1.0 | 1.0 | 1.5 | 0.9 | 0.35 |
| YGL242C | 0.8 | 0.9 | 0.6 | 0.8 | 1.4 | 1.9 | 2.0 | 1.5 | 0.9 | 1.3 | 1.4 | 0.9 | 1.6 | 1.6 | 1.2 | 1.1 | 1.5 | 1.4 | 1.32 |
| YGR052W | 1.6 | 0.8 | 3.9 | 4.6 | 1.4 | 2.6 | 0.8 | 1.1 | 0.6 | 1.7 | 0.6 | 0.7 | 1.5 | 1.3 | 0.6 | 7.6 | 1.8 | 4.0 | 0.48 |
| YGR106C | 1.2 | 0.8 | 1.9 | 1.3 | 0.9 | 2.0 | 1.1 | 1.0 | 0.7 | 1.0 | 1.2 | 1.1 | 1.0 | 1.2 | 0.9 | 1.1 | 1.0 | 1.7 | 4.60 |
| YGR111W | 1.1 | 2.0 | 1.0 | 1.7 | 1.0 | 2.4 | 1.8 | 1.8 | 3.3 | 2.5 | 1.6 | 0.9 | 2.2 | 1.5 | 0.9 | 2.2 | 1.1 | 2.1 | 0.79 |
| YHL023C | 0.7 | 0.6 | 1.3 | 0.7 | 1.1 | 1.8 | 1.0 | 1.0 | 1.3 | 1.1 | 1.0 | 1.1 | 1.9 | 1.0 | 1.0 | 1.1 | 0.9 | 0.9 | 0.32 |
| YHL048W | 1.3 | 1.6 | 2.8 | 1.3 | 1.1 | 3.7 | 2.3 | 1.2 | 0.8 | 1.2 | 0.9 | 1.1 | 1.7 | 1.0 | 2.3 | 2.5 | 1.9 | 3.2 | 4.10 |
| YIL007C | 1.2 | 1.0 | 1.1 | 1.0 | 1.4 | 2.0 | 1.3 | 1.2 | 1.1 | 1.4 | 1.2 | 1.0 | 1.1 | 1.7 | 1.1 | 1.9 | 1.2 | 1.5 | 0.69 |
| YIR016W | 1.1 | 1.1 | 1.4 | 1.6 | 1.2 | 2.3 | 1.7 | 0.8 | 1.4 | 1.3 | 1.4 | 1.3 | 1.6 | 1.3 | 1.0 | 2.7 | 1.9 | 2.0 | 0.67 |
| YIR043C | 1.2 | 1.6 | 2.2 | 1.8 | 1.3 | 2.4 | 2.2 | 1.0 | 0.8 | 1.3 | 1.4 | 1.3 | 2.0 | 1.4 | 2.5 | 2.6 | 1.4 | 2.5 | 3.90 |
| YJL012C | 0.7 | 0.6 | 1.6 | 0.7 | 1.5 | 1.9 | 0.5 | 1.0 | 0.6 | 1.0 | 0.7 | 1.2 | 1.3 | 1.0 | 1.1 | 0.3 | 0.9 | 0.8 | 1.77 |
| YJL083W | 0.9 | 1.3 | 0.8 | 0.9 | 1.1 | 2.8 | 0.7 | 0.7 | 0.0 | 0.4 | 0.3 | 0.9 | 0.8 | 1.1 | 0.9 | 0.6 | 1.4 | 1.0 | 0.34 |
| YJL131C | 0.9 | 0.8 | 0.5 | 1.1 | 1.7 | 2.2 | 1.9 | 1.2 | 0.5 | 1.0 | 0.3 | 0.8 | 1.8 | 1.5 | 1.0 | 2.2 | 1.3 | 1.2 | 0.54 |
| YJR061W | 1.0 | 2.1 | 1.0 | 0.7 | 1.1 | 2.0 | 0.9 | 1.9 | 1.6 | 1.4 | 0.8 | 0.9 | 0.8 | 1.5 | 1.6 | 1.1 | 1.3 | 1.1 | 0.34 |
| YJR161C | 1.2 | 2.0 | 2.8 | 1.8 | 0.8 | 2.2 | 2.6 | 1.0 | 0.5 | 1.2 | 1.7 | 1.2 | 2.1 | 1.0 | 2.9 | 2.6 | 2.1 | 3.4 | 3.62 |
| YKL175W | 0.7 | 1.1 | 2.1 | 0.9 | 1.4 | 2.2 | 1.3 | 0.9 | 1.1 | 1.9 | 0.8 | 0.9 | 1.7 | 1.0 | 1.6 | 1.1 | 0.9 | 1.0 | 1.71 |
| YKR070W | 1.3 | 0.8 | 1.4 | 1.4 | 1.1 | 2.2 | 1.4 | 1.3 | 3.4 | 2.0 | 1.6 | 0.9 | 1.7 | 1.2 | 0.8 | 2.1 | 1.0 | 1.4 | 0.99 |
| YLL023C | 0.7 | 1.3 | 5.9 | 0.9 | 1.0 | 2.6 | 1.5 | 0.5 | 1.1 | 1.5 | 1.6 | 1.2 | 1.6 | 1.1 | 2.5 | 2.8 | 1.6 | 1.4 | 1.66 |
| YLR023C | 1.3 | 1.5 | 0.9 | 1.2 | 1.1 | 2.6 | 1.2 | 0.9 | 0.7 | 1.7 | 1.1 | 0.6 | 1.0 | 1.1 | 0.9 | 0.8 | 0.7 | 0.8 | 0.92 |
| YLR225C | 0.8 | 0.7 | 0.4 | 0.7 | 1.6 | 2.4 | 1.6 | 1.2 | 1.0 | 1.2 | 0.8 | 1.2 | 1.3 | 1.4 | 1.6 | 0.8 | 1.2 | 1.2 | 1.41 |
| YLR241W | 0.9 | 1.7 | 1.6 | 2.1 | 0.8 | 1.7 | 0.9 | 1.0 | 1.8 | 1.5 | 0.7 | 0.9 | 2.0 | 0.9 | 2.4 | 1.1 | 1.4 | 1.6 | 1.03 |
| YLR252W | 1.3 | 1.3 | 4.6 | 3.0 | 1.0 | 2.8 | 1.6 | 1.1 | 3.1 | 1.4 | 2.5 | 0.7 | 1.3 | 1.3 | 0.9 | 2.7 | 2.2 | 2.9 | 0.96 |
| YLR270W | 1.2 | 1.1 | 2.3 | 1.2 | 1.1 | 3.5 | 3.9 | 1.1 | 3.3 | 2.1 | 2.8 | 1.1 | 2.1 | 1.3 | 1.4 | 2.6 | 1.4 | 2.3 | 1.00 |
| YML030W | 1.3 | 0.8 | 0.8 | 1.4 | 1.9 | 3.0 | 2.1 | 1.4 | 0.8 | 0.8 | 1.2 | 0.7 | 1.0 | 1.3 | 0.6 | 2.7 | 1.2 | 1.3 | 1.35 |
| YMR148W | 1.0 | 1.0 | 1.6 | 0.9 | 1.2 | 2.9 | 0.8 | 0.6 | 1.0 | 0.8 | 1.0 | 0.5 | 1.0 | 1.0 | 0.7 | 1.4 | 1.1 | 0.9 | 0.28 |
| YMR181C | 1.3 | 1.6 | 1.8 | 1.1 | 1.2 | 2.1 | 2.0 | 0.9 | 1.9 | 1.4 | 1.7 | 1.4 | 2.8 | 1.0 | 1.4 | 2.8 | 1.1 | 2.0 | 0.79 |
| YMR298W | 1.3 | 1.3 | 2.9 | 0.8 | 1.5 | 2.3 | 1.3 | 1.1 | 2.4 | 2.6 | 1.7 | 0.9 | 1.0 | 2.0 | 2.0 | 1.7 | 1.4 | 1.6 | 1.37 |
| YNL011C | 1.1 | 1.5 | 0.8 | 0.9 | 0.9 | 2.9 | 2.9 | 1.5 | 3.8 | 1.6 | 1.4 | 0.6 | 1.2 | 1.2 | 0.8 | 3.1 | 1.2 | 1.8 | 0.68 |
| YOL129W | 1.0 | 1.1 | 2.5 | 1.2 | 1.6 | 2.2 | 1.6 | 0.7 | 1.2 | 1.2 | 0.7 | 0.8 | 1.8 | 1.5 | 1.0 | 1.9 | 2.6 | 2.4 | 2.61 |
| YOR042W | 0.6 | 1.4 | 0.7 | 0.7 | 1.3 | 2.1 | 1.1 | 0.9 | 2.1 | 1.2 | 0.9 | 0.8 | 1.3 | 1.2 | 1.1 | 1.1 | 1.4 | 1.1 | 0.54 |
| YOR052C | 1.5 | 1.3 | 0.5 | 2.4 | 2.3 | 2.4 | 1.4 | 1.4 | 1.4 | 1.3 | 0.8 | 1.0 | 2.6 | 1.6 | 1.2 | 1.3 | 1.2 | 2.3 | 2.64 |
| YOR137C | 1.1 | 1.0 | 0.9 | 1.2 | 1.3 | 2.1 | 1.6 | 0.6 | 0.7 | 0.7 | 0.9 | 0.8 | 1.6 | 0.9 | 1.4 | 1.0 | 1.6 | 1.5 | 0.78 |
| YPL156C | 1.4 | 1.4 | 0.9 | 2.9 | 1.2 | 2.4 | 3.2 | 1.2 | 0.5 | 2.8 | 0.9 | 1.5 | 1.7 | 1.6 | 0.8 | 2.4 | 1.6 | 1.9 | 0.70 |
| YPL186C | 1.6 | 1.7 | 1.4 | 4.5 | 1.3 | 3.2 | 3.2 | 1.9 | 2.4 | 1.5 | 3.7 | 1.0 | 1.4 | 2.0 | 0.7 | 6.2 | 1.2 | 2.9 | 0.60 |
| YPL216W | 0.7 | 0.8 | 1.3 | 0.9 | 0.9 | 1.8 | 0.8 | 0.9 | 1.8 | 1.9 | 0.9 | 0.9 | 1.1 | 1.2 | 1.0 | 1.1 | 1.1 | 0.9 | 0.47 |
| YPR098C | 1.6 | 2.6 | 1.7 | 1.9 | 2.0 | 3.0 | 2.9 | 1.1 | 1.4 | 1.7 | 1.9 | 0.9 | 1.2 | 1.8 | 0.9 | 2.9 | 2.2 | 2.2 | 1.10 |
| YFL062W | 1.3 | 2.7 | 1.7 | 2.4 | 1.6 | 1.8 | 3.5 | 1.1 | 0.6 | 1.2 | 2.2 | 1.1 | 1.6 | 1.1 | 1.6 | 2.7 | 2.1 | 3.0 | 3.43 |
| YJL217W | 0.6 | 0.7 | 3.1 | 2.5 | 0.7 | 1.7 | 3.2 | 4.5 | 0.7 | 1.7 | 4.6 | 0.7 | 0.5 | 0.7 | 0.8 | 5.1 | 1.4 | 2.5 | 2.19 |
| YLR126C | 0.9 | 1.5 | 0.4 | 0.8 | 1.3 | 1.7 | 3.4 | 1.8 | 1.3 | 3.4 | 1.3 | 1.4 | 1.6 | 1.4 | 1.4 | 1.4 | 1.3 | 1.2 | 0.61 |
| YNL249C | 1.3 | 1.7 | 0.3 | 1.5 | 1.2 | 0.7 | 2.3 | 1.7 | 2.6 | 1.7 | 1.4 | 1.0 | 1.4 | 1.3 | 0.9 | 1.3 | 1.1 | 1.1 | 0.56 |
| YNL336W | 1.2 | 1.0 | 3.4 | 1.3 | 1.3 | 1.4 | 2.5 | 0.9 | 0.7 | 1.9 | 0.8 | 1.1 | 1.3 | 1.1 | 1.9 | 2.8 | 2.0 | 3.1 | 3.74 |
| YBR074W | 0.7 | 1.3 | 0.9 | 0.8 | 1.3 | 1.5 | 2.2 | 0.7 | 0.4 | 0.5 | 0.5 | 1.0 | 1.2 | 1.5 | 0.8 | 1.3 | 1.4 | 1.0 | 0.52 |
| YDL248W | 1.1 | 1.7 | 2.8 | 1.7 | 1.5 | 2.0 | 2.5 | 1.1 | 0.9 | 1.4 | 1.4 | 1.2 | 2.0 | 0.9 | 1.8 | 1.9 | 1.6 | 2.4 | 4.35 |
| YDR105C | 0.7 | 1.1 | 2.3 | 1.0 | 0.8 | 1.4 | 2.1 | 0.8 | 2.3 | 1.7 | 1.4 | 0.9 | 1.8 | 1.1 | 0.9 | 1.4 | 1.1 | 1.4 | 1.09 |
| YEL075C | 1.1 | 1.1 | 0.7 | 1.0 | 1.1 | 1.4 | 1.9 | 0.7 | 0.6 | 0.7 | 0.7 | 1.1 | 1.7 | 1.9 | 0.8 | 1.2 | 1.4 | 1.1 | 1.19 |
| YER046W | 1.3 | 1.9 | 0.5 | 1.1 | 1.6 | 1.8 | 2.6 | 1.5 | 1.5 | 1.7 | 1.1 | 1.0 | 1.0 | 1.4 | 0.7 | 2.7 | 2.0 | 1.9 | 0.53 |
| YER050C | 1.3 | 0.9 | 0.5 | 2.3 | 2.2 | 1.0 | 2.2 | 1.5 | 1.4 | 1.6 | 1.4 | 0.7 | 1.0 | 2.1 | 1.0 | 2.1 | 1.0 | 1.8 | 1.40 |
| YGL250W | 1.1 | 1.4 | 1.9 | 1.8 | 1.6 | 1.4 | 2.0 | 1.1 | 1.5 | 1.2 | 2.4 | 0.7 | 1.2 | 1.0 | 1.1 | 2.3 | 1.2 | 1.9 | 0.45 |
| YGR042W | 1.5 | 1.1 | 1.2 | 2.0 | 1.5 | 2.2 | 2.2 | 1.8 | 1.3 | 3.2 | 1.6 | 1.1 | 1.6 | 2.2 | 1.4 | 2.6 | 1.3 | 1.9 | 0.79 |
| YGR053C | 1.3 | 0.7 | 0.6 | 2.7 | 2.1 | 1.9 | 3.4 | 1.5 | 4.5 | 2.2 | 1.6 | 0.7 | 1.2 | 1.7 | 0.7 | 2.7 | 1.5 | 2.4 | 0.39 |
| YGR066C | 1.6 | 2.5 | 1.3 | 2.5 | 1.2 | 1.7 | 2.1 | 1.3 | 2.3 | 5.0 | 2.6 | 0.8 | 1.2 | 1.9 | 0.7 | 2.6 | 1.0 | 1.1 | 0.17 |
| YGR247W | 0.8 | 1.1 | 0.8 | 1.0 | 1.0 | 0.9 | 1.8 | 0.9 | 1.1 | 1.1 | 1.1 | 0.6 | 0.8 | 1.0 | 0.4 | 1.2 | 1.0 | 1.0 | 0.45 |
| YGR295C | 1.0 | 1.6 | 2.8 | 2.0 | 0.9 | 1.9 | 2.8 | 1.1 | 0.5 | 1.2 | 1.1 | 1.1 | 1.9 | 1.0 | 2.4 | 2.4 | 1.6 | 2.8 | 4.78 |
| YHL044W | 0.9 | 1.3 | 1.5 | 1.4 | 0.7 | 1.9 | 2.0 | 1.4 | 1.1 | 1.9 | 0.7 | 1.2 | 1.6 | 1.7 | 1.1 | 3.9 | 0.8 | 1.2 | 0.45 |
| YHR145C | 1.5 | 1.6 | 0.6 | 0.9 | 1.4 | 1.3 | 2.1 | 1.6 | 0.8 | 2.4 | 2.0 | 0.9 | 1.5 | 1.8 | 1.3 | 1.1 | 1.3 | 1.1 | 1.70 |
| YIL058W | 2.0 | 0.9 | 1.4 | 0.5 | 1.3 | 1.4 | 2.2 | 0.8 | 1.1 | 1.0 | 0.0 | 0.6 | 1.0 | 2.0 | 0.9 | 2.1 | 1.4 | 1.3 | 0.39 |
| YIL065C | 1.0 | 1.3 | 0.8 | 1.6 | 1.9 | 1.6 | 2.0 | 1.4 | 3.8 | 2.1 | 1.1 | 0.9 | 1.8 | 1.7 | 0.8 | 2.4 | 2.1 | 1.4 | 0.94 |
| YIL083C | 0.8 | 1.0 | 0.9 | 0.8 | 1.2 | 1.4 | 2.2 | 1.0 | 1.0 | 1.1 | 1.2 | 1.0 | 1.0 | 1.0 | 0.9 | 1.4 | 0.9 | 1.1 | 0.76 |
| YJL185C | 0.9 | 1.0 | 1.9 | 1.6 | 1.0 | 1.4 | 2.3 | 1.0 | 0.9 | 1.2 | 1.2 | 0.8 | 1.0 | 1.4 | 1.6 | 2.3 | 1.0 | 1.1 | 0.30 |
| YJL213W | 0.8 | 1.1 | 0.2 | 1.2 | 1.5 | 2.6 | 2.1 | 1.2 | 1.3 | 1.5 | 0.7 | 0.7 | 1.0 | 0.8 | 2.8 | 3.6 | 2.1 | 2.8 | 0.33 |
| YKR020W | 1.1 | 1.8 | 0.9 | 1.6 | 1.0 | 4.5 | 2.2 | 1.5 | 1.9 | 2.8 | 1.0 | 0.8 | 1.7 | 1.4 | 1.2 | 2.5 | 1.3 | 2.0 | 0.44 |
| YLL025W | 0.8 | 1.3 | 1.8 | 0.8 | 1.1 | 1.5 | 1.9 | 1.0 | 1.5 | 2.0 | 1.4 | 1.0 | 1.3 | 1.0 | 1.2 | 1.7 | 1.0 | 0.9 | 0.40 |
| YLR108C | 3.2 | 1.8 | 4.7 | 1.8 | 1.9 | 1.3 | 2.6 | 2.0 | 7.4 | 10.4 | 2.6 | 0.8 | 1.9 | 3.4 | 1.2 | 2.3 | 1.0 | 1.5 | 0.39 |
| YLR290C | 1.3 | 1.2 | 0.8 | 1.9 | 2.1 | 1.7 | 2.3 | 1.1 | 1.2 | 1.1 | 1.5 | 0.8 | 1.0 | 1.3 | 0.8 | 3.2 | 1.3 | 2.4 | 1.49 |
| YML068W | 1.0 | 3.7 | 1.0 | 1.2 | 1.0 | 0.9 | 4.1 | 1.3 | 1.3 | 1.4 | 0.6 | 0.4 | 0.8 | 1.1 | 1.1 | 1.9 | 1.0 | 1.3 | 0.41 |
| YMR178W | 1.6 | 1.0 | 0.8 | 0.9 | 1.1 | 1.6 | 2.1 | 1.6 | 1.6 | 1.6 | 1.0 | 0.9 | 1.0 | 1.2 | 0.9 | 2.1 | 1.1 | 2.3 | 1.36 |
| YNL122C | 1.2 | 0.9 | 1.2 | 1.3 | 1.6 | 1.8 | 2.1 | 1.4 | 1.0 | 1.2 | 1.3 | 0.7 | 1.0 | 1.2 | 0.6 | 1.8 | 1.0 | 1.4 | 1.07 |
| YNL285W | 1.1 | 1.2 | 0.6 | 1.1 | 1.4 | 0.8 | 2.3 | 1.2 | 1.4 | 1.7 | 1.2 | 0.6 | 1.1 | 1.8 | 0.9 | 1.3 | 1.2 | 1.3 | 0.43 |
| YNL293W | 1.3 | 0.8 | 0.8 | 1.8 | 1.3 | 0.9 | 2.1 | 1.0 | 1.6 | 2.4 | 0.8 | 0.7 | 1.4 | 1.1 | 1.2 | 2.1 | 1.0 | 1.4 | 0.58 |
| YNR061C | 0.8 | 1.1 | 2.5 | 0.8 | 1.0 | 1.3 | 2.6 | 0.8 | 0.8 | 0.9 | 1.2 | 0.9 | 1.1 | 1.3 | 0.9 | 1.1 | 1.6 | 1.1 | 1.52 |
| YOR220W | 1.5 | 1.4 | 1.7 | 2.4 | 1.3 | 1.2 | 2.1 | 0.9 | 1.8 | 2.5 | 1.2 | 1.2 | 2.9 | 0.8 | 0.7 | 2.0 | 3.5 | 3.4 | 1.44 |
| YPR077C | 1.4 | 1.4 | 1.2 | 2.7 | 1.4 | 1.2 | 2.0 | 1.5 | 0.6 | 2.5 | 0.8 | 1.0 | 0.8 | 1.6 | 2.9 | 0.6 | 1.4 | 1.2 | 0.24 |
| YPR147C | 0.9 | 0.8 | 1.1 | 1.4 | 1.6 | 2.1 | 1.8 | 1.1 | 1.2 | 2.4 | 1.3 | 0.9 | 1.6 | 1.1 | 1.4 | 1.9 | 1.1 | 1.3 | 1.18 |
| YEL041W | 1.4 | 1.4 | 0.8 | 2.5 | 0.8 | 1.2 | 1.9 | 1.9 | 2.4 | 3.6 | 3.5 | 0.9 | 1.9 | 2.0 | 1.4 | 1.9 | 1.5 | 1.9 | 0.39 |
| YKL187C | 0.9 | 1.0 | 1.4 | 1.5 | 0.8 | 0.8 | 0.7 | 1.1 | 5.2 | 8.6 | 3.3 | 0.9 | 1.4 | 1.6 | 1.2 | 4.8 | 0.7 | 1.2 | 0.36 |
| YBR285W | 1.6 | 1.6 | 0.9 | 6.2 | 1.3 | 2.2 | 1.5 | 1.0 | 4.0 | 3.1 | 4.6 | 0.5 | 0.9 | 1.6 | 0.4 | 7.8 | 2.5 | 2.3 | 0.27 |
| YBR292C | 0.8 | 4.3 | 1.2 | 0.6 | 1.0 | 0.8 | 0.7 | 0.8 | 2.1 | 1.3 | 1.8 | 0.4 | 0.7 | 1.1 | 1.0 | 1.0 | 0.9 | 0.9 | 0.30 |
| YDL123W | 1.0 | 1.3 | 3.7 | 1.4 | 0.8 | 0.7 | 1.0 | 0.8 | 2.0 | 2.0 | 4.6 | 1.1 | 1.5 | 0.9 | 3.3 | 1.5 | 1.1 | 1.0 | 0.52 |
| YDR056C | 1.4 | 1.3 | 2.0 | 2.0 | 0.9 | 1.0 | 1.4 | 1.4 | 1.3 | 2.3 | 2.3 | 1.2 | 1.0 | 2.3 | 1.1 | 2.5 | 1.2 | 2.0 | 1.83 |
| YDR132C | 3.7 | 1.6 | 1.4 | 2.1 | 1.2 | 1.9 | 1.7 | 2.3 | 5.9 | 7.3 | 3.3 | 0.9 | 1.5 | 1.4 | 1.2 | 1.2 | 1.0 | 1.3 | 0.40 |
| YDR154C | 1.0 | 2.1 | 2.5 | 1.7 | 0.8 | 1.5 | 0.9 | 1.1 | 1.5 | 1.2 | 3.2 | 1.0 | 2.6 | 1.0 | 1.8 | 1.3 | 1.5 | 2.0 | 2.69 |
| YDR295C | 0.8 | 1.0 | 0.6 | 1.0 | 0.9 | 1.0 | 0.8 | 1.3 | 2.6 | 1.3 | 1.9 | 0.7 | 1.8 | 1.1 | 1.4 | 0.6 | 0.8 | 1.0 | 0.49 |
| YDR494W | 0.9 | 0.9 | 0.2 | 1.3 | 1.3 | 1.4 | 1.7 | 1.2 | 1.2 | 0.9 | 1.6 | 0.8 | 1.0 | 1.0 | 0.6 | 1.9 | 1.1 | 1.3 | 1.39 |
| YEL072W | 3.2 | 3.5 | 1.6 | 1.8 | 1.5 | 0.6 | 1.3 | 1.6 | 4.7 | 4.3 | 1.7 | 0.9 | 1.0 | 2.0 | 0.5 | 1.5 | 0.8 | 1.1 | 0.39 |
| YER045C | 1.6 | 2.1 | 1.2 | 1.0 | 0.9 | 0.8 | 1.1 | 1.4 | 2.0 | 1.5 | 2.3 | 0.8 | 1.2 | 1.3 | 1.4 | 1.6 | 1.1 | 1.1 | 0.34 |
| YER181C | 1.1 | 0.4 | 1.7 | 2.2 | 1.0 | 0.7 | 1.0 | 0.9 | | 0.8 | 0.1 | 0.6 | 1.3 | 1.0 | 0.7 | 0.7 | 0.7 | 1.1 | 0.42 |
| YGL114W | 1.5 | 0.8 | 3.6 | 1.3 | 1.3 | 0.7 | 1.0 | 1.1 | 2.7 | 5.6 | 3.2 | 0.6 | 1.2 | 1.1 | 1.2 | 1.4 | 0.9 | 0.8 | 0.60 |
| YGL193C | 1.1 | 0.9 | 0.9 | 1.4 | 0.7 | 0.7 | 1.0 | 0.7 | 0.8 | 1.0 | 2.1 | 0.7 | 1.1 | 1.0 | 1.0 | 1.7 | 1.1 | 0.9 | 0.62 |
| YGL204C | 0.9 | 2.2 | 1.2 | 1.2 | 0.7 | 0.3 | 1.3 | 0.8 | 1.0 | 0.9 | 1.9 | 0.8 | 1.4 | 1.8 | 0.8 | 1.1 | 0.9 | 0.9 | 0.38 |
| YGL259W | 1.1 | 1.6 | 1.4 | 1.5 | 1.1 | 0.8 | 1.6 | 1.3 | 2.6 | 2.2 | 2.7 | 1.1 | 1.9 | 1.1 | 1.3 | 4.1 | 0.9 | 0.9 | 0.49 |
| YIL060W | 1.0 | 2.3 | 0.8 | 1.0 | 1.2 | 0.7 | 0.9 | 1.8 | 0.9 | 3.0 | 1.9 | 0.7 | 0.6 | 0.9 | 1.3 | 1.1 | 1.2 | 0.9 | 1.22 |
| YJL036W | 1.3 | 1.0 | 0.6 | 1.6 | 0.7 | 1.1 | 1.5 | 1.5 | 6.6 | 3.5 | 1.7 | 1.2 | 2.2 | 1.5 | 1.1 | 2.1 | 1.4 | 1.6 | 0.88 |
| YJR085C | 1.0 | 1.9 | 2.8 | 4.5 | 0.6 | 1.5 | 1.4 | 0.9 | 3.7 | 2.6 | 2.9 | 1.1 | 1.9 | 1.9 | 0.5 | 2.0 | 1.4 | 1.9 | 2.19 |
| YKR071C | 3.6 | 1.6 | 1.0 | 1.1 | 1.9 | 0.6 | 0.9 | 2.1 | 4.7 | 4.9 | 2.3 | 0.8 | 1.5 | 1.6 | 0.7 | 0.8 | 0.9 | 1.0 | 1.05 |
| YLR145W | 0.9 | 1.1 | 0.6 | 1.5 | 1.1 | 0.6 | 0.9 | 1.1 | 1.9 | 1.2 | 2.4 | 0.8 | 0.7 | 1.6 | 1.0 | 2.5 | 1.0 | 0.8 | 0.62 |
| YLR156W | 1.3 | 1.0 | 0.7 | 0.8 | 0.8 | 0.6 | 1.3 | 1.0 | 1.1 | 2.2 | 1.9 | 0.8 | 1.9 | 1.0 | 1.0 | 1.7 | 1.1 | 0.9 | 0.31 |
| YLR280C | 0.6 | 1.4 | 0.4 | 0.6 | 1.3 | 0.9 | 1.5 | 1.0 | 0.5 | 1.3 | 2.1 | 0.8 | 1.6 | 0.5 | 0.6 | 1.2 | 1.2 | 0.9 | 0.19 |
| YLR311C | 1.5 | 1.9 | 2.8 | 2.9 | 0.9 | 0.4 | 1.1 | 1.0 | 0.9 | 3.0 | 7.8 | 0.9 | 0.9 | 1.6 | 0.5 | 1.9 | 1.3 | 1.1 | 0.33 |
| YMR034C | 1.5 | 1.3 | 1.0 | 6.5 | 1.3 | 1.1 | 1.9 | 1.2 | 0.7 | 2.7 | 2.1 | 0.9 | 1.8 | 1.4 | 1.7 | 1.3 | 1.8 | 1.6 | 0.43 |
| YNL240C | 0.8 | 0.8 | 0.8 | 0.6 | 0.8 | 0.7 | 0.7 | 0.9 | 5.4 | 3.1 | 3.4 | 0.7 | 1.1 | 0.8 | 1.4 | 0.8 | 0.8 | 0.7 | 0.37 |
| YNL260C | 1.7 | 1.8 | 0.5 | 1.0 | 1.6 | 0.8 | 1.1 | 1.2 | 3.8 | 2.0 | 2.5 | 0.6 | 0.7 | 1.4 | 0.7 | 1.2 | 0.9 | 1.0 | 0.75 |
| YNR074C | 1.3 | 1.7 | 3.1 | 1.4 | 1.2 | 0.7 | 1.4 | 1.5 | 3.9 | 3.0 | 2.7 | 0.6 | 1.5 | 1.7 | 0.8 | 0.9 | 0.9 | 0.8 | 0.66 |
| YOL084W | 1.0 | 1.4 | 1.6 | 3.6 | 0.6 | 1.6 | 0.8 | 1.0 | 2.6 | 3.4 | 6.1 | 1.7 | 6.4 | 0.9 | 1.6 | 6.6 | 1.3 | 1.6 | 0.28 |
| YOL159C | 1.9 | 2.4 | 1.7 | 2.0 | 0.9 | 1.0 | 2.1 | 1.2 | 1.1 | 2.4 | 6.6 | 1.2 | 1.4 | 1.8 | 1.1 | 2.3 | 2.0 | 1.6 | 0.61 |
| YOR228C | 1.3 | 1.1 | 1.8 | 1.4 | 1.2 | 1.0 | 1.2 | 0.7 | 1.4 | 1.1 | 2.8 | 0.9 | 1.1 | 1.0 | 0.6 | 3.4 | 1.0 | 1.4 | 0.44 |
| YOR255W | 2.5 | 2.7 | 1.0 | 1.5 | 1.0 | 0.5 | 0.9 | 1.2 | 0.3 | 0.8 | 2.0 | 0.7 | 1.6 | 1.3 | 4.9 | 1.2 | 1.0 | 1.0 | 0.19 |
| YBR047W | 2.7 | 2.4 | 1.1 | 1.5 | 0.9 | 1.1 | | 2.2 | 11.0 | 10.0 | 3.4 | 0.8 | 1.7 | 1.0 | 0.8 | 1.1 | 1.3 | 1.0 | 0.25 |
| YER124C | 0.5 | 17.3 | 0.7 | 1.2 | 1.4 | 2.1 | 1.0 | 1.8 | 0.4 | 1.4 | 1.5 | 0.6 | 0.7 | 0.5 | 0.5 | 1.5 | 0.9 | 1.1 | 2.22 |
| YKR007W | 0.8 | 0.9 | 0.8 | 0.9 | 1.1 | 1.6 | 1.5 | 1.9 | 1.1 | 1.8 | 1.3 | 0.8 | 0.9 | 1.2 | 1.2 | 1.1 | 0.8 | 0.9 | 0.73 |
| YOR007C | 1.1 | 3.3 | 1.4 | 0.9 | 1.1 | 1.2 | 0.4 | 2.1 | 5.6 | 2.5 | 1.8 | 0.7 | 0.7 | 0.8 | 1.3 | 1.0 | 1.2 | 0.8 | 2.26 |
| YBL065W | 1.2 | 2.9 | 1.0 | 3.5 | 1.1 | 0.7 | 1.0 | 1.9 | 20.8 | 4.2 | 1.3 | 0.7 | 1.8 | 1.9 | 3.1 | 0.9 | 1.1 | 0.9 | 0.15 |
| YDL113C | 1.0 | 1.4 | 1.2 | 1.2 | 1.0 | 1.2 | 2.2 | 1.7 | 3.8 | 3.0 | 1.1 | 1.3 | 1.5 | 1.2 | 1.2 | 1.7 | 1.2 | 1.5 | 0.58 |
| YDR018C | 1.5 | 1.3 | 3.3 | 2.6 | 1.3 | 1.5 | 1.4 | 1.4 | 2.3 | 4.4 | 1.4 | 1.1 | 1.8 | 1.7 | 1.0 | 2.6 | 0.9 | 1.2 | 0.22 |
| YDR202C | 1.1 | 1.2 | 1.1 | 1.3 | 1.3 | 1.7 | 1.6 | 1.4 | 4.8 | 2.4 | 1.3 | 0.8 | 1.2 | 2.0 | 0.5 | 2.8 | 1.0 | 1.6 | 0.79 |
| YDR223W | 1.3 | 4.6 | 2.0 | 1.9 | 1.1 | 1.4 | 1.0 | 1.0 | 2.7 | 4.6 | 1.4 | 1.0 | 1.5 | 1.2 | 1.2 | 3.5 | 0.9 | 1.0 | 0.29 |
| YDR350C | 0.9 | 1.5 | 0.4 | 1.3 | 1.3 | 1.3 | 1.5 | 0.9 | 2.8 | 2.5 | 0.9 | 0.9 | 1.0 | 1.1 | 1.6 | 1.3 | 1.4 | 1.2 | 0.53 |
| YDR374C | 1.9 | 2.9 | 1.5 | 0.8 | 1.3 | 1.1 | 0.9 | 1.8 | 10.4 | 3.9 | 1.4 | 1.0 | 2.5 | 4.0 | 2.0 | 1.3 | 1.0 | 1.0 | 0.36 |
| YDR512C | 1.8 | 4.3 | 2.5 | 2.3 | 0.7 | 2.1 | 2.0 | 1.6 | 3.6 | 4.0 | 1.8 | 1.1 | 1.9 | 2.7 | 1.1 | 3.0 | 1.3 | 2.0 | 0.82 |
| YFR017C | 1.1 | 1.4 | 4.4 | 4.4 | 1.2 | 1.2 | 1.3 | 0.8 | 1.0 | 7.1 | 3.1 | 1.8 | 1.7 | 1.3 | 1.4 | 3.7 | 0.9 | 1.1 | 0.49 |
| YGL046W | 1.6 | 1.2 | 1.5 | 1.4 | 0.9 | 1.2 | 1.0 | 1.2 | 0.8 | 4.5 | 1.5 | 1.1 | 1.6 | 0.9 | 2.0 | 1.2 | 1.5 | 1.3 | 0.42 |
| YGL067W | 1.6 | 1.3 | 1.7 | 1.1 | 0.6 | 1.0 | 1.1 | 1.2 | 1.0 | 2.3 | 1.7 | 0.9 | 3.7 | 1.6 | 0.7 | 1.0 | 0.9 | 1.1 | 0.63 |
| YGL098W | 1.2 | 0.8 | 0.5 | 1.7 | 0.8 | 0.7 | 1.2 | 1.3 | 1.2 | 2.4 | 1.3 | 0.8 | 0.8 | 1.1 | 0.7 | 1.3 | 1.1 | 1.5 | 0.77 |
| YGL117W | 2.3 | 0.6 | 0.5 | 2.0 | 0.9 | 0.9 | 1.3 | 1.9 | 0.5 | 5.8 | 1.4 | 1.5 | 1.4 | 2.0 | 1.4 | 0.6 | 2.1 | 1.1 | 1.05 |
| YGL146C | 0.9 | 1.0 | 1.1 | 3.3 | 1.3 | 1.2 | 1.3 | 1.2 | 0.9 | 3.2 | 1.3 | 0.9 | 1.1 | 1.2 | 1.1 | 2.2 | 1.0 | 1.0 | 0.43 |
| YGR011W | 1.1 | 1.5 | 1.0 | 1.3 | 0.9 | 1.1 | 1.8 | 1.4 | 7.4 | 9.4 | 2.3 | 0.8 | 1.6 | 3.9 | 1.8 | 1.6 | 0.9 | 1.0 | 0.40 |
| YGR153W | 1.5 | 0.9 | 1.0 | 1.1 | 1.6 | 0.9 | 1.4 | 1.8 | 2.7 | 2.5 | 1.0 | 0.7 | 1.1 | 1.9 | 1.3 | 1.1 | 1.3 | 1.7 | 0.39 |
| YGR223C | 1.8 | 1.2 | 1.5 | 1.3 | 1.3 | 1.2 | 1.2 | 1.4 | 6.2 | 3.8 | 1.9 | 0.8 | 1.8 | 1.6 | 1.2 | 1.8 | 1.1 | 1.8 | 0.66 |
| YHR116W | 0.9 | 1.2 | 0.9 | 2.1 | 1.6 | 1.5 | 1.8 | 1.1 | 1.5 | 2.7 | 0.9 | 0.8 | 1.9 | 1.4 | 1.2 | 1.9 | 0.9 | 1.5 | 0.60 |
| YIL097W | 1.2 | 1.0 | 1.3 | 1.2 | 1.2 | 2.0 | 1.6 | 1.7 | 4.9 | 3.4 | 1.4 | 1.1 | 1.4 | 1.4 | 1.2 | 2.1 | 1.1 | 1.7 | 0.52 |
| YKL133C | 1.3 | 12.7 | 2.4 | 2.0 | 1.0 | 1.7 | 3.2 | 1.5 | 4.1 | 5.9 | 1.4 | 1.3 | 1.4 | 1.6 | 1.7 | 3.8 | 1.2 | 2.4 | 0.35 |
| YKL162C | 1.3 | 1.0 | 1.9 | 0.9 | 1.1 | 1.7 | 1.4 | 1.5 | 6.7 | 4.7 | 1.6 | 1.1 | 1.1 | 1.9 | 1.6 | 2.0 | 0.8 | 1.1 | 0.25 |
| YLL062C | 6.5 | 6.7 | 4.6 | 1.1 | 0.8 | 0.9 | 0.7 | 1.8 | 14.9 | 5.4 | 1.7 | 0.6 | 1.4 | 1.7 | 1.6 | 1.0 | 0.9 | 0.9 | 0.29 |
| YLR247C | 0.8 | 1.5 | 1.0 | 1.0 | 1.2 | 1.2 | 1.2 | 1.3 | 2.5 | 2.9 | 1.6 | 1.0 | 1.8 | 1.0 | 1.4 | 1.4 | 1.2 | 1.3 | 0.48 |
| YLR267W | 0.9 | 1.7 | 1.3 | 1.4 | 1.2 | 1.3 | 1.4 | 1.4 | 1.1 | 4.6 | 1.7 | 0.8 | 1.5 | 2.3 | 0.7 | 3.6 | 1.2 | 2.8 | 0.24 |
| YMR041C | 1.2 | 2.8 | 1.4 | 1.2 | 1.1 | 0.5 | 0.5 | 1.4 | 6.5 | 6.0 | 1.6 | 1.0 | 1.1 | 0.6 | 0.5 | 0.3 | 0.8 | 0.4 | 0.84 |
| YMR253C | 0.9 | 1.7 | 2.0 | 1.3 | 0.8 | 1.7 | 2.3 | 1.0 | 1.5 | 2.8 | 1.0 | 0.5 | 1.2 | 1.3 | 0.9 | 1.3 | 1.2 | 1.4 | 0.42 |
| YOR225W | 1.1 | 2.8 | 1.4 | 1.0 | 1.3 | 0.7 | 0.8 | 0.8 | 0.8 | 3.3 | 1.2 | 0.5 | 0.6 | 2.2 | 0.7 | 0.6 | 0.9 | 0.9 | 0.26 |
| YPL166W | 1.0 | 1.4 | 1.4 | 1.4 | 1.2 | 0.7 | 1.8 | 1.2 | 1.5 | 3.1 | 1.4 | 0.8 | 1.4 | 0.8 | 1.2 | 2.3 | 1.1 | 1.6 | 0.39 |
| YPL202C | 1.1 | 1.6 | 1.0 | 0.7 | 1.9 | 1.4 | 1.6 | 1.1 | 1.8 | 3.5 | 1.0 | 0.7 | 1.1 | 0.9 | 1.0 | 1.2 | 1.1 | 1.0 | 0.56 |
| YBR101C | 1.3 | 2.2 | 1.7 | 1.1 | 1.2 | 0.8 | 0.6 | 1.0 | 6.9 | 3.0 | 1.3 | 0.7 | 1.6 | 0.7 | 0.5 | 0.3 | 0.9 | 1.0 | 1.83 |
| YBR269C | 1.2 | 5.7 | 2.2 | 1.1 | 1.3 | 1.8 | 1.3 | 0.8 | 2.7 | 2.6 | 1.2 | 1.0 | 1.3 | 1.4 | 0.4 | 1.6 | 1.5 | 1.6 | 0.58 |
| YBR280C | 1.2 | 1.0 | 2.7 | 2.0 | 1.3 | 1.6 | 1.7 | 1.0 | 5.3 | 1.9 | 1.3 | 1.3 | 2.8 | 1.3 | 1.0 | 3.2 | 1.1 | 1.7 | 0.33 |
| YDL234C | 1.9 | 0.9 | 0.5 | 1.4 | 1.0 | 1.1 | 1.9 | 1.3 | 4.6 | 2.1 | 1.5 | 0.9 | 3.5 | 0.7 | 1.0 | 2.5 | 2.3 | 3.6 | 0.94 |
| YDL242W | 1.3 | 1.3 | 1.6 | 1.5 | 1.3 | 0.7 | 1.1 | 0.5 | 4.0 | 4.1 | 1.3 | 1.0 | 1.0 | 1.0 | 1.2 | 1.3 | 0.9 | 1.0 | 0.38 |
| YDR531W | 0.8 | 0.9 | 0.7 | 1.0 | 2.0 | 1.2 | 1.6 | 1.4 | 2.9 | 1.5 | 1.3 | 0.9 | 1.4 | 1.2 | 0.9 | 1.2 | 1.5 | 1.2 | 1.32 |
| YFR042W | 1.5 | 1.2 | 1.9 | 1.9 | 1.8 | 0.8 | 1.6 | 1.0 | 2.8 | 2.6 | 1.7 | 1.1 | 1.9 | 1.7 | 1.4 | 1.4 | 1.7 | 1.7 | 1.17 |
| YFR046C | 1.1 | 2.3 | 1.1 | 1.6 | 1.2 | 1.0 | 1.0 | 1.5 | 5.8 | 1.5 | 1.0 | 0.9 | 1.1 | 1.4 | 0.9 | 1.3 | 1.2 | 1.2 | 0.29 |
| YGL227W | 1.0 | 0.8 | 0.9 | 0.7 | 1.2 | 1.1 | 1.2 | 1.0 | 2.4 | 1.9 | 1.1 | 0.8 | 1.0 | 1.3 | 0.9 | 1.5 | 1.1 | 1.3 | 0.59 |
| YGR089W | 0.8 | 0.8 | 0.9 | 0.5 | 1.1 | 1.2 | 0.9 | 0.8 | 4.0 | 1.4 | 0.9 | 0.7 | 0.8 | 1.1 | 0.8 | 0.9 | 0.9 | 1.1 | 0.50 |
| YGR134W | 1.1 | 1.5 | 0.5 | 1.3 | 1.3 | 1.0 | 1.0 | 1.5 | 2.4 | 1.9 | 0.9 | 0.7 | 1.5 | 1.1 | 1.1 | 0.7 | 0.9 | 0.8 | 0.33 |
| YHR017W | 0.8 | 1.0 | 1.3 | 1.3 | 1.4 | 1.1 | 1.5 | 1.5 | 2.9 | 1.7 | 1.5 | 1.0 | 1.2 | 1.2 | 0.8 | 2.5 | 1.1 | 1.7 | 0.90 |
| YIL152W | 1.1 | 1.3 | 1.0 | 1.7 | 1.0 | 0.8 | 1.2 | 1.0 | 3.1 | 1.8 | 0.9 | 1.1 | 1.4 | 1.7 | 0.9 | 1.5 | 1.1 | 1.0 | 0.81 |
| YIL164C | 1.2 | 1.2 | 1.0 | 1.2 | 1.0 | 1.1 | 2.0 | 1.2 | 4.2 | 3.0 | 1.0 | 1.0 | 1.8 | 2.0 | 0.9 | 2.5 | 1.4 | 1.4 | 0.54 |
| YJR056C | 1.0 | 2.4 | 0.8 | 1.2 | 0.9 | 0.5 | 0.9 | 1.2 | 3.5 | 1.4 | 0.8 | 0.8 | 0.9 | 1.4 | 1.6 | 1.3 | 0.9 | 1.1 | 0.44 |
| YJR072C | 0.7 | 2.5 | 1.0 | 0.8 | 1.1 | 0.7 | 0.8 | 1.3 | 3.1 | 1.6 | 0.9 | 1.0 | 1.1 | 1.2 | 0.6 | 0.9 | 0.8 | 0.8 | 0.78 |
| YKL034W | 0.8 | 1.4 | 1.0 | 0.9 | 1.1 | 1.9 | 1.4 | 0.7 | 2.6 | 3.7 | 1.2 | 1.0 | 1.9 | 1.0 | 1.6 | 1.5 | 1.3 | 1.2 | 0.41 |
| YKR012C | 0.7 | 1.4 | 1.0 | 1.0 | 1.2 | 1.4 | 1.0 | 1.0 | 3.9 | 1.2 | 0.8 | 0.7 | 1.5 | 1.4 | 1.2 | 0.9 | 1.0 | 0.9 | 0.61 |
| YLR064W | 1.1 | 1.1 | 2.9 | 1.3 | 0.8 | 1.2 | 0.8 | 1.0 | 3.3 | 2.1 | 1.6 | 0.7 | 1.6 | 1.4 | 1.5 | 1.0 | 0.9 | 1.0 | 1.49 |
| YLR364W | 3.3 | 8.0 | 1.2 | 1.5 | 1.3 | 1.0 | 1.0 | 1.1 | 8.3 | 1.3 | 1.5 | 0.7 | 1.0 | 3.0 | 1.9 | 1.0 | 1.0 | 0.9 | 0.37 |
| YLR421C | 1.1 | 1.3 | 0.9 | 1.2 | 0.9 | 2.6 | 1.5 | 1.5 | 4.5 | 3.3 | 1.2 | 0.9 | 1.6 | 1.6 | 1.6 | 1.9 | 1.2 | 1.8 | 2.07 |
| YML118W | 1.5 | 0.5 | 0.7 | 2.7 | 0.7 | 0.8 | 1.5 | 0.9 | 6.6 | 1.6 | 1.3 | 0.9 | 1.7 | 1.0 | 2.2 | 1.6 | 1.5 | 1.5 | 0.26 |
| YMR114C | 1.2 | 1.4 | 0.5 | 2.4 | 1.2 | 1.2 | 1.7 | 1.1 | 2.5 | 1.7 | 1.0 | 0.9 | 1.1 | 1.5 | 1.2 | 3.0 | 1.0 | 1.3 | 0.62 |
| YMR115W | 1.0 | 0.6 | 1.3 | 1.1 | 0.9 | 1.5 | 1.0 | 1.2 | 5.8 | 2.1 | 1.1 | 1.0 | 1.3 | 1.3 | 1.2 | 1.1 | 1.0 | 1.2 | 0.61 |
| YMR258C | 0.9 | 1.4 | 1.2 | 1.1 | 0.7 | 1.2 | 1.5 | 0.9 | 2.3 | 1.8 | 1.4 | 0.9 | 1.6 | 1.0 | 0.6 | 1.2 | 1.1 | 1.3 | 0.60 |
| YNL181W | 1.2 | 1.2 | 0.6 | 1.2 | 1.4 | 1.6 | 0.9 | 1.9 | 5.3 | 2.8 | 1.2 | 0.8 | 1.8 | 1.9 | 1.0 | 1.0 | 0.8 | 1.3 | 0.86 |
| YNL191W | 1.5 | 5.1 | 4.7 | 0.7 | 0.9 | 0.6 | 3.0 | 1.2 | 3.9 | 3.2 | 1.0 | 0.5 | 0.6 | 1.0 | 1.0 | 0.6 | 0.5 | 0.6 | 0.45 |
| YNL212W | 1.1 | 1.1 | 1.1 | 0.5 | 0.9 | 0.8 | 1.3 | 1.3 | 4.6 | 2.3 | 1.3 | 0.8 | 1.3 | 1.0 | 0.9 | 1.3 | 0.9 | 1.2 | 0.67 |
| YNL265C | 1.0 | 1.3 | 0.5 | 1.3 | 0.9 | 1.9 | 1.5 | 1.4 | 5.0 | 2.6 | 0.9 | 0.8 | 2.0 | 1.9 | 0.6 | 1.7 | 0.9 | 1.8 | 0.85 |
| YOR088W | 0.7 | 0.9 | 1.7 | 0.9 | 1.2 | 0.7 | 0.3 | 0.7 | 2.5 | 1.3 | 0.8 | 0.6 | 0.4 | 0.4 | 0.8 | 0.4 | 0.7 | 0.7 | 3.56 |
| YOR155C | 0.8 | 1.2 | 1.7 | 1.0 | 1.4 | | | 0.8 | 3.6 | 1.7 | 1.6 | 0.5 | | 1.5 | 0.9 | | 0.8 | 0.9 | 0.46 |
| YPL151C | 1.0 | 0.8 | 1.2 | 1.2 | 1.0 | 0.7 | 0.7 | 1.0 | 4.2 | 2.0 | 0.9 | 0.8 | 1.4 | 0.9 | 1.8 | 0.9 | 0.8 | 1.0 | 0.51 |
| YPL249C | 0.8 | 0.9 | 1.1 | 0.6 | 0.8 | 2.5 | 1.0 | 1.0 | 3.0 | 1.2 | 0.9 | 0.9 | 1.8 | 1.0 | 1.0 | 1.0 | 0.8 | 1.2 | 0.36 |
| YPL260W | 0.9 | 3.9 | 1.4 | 0.8 | 0.8 | 1.4 | 1.2 | 1.1 | 2.6 | 2.1 | 1.0 | 0.8 | 1.4 | 1.1 | 1.3 | 1.2 | 0.8 | 1.3 | 0.82 |
| YPR061C | 1.3 | 3.2 | 1.2 | 4.9 | 1.4 | 0.5 | 1.8 | 1.2 | 3.2 | 1.9 | 1.6 | 0.8 | 1.1 | 1.7 | 0.4 | 2.7 | 1.1 | 0.9 | 0.40 |
| YPR093C | 1.1 | 1.1 | 0.7 | 1.3 | 1.0 | 0.7 | 0.8 | 1.1 | 6.6 | 2.3 | 1.2 | 0.9 | 1.4 | 1.1 | 0.8 | 1.4 | 1.4 | 1.1 | 0.39 |
| YPR158W | 1.5 | 1.5 | 3.7 | 0.9 | 1.6 | 0.7 | 1.2 | 1.2 | 5.0 | 3.5 | 1.5 | 0.6 | 1.6 | 1.1 | 0.9 | 0.6 | 0.9 | 0.7 | 0.96 |
| YPR169W | 0.9 | 0.9 | 0.5 | 0.6 | 1.0 | 1.5 | 0.9 | 1.0 | 3.5 | 1.4 | 1.0 | 0.5 | 1.0 | 1.0 | 0.9 | 1.4 | 1.1 | 1.1 | 0.86 |
| YPR174C | 0.8 | 0.9 | 0.8 | 0.6 | 1.3 | 1.1 | 0.8 | 1.2 | 3.2 | 1.0 | 0.7 | 1.1 | 1.4 | 0.9 | 1.2 | 0.4 | 1.4 | 0.9 | 0.55 |
| YAL014C | 1.0 | 1.6 | 1.4 | 1.2 | 0.8 | 0.6 | 1.2 | 1.2 | 3.1 | 2.0 | 1.7 | 1.2 | 1.5 | 1.8 | 2.3 | 0.9 | 1.1 | 1.1 | 0.56 |
| YAL017W | 0.6 | 1.4 | 2.3 | 1.2 | 1.0 | 1.1 | 1.3 | 0.6 | 1.9 | 1.6 | 0.7 | 1.0 | 2.2 | 1.1 | 1.0 | 1.3 | 1.0 | 1.2 | 0.79 |
| YAL049C | 1.0 | 2.0 | 1.7 | 1.0 | 0.7 | 1.5 | 3.4 | 1.4 | 3.0 | 1.4 | 1.0 | 1.1 | 1.6 | 2.0 | 1.4 | 2.9 | 1.6 | 1.8 | 1.13 |
| YBR013C | 1.1 | 2.7 | 1.8 | 1.4 | 0.9 | 1.8 | 1.0 | 1.4 | 2.2 | 2.2 | 1.2 | 1.2 | 2.0 | 2.5 | 1.1 | 1.5 | 1.4 | 1.1 | 0.68 |
| YBR051W | 1.2 | 0.8 | 1.5 | 0.3 | 1.1 | | 1.1 | 1.1 | 1.5 | 1.5 | 0.1 | 0.7 | 1.2 | 1.0 | 0.8 | 1.2 | 1.1 | 0.8 | 0.40 |
| YBR063C | 0.8 | 0.8 | 0.4 | 0.6 | 1.2 | 1.6 | 1.6 | 1.3 | 2.2 | 1.6 | 0.6 | 1.1 | 1.7 | 1.7 | 1.6 | 1.0 | 1.2 | 1.2 | 0.38 |
| YBR129C | 1.1 | 0.8 | 0.6 | 1.0 | 1.2 | 1.9 | 1.0 | 1.4 | 2.3 | 1.4 | 1.4 | 1.0 | 1.3 | 1.6 | 0.9 | 1.7 | 1.0 | 1.5 | 1.25 |
| YBR255W | 1.1 | 1.6 | 0.4 | 0.7 | 1.2 | 1.0 | 1.1 | 1.8 | 1.8 | 1.5 | 1.5 | 0.7 | 1.4 | 1.1 | 1.0 | 1.5 | 1.0 | 1.0 | 0.27 |
| YBR281C | 0.8 | 1.1 | 3.0 | 0.5 | 0.8 | | 0.6 | 1.2 | 1.8 | 1.4 | 1.4 | 0.5 | 1.2 | 0.9 | 1.0 | 0.8 | 0.7 | 0.8 | 0.44 |
| YCL044C | 0.8 | 1.4 | 2.4 | 0.6 | 1.1 | 1.0 | 1.0 | 1.0 | 3.3 | 4.1 | 1.1 | 0.9 | 1.7 | 1.0 | 4.4 | 0.8 | 0.9 | 0.6 | 0.21 |
| YDL089W | 1.1 | 1.3 | 1.4 | 0.9 | 1.2 | 2.0 | 2.0 | 1.1 | 2.3 | 2.5 | 2.0 | 0.9 | 1.9 | 1.2 | 1.6 | 1.3 | 1.0 | 1.1 | 0.45 |
| YDL173W | 1.0 | 1.2 | 1.0 | 0.8 | 1.3 | 1.1 | 1.6 | 1.5 | 1.8 | 1.6 | 1.2 | 1.1 | 1.7 | 2.1 | 1.1 | 1.3 | 1.3 | 1.8 | 1.19 |
| YDL193W | 0.9 | 1.5 | 1.4 | 0.8 | 1.1 | 2.0 | 1.5 | 1.6 | 2.5 | 1.4 | 1.1 | 1.1 | 1.4 | 1.1 | 1.2 | 1.8 | 0.9 | 1.4 | 0.93 |
| YDL233W | 0.8 | 2.0 | 1.0 | 3.8 | 0.9 | 1.0 | 0.8 | 1.0 | 2.1 | 1.1 | 0.7 | 0.9 | 1.8 | 1.1 | 1.3 | 0.9 | 1.1 | 1.4 | 0.33 |
| YDR071C | 1.4 | 1.1 | 0.7 | 1.3 | 1.1 | 1.5 | 1.3 | 1.6 | 2.6 | 1.6 | 1.4 | 0.9 | 1.3 | 2.0 | 0.9 | 1.2 | 0.9 | 1.6 | 3.08 |
| YDR078C | 1.1 | 1.6 | 3.5 | 0.8 | 0.9 | 0.8 | 2.8 | 1.4 | 2.1 | 1.4 | 1.1 | 0.7 | 1.1 | 1.8 | 0.5 | 1.3 | 0.9 | 1.2 | 0.53 |
| YDR109C | 0.8 | 1.0 | 1.0 | 1.3 | 1.0 | 0.9 | 1.2 | 1.5 | 1.9 | 1.3 | 0.9 | 1.0 | 1.4 | 1.0 | 1.7 | 1.5 | 1.2 | 0.9 | 0.38 |
| YDR140W | 1.7 | 1.4 | 0.9 | 1.8 | 0.7 | 0.9 | 1.2 | 1.5 | 2.2 | 2.5 | 1.7 | 1.1 | 2.0 | 3.2 | 0.9 | 1.9 | 1.3 | 1.3 | 0.78 |
| YDR221W | 0.8 | 1.3 | 0.3 | 0.8 | 1.1 | 1.5 | 0.9 | 1.0 | 2.3 | 1.4 | 0.7 | 0.6 | 1.0 | 1.0 | 1.0 | 1.0 | 1.1 | 1.0 | 0.34 |
| YDR271C | 1.0 | 0.8 | 0.7 | 1.7 | 0.8 | 1.4 | 1.3 | 0.8 | 5.4 | 0.9 | 0.6 | 0.7 | 1.5 | 1.4 | 1.3 | 0.6 | 1.2 | 0.9 | 0.32 |
| YDR316W | 1.0 | 0.8 | 0.6 | 0.9 | 1.5 | 0.7 | 0.3 | 1.4 | 2.5 | 1.0 | 1.3 | 0.6 | 0.6 | 0.7 | 1.3 | 0.3 | 0.6 | 0.5 | 0.69 |
| YDR338C | 1.1 | 1.3 | 1.4 | 1.0 | 0.7 | 0.9 | 1.1 | 0.9 | 2.7 | 1.7 | 1.9 | 0.8 | 1.3 | 1.0 | 0.8 | 0.8 | 0.9 | 1.0 | 0.40 |
| YDR421W | 1.1 | 1.0 | 1.0 | 1.1 | 0.9 | 0.5 | 0.2 | 0.9 | 3.8 | 1.2 | 1.0 | 0.7 | 0.6 | 1.2 | 1.9 | 1.0 | 0.7 | 0.8 | 0.42 |
| YDR425W | 1.3 | 1.7 | 1.0 | 2.2 | 1.4 | 1.7 | 1.8 | 1.2 | 2.6 | 2.2 | 0.9 | 1.1 | 1.4 | 1.3 | 1.3 | 1.1 | 1.2 | 1.2 | 0.31 |
| YDR485C | 0.8 | 0.9 | 0.7 | 0.9 | 1.6 | 1.2 | 1.2 | 1.1 | 2.7 | 2.0 | 0.9 | 0.9 | 1.2 | 1.1 | 1.3 | 1.5 | 0.8 | 1.0 | 0.59 |
| YDR504C | 1.0 | 1.0 | 0.9 | 0.8 | 1.1 | 1.2 | 1.1 | 0.8 | 2.3 | 1.1 | 1.2 | 0.7 | 2.2 | 1.3 | 1.2 | 1.2 | 1.2 | 1.2 | 0.70 |
| YEL044W | 0.8 | 1.5 | 0.7 | 0.9 | 1.2 | 0.8 | 0.8 | 0.8 | 2.1 | 1.2 | 1.6 | 0.6 | 1.3 | 0.6 | 0.6 | 0.7 | 1.4 | 1.0 | 0.93 |
| YER092W | 1.3 | 1.3 | 1.2 | 1.1 | 1.2 | 1.5 | 1.3 | 1.1 | 1.9 | 1.6 | 0.9 | 0.9 | 1.2 | 1.2 | 1.0 | 1.9 | 1.4 | 1.7 | 1.13 |
| YER182W | 1.2 | 0.8 | 1.1 | 1.9 | 1.2 | 0.6 | 0.8 | 1.4 | 2.2 | 1.2 | 1.5 | 1.0 | 0.9 | 1.9 | 0.5 | 3.6 | 0.8 | 1.2 | 0.87 |
| YFL042C | 0.8 | 0.9 | 0.6 | 1.1 | 1.4 | 1.5 | 1.1 | 0.8 | 2.0 | 2.0 | 5.4 | 0.9 | 1.7 | 1.2 | 1.1 | 1.0 | 1.1 | 1.2 | 0.43 |
| YFR056C | 0.9 | 1.5 | 0.7 | 0.5 | 1.2 | 0.3 | 0.9 | 1.0 | 2.4 | 2.7 | 1.3 | 0.5 | 0.3 | 0.6 | 0.9 | 0.5 | 1.1 | 0.8 | 0.59 |
| YGL041C | 0.9 | 0.6 | 1.3 | 1.0 | 0.6 | 1.4 | 1.0 | 0.8 | 1.3 | 0.4 | 0.6 | 0.6 | 1.0 | 1.2 | 0.7 | 0.8 | 1.0 | 1.1 | 0.51 |
| YGL045W | 1.4 | 1.1 | 1.9 | 3.4 | 1.0 | 0.8 | 1.7 | 1.2 | 2.8 | 4.2 | 1.2 | 1.1 | 1.3 | 1.1 | 1.5 | 3.4 | 1.3 | 1.2 | 0.47 |
| YGL057C | 1.3 | 1.1 | 1.4 | 1.4 | 1.3 | 1.3 | 1.1 | 1.1 | 2.1 | 1.1 | 1.3 | 1.1 | 1.3 | 1.5 | 0.7 | 1.2 | 0.9 | 1.1 | 0.56 |
| YGL183C | 1.1 | 1.4 | 0.8 | 0.5 | 1.6 | 1.2 | 1.2 | 0.8 | 3.4 | 2.5 | 0.8 | 0.9 | 1.5 | 1.7 | 1.7 | 0.6 | 1.4 | 1.0 | 0.18 |
| YGL223C | 1.1 | 1.7 | 1.0 | 0.7 | 1.4 | 1.1 | 1.2 | 1.0 | 2.6 | 2.0 | 1.3 | 0.9 | 1.7 | 0.9 | 0.7 | 1.1 | 0.8 | 1.0 | 0.60 |
| YGR156W | 0.9 | 1.3 | 1.7 | 1.2 | 1.0 | 1.2 | 1.3 | 1.3 | 4.0 | 2.4 | 0.5 | 1.2 | 1.8 | 1.6 | 1.3 | 0.5 | 0.8 | 0.8 | 0.26 |
| YGR198W | 0.7 | 0.9 | 0.7 | 1.0 | 0.8 | 0.8 | 0.8 | 1.0 | 2.9 | 1.8 | 1.0 | 0.7 | 1.3 | 0.8 | 0.9 | 1.2 | 1.1 | 1.0 | 0.87 |
| YGR210C | 0.9 | 1.4 | 1.1 | 0.9 | 0.8 | 0.8 | 0.8 | 1.0 | 3.5 | 1.8 | 1.0 | 0.6 | 1.0 | 0.8 | 1.3 | 0.7 | 1.1 | 0.7 | 0.56 |
| YGR211W | 0.7 | 1.0 | 2.0 | 1.0 | 0.9 | 0.6 | 0.5 | 1.0 | 2.9 | 2.5 | 0.8 | 0.6 | 0.8 | 0.4 | 0.5 | 0.3 | 0.5 | 0.5 | 1.99 |
| YGR237C | 0.8 | 1.3 | 2.5 | 0.8 | 1.2 | 0.7 | 1.4 | 1.0 | 1.8 | 1.4 | 1.6 | 0.8 | 1.6 | 0.6 | 1.1 | 1.2 | 0.9 | 1.0 | 0.50 |
| YGR250C | 1.3 | 1.4 | 1.4 | 1.5 | 1.4 | 1.6 | 1.2 | 1.1 | 2.4 | 2.8 | 1.6 | 0.6 | 1.7 | 1.3 | 1.2 | 1.2 | 1.3 | 2.1 | 1.21 |
| YGR266W | 0.7 | 0.5 | 1.0 | 0.9 | 0.8 | 1.2 | 0.9 | 0.9 | 2.0 | 1.1 | 1.2 | 0.6 | 1.1 | 0.8 | 1.1 | 1.3 | 0.7 | 1.0 | 0.59 |
| YGR277C | 1.0 | 1.9 | 1.1 | 0.8 | 0.8 | 0.8 | 1.9 | 1.3 | 2.8 | 1.5 | 1.2 | 0.9 | 1.6 | 1.5 | 0.9 | 1.0 | 1.3 | 1.1 | 0.81 |
| YHL021C | 1.6 | 3.9 | 5.9 | 3.2 | 0.9 | 1.4 | 2.7 | 1.0 | 2.7 | 1.1 | 1.4 | 1.1 | 2.6 | 1.1 | 1.2 | 2.4 | 1.3 | 3.6 | 1.27 |
| YHL037C | 1.2 | 1.4 | 0.9 | | 1.0 | 0.7 | 1.0 | 1.1 | 0.4 | 1.1 | 1.0 | 0.6 | 1.0 | 0.9 | 0.8 | 0.7 | 0.9 | 0.9 | 0.28 |
| YHR083W | 1.0 | 0.9 | 1.4 | 1.3 | 0.9 | 0.9 | 1.0 | 1.0 | 2.0 | 2.1 | 1.3 | 0.9 | 1.1 | 1.6 | 1.3 | 2.4 | 0.8 | 1.0 | 0.90 |
| YHR134W | 1.1 | 0.7 | 0.4 | 1.1 | 1.1 | 2.0 | 1.3 | 1.2 | 3.5 | 1.5 | 0.7 | 1.0 | 1.2 | 1.6 | 1.0 | 1.2 | 1.3 | 1.3 | 0.81 |
| YHR180W | 1.5 | 0.7 | 1.1 | 1.5 | 0.9 | 1.1 | 1.1 | 1.2 | 4.8 | 3.5 | 1.4 | 0.7 | 1.3 | 1.5 | 0.8 | 1.3 | 0.8 | 1.0 | 0.36 |
| YIL108W | 1.0 | 1.7 | 2.2 | 0.7 | 1.3 | 0.6 | 0.9 | 0.8 | 2.4 | 2.0 | 1.2 | 0.7 | 1.3 | 1.0 | 1.8 | 0.7 | 1.2 | 0.7 | 0.51 |
| YIL165C | 1.2 | 2.3 | 0.9 | 1.6 | 1.6 | 0.8 | 1.5 | 1.1 | 3.2 | 3.4 | 1.1 | 0.9 | 1.5 | 1.3 | 0.8 | 1.9 | 1.4 | 1.3 | 0.74 |
| YJL032W | 1.1 | 1.0 | 0.8 | 0.9 | 0.9 | 0.6 | 0.9 | 1.3 | 3.3 | 2.0 | 1.8 | 0.5 | 1.4 | 1.6 | 1.0 | 1.2 | 1.1 | 1.1 | 0.33 |
| YJL049W | 1.3 | 0.8 | 0.5 | 1.8 | 1.6 | 1.5 | 1.2 | 1.5 | 2.0 | 1.9 | 1.5 | 0.8 | 0.9 | 1.8 | 0.8 | 1.6 | 0.9 | 1.1 | 0.75 |
| YJR044C | 1.1 | 1.2 | 5.1 | 1.0 | 1.0 | 1.5 | 2.0 | 1.0 | 2.2 | 1.6 | 1.9 | 0.7 | 2.0 | 1.3 | 1.6 | 2.7 | 1.8 | 1.6 | 1.18 |
| YKL059C | 0.8 | 1.0 | 0.9 | 0.7 | 1.3 | 0.9 | 1.0 | 1.0 | 2.6 | 1.2 | 0.9 | 0.9 | 1.3 | 1.1 | 1.1 | 0.8 | 1.1 | 0.9 | 0.53 |
| YKL090W | 1.0 | 0.9 | 1.0 | 1.2 | 1.1 | 1.0 | 1.1 | 1.2 | 3.9 | 1.6 | 2.3 | 0.6 | 0.8 | 1.4 | 0.8 | 1.6 | 0.7 | 1.0 | 0.37 |
| YKL094W | 1.2 | 1.6 | 1.2 | 1.9 | 0.9 | 1.0 | 1.1 | 1.3 | 3.1 | 1.8 | 1.3 | 0.9 | 1.7 | 1.3 | 1.0 | 2.0 | 1.2 | 1.9 | 1.60 |
| YLR097C | 1.1 | 1.5 | 1.0 | 1.6 | 1.6 | 1.7 | 1.8 | 1.4 | 2.2 | 1.1 | 1.7 | 0.8 | 1.1 | 1.7 | 0.9 | 2.5 | 1.1 | 1.7 | 0.76 |
| YLR226W | 1.0 | 1.8 | 0.3 | 1.0 | 1.7 | 1.1 | 1.9 | 1.3 | 2.1 | 1.4 | 0.8 | 0.7 | 0.7 | 1.1 | 1.3 | 0.9 | 1.1 | 1.0 | 0.56 |
| YLR392C | 1.1 | 0.9 | 0.8 | 1.9 | 1.0 | 1.2 | 1.0 | 1.3 | 2.4 | 2.1 | 1.4 | 0.6 | 1.4 | 1.1 | 0.5 | 2.1 | 0.8 | 1.6 | 0.46 |
| YLR427W | 0.6 | 1.0 | 0.2 | 0.5 | 1.6 | 1.0 | 1.2 | 1.1 | 2.1 | 1.3 | 0.8 | 0.9 | 2.5 | 0.8 | 0.8 | 1.3 | 1.0 | 1.0 | 0.49 |
| YML013W | 0.8 | 1.0 | 0.4 | 0.6 | 1.3 | 0.6 | 1.3 | 0.6 | 2.1 | 1.2 | 1.1 | 0.9 | 1.2 | 1.1 | 2.2 | 1.3 | 1.1 | 0.8 | 0.27 |
| YML029W | 0.6 | 1.5 | 1.3 | 1.0 | 1.2 | 1.6 | 1.1 | 0.9 | 2.3 | 1.8 | 1.2 | 1.1 | 2.0 | 1.6 | 0.8 | 1.1 | 1.2 | 1.0 | 0.46 |
| YML041C | 1.2 | 1.0 | 0.6 | 1.0 | 1.3 | 1.9 | 1.4 | 1.8 | 3.0 | 1.5 | 0.9 | 0.8 | 1.2 | 2.2 | 1.0 | 1.4 | 1.3 | 1.3 | 0.59 |
| YML079W | 1.0 | 1.7 | 1.1 | 1.5 | 0.8 | 1.4 | 1.2 | 1.2 | 1.9 | 1.4 | 1.2 | 1.1 | 1.6 | 1.4 | 0.3 | 2.0 | 1.4 | 1.4 | 1.18 |
| YMR068W | 0.9 | 0.7 | 0.8 | 1.5 | 1.6 | 1.1 | 1.5 | 0.8 | 2.4 | 1.4 | 1.1 | 0.7 | 1.4 | 1.1 | 2.2 | 0.8 | 1.3 | 1.1 | 0.24 |
| YMR160W | 0.8 | 1.3 | 1.2 | 1.2 | 1.4 | 2.2 | 1.0 | 0.9 | 2.2 | 1.8 | 1.0 | 1.0 | 1.6 | 1.1 | 1.5 | 1.4 | 0.9 | 1.3 | 0.37 |
| YNL026W | 0.7 | 1.0 | 1.3 | 0.8 | 1.4 | 1.1 | 1.5 | 1.0 | 2.7 | 1.8 | 1.1 | 0.9 | 1.7 | 0.8 | 0.8 | 1.5 | 1.0 | 1.4 | 0.86 |
| YNL063W | 0.9 | 1.9 | 0.9 | 1.4 | 1.1 | 1.1 | 1.4 | 1.5 | 3.0 | 1.8 | 1.1 | 1.0 | 1.5 | 1.1 | 0.7 | 1.8 | 0.9 | 1.0 | 0.54 |
| YNL176C | 1.1 | 1.3 | 2.9 | 1.0 | 0.8 | 0.7 | 0.7 | 0.9 | 2.4 | 2.1 | 1.2 | 0.6 | 0.6 | 0.9 | 0.8 | 1.3 | 0.6 | 0.8 | 0.66 |
| YNL194C | 1.6 | 0.7 | 2.0 | 15.2 | 0.7 | 0.7 | 1.5 | 0.9 | 2.2 | 6.4 | 17.1 | 0.5 | 1.5 | 3.0 | 0.5 | 4.3 | 0.8 | 2.4 | 0.34 |
| YNL253W | 1.3 | 1.3 | 0.6 | 1.0 | 1.5 | 1.3 | 1.3 | 1.1 | 2.8 | 2.6 | 0.8 | 0.8 | 1.1 | 1.4 | 0.5 | 1.1 | 1.1 | 1.2 | 0.54 |
| YNL276C | 1.3 | 13.1 | 1.9 | 0.3 | 1.1 | 1.3 | 0.9 | 0.7 | 3.1 | 1.3 | 0.7 | 0.9 | 1.5 | 1.5 | 0.5 | 0.8 | 1.1 | 0.8 | 0.22 |
| YNR051C | 0.7 | 0.5 | 1.1 | 0.6 | 1.3 | 1.8 | 1.3 | 0.7 | 2.4 | 1.4 | 1.5 | 0.9 | 2.1 | 1.0 | 1.2 | 0.6 | 0.7 | 0.8 | 1.58 |
| YOR022C | 1.3 | 1.1 | 1.1 | 1.2 | 1.4 | 1.2 | 1.6 | 1.2 | 2.0 | 1.4 | 1.7 | 0.8 | 1.7 | 1.0 | 0.8 | 1.8 | 1.1 | 1.1 | 0.36 |
| YOR087W | 0.7 | 1.0 | 1.8 | 1.3 | 0.8 | 0.3 | 0.9 | 0.8 | 2.8 | 1.3 | 1.3 | 0.8 | 1.2 | 0.9 | 0.8 | 1.2 | 0.8 | 1.1 | 0.61 |
| YOR138C | 0.8 | 1.5 | 0.7 | 0.8 | 0.8 | 1.0 | 1.1 | 0.8 | 2.1 | 3.7 | 0.7 | 0.9 | 1.8 | 0.9 | 1.0 | 1.1 | 1.1 | 1.1 | 0.47 |
| YOR267C | 0.8 | 0.7 | 1.0 | 0.9 | 1.2 | 1.6 | 0.9 | 0.7 | 2.4 | 2.2 | 0.4 | 0.9 | 1.4 | 0.9 | 1.3 | 0.8 | 1.3 | 1.0 | 0.58 |
| YPL005W | 1.0 | 1.0 | 1.0 | 1.0 | 1.4 | 0.8 | 2.0 | 1.0 | 2.6 | 2.8 | 1.1 | 0.7 | 1.2 | 1.0 | 1.2 | 1.6 | 1.2 | 1.1 | 0.32 |
| YPL150W | 0.7 | 0.9 | 1.6 | 0.9 | 1.0 | 0.6 | 1.2 | 1.1 | 3.1 | 2.0 | 1.1 | 1.0 | 2.1 | 1.0 | 1.1 | 1.2 | 1.4 | 1.1 | 0.47 |
| YPL152W | 1.4 | 1.1 | 1.4 | 1.8 | 1.4 | 1.2 | 1.3 | 1.1 | 3.6 | 2.7 | 1.4 | 0.9 | 1.4 | 1.2 | 2.3 | 1.4 | 1.2 | 1.9 | 0.42 |
| YPL168W | 1.1 | 1.5 | 0.5 | 1.4 | 1.0 | 0.8 | 1.2 | 1.2 | 2.1 | 1.6 | 0.7 | 0.7 | 1.0 | 1.0 | 0.8 | 1.3 | 1.1 | 1.2 | 0.41 |
| YPL180W | 0.9 | 2.0 | 0.7 | 0.6 | 1.2 | 1.5 | 1.0 | 1.0 | 1.9 | 1.8 | 1.0 | 0.8 | 1.5 | 1.0 | 0.7 | 1.0 | 1.0 | 0.9 | 0.30 |
| YPL188W | 1.3 | 1.2 | 2.9 | 1.0 | 0.9 | 0.6 | 1.3 | 1.6 | 2.5 | 2.6 | 1.0 | 0.6 | 0.9 | 1.4 | 0.8 | 1.7 | 0.7 | 0.9 | 0.55 |
| YPR049C | 1.0 | 2.5 | 1.5 | 1.0 | 0.7 | 1.2 | 1.1 | 1.1 | 2.4 | 2.1 | 0.9 | 0.9 | 1.6 | 1.4 | 1.5 | 1.7 | 1.0 | 1.1 | 0.34 |
| YPR148C | 1.1 | 1.2 | 0.8 | 1.3 | 0.9 | 1.0 | 1.6 | 1.5 | 2.7 | 1.7 | 0.9 | 1.2 | 1.2 | 1.1 | 1.7 | 1.7 | 0.9 | 2.0 | 1.25 |
| YPR172W | 1.1 | 1.6 | 2.2 | 1.2 | 1.0 | 1.7 | 1.2 | 1.0 | 2.1 | 1.3 | 1.9 | 0.8 | 1.3 | 1.6 | 1.5 | 1.9 | 1.1 | 1.4 | 0.45 |
| YAL018C | 2.0 | 1.9 | 1.1 | 11.3 | 0.8 | 0.7 | 1.0 | 1.6 | | 0.4 | 0.1 | 0.9 | 1.3 | 1.0 | 6.9 | 1.1 | 0.9 | 0.9 | 0.21 |
| YAR064W | 2.1 | 0.9 | 0.9 | 2.8 | 0.8 | 0.5 | 1.1 | 1.1 | 0.0 | 0.7 | 0.7 | 0.8 | 1.1 | 1.1 | 1.9 | 1.0 | 0.9 | 0.9 | 0.30 |
| YBR012C | 2.5 | 1.7 | 1.3 | 0.8 | 0.9 | 1.8 | 1.5 | 1.1 | 1.4 | 2.6 | 0.7 | 1.0 | 2.1 | 0.9 | 1.1 | 1.3 | 1.4 | 1.2 | 0.46 |
| YBR287W | 1.7 | 1.9 | 3.1 | 2.4 | 1.3 | 1.4 | 1.1 | 1.0 | 1.2 | 1.6 | 2.0 | 1.2 | 3.4 | 0.8 | 1.3 | 1.6 | 3.2 | 1.9 | 2.73 |
| YDR250C | 1.9 | 1.4 | 0.9 | 3.0 | 1.0 | 0.4 | 1.0 | 1.1 | | 0.5 | 0.6 | 0.8 | 1.2 | 0.9 | 0.7 | 1.7 | 0.9 | 0.9 | 0.32 |
| YJL037W | 1.8 | 1.4 | 1.4 | 2.4 | 1.5 | 1.8 | 1.2 | 1.2 | 1.8 | 2.4 | 1.6 | 0.8 | 1.1 | 2.0 | 1.6 | 1.0 | 1.3 | 0.9 | 0.26 |
| YNL058C | 2.0 | 1.0 | 2.1 | 1.7 | 1.0 | 0.4 | 0.7 | | 0.3 | 0.7 | 1.0 | 1.0 | 0.6 | 0.8 | 2.2 | 0.9 | 0.8 | 1.0 | 0.60 |
| YJR030C | 0.7 | 4.4 | 0.9 | 1.2 | 0.7 | 0.6 | 0.7 | 0.9 | 0.4 | 0.5 | 0.8 | 0.9 | 0.7 | 1.0 | 0.8 | 0.9 | 0.7 | 0.9 | 0.27 |
| YKR040C | 1.2 | 3.7 | 2.4 | 2.9 | 0.9 | 0.9 | 0.9 | 1.0 | 1.3 | 1.2 | 0.8 | 0.8 | 0.6 | 3.8 | 1.1 | 1.2 | 0.7 | 0.8 | 0.53 |
| YDR128W | 0.6 | 3.9 | 1.4 | 0.9 | 0.8 | 0.9 | 0.8 | 1.1 | 0.9 | 1.5 | 0.3 | 0.6 | 1.1 | 1.0 | 1.4 | 1.3 | 0.9 | 0.9 | 0.40 |
| YGR139W | 1.3 | 3.7 | 0.8 | 0.7 | 1.3 | 0.6 | 0.8 | 0.6 | | 0.6 | 0.9 | 0.5 | 0.4 | 0.9 | 2.3 | 0.8 | 1.0 | 0.8 | 0.22 |
| YOR253W | 0.9 | 2.8 | 1.0 | 1.4 | 0.8 | 0.5 | 1.3 | 1.2 | 0.4 | 1.0 | 0.9 | 0.7 | 0.5 | 1.3 | 1.1 | 0.9 | 0.9 | 0.9 | 1.01 |
| YOL026C | 1.2 | 2.7 | 0.7 | 1.4 | 1.9 | 1.0 | 1.8 | 1.1 | 1.2 | 1.1 | 1.2 | 0.9 | 1.2 | 1.3 | 0.9 | 2.4 | 2.1 | 1.4 | 0.65 |
| YDR278C | 1.0 | 3.2 | 0.9 | 1.0 | 1.1 | 1.0 | 0.7 | 0.5 | 0.6 | 0.7 | 0.4 | 0.6 | 0.7 | 0.9 | 0.7 | 0.5 | 0.7 | 0.8 | 0.78 |
| YHR095W | 1.1 | 2.6 | 1.5 | 2.1 | 1.1 | 1.0 | 0.8 | 1.0 | 1.5 | 1.2 | 1.3 | 0.6 | 0.7 | 0.9 | 0.8 | 0.9 | 0.8 | 0.7 | 0.67 |
| YCL042W | | 2.5 | 7.2 | | 1.1 | 0.6 | | 0.6 | 0.7 | 2.2 | 3.2 | | 7.5 | | | | 0.8 | | 1.94 |
| YNL200C | 1.0 | 3.0 | 3.6 | 2.0 | 1.0 | 0.4 | 1.2 | 0.7 | 1.9 | 1.2 | 3.1 | 0.9 | 1.1 | 0.6 | 0.8 | 0.9 | 1.2 | 0.9 | 1.40 |
| YPL221W | 0.9 | 3.0 | 1.0 | 1.8 | 1.4 | 1.1 | 1.3 | 0.9 | 0.3 | 1.3 | 1.0 | 0.8 | 1.0 | 0.8 | 1.0 | 1.2 | 1.5 | 1.2 | 1.51 |
| YLR415C | 1.1 | 2.4 | 6.7 | | 0.9 | 0.3 | 1.2 | 0.9 | | 0.3 | 0.8 | 0.8 | 1.0 | 0.8 | 1.0 | 1.9 | 0.8 | 1.0 | 0.19 |
| YOR325W | 1.0 | 2.6 | 1.5 | 0.3 | 0.9 | 0.3 | 1.2 | 0.9 | | | 0.6 | 0.9 | 0.6 | 1.3 | 0.7 | 1.4 | 0.8 | 1.0 | 0.19 |
| YGL088W | 0.9 | 2.1 | 0.9 | 1.1 | 0.6 | 0.8 | 1.0 | 0.5 | 0.5 | 0.8 | 0.8 | 0.6 | 0.7 | 1.4 | 0.4 | 0.3 | 0.5 | 0.7 | 1.80 |
| YDR090C | 0.9 | 3.0 | 0.8 | 1.2 | 1.8 | 1.4 | 1.5 | 0.8 | 1.0 | 1.5 | 0.7 | 0.6 | 0.8 | 0.9 | 1.1 | 0.9 | 1.1 | 1.1 | 0.79 |
| YMR071C | 1.0 | 2.0 | 1.1 | 1.9 | 1.4 | 1.2 | 1.8 | 1.1 | 1.6 | 1.8 | 1.2 | 0.7 | 1.3 | 1.2 | 0.8 | 1.4 | 1.2 | 1.9 | 2.39 |
| YGR293C | 0.5 | 1.9 | 1.6 | 0.0 | 1.1 | 0.7 | 1.3 | 1.3 | | 1.1 | 0.6 | 1.0 | 0.7 | 1.0 | 0.7 | 0.9 | 0.8 | 0.9 | 0.23 |
| YJL017W | 1.1 | 1.7 | 3.5 | 1.1 | 1.3 | 1.2 | 0.2 | 1.0 | 0.8 | 1.4 | 1.4 | 1.3 | 1.5 | 1.0 | 1.1 | 0.6 | 1.0 | 1.0 | 0.85 |
| YIL127C | 1.1 | 4.8 | 0.2 | 1.2 | 1.0 | 0.4 | 0.6 | 1.2 | 0.3 | 0.5 | 0.9 | 0.7 | 0.4 | 1.3 | 0.7 | 0.2 | 0.7 | 0.6 | 1.57 |
| YDR281C | 2.1 | 2.6 | 0.4 | 2.1 | 1.3 | 1.5 | 0.6 | 1.2 | 0.2 | 0.5 | 0.8 | 0.9 | 0.7 | 1.2 | 0.3 | 0.6 | 1.5 | 1.2 | 1.56 |
| YDR366C | 0.9 | 2.0 | 0.8 | 0.9 | 1.0 | 1.5 | 1.5 | 1.1 | 1.0 | 1.5 | 1.7 | 0.6 | 0.8 | 1.4 | 1.4 | 1.0 | 1.2 | 0.9 | 1.14 |
| YFR026C | 0.9 | 2.0 | 1.0 | 0.8 | 1.0 | 1.2 | 1.3 | 1.3 | 1.8 | 0.6 | 0.8 | 0.6 | 1.1 | 1.0 | 2.3 | 2.2 | 1.1 | 1.1 | 0.39 |
| YAR047C | 1.1 | 1.7 | 1.5 | 0.7 | 1.1 | 1.3 | 1.2 | 0.8 | 0.5 | 2.3 | 1.1 | 0.9 | 1.4 | 1.3 | 0.5 | 1.0 | 1.7 | 1.0 | 0.35 |
| YHL006C | 0.7 | 1.4 | 0.9 | 0.6 | 1.1 | 1.1 | 1.2 | 1.1 | 0.9 | 0.9 | 0.7 | 1.0 | 1.1 | 1.1 | 0.6 | 0.9 | 1.1 | 1.0 | 0.37 |
| YPL225W | 1.3 | 1.7 | 1.0 | 1.6 | 1.1 | 1.8 | 1.7 | 1.7 | 1.0 | 1.6 | 0.9 | 0.7 | 0.8 | 1.4 | 0.9 | 1.8 | 1.0 | 1.7 | 2.87 |
| YBR124W | 0.9 | 1.4 | 1.5 | 0.4 | 1.0 | 0.6 | 1.1 | 1.2 | 1.1 | 0.7 | 0.4 | 0.9 | 0.9 | 1.0 | 1.0 | 0.8 | 0.9 | 0.9 | 0.29 |
| YBL044W | 0.9 | 2.7 | 2.1 | 0.8 | 1.2 | 0.7 | 1.1 | 0.7 | 0.2 | 0.5 | 0.6 | 0.9 | 0.7 | 0.9 | 0.7 | 0.6 | 0.8 | 0.8 | 0.39 |
| YCL056C | 1.3 | 1.4 | 0.7 | 1.0 | 1.5 | 1.4 | 1.6 | 1.0 | 1.5 | 1.6 | 1.5 | 1.0 | 1.3 | 2.2 | 1.7 | 2.3 | 1.2 | 1.5 | 0.82 |
| YCR007C | 2.2 | 1.6 | 0.9 | 2.9 | 1.5 | 1.4 | 2.0 | 1.0 | 1.4 | 2.6 | 1.1 | 0.8 | 1.5 | 1.7 | 0.8 | 1.7 | 2.6 | 2.1 | 0.39 |
| YPR146C | 0.8 | 1.6 | 1.3 | 0.9 | 1.2 | 1.1 | 1.0 | 0.8 | 0.9 | 1.2 | 1.4 | 0.8 | 1.3 | 0.9 | 0.9 | 1.3 | 1.0 | 1.1 | 0.80 |
| YKL097C | 0.9 | 1.6 | 0.6 | 1.4 | 1.8 | 0.7 | 0.8 | 0.4 | 0.1 | | 0.6 | 0.6 | 0.9 | 1.0 | 0.8 | 0.5 | 1.1 | 0.5 | 0.21 |
| YBR066C | 1.9 | 1.3 | 1.6 | 1.6 | 1.3 | 1.3 | 1.3 | 1.2 | 0.3 | 0.9 | 1.2 | 0.7 | 0.6 | 1.1 | 0.4 | 3.2 | 1.2 | 1.2 | 0.83 |
| YLR338W | 1.1 | 1.6 | 1.4 | 1.1 | 0.9 | 1.1 | 0.9 | 0.8 | 7.9 | 0.5 | 0.9 | 0.6 | 1.2 | 0.8 | 1.5 | 0.9 | 0.8 | 0.6 | 0.33 |
| YBR162C | 0.6 | 0.5 | 3.1 | 0.8 | 1.3 | 0.6 | 0.3 | 0.8 | 0.1 | 0.4 | 0.8 | 0.7 | 0.7 | 0.5 | 1.9 | 0.4 | 1.2 | 1.3 | 3.96 |
| YDL046W | 1.2 | 1.4 | 5.1 | 1.3 | 0.9 | 1.8 | 1.2 | 0.7 | 1.6 | 2.3 | 2.6 | 1.0 | 1.5 | 1.2 | 2.3 | 2.6 | 1.2 | 1.6 | 1.94 |
| YDR133C | 0.9 | 1.1 | 2.9 | 2.0 | 1.2 | 0.7 | 0.2 | 0.5 | 0.1 | 0.4 | 1.3 | 0.3 | 0.1 | 0.6 | 0.6 | 1.1 | 0.6 | 0.9 | 4.02 |
| YGR038W | 0.9 | 1.2 | 3.5 | 0.8 | 1.3 | 1.3 | 1.1 | 0.7 | 1.4 | 1.0 | 1.4 | 1.0 | 1.2 | 1.4 | 1.1 | 1.3 | 1.1 | 1.1 | 1.27 |
| YGR243W | 1.4 | 2.2 | 9.4 | 5.0 | 1.0 | 1.5 | 1.9 | 1.3 | 0.7 | 3.0 | 1.7 | 1.2 | 1.1 | 1.9 | 0.8 | 2.8 | 2.1 | 4.4 | 1.11 |
| YHR105W | 0.9 | 0.8 | 2.4 | 6.3 | 1.5 | 0.2 | 0.9 | 1.0 | 1.1 | 3.4 | 0.7 | 0.7 | 1.4 | 1.2 | 0.9 | 1.8 | 1.0 | 0.9 | 0.32 |
| YHR181W | 0.9 | 0.7 | 2.4 | 1.4 | 1.0 | 0.9 | 0.8 | 0.9 | 0.5 | 1.3 | 1.3 | 0.8 | 0.7 | 1.6 | 1.4 | 1.5 | 0.9 | 0.9 | 1.40 |
| YJL097W | 1.0 | 1.1 | 3.7 | 1.6 | 0.9 | 1.6 | 1.0 | 0.8 | 0.3 | 0.7 | 1.5 | 1.0 | 0.7 | 1.0 | 1.1 | 1.2 | 1.2 | 1.1 | 1.69 |
| YKL051W | 1.0 | 1.1 | 4.7 | 0.8 | 0.8 | 1.0 | 0.6 | 0.8 | 1.3 | 1.1 | 1.8 | 0.8 | 1.2 | 0.7 | 1.5 | 0.9 | 1.5 | 1.4 | 1.07 |
| YKL100C | 0.7 | 1.1 | 5.0 | 1.6 | 0.6 | 1.8 | 1.2 | 0.7 | 1.3 | 1.3 | 1.6 | 0.9 | 1.3 | 1.0 | 1.7 | 2.0 | 0.7 | 1.5 | 1.14 |
| YLR339C | 0.7 | 0.8 | 3.1 | 0.8 | 1.3 | 0.4 | 0.4 | 0.5 | | 0.4 | 1.6 | 0.6 | 0.1 | 0.5 | 1.0 | 0.5 | 0.7 | 0.6 | 1.37 |
| YOL030W | 1.0 | 0.8 | 4.4 | 1.0 | 0.9 | 0.9 | 1.3 | 0.6 | 1.2 | 0.6 | 1.3 | 0.9 | 1.7 | 0.6 | 0.9 | 0.7 | 0.9 | 1.0 | 1.93 |
| YPR150W | 1.3 | 1.1 | 4.3 | 3.0 | 1.3 | 0.9 | 0.9 | 0.8 | 1.2 | 3.3 | 0.9 | 0.6 | 1.4 | 1.4 | 0.9 | 1.8 | 0.9 | 1.0 | 0.31 |
| YBL100C | 0.8 | 1.8 | 1.6 | 0.5 | 0.6 | 0.4 | 1.1 | 0.6 | 0.2 | 0.8 | 0.9 | 0.7 | 0.9 | 0.9 | 1.0 | 0.5 | 0.8 | 0.8 | 0.58 |
| YBR096W | 1.1 | 1.5 | 2.8 | 1.6 | 0.9 | 1.7 | 1.9 | 1.0 | 1.2 | 1.6 | 1.2 | 0.9 | 1.9 | 1.7 | 1.1 | 1.1 | 1.1 | 1.5 | 1.43 |
| YBR100W | 1.0 | 1.5 | 4.8 | 1.1 | 0.9 | 0.5 | 1.0 | 0.9 | 8.5 | 3.0 | 0.2 | 0.7 | 1.2 | 1.2 | 1.3 | 1.2 | 0.8 | 0.9 | 0.35 |
| YCL058C | 1.1 | 1.6 | 2.2 | 1.2 | 1.7 | 1.1 | 0.8 | 0.9 | 0.6 | 1.0 | 1.0 | 1.0 | 0.9 | 1.9 | 1.2 | 0.8 | 1.4 | 0.9 | 0.72 |
| YCR030C | 1.1 | 0.9 | 3.3 | 1.0 | 1.0 | 1.2 | 1.0 | 0.7 | 0.9 | 2.0 | 1.1 | 0.9 | 1.7 | 0.8 | 1.3 | 1.0 | 1.2 | 1.0 | 0.63 |
| YDL015C | 0.9 | 0.7 | 3.6 | 1.1 | 1.2 | 2.5 | 1.1 | 0.9 | 0.7 | 1.1 | 0.8 | 1.0 | 0.9 | 1.1 | 1.1 | 2.1 | 1.0 | 1.5 | 2.99 |
| YDL023C | 0.8 | 1.6 | 2.4 | 1.1 | 0.6 | 0.3 | 1.5 | 0.7 | 0.7 | 1.5 | 3.5 | 0.9 | 1.4 | 0.7 | 0.6 | 0.9 | 0.8 | 1.1 | 0.78 |
| YDL086W | 0.9 | 1.2 | 1.9 | 0.9 | 1.3 | 0.9 | 0.8 | 0.8 | 1.3 | 1.1 | 1.4 | 0.8 | 1.7 | 0.9 | 0.9 | 1.1 | 0.8 | 0.8 | 0.80 |
| YDR233C | 1.1 | 1.1 | 3.2 | 1.9 | 0.7 | 0.8 | 1.2 | 1.1 | 0.2 | 0.7 | 3.7 | 1.3 | 0.5 | 0.9 | 1.5 | 2.8 | 0.9 | 0.9 | 3.47 |
| YDR359C | 0.8 | 0.7 | 1.7 | 1.9 | 1.3 | 0.8 | 1.0 | 0.8 | 0.7 | 1.0 | 0.5 | 1.0 | 1.1 | 1.3 | 1.4 | 0.9 | 0.8 | 0.9 | 0.34 |
| YEL033W | 0.9 | 1.2 | 2.7 | 1.1 | 1.0 | 0.7 | 0.3 | 0.7 | 0.6 | 0.4 | 1.2 | 0.6 | 0.4 | 1.0 | 0.6 | 0.9 | 0.9 | 0.9 | 2.96 |
| YGR022C | 1.3 | 1.1 | 1.7 | 2.2 | 1.1 | 1.3 | 0.9 | 0.9 | 0.2 | | 0.4 | 0.7 | 1.2 | 1.1 | 0.7 | 0.6 | 0.9 | 0.8 | 0.50 |
| YGR026W | 0.8 | 1.1 | 2.9 | 0.9 | 0.9 | 1.5 | 0.9 | 0.9 | 0.8 | 0.7 | 1.3 | 0.5 | 0.6 | 1.0 | 1.2 | 1.4 | 1.0 | 1.2 | 2.19 |
| YGR107W | 1.0 | 0.8 | 1.7 | 0.9 | 1.1 | 0.8 | 1.0 | 0.9 | 0.1 | 0.0 | 0.6 | 0.6 | 0.9 | 1.0 | 1.1 | 0.8 | 1.2 | 0.8 | 0.43 |
| YHL005C | 0.9 | 2.7 | 2.7 | 0.2 | 1.2 | 1.1 | 0.8 | 0.9 | | 0.9 | | 0.7 | 0.7 | 1.0 | 0.7 | 0.7 | 1.0 | 0.8 | 0.33 |
| YHR126C | 1.1 | 0.9 | 2.8 | 0.6 | 1.0 | 1.2 | 1.1 | 0.6 | 0.4 | 0.7 | 1.9 | 1.0 | 0.8 | 0.8 | 1.4 | 1.7 | 1.1 | 1.3 | 1.31 |
| YHR143W | 0.5 | 0.9 | 4.9 | 1.0 | 1.2 | 2.1 | 0.5 | 1.5 | 0.2 | 0.7 | 1.2 | 0.5 | 0.5 | 0.8 | 0.3 | 0.9 | 0.9 | 0.7 | 3.58 |
| YIL157C | 1.1 | 1.3 | 2.1 | 1.7 | 0.8 | 1.3 | 1.2 | 1.0 | 0.9 | 0.9 | 1.3 | 0.8 | 1.0 | 1.0 | 0.5 | 1.8 | 1.1 | 1.5 | 1.46 |
| YIR041W | 1.6 | 1.0 | 2.4 | 1.4 | 1.3 | 1.0 | 1.6 | 0.9 | 1.5 | 2.5 | 1.2 | 1.3 | 1.1 | 1.1 | 1.3 | 1.3 | 1.1 | 0.9 | 0.66 |
| YJL016W | 1.3 | 1.6 | 2.7 | 1.3 | 1.4 | 1.5 | 1.4 | 0.9 | 0.6 | 0.9 | 1.3 | 0.9 | 1.3 | 1.6 | 1.1 | 2.5 | 1.2 | 1.5 | 0.88 |
| YJR018W | 0.8 | 1.2 | 1.8 | 0.7 | 1.1 | 1.0 | 0.9 | 0.7 | 0.4 | 0.7 | 0.9 | 0.8 | 1.5 | 0.9 | 1.4 | 0.8 | 1.0 | 0.7 | 0.35 |
| YKL147C | 1.0 | 1.3 | 1.9 | 0.9 | 0.8 | 0.7 | 0.8 | 0.7 | 1.0 | 0.5 | 0.3 | 0.9 | 1.0 | 1.0 | 0.7 | 1.1 | 0.9 | 0.9 | 0.28 |
| YKL169C | 1.0 | 1.0 | 1.6 | 1.8 | 0.8 | 1.2 | 1.5 | 1.2 | 0.3 | 0.5 | | 0.7 | 1.0 | 1.3 | 0.8 | 2.6 | 0.9 | 1.1 | 0.45 |
| YKR033C | 0.9 | 1.2 | 2.1 | 1.0 | 1.0 | 0.6 | 1.0 | 1.0 | 0.1 | 1.4 | 0.3 | 0.4 | 0.9 | 0.9 | 1.1 | 0.6 | 1.0 | 0.9 | 0.28 |
| YLL064C | 0.8 | 1.7 | 1.7 | 1.9 | 0.9 | 1.7 | 1.1 | 0.9 | 1.1 | 2.5 | 1.0 | 1.1 | 1.3 | 1.1 | 1.2 | 1.6 | 1.2 | 1.0 | 0.68 |
| YLR041W | 0.8 | 1.2 | 2.2 | 1.5 | 1.6 | 0.5 | 1.2 | 0.6 | | 0.4 | 1.1 | 0.4 | 0.3 | 0.5 | 1.3 | 0.5 | 0.7 | 0.9 | 0.69 |
| YLR177W | 0.8 | 1.2 | 1.8 | 2.2 | 0.8 | 1.1 | 1.3 | 0.9 | 1.0 | 2.3 | 0.9 | 1.1 | 1.5 | 1.0 | 0.5 | 1.3 | 1.1 | 1.5 | 1.25 |
| YMR007W | 1.1 | 0.9 | 2.0 | 0.7 | 1.3 | 1.7 | 3.1 | 0.7 | | 0.1 | 0.9 | 0.8 | 1.3 | 1.2 | 1.8 | 1.1 | 2.5 | 1.0 | 0.30 |
| YMR156C | 1.0 | 0.9 | 2.7 | 1.7 | 1.2 | 1.4 | 1.4 | 0.9 | 0.6 | 1.3 | 1.1 | 0.7 | 1.0 | 1.2 | 0.7 | 1.3 | 1.2 | 1.1 | 0.40 |
| YMR215W | 0.8 | 0.5 | 2.1 | 1.2 | 1.1 | 0.3 | 0.3 | 0.5 | 0.1 | 0.2 | 1.1 | 0.4 | 0.2 | 0.5 | 0.7 | 0.2 | 0.7 | 0.7 | 1.59 |
| YNL195C | 0.9 | 1.1 | 3.5 | 4.7 | 0.9 | 0.8 | 1.1 | 0.9 | 2.6 | 11.7 | 2.9 | 0.6 | 1.6 | 1.5 | 1.4 | 6.4 | 0.8 | 0.9 | 0.32 |
| YOL073C | 0.7 | 0.9 | 2.5 | 0.6 | 1.1 | 1.2 | 1.3 | 0.7 | 1.9 | 1.1 | 1.7 | 0.8 | 1.6 | 0.8 | 1.6 | 1.7 | 1.2 | 1.2 | 0.54 |
| YOR129C | 0.6 | 0.8 | 3.3 | 0.8 | 1.3 | 0.4 | 0.4 | 0.6 | 0.2 | 0.4 | 1.1 | 0.6 | 0.2 | 0.5 | 0.6 | 0.3 | 0.6 | 0.4 | 5.22 |
| YOR161C | 0.6 | 1.0 | 6.0 | 2.2 | 1.1 | 0.9 | 1.2 | 0.8 | 1.0 | 1.7 | 4.6 | 0.5 | 0.7 | 0.9 | 1.4 | 2.0 | 0.7 | 1.2 | 0.82 |
| YPL004C | 0.7 | 1.2 | 3.7 | 1.9 | 1.4 | 1.3 | 2.3 | 0.9 | 1.1 | 1.6 | 1.7 | 0.9 | 1.3 | 1.0 | 1.1 | 2.0 | 1.2 | 2.2 | 4.52 |
| YPL246C | 0.7 | 0.8 | 2.6 | 0.9 | 1.4 | 1.3 | 0.8 | 0.6 | 0.4 | 0.5 | 1.1 | 0.8 | 0.9 | 0.7 | 1.2 | 1.1 | 1.2 | 0.9 | 0.98 |
| YPL272C | 1.1 | 0.9 | 2.2 | 0.6 | 1.3 | 1.1 | 0.7 | 0.8 | 1.2 | 2.5 | 1.1 | 0.7 | 1.1 | 1.1 | 0.6 | 2.5 | 0.9 | 1.2 | 0.25 |
| YPR063C | 1.0 | 0.8 | 2.6 | 0.8 | 1.2 | 1.0 | 0.6 | 0.7 | 0.5 | 0.6 | 1.0 | 0.7 | 0.8 | 1.0 | 1.0 | 1.2 | 1.0 | 0.9 | 1.41 |
| YDL129W | 1.0 | 0.7 | 1.0 | 2.3 | 1.3 | 0.9 | 0.9 | 1.0 | 0.5 | 0.9 | 0.6 | 0.9 | 0.5 | 0.8 | 0.5 | 1.4 | 0.6 | 0.8 | 0.69 |
| YDR066C | 1.3 | 1.5 | 0.9 | 2.2 | 1.0 | 1.4 | 1.1 | 1.3 | 1.5 | 1.1 | 0.6 | 0.8 | 1.4 | 1.3 | 0.9 | 1.4 | 1.1 | 1.1 | 0.57 |
| YGL059W | 1.3 | 1.5 | 0.9 | 3.1 | 0.7 | 0.7 | 1.0 | 1.4 | 1.6 | 1.7 | 1.4 | 1.0 | 2.0 | 1.2 | 1.6 | 1.7 | 1.4 | 1.4 | 0.49 |
| YNL144C | 1.8 | 0.8 | 1.2 | 4.5 | 0.9 | 1.1 | 1.1 | 1.1 | 0.7 | 1.9 | 1.7 | 1.4 | 1.0 | 1.1 | 0.1 | 1.7 | 2.4 | 2.8 | 0.51 |
| YAL037W | 1.6 | 0.9 | 1.1 | 3.5 | 1.2 | 1.1 | 1.0 | 1.3 | 0.6 | 1.9 | 0.5 | 0.8 | 1.1 | 1.2 | 0.8 | 0.6 | 0.9 | 0.9 | 0.55 |
| YAR023C | 1.1 | 1.0 | 1.7 | 2.1 | 1.3 | 1.0 | 1.1 | 0.8 | 0.9 | 1.4 | 1.0 | 1.2 | 1.1 | 1.0 | 3.1 | 1.6 | 0.9 | 1.1 | 0.62 |
| YCR015C | 1.1 | 0.9 | 0.5 | 2.1 | 1.7 | 1.0 | 1.5 | 1.2 | 0.5 | 1.0 | 1.1 | 0.9 | 0.6 | 1.2 | 0.9 | 1.1 | 1.1 | 1.2 | 0.58 |
| YCR043C | 1.4 | 0.5 | 0.8 | 2.3 | 0.9 | 0.8 | 0.7 | 1.2 | 0.3 | 0.7 | 1.0 | 0.6 | 0.8 | 1.6 | 1.2 | 0.8 | 1.0 | 1.1 | 1.25 |
| YDL146W | 1.0 | 1.2 | 1.2 | 2.0 | 0.7 | 0.7 | 1.4 | 0.9 | 0.8 | 2.3 | 1.4 | 1.0 | 1.4 | 1.0 | 1.7 | 1.8 | 1.0 | 1.2 | 0.62 |
| YDR057W | 0.9 | 1.4 | 1.6 | 2.6 | 0.8 | 0.7 | 1.4 | 1.2 | 1.5 | 1.1 | 1.0 | 0.7 | 0.9 | 1.2 | 1.1 | 1.7 | 0.7 | 1.1 | 0.70 |
| YDR222W | 1.0 | 1.1 | 1.7 | 1.7 | 1.4 | 1.3 | 0.5 | 1.0 | 0.6 | 1.0 | 1.0 | 0.5 | 0.8 | 1.3 | 0.5 | 0.6 | 0.7 | 0.8 | 0.52 |
| YDR286C | 1.6 | 0.9 | 1.1 | 2.1 | 1.0 | 1.7 | 1.3 | 1.9 | 0.8 | 1.2 | 1.1 | 0.9 | 1.4 | 1.3 | 0.8 | 2.1 | 1.1 | 1.4 | 0.90 |
| YDR438W | 1.2 | 1.8 | 0.8 | 2.1 | 1.6 | 1.3 | 1.4 | 1.0 | 1.1 | 0.8 | 1.1 | 0.8 | 1.2 | 1.3 | 1.0 | 1.2 | 1.6 | 1.2 | 0.48 |
| YDR479C | 1.1 | 1.8 | 1.0 | 2.3 | 0.9 | 1.6 | 1.2 | 0.9 | 1.2 | 1.6 | 1.0 | 1.0 | 1.2 | 1.4 | 1.6 | 2.1 | 1.1 | 1.5 | 0.51 |
| YEL057C | 1.1 | 0.6 | 1.3 | 2.4 | 1.0 | 0.9 | 1.1 | 1.2 | 1.2 | 1.9 | 0.7 | 0.8 | 1.5 | 1.5 | 1.0 | 1.8 | 0.9 | 1.0 | 0.44 |
| YEL073C | 1.6 | 0.8 | 1.0 | 2.8 | 1.2 | 1.5 | 0.8 | 0.9 | 0.7 | 1.2 | 1.1 | 0.5 | 1.0 | 1.1 | 2.8 | 1.0 | 1.3 | 0.8 | 0.38 |
| YER084W | 0.8 | 1.1 | 1.0 | 3.9 | 0.9 | 0.6 | 0.8 | 0.9 | | 0.8 | | 0.8 | 0.7 | 0.9 | 2.6 | 1.5 | 0.8 | 0.9 | 0.30 |
| YER121W | 1.1 | 1.2 | 0.7 | 7.7 | 1.3 | 2.5 | 1.3 | 1.3 | 0.6 | 0.5 | 1.2 | 0.7 | 0.6 | 1.3 | 0.9 | 4.6 | 1.2 | 1.8 | 0.73 |
| YER189W | 0.9 | 0.8 | 0.9 | 1.9 | 1.3 | 0.5 | 0.9 | 0.7 | 0.4 | 0.5 | 0.7 | 0.8 | 0.7 | 0.9 | 0.9 | 0.8 | 0.8 | 0.8 | 0.43 |
| YFL017C | 1.3 | 1.2 | 0.9 | 2.3 | 1.5 | 1.3 | 1.5 | 1.4 | 0.8 | 0.6 | 1.1 | 1.0 | 0.7 | 1.1 | 0.6 | 1.8 | 1.5 | 1.8 | 1.21 |
| YFL046W | 1.3 | 0.9 | 0.7 | 2.1 | 1.7 | 0.7 | 1.3 | 1.3 | 0.7 | 0.7 | 1.3 | 0.8 | 0.7 | 1.3 | 1.0 | 1.1 | 1.4 | 1.6 | 0.68 |
| YFR008W | 1.1 | 1.0 | 0.8 | 2.2 | 1.1 | 1.0 | 1.1 | 0.9 | 1.5 | 1.4 | 0.7 | 0.9 | 0.8 | 1.1 | 0.8 | 1.5 | 1.0 | 1.0 | 1.26 |
| YGL214W | 0.9 | 1.0 | 1.2 | 2.6 | 1.2 | 0.9 | 0.8 | 0.9 | 0.4 | 0.7 | 0.4 | 0.8 | 0.6 | 1.1 | 0.9 | 0.8 | 1.0 | 0.8 | 0.61 |
| YGL218W | 1.4 | 0.9 | 1.4 | 3.4 | 1.2 | 1.1 | 0.8 | 1.1 | 1.3 | 0.4 | 0.1 | 0.8 | 1.3 | 0.9 | 1.1 | 0.7 | 1.0 | 1.0 | 0.53 |
| YGR021W | 0.9 | 1.5 | 1.0 | 8.6 | 1.0 | 0.9 | 1.3 | 1.4 | 0.7 | 0.6 | 0.6 | 0.9 | 0.9 | 1.2 | 0.8 | 2.1 | 1.0 | 1.0 | 0.58 |
| YGR024C | 1.2 | 0.9 | 0.7 | 2.6 | 1.0 | 0.7 | 1.3 | 1.4 | 0.4 | 1.2 | 1.0 | 0.7 | 0.5 | 1.4 | 1.0 | 1.2 | 0.8 | 1.3 | 1.61 |
| YGR064W | 1.1 | 1.2 | 1.6 | 2.3 | 0.7 | 0.6 | 1.1 | 1.0 | 0.1 | 0.8 | 1.7 | 0.8 | 0.9 | 2.1 | 1.0 | 1.3 | 0.8 | 0.9 | 0.50 |
| YGR182C | 1.6 | 1.2 | 1.2 | 2.3 | 1.1 | 0.9 | 0.7 | 1.1 | 0.5 | 0.7 | 0.9 | 0.7 | 0.7 | 2.2 | 0.4 | 2.0 | 1.0 | 1.7 | 1.95 |
| YGR236C | 1.6 | 1.6 | 0.4 | 4.1 | 1.2 | 1.2 | 1.0 | 1.2 | 1.5 | 0.7 | 1.0 | 0.9 | 1.2 | 1.2 | 0.7 | 7.9 | 1.5 | 1.9 | 0.30 |
| YHL042W | 1.2 | 0.8 | 1.3 | 51.1 | 1.0 | 0.7 | 1.2 | 1.0 | 0.8 | 1.8 | 0.7 | 0.6 | 1.3 | 1.2 | 1.1 | 1.9 | 1.1 | 0.9 | 0.37 |
| YIL012W | 1.2 | 5.8 | 1.5 | 2.8 | 1.5 | 1.5 | 0.9 | 0.9 | 1.7 | 2.8 | 0.9 | 0.6 | 0.4 | 1.1 | 1.1 | 0.9 | 1.0 | 0.9 | 0.28 |
| YIL028W | 1.0 | 1.1 | 0.8 | 5.4 | 1.4 | 0.7 | 1.1 | 0.8 | 0.8 | 1.7 | 1.1 | 0.5 | 1.0 | 1.1 | 0.9 | 1.1 | 1.3 | 1.0 | 0.27 |
| YIL057C | 1.3 | 1.8 | 1.1 | 4.6 | 1.3 | 1.2 | 1.2 | 1.4 | 0.1 | 5.0 | 0.8 | 0.6 | 0.9 | 1.5 | 0.8 | 11.1 | 1.1 | 3.6 | 0.26 |
| YIL089W | 1.3 | 1.3 | 0.7 | 3.9 | 1.6 | 1.6 | 1.6 | 1.1 | | 0.6 | 0.7 | 0.6 | 0.7 | 1.4 | 0.6 | 1.3 | 1.4 | 1.3 | 0.31 |
| YIL102C | 1.4 | 1.6 | 1.0 | 6.1 | 1.5 | 0.8 | 1.1 | 0.7 | | 0.3 | | 0.6 | 0.7 | 1.0 | 0.8 | 0.6 | 1.2 | 1.0 | 0.18 |
| YIL113W | 0.9 | 1.0 | 0.9 | 2.6 | 0.6 | 1.0 | 1.9 | 0.7 | 1.9 | 2.2 | 2.5 | 0.8 | 1.3 | 1.2 | 0.9 | 1.6 | 1.4 | 1.3 | 0.48 |
| YIL122W | 1.2 | 1.1 | 1.1 | 2.1 | 0.9 | 0.2 | 0.8 | 0.7 | 0.4 | 0.8 | 2.1 | 1.0 | 0.7 | 1.0 | 1.6 | 1.4 | 0.9 | 0.9 | 0.35 |
| YJL100W | 1.4 | 2.0 | 0.9 | 2.3 | 1.3 | 1.1 | 1.2 | 1.2 | 1.8 | 2.7 | 1.5 | 0.9 | 1.4 | 1.2 | 1.2 | 1.4 | 1.5 | 1.1 | 0.38 |
| YJL169W | 0.8 | 1.1 | 1.5 | 2.8 | 1.4 | 0.8 | 0.9 | 0.7 | 0.3 | 0.5 | 0.4 | 0.5 | 0.7 | 1.0 | 0.9 | 0.6 | 1.1 | 0.9 | 0.35 |
| YJL199C | 1.1 | 0.7 | 0.9 | 4.5 | 1.0 | 1.7 | 1.0 | 1.0 | 1.0 | 1.6 | 1.0 | 0.8 | 0.9 | 1.9 | 0.9 | 3.7 | 1.0 | 1.0 | 0.59 |
| YJR039W | 0.9 | 0.6 | 0.8 | 3.0 | 1.8 | 1.0 | 1.6 | 1.2 | 1.8 | 1.6 | 1.8 | 0.7 | 1.5 | 0.6 | 1.2 | 1.5 | 1.3 | 1.3 | 0.24 |
| YJR101W | 0.8 | 0.6 | 0.7 | 2.9 | 1.8 | 0.9 | 1.5 | 1.1 | 0.4 | 0.8 | 1.2 | 0.5 | 0.5 | 0.9 | 0.5 | 1.2 | 1.0 | 1.1 | 2.04 |
| YKL061W | 1.3 | 0.7 | 0.7 | 1.8 | 0.8 | 1.0 | 0.9 | 1.2 | 0.8 | 1.0 | 1.1 | 0.7 | 0.8 | 1.2 | 0.6 | 1.5 | 1.3 | 1.6 | 0.92 |
| YKL121W | 0.9 | 7.8 | 0.8 | 11.8 | 1.6 | 1.2 | 1.7 | 1.0 | 0.8 | 0.9 | 1.6 | 0.8 | 1.0 | 1.2 | 0.8 | 1.6 | 1.6 | 1.1 | 0.31 |
| YKL160W | 1.5 | 1.0 | 0.4 | 1.8 | 1.1 | 1.5 | 1.5 | 2.1 | 1.3 | 1.9 | 1.4 | 0.9 | 1.6 | 1.5 | 0.8 | 1.6 | 1.5 | 2.2 | 2.19 |
| YLR016C | 0.7 | 1.3 | 0.5 | 2.0 | 1.5 | 0.8 | 1.1 | 1.2 | 0.8 | 1.1 | 1.0 | 0.7 | 0.8 | 0.9 | 0.7 | 1.3 | 1.0 | 1.3 | 1.15 |
| YLR030W | 1.4 | 1.5 | 0.8 | 2.5 | 1.2 | 1.5 | 1.5 | 0.9 | 0.3 | 2.7 | 0.2 | 0.8 | 1.5 | 1.2 | 0.6 | 2.1 | 1.0 | 1.0 | 0.29 |
| YLR036C | 1.3 | 0.8 | 0.7 | 2.0 | 1.5 | 1.3 | 1.7 | 1.2 | 0.4 | 1.6 | 0.8 | 0.7 | 0.7 | 1.3 | 0.6 | 1.4 | 1.0 | 1.4 | 1.07 |
| YLR112W | 1.1 | 0.9 | 1.1 | 1.7 | 1.3 | 1.3 | 1.0 | 0.7 | | 0.7 | 0.5 | 0.5 | 0.7 | 0.8 | 0.4 | 0.4 | 1.0 | 0.8 | 0.39 |
| YLR125W | 1.4 | 2.0 | 0.5 | 3.1 | 1.8 | 0.7 | 1.3 | 1.2 | 0.5 | 1.7 | 1.1 | 0.5 | 0.8 | 1.3 | 0.7 | 1.6 | 1.2 | 1.2 | 0.29 |
| YLR128W | 1.3 | 1.4 | 0.8 | 1.8 | | 1.3 | 1.3 | 1.1 | 0.8 | 1.7 | 1.4 | 0.7 | 1.2 | 1.4 | 0.9 | 1.5 | 1.2 | 1.2 | 0.25 |
| YLR204W | 1.1 | 0.9 | 0.5 | 2.1 | 2.0 | 1.5 | 2.0 | 1.3 | 1.4 | 1.6 | 1.6 | 0.8 | 1.1 | 1.8 | 1.0 | 2.5 | 1.2 | 1.3 | 1.26 |
| YLR211C | 1.3 | 1.0 | 0.8 | 7.7 | 1.7 | 1.2 | 1.8 | 1.1 | 0.9 | 1.2 | 0.8 | 0.7 | 1.1 | 1.2 | 3.6 | 1.9 | 1.3 | 1.3 | 0.35 |
| YLR257W | 1.5 | 1.0 | 0.7 | 2.4 | 1.4 | 1.2 | 0.8 | 1.3 | 1.0 | 1.1 | 1.2 | 1.0 | 1.0 | 0.9 | 1.0 | 1.7 | 1.8 | 2.6 | 5.20 |
| YLR326W | 1.2 | 1.0 | 1.5 | 2.2 | 1.3 | 0.4 | 1.2 | 0.9 | 0.7 | 1.5 | 6.6 | 0.5 | 0.4 | 1.1 | 0.7 | 0.9 | 0.8 | 1.0 | 0.33 |
| YLR334C | 1.2 | 1.1 | 1.1 | 2.3 | 1.8 | 1.0 | 1.4 | 0.9 | 1.0 | 0.8 | 0.8 | 0.8 | 0.9 | 2.2 | 2.5 | 0.7 | 0.8 | 1.0 | 0.30 |
| YLR408C | 1.3 | 2.3 | 0.6 | 2.3 | 1.4 | 1.0 | 1.0 | 1.4 | 0.9 | 1.1 | 0.7 | 0.8 | 1.1 | 2.1 | 0.9 | 1.1 | 1.2 | 1.2 | 0.79 |
| YLR414C | 1.5 | 1.6 | 1.4 | 4.8 | 1.3 | 0.6 | 0.7 | 0.9 | 0.2 | 1.2 | 1.2 | 0.6 | 1.0 | 1.0 | 2.1 | 0.9 | 3.9 | 3.6 | 2.21 |
| YLR444C | 0.9 | 0.8 | 1.7 | 5.9 | 0.7 | 0.7 | 0.9 | 0.9 | 0.2 | 0.2 | 1.1 | 0.6 | 0.5 | 1.1 | 1.3 | 0.7 | 0.9 | 0.9 | 0.50 |
| YML050W | 1.5 | 1.2 | 1.3 | 2.1 | 0.8 | 0.5 | 0.9 | 1.1 | 1.3 | 1.6 | 1.2 | 0.8 | 0.9 | 1.9 | 1.0 | 1.5 | 0.8 | 1.1 | 0.42 |
| YML107C | 1.1 | 0.7 | 0.4 | 1.8 | 2.2 | 1.1 | 1.5 | 1.3 | 0.6 | 0.9 | 0.9 | 0.7 | 0.6 | 1.3 | 1.0 | 1.3 | 1.2 | 1.5 | 0.67 |
| YMR031C | 0.7 | 0.8 | 1.2 | 2.1 | 0.6 | 0.7 | 0.8 | 0.9 | 1.5 | 1.1 | 0.8 | 0.7 | 1.0 | 1.0 | 0.7 | 1.6 | 0.9 | 1.3 | 1.26 |
| YMR204C | 1.1 | 1.9 | 1.3 | 2.3 | 1.0 | 0.6 | 0.7 | 1.0 | 1.0 | 2.2 | 0.7 | 0.7 | 1.1 | 1.2 | 1.5 | 1.1 | 0.9 | 1.1 | 0.31 |
| YMR206W | 1.4 | 1.4 | 2.3 | 3.9 | 1.1 | 1.0 | 1.3 | 0.9 | 1.0 | 9.0 | 1.8 | 1.0 | 1.2 | 1.4 | 0.3 | 2.2 | 1.0 | 1.2 | 0.17 |
| YNL010W | 1.5 | 1.0 | 0.7 | 1.9 | 1.0 | 1.2 | 1.1 | 1.8 | 0.5 | 0.6 | 1.1 | 0.9 | 0.7 | 1.2 | 0.9 | 1.2 | 1.0 | 1.7 | 4.53 |
| YNL127W | 1.1 | 1.2 | 0.7 | 3.6 | 0.7 | 1.2 | 0.8 | 1.0 | 2.7 | 2.2 | 1.0 | 1.0 | 1.7 | 1.0 | 1.5 | 1.4 | 0.9 | 1.4 | 0.36 |
| YNL217W | 1.1 | 1.2 | 0.5 | 1.7 | 2.1 | 1.5 | 1.3 | 1.4 | 0.3 | 0.9 | 0.7 | 1.0 | 1.0 | 0.9 | 0.5 | 1.0 | 1.3 | 2.1 | 2.35 |
| YOL118C | 2.6 | 2.2 | 1.3 | 2.2 | | 1.5 | 3.2 | 1.1 | | 3.2 | 1.4 | 0.6 | 1.7 | 3.0 | 1.0 | 0.5 | 0.9 | 1.0 | 0.24 |
| YOR053W | 1.0 | 1.1 | 0.5 | 1.7 | 1.7 | 0.9 | 1.6 | 0.9 | 1.3 | 0.8 | 1.3 | 0.6 | 0.9 | 1.2 | 1.0 | 1.3 | 1.2 | 1.2 | 0.77 |
| YOR352W | 1.3 | 0.9 | 1.0 | 2.2 | 1.6 | 0.9 | 1.8 | 1.1 | 1.7 | 2.0 | 1.1 | 1.0 | 1.2 | 1.3 | 0.7 | 2.5 | 1.3 | 1.7 | 0.59 |
| YOR394W | 1.2 | 0.8 | 2.2 | 1.6 | 1.6 | 1.7 | 1.8 | 1.0 | 1.1 | 2.3 | 1.9 | 0.9 | 1.3 | 0.9 | 0.9 | 1.7 | 1.6 | 1.1 | 0.51 |
| YPL033C | 0.8 | 1.0 | 0.9 | 5.1 | 1.3 | 0.7 | 1.0 | 0.7 | 0.4 | 1.6 | 0.7 | 0.6 | 0.7 | 1.1 | 0.8 | 0.4 | 0.9 | 0.8 | 0.23 |
| YPL066W | 0.9 | 1.0 | 1.1 | 2.0 | 1.3 | 1.3 | 1.3 | 0.9 | 0.5 | 0.8 | 1.0 | 1.1 | 0.9 | 0.7 | 0.7 | 1.2 | 1.3 | 1.3 | 0.77 |
| YPR014C | 1.0 | 0.7 | 1.1 | 7.9 | 0.8 | 0.9 | 0.8 | 0.9 | | 0.9 | 0.5 | 0.7 | 0.7 | 1.0 | 0.9 | 0.8 | 0.9 | 1.1 | 0.29 |
| YBR005W | 1.9 | 2.6 | 1.1 | 2.9 | 0.8 | 0.7 | 1.2 | 0.9 | 0.4 | 1.3 | 0.8 | 1.7 | 1.9 | 0.9 | 1.1 | 1.7 | 2.9 | 4.6 | 1.00 |
| YFL027C | 0.8 | 0.8 | 1.1 | 1.3 | 1.1 | 0.3 | 0.4 | 1.1 | 1.8 | 1.3 | 1.3 | 0.6 | 1.1 | 0.3 | 1.4 | 1.2 | 5.1 | 5.2 | 1.74 |
| YGL080W | 1.3 | 1.5 | 1.1 | 1.3 | 1.5 | 1.4 | 1.5 | 1.2 | 1.1 | 1.2 | 0.8 | 1.0 | 0.7 | 1.6 | 0.9 | 1.7 | 1.3 | 2.9 | 1.70 |
| YMR252C | 1.1 | 1.0 | 1.5 | 1.3 | 1.3 | 1.0 | 1.3 | 1.1 | 0.9 | 1.0 | 1.4 | 0.8 | 1.1 | 1.1 | 1.1 | 2.1 | 1.5 | 2.2 | 0.86 |
| YOR385W | 2.8 | 1.2 | 0.2 | 2.5 | 1.5 | 1.0 | 1.2 | 1.0 | 1.5 | 2.2 | 1.2 | 0.6 | 2.8 | 0.8 | 0.8 | 1.2 | 4.6 | 5.9 | 1.17 |
| YAR033W | 1.0 | 0.8 | 1.5 | 1.1 | 1.1 | 1.4 | 2.1 | 1.2 | 1.3 | 1.6 | 1.5 | 0.9 | 1.5 | 1.3 | 0.9 | 2.0 | 1.1 | 3.0 | 1.15 |
| YBR151W | 0.8 | 0.8 | 1.3 | 1.2 | 1.2 | 1.7 | 1.3 | 0.8 | 0.8 | 0.9 | 0.9 | 0.9 | 1.1 | 0.8 | 1.0 | 3.0 | 1.4 | 2.4 | 1.83 |
| YHR162W | 1.2 | 1.2 | 0.9 | 1.8 | 1.2 | 1.3 | 1.7 | 1.4 | 1.0 | 1.7 | 1.3 | 1.2 | 1.5 | 2.0 | 1.1 | 1.4 | 1.3 | 2.7 | 3.64 |
| YLR165C | 1.4 | 0.8 | 1.3 | 1.1 | 1.1 | 1.5 | 1.4 | 1.1 | 1.7 | 1.0 | 1.4 | 0.9 | 1.2 | 1.4 | 0.5 | 1.6 | 1.1 | 2.1 | 0.78 |
| YNL157W | 1.4 | 1.1 | 0.7 | 1.5 | 1.9 | 0.9 | 1.3 | 1.1 | 1.7 | 1.4 | 1.3 | 0.9 | 1.6 | 1.5 | 1.0 | 1.0 | 1.9 | 2.3 | 1.41 |
| YOL002C | 0.9 | 0.7 | 0.5 | 1.0 | 2.0 | 0.9 | 0.4 | 0.8 | 0.1 | 0.3 | 0.8 | 1.1 | 0.2 | 0.5 | 0.6 | 1.7 | 1.5 | 2.1 | 5.33 |
| YPL233W | 1.2 | 0.9 | 0.5 | 1.1 | 1.4 | 0.6 | 1.0 | 1.0 | 0.3 | 0.6 | 0.9 | 0.6 | 1.0 | 1.0 | 0.4 | 1.0 | 1.9 | 1.9 | 0.48 |
| YGR149W | 1.4 | 1.3 | 1.8 | 1.9 | 1.0 | 1.7 | 1.8 | 0.8 | 0.6 | 0.6 | 1.8 | 1.2 | 2.6 | 1.0 | 2.4 | 1.3 | 2.0 | 1.7 | 0.71 |
| YNL043C | 1.0 | 4.4 | 1.6 | 1.3 | 0.8 | 0.6 | 0.9 | 0.9 | 0.4 | 0.4 | 0.7 | 0.9 | 2.1 | 1.2 | 1.6 | 0.8 | 2.7 | 1.6 | 0.68 |
| YPL067C | 1.2 | 1.6 | 2.8 | 1.4 | 1.0 | 0.8 | 1.2 | 1.2 | | 0.8 | 0.9 | 1.4 | 0.9 | 0.7 | 1.5 | 1.1 | 2.9 | 1.7 | 0.40 |
| YPL170W | 1.2 | 1.3 | 0.9 | 1.1 | 1.9 | 1.1 | 0.6 | 1.1 | 1.4 | 1.0 | 1.1 | 0.9 | 1.1 | 1.4 | 1.2 | 1.6 | 2.2 | 1.4 | 1.05 |
| YGL051W | 1.1 | 1.0 | 1.6 | 1.7 | 1.3 | 1.0 | 1.3 | 0.8 | 1.1 | 1.8 | 1.4 | 1.0 | 1.5 | 1.2 | 0.8 | 2.6 | 1.4 | 1.9 | 1.06 |
| YIL112W | 1.1 | 1.4 | 2.8 | 1.9 | 0.6 | 1.3 | 1.1 | 0.9 | 1.3 | 2.0 | 1.4 | 1.2 | 1.1 | 1.3 | 1.8 | 2.4 | 1.2 | 1.3 | 0.45 |
| YLR052W | 1.0 | 1.5 | 0.8 | 1.3 | 1.0 | 0.8 | 1.0 | 1.2 | 0.4 | 1.1 | 1.1 | 1.0 | 0.8 | 1.2 | 1.0 | 2.4 | 0.7 | 0.9 | 0.77 |
| YNL203C | 0.9 | 1.3 | 0.3 | 3.2 | 1.0 | 1.7 | 1.6 | 0.8 | | 0.3 | 1.1 | 0.9 | 1.2 | 2.7 | 1.2 | 5.6 | 1.3 | 1.7 | 0.21 |
| YNR014W | 1.0 | 0.8 | 1.7 | 1.2 | 1.2 | 0.7 | 1.7 | 0.9 | 0.3 | 2.3 | 4.4 | 0.9 | 1.5 | 4.9 | 1.0 | 2.4 | 1.1 | 1.0 | 0.31 |
| YAL028W | 0.9 | 0.7 | 1.1 | 2.0 | 1.0 | 0.6 | 1.2 | 0.8 | 1.6 | 1.3 | 1.0 | 1.0 | 1.5 | 1.6 | 2.2 | 1.7 | 1.0 | 1.0 | 0.30 |
| YBL095W | 0.8 | 1.4 | 1.2 | 1.4 | 1.1 | 0.9 | 1.1 | 0.9 | 1.0 | 0.6 | 1.2 | 0.8 | 1.0 | 1.0 | 0.4 | 2.2 | 1.3 | 1.9 | 0.72 |
| YBR157C | 0.7 | 1.0 | 0.4 | 1.4 | 1.3 | 1.2 | 1.6 | 0.7 | 0.6 | 0.3 | 0.6 | 0.6 | 0.5 | 0.9 | 0.2 | 1.9 | 1.3 | 0.8 | 0.69 |
| YDL091C | 1.1 | 1.3 | 1.8 | 1.4 | 0.7 | 0.8 | 1.3 | 1.3 | 1.6 | 1.5 | 1.5 | 1.0 | 1.3 | 1.5 | 1.9 | 2.4 | 1.1 | 1.6 | 0.47 |
| YDL216C | 1.1 | 1.1 | 0.7 | 2.0 | 1.6 | 1.6 | 1.2 | 1.6 | 1.3 | 1.3 | 0.7 | 0.9 | 1.2 | 1.1 | 1.0 | 1.8 | 1.0 | 1.6 | 0.42 |
| YDR067C | 1.3 | 1.2 | 0.7 | 2.7 | 0.9 | 1.1 | 1.3 | 1.8 | 1.0 | 1.3 | 0.9 | 0.9 | 1.2 | 1.5 | 1.5 | 2.4 | 1.3 | 1.4 | 0.76 |
| YDR186C | 0.7 | 0.9 | 0.7 | 0.7 | 1.3 | 0.7 | 1.4 | 1.2 | 1.2 | 1.6 | 0.7 | 1.1 | 1.1 | 0.8 | 0.8 | 2.3 | 1.1 | 0.9 | 0.50 |
| YDR196C | 0.9 | 0.9 | 0.7 | 1.1 | 1.0 | 1.7 | 1.7 | 1.5 | 1.6 | 1.1 | 1.3 | 0.7 | 1.2 | 1.0 | 0.7 | 2.7 | 1.2 | 1.9 | 1.54 |
| YDR262W | 1.3 | 1.8 | 0.8 | 1.2 | 1.7 | 1.3 | 1.7 | 1.2 | 0.9 | 1.7 | 1.6 | 0.8 | 0.9 | 1.7 | 1.5 | 3.8 | 1.0 | 1.4 | 1.11 |
| YDR306C | 0.9 | 1.2 | 0.7 | 1.8 | 1.7 | 1.2 | 1.6 | 1.3 | 2.0 | 1.4 | 2.1 | 0.9 | 1.2 | 1.1 | 1.2 | 2.4 | 1.0 | 1.5 | 0.77 |
| YDR319C | 1.3 | 0.9 | 0.8 | 1.1 | 1.2 | 1.2 | 1.7 | 1.3 | 1.1 | 1.1 | 1.3 | 1.0 | | 1.3 | 0.8 | 2.0 | 2.1 | 2.2 | 1.23 |
| YER188W | 1.3 | 0.9 | 1.2 | 1.9 | 0.9 | 1.5 | 1.2 | 0.7 | 0.4 | 0.9 | 1.2 | 0.9 | 1.1 | 1.1 | 0.3 | 2.5 | 1.0 | 1.1 | 0.61 |
| YGL004C | 0.8 | 1.4 | 1.3 | 1.0 | 1.0 | 1.4 | 2.9 | 1.4 | 1.8 | 2.2 | 1.0 | 0.8 | 1.3 | 0.9 | 0.6 | 2.2 | 1.1 | 1.4 | 0.48 |
| YGR141W | 0.8 | 1.6 | 1.6 | 1.7 | 1.1 | 1.5 | 1.5 | 0.9 | 0.3 | 1.0 | 0.6 | 0.6 | 1.0 | 0.7 | 2.6 | 2.1 | 1.1 | 1.3 | 0.65 |
| YHR080C | 0.6 | 1.8 | 1.9 | 0.7 | 0.8 | 1.8 | 1.1 | 1.1 | 1.9 | 1.2 | 0.7 | 1.2 | 2.0 | 0.9 | 0.9 | 2.3 | 1.1 | 1.3 | 0.49 |
| YHR097C | 1.6 | 0.7 | 0.7 | 0.9 | | 0.8 | 0.8 | 0.7 | 0.4 | 0.6 | 0.7 | 0.8 | 1.3 | 0.9 | 2.7 | 2.0 | 1.7 | 1.4 | 0.24 |
| YIL077C | 0.9 | 0.8 | 1.2 | 1.2 | 1.3 | 1.8 | 1.2 | 0.9 | 1.5 | 1.4 | 1.3 | 1.6 | 1.3 | 1.3 | 0.9 | 2.4 | 1.3 | 1.1 | 0.75 |
| YJL046W | 0.9 | 0.9 | 0.6 | 1.8 | 1.0 | 0.7 | 1.5 | 1.1 | 1.8 | 2.7 | 4.2 | 0.9 | 1.3 | 1.3 | 1.0 | 2.5 | 1.0 | 1.3 | 0.68 |
| YLL005C | 1.0 | 1.1 | 1.1 | 1.2 | 1.6 | 1.9 | 1.3 | 1.0 | 0.4 | 1.0 | 0.9 | 0.8 | 1.1 | 1.7 | 1.0 | 2.1 | 1.1 | 1.2 | 0.44 |
| YLR151C | 1.4 | 2.0 | 1.4 | 1.0 | 1.0 | | 1.4 | 1.4 | 1.5 | 0.9 | 1.1 | 0.5 | 0.7 | 1.3 | 0.7 | 2.4 | 1.0 | 1.4 | 0.30 |
| YLR271W | 1.6 | 1.2 | 0.8 | 1.8 | 1.4 | 1.4 | 1.7 | 1.5 | 1.0 | 1.0 | 1.3 | 0.8 | 1.2 | 1.2 | 1.0 | 3.0 | 1.5 | 1.8 | 0.78 |
| YMR025W | 1.2 | 1.2 | 0.7 | 1.5 | 1.3 | 1.3 | 1.4 | 1.2 | 1.0 | 1.6 | 1.2 | 0.9 | 1.2 | 1.8 | 1.1 | 2.4 | 1.0 | 1.5 | 0.42 |
| YMR135C | 1.0 | 1.0 | 1.1 | 1.9 | 1.8 | 1.0 | 1.0 | 1.0 | 1.1 | 1.4 | 1.1 | 1.0 | 1.3 | 1.0 | 1.4 | 2.3 | 1.5 | 1.3 | 1.06 |
| YMR210W | 1.0 | 1.1 | 1.8 | 1.9 | 0.6 | 0.8 | 1.1 | 1.1 | 1.5 | 2.2 | 0.8 | 0.9 | 1.0 | 1.0 | 1.0 | 2.2 | 0.8 | 1.5 | 0.56 |
| YNR040W | 0.8 | 4.9 | 0.5 | 0.9 | 0.9 | 1.6 | 2.1 | 1.3 | 1.1 | 0.8 | 1.2 | 0.6 | 0.8 | 1.5 | 0.7 | 2.1 | 1.1 | 1.0 | 0.30 |
| YPL039W | 1.2 | 0.9 | 0.6 | 1.3 | 1.4 | 1.5 | 1.5 | 1.4 | 1.1 | 1.0 | 0.9 | 0.9 | 1.1 | 1.1 | 0.6 | 2.4 | 1.1 | 1.1 | 0.52 |
| YPL099C | 1.1 | 1.9 | 0.7 | 2.0 | 0.9 | 1.2 | 1.4 | 1.4 | 2.4 | 1.7 | 0.8 | 0.9 | 1.1 | 1.3 | 0.5 | 2.1 | 1.0 | 1.7 | 0.92 |
| YPL107W | 1.2 | 0.8 | 1.1 | 1.3 | 1.0 | 1.1 | 1.2 | 1.5 | 1.4 | 1.3 | 1.1 | 0.7 | 0.8 | 1.7 | 0.7 | 1.8 | 1.0 | 1.1 | 0.59 |
| YPL138C | 1.1 | 1.7 | 1.1 | 1.8 | 0.7 | 0.5 | 1.1 | 1.3 | 1.1 | 1.5 | 0.5 | 0.9 | 0.6 | 1.3 | 2.7 | 2.6 | 0.8 | 1.1 | 0.38 |

TABLE 2
Mitochondria-located protein genes

| | The level of expressed mRNA in the presence of chemical substance/The | | | | | | | | | | | | | | | | | | |
|---|---|---|---|---|---|---|---|---|---|---|---|---|---|---|---|---|---|---|---|
| | level of expressed mRNA in the absence of chemical substance | | | | | | | | | | | | | | | | | | |
| yeast genes | (1) | (2) | (3) | (4) | (5) | (6) | (7) | (8) | (9) | (10) | (11) | (12) | (13) | (14) | (15) | (16) | (17) | (18) | Intensity |
| YJR048W | 1.3 | 0.9 | 2.2 | 1.4 | 0.8 | 0.5 | 0.3 | 0.9 | 0.5 | 1.5 | 1.0 | 0.8 | 0.4 | 2.5 | 0.6 | 1.5 | 1.2 | 1.2 | 1.19 |
| YOR226C | 1.3 | 2.2 | 1.7 | 1.1 | 1.3 | 1.2 | 1.2 | 1.2 | 1.3 | 5.9 | 1.5 | 0.6 | 0.9 | 2.7 | 1.0 | 0.7 | 1.0 | 0.7 | 0.58 |
| YDL174C | 0.9 | 1.8 | 0.9 | 1.3 | 0.5 | 1.7 | 3.6 | 0.8 | 0.5 | 1.7 | 0.9 | 2.8 | 5.9 | 1.1 | 0.6 | 5.1 | 2.2 | 4.0 | 0.63 |
| YBL022C | 0.4 | 0.8 | 1.2 | 0.7 | 1.2 | 1.2 | 1.0 | 0.4 | 2.8 | 1.2 | 1.1 | 0.9 | 2.6 | 1.0 | 1.5 | 1.0 | 0.9 | 0.7 | 0.76 |
| YCL057W | 0.6 | 1.5 | 2.3 | 0.5 | 1.1 | 1.8 | 1.4 | 1.8 | 4.2 | 2.1 | 1.6 | 1.0 | 3.8 | 1.0 | 2.8 | 1.5 | 0.9 | 1.1 | 0.76 |
| YDR258C | 1.6 | 2.3 | 2.8 | 1.4 | 1.3 | 2.4 | 3.9 | 1.9 | 4.9 | 13.3 | 1.7 | 1.8 | 5.2 | 1.5 | 0.6 | 1.0 | 1.3 | 1.3 | 0.87 |
| YGR028W | 1.4 | 3.5 | 1.0 | 1.8 | 0.9 | 1.5 | 2.1 | 1.4 | 1.7 | 2.0 | 1.1 | 1.5 | 2.6 | 1.2 | 1.0 | 2.2 | 1.5 | 2.6 | 0.91 |
| YGR244C | 1.0 | 1.2 | 1.6 | 1.6 | 0.8 | 2.6 | 3.9 | 1.8 | 1.4 | 1.1 | 1.8 | 1.9 | 2.6 | 1.2 | 2.0 | 2.6 | 1.9 | 3.1 | 1.12 |
| YKL142W | 1.8 | 2.7 | 3.1 | 1.4 | 0.7 | 3.4 | 6.9 | 2.3 | 5.8 | 5.9 | 2.0 | 1.9 | 3.3 | 2.3 | 1.3 | 3.3 | 2.4 | 3.6 | 1.68 |
| YNL055C | 1.1 | 1.8 | 5.5 | 2.0 | 0.9 | 2.6 | 1.2 | 0.7 | 1.1 | 1.0 | 1.9 | 1.7 | 2.4 | 0.9 | 1.1 | 2.6 | 1.2 | 1.7 | 4.10 |
| YNL071W | 0.9 | 0.9 | 1.7 | 1.5 | 0.6 | 1.3 | 1.4 | 0.9 | 1.1 | 1.2 | 1.6 | 1.3 | 2.5 | 0.8 | 1.1 | 1.1 | 1.0 | 1.2 | 1.61 |
| YOR020C | 1.4 | 2.1 | 1.7 | 1.7 | 1.5 | 2.3 | 1.7 | 1.7 | 5.8 | 2.6 | 2.5 | 0.8 | 3.0 | 1.6 | 1.5 | 1.4 | 1.4 | 1.7 | 1.66 |
| YOR037W | 1.0 | 1.5 | 0.6 | 1.4 | 1.5 | 1.6 | 2.5 | 1.5 | 1.3 | 1.0 | 1.2 | 1.2 | 2.1 | 1.0 | 0.9 | 1.4 | 1.2 | 1.6 | 0.61 |
| YDL198C | 1.4 | 1.0 | 2.1 | 1.4 | 0.7 | 0.8 | 1.0 | 1.1 | 1.9 | 2.7 | 2.5 | 1.2 | 2.0 | 1.7 | 1.2 | 1.4 | 1.5 | 1.1 | 1.19 |
| YDR231C | 1.1 | 1.6 | 1.0 | 1.5 | 1.1 | 2.2 | 1.7 | 1.3 | 1.5 | 1.4 | 1.3 | 1.1 | 2.4 | 1.5 | 1.0 | 3.0 | 1.2 | 1.3 | 0.92 |
| YER178W | 0.8 | 0.9 | 3.6 | 1.0 | 0.7 | 2.5 | 1.6 | 0.8 | 1.7 | 1.3 | 2.5 | 1.3 | 2.4 | 0.9 | 1.8 | 1.0 | 1.1 | 0.9 | 2.18 |
| YFL016C | 0.8 | 1.0 | 1.6 | 0.9 | 1.3 | 1.3 | 1.4 | 0.7 | 6.4 | 2.9 | 1.2 | 1.0 | 2.8 | 0.8 | 0.8 | 0.6 | 1.2 | 0.9 | 1.25 |
| YGR008C | 2.1 | 3.0 | 1.7 | 3.2 | 0.9 | 2.9 | 3.7 | 1.9 | 3.1 | 2.4 | 2.6 | 2.0 | 3.4 | 1.3 | 1.5 | 2.3 | 1.7 | 4.2 | 3.03 |
| YIL155C | 1.0 | 0.7 | 1.4 | 3.7 | 1.3 | 1.4 | 2.2 | 1.0 | 2.5 | 2.9 | 1.3 | 1.4 | 2.0 | 1.3 | 1.4 | 3.8 | 1.1 | 1.4 | 0.51 |
| YJL102W | 1.1 | 0.7 | 0.4 | 0.8 | 1.3 | 1.6 | 0.6 | 0.9 | 3.1 | 2.2 | 0.7 | 0.8 | 2.6 | 1.5 | 1.5 | 0.6 | 1.0 | 1.1 | 0.22 |
| YJR045C | 0.5 | 1.9 | 3.9 | | 0.5 | 1.2 | 1.0 | 0.8 | 3.2 | 3.0 | 1.4 | 1.0 | 2.3 | 0.5 | 2.3 | 0.8 | 0.7 | 0.9 | 3.78 |
| YLR259C | 0.7 | 1.0 | 3.1 | 1.6 | 1.1 | 0.9 | 1.8 | 0.8 | 1.8 | 2.6 | 3.0 | 0.7 | 2.5 | 0.8 | 2.1 | 1.3 | 0.8 | 1.0 | 2.09 |
| YLR348C | 1.1 | 4.5 | 1.2 | 1.2 | 0.9 | 1.9 | 0.9 | 0.9 | 2.1 | 1.3 | 1.3 | 0.9 | 1.9 | 1.0 | 2.4 | 1.1 | 1.1 | 1.0 | 0.64 |
| YML054C | 1.5 | 1.8 | 1.3 | 3.4 | 1.3 | 1.8 | 1.2 | 1.5 | 4.1 | 1.8 | 1.4 | 1.8 | 2.8 | 2.1 | 1.1 | 7.8 | 1.1 | 1.6 | 0.25 |
| YMR089C | 0.8 | 1.1 | 0.9 | 0.9 | 0.8 | 1.2 | 1.2 | 1.3 | 5.6 | 2.5 | 1.3 | 0.8 | 2.3 | 0.9 | 1.8 | 1.1 | 0.9 | 1.1 | 0.69 |
| YMR152W | 0.9 | 1.1 | 1.8 | 0.9 | 0.8 | 7.5 | 0.8 | 0.8 | 2.7 | 1.3 | 1.2 | 1.3 | 1.9 | 1.1 | 0.6 | 1.0 | 1.5 | 0.9 | 0.71 |
| YNL104C | 1.1 | 1.3 | 3.0 | 0.9 | 0.8 | 0.8 | 0.6 | 0.9 | 1.1 | 2.0 | 2.1 | 1.1 | 2.1 | 0.9 | 2.5 | 1.5 | 0.9 | 0.9 | 1.69 |
| YOR130C | 0.8 | 1.7 | 1.4 | 1.0 | 1.8 | 1.6 | 1.3 | 0.8 | 2.0 | 1.7 | 0.6 | 0.9 | 2.1 | 1.3 | 1.0 | 0.8 | 1.5 | 1.1 | 0.51 |
| YPR024W | 0.7 | 1.0 | 1.3 | 1.0 | 1.3 | 1.0 | 1.4 | 0.7 | 3.0 | 1.7 | 0.9 | 0.8 | 2.5 | 0.7 | 1.0 | 1.6 | 1.1 | 0.8 | 0.84 |
| YPR067W | 1.6 | 1.2 | 1.7 | 0.9 | 1.3 | 1.3 | 1.5 | 1.4 | 3.5 | 3.4 | 1.8 | 1.0 | 3.0 | 1.6 | 1.3 | 0.8 | 1.0 | 1.0 | 0.66 |
| YBR029C | 0.7 | 0.2 | 1.8 | 1.0 | 1.4 | 0.8 | 0.4 | 0.7 | 2.0 | 1.1 | 0.7 | 1.3 | 1.7 | 0.9 | 3.5 | 0.9 | 0.6 | 0.8 | 1.56 |
| YCL009C | 0.6 | 0.9 | 2.0 | 0.5 | 0.7 | 1.8 | 1.3 | 1.1 | 0.9 | 1.6 | 1.5 | 1.7 | 1.6 | 0.7 | 6.5 | 0.9 | 1.1 | 0.8 | 0.68 |
| YER026C | 0.8 | 0.6 | 3.3 | 1.0 | 1.2 | 2.5 | 1.6 | 0.8 | 0.9 | 1.5 | 0.9 | 1.7 | 1.9 | 1.1 | 4.0 | 1.4 | 1.7 | 3.2 | 4.48 |
| YLR109W | 0.8 | 2.9 | 6.0 | 0.9 | 1.3 | 2.1 | 2.6 | 0.9 | 2.4 | 2.7 | 1.5 | 1.6 | 3.2 | 2.3 | 3.9 | 1.8 | 1.1 | 1.2 | 3.86 |
| YMR189W | 0.7 | 0.8 | 1.9 | 2.8 | 0.4 | 1.9 | 0.8 | 0.4 | 0.7 | 0.9 | 8.2 | 0.6 | 0.9 | 1.6 | 5.6 | 0.9 | 1.0 | 0.8 | 0.88 |
| YNL169C | 0.7 | 1.4 | 1.4 | 1.2 | 0.7 | 1.4 | 0.7 | 0.8 | 1.1 | 1.1 | 0.8 | 1.0 | 1.7 | 0.9 | 3.4 | 0.8 | 1.1 | 1.3 | 1.05 |
| YER069W | 1.3 | 0.8 | 3.3 | 1.4 | 1.5 | 1.3 | 1.0 | 1.5 | 0.8 | 10.4 | 1.5 | 0.8 | 1.4 | 1.3 | 2.4 | 1.1 | 1.1 | 0.9 | 0.25 |
| YIL022W | 0.8 | 0.7 | 1.1 | 1.6 | 0.8 | 1.2 | 0.7 | 0.8 | 1.1 | 1.3 | 1.0 | 0.9 | 1.2 | 0.6 | 2.9 | 1.1 | 0.8 | 0.7 | 0.52 |
| YBR146W | 0.9 | 0.8 | 0.7 | 1.5 | 1.3 | 2.6 | 1.5 | 1.0 | 0.9 | 1.1 | 1.2 | 1.0 | 1.7 | 1.2 | 1.0 | 2.9 | 1.0 | 1.2 | 1.61 |
| YDR019C | 1.4 | 0.5 | 1.7 | 4.0 | 0.9 | 4.8 | 1.4 | 0.7 | 0.4 | 0.7 | 1.8 | 0.7 | 1.0 | 1.6 | 1.8 | 1.8 | 1.2 | 1.6 | 2.48 |
| YGR207C | 1.5 | 1.1 | 1.0 | 1.1 | 1.5 | 2.8 | 1.7 | 1.9 | 2.1 | 2.6 | 1.4 | 1.1 | 1.0 | 2.2 | 0.6 | 1.8 | 1.2 | 1.8 | 1.49 |
| YMR072W | 1.1 | 1.0 | 1.4 | 1.0 | 1.7 | 2.4 | 1.4 | 1.0 | 3.0 | 1.2 | 1.9 | 0.8 | 2.1 | 1.2 | 0.8 | 1.7 | 0.9 | 1.9 | 2.06 |
| YNL037C | 1.4 | 1.8 | 1.7 | 1.1 | 1.7 | 2.8 | 2.8 | 1.0 | 0.6 | 3.2 | 1.4 | 1.3 | 1.9 | 1.3 | 0.9 | 1.4 | 0.9 | 1.3 | 2.05 |
| YOR136W | 1.0 | 0.9 | 3.5 | 1.0 | 1.5 | 3.9 | 3.2 | 1.1 | 0.3 | 3.4 | 1.4 | 1.2 | 1.5 | 0.9 | 1.2 | 1.3 | 0.8 | 1.3 | 3.20 |
| YPL271W | 1.2 | 3.2 | 4.1 | 1.2 | 1.4 | 3.3 | 1.2 | 0.9 | 0.6 | 1.2 | 1.7 | 0.8 | 1.2 | 1.3 | 0.8 | 1.4 | 1.2 | 1.3 | 1.34 |
| YAL044C | 1.5 | 1.1 | 1.0 | 3.0 | 0.8 | 2.2 | 1.0 | 0.8 | 0.4 | 0.6 | 1.4 | 0.6 | 1.1 | 1.4 | 0.7 | 1.0 | 1.4 | 1.8 | 4.07 |
| YAL054C | 1.2 | 1.1 | 1.4 | 1.6 | 1.1 | 2.2 | 1.1 | 1.1 | 8.9 | 4.1 | 1.3 | 1.0 | 1.8 | 1.3 | 0.9 | 2.5 | 1.5 | 1.4 | 0.24 |
| YBR024W | 1.0 | 1.0 | 1.1 | 2.4 | 1.0 | 2.2 | 1.9 | 1.4 | 1.3 | 1.4 | 1.3 | 1.1 | 1.7 | 1.9 | 0.8 | 2.3 | 1.0 | 1.2 | 1.14 |
| YDR405W | 0.9 | 1.1 | 4.7 | 1.0 | 1.1 | 2.4 | 1.2 | 0.9 | 0.6 | 1.0 | 1.0 | 1.2 | 1.3 | 1.1 | 2.5 | 1.3 | 1.3 | 0.9 | 0.44 |
| YGL068W | 1.0 | 0.6 | 1.1 | 1.4 | 0.9 | 2.6 | 1.5 | 0.9 | 0.7 | 0.8 | 1.0 | 1.1 | 1.0 | 0.9 | 0.6 | 1.8 | 1.8 | 1.0 | 3.35 |
| YGR220C | 1.1 | 0.6 | 0.9 | 1.1 | 1.3 | 2.1 | 1.4 | 1.5 | 0.8 | 1.5 | 1.1 | 0.9 | 1.1 | 1.5 | 1.1 | 2.3 | 0.9 | 1.1 | 1.42 |
| YHR037W | 0.7 | 0.9 | 1.3 | 1.6 | 0.9 | 1.8 | 1.2 | 1.0 | 0.9 | 2.2 | 1.7 | 1.1 | 1.1 | 1.1 | 1.1 | 2.0 | 1.0 | 1.4 | 1.15 |
| YIL070C | 1.2 | 1.0 | 0.8 | 1.4 | 1.3 | 2.3 | 1.6 | 1.1 | 0.3 | 0.6 | 1.0 | 0.8 | 0.7 | 1.3 | 1.0 | 2.4 | 0.7 | 1.1 | 1.69 |
| YIL111W | 1.6 | 1.0 | 1.8 | 2.7 | | 2.6 | 3.2 | 1.2 | 1.6 | 1.1 | 2.1 | 0.9 | 1.7 | 1.5 | 1.1 | 2.5 | 1.9 | 3.9 | 1.52 |
| YKL138C | 1.2 | 0.9 | 0.6 | 0.9 | 1.3 | 2.3 | 1.9 | 1.7 | 1.1 | 2.1 | 1.2 | 0.8 | 0.8 | 1.6 | 0.6 | 2.5 | 1.0 | 1.3 | 1.29 |
| YKL150W | 1.1 | 2.0 | 3.0 | 1.4 | 1.4 | 2.3 | 2.4 | 1.3 | 1.1 | 1.3 | 1.9 | 1.2 | 1.8 | 1.4 | 0.9 | 6.1 | 1.2 | 2.7 | 2.20 |
| YKR006C | 1.0 | 0.7 | 1.2 | 1.2 | 1.4 | 2.4 | 1.6 | 1.2 | 1.0 | 1.5 | 1.2 | 0.8 | 0.9 | 1.6 | 0.9 | 2.3 | 0.8 | 1.1 | 1.39 |
| YLR142W | 4.4 | 2.7 | 1.1 | 6.1 | 1.3 | 3.1 | 1.2 | 2.1 | 3.1 | 4.4 | 3.8 | 0.8 | 1.2 | 2.1 | 1.6 | 3.4 | 1.9 | 3.2 | 0.28 |
| YML110C | 1.1 | 0.8 | 1.9 | 1.6 | 0.8 | 2.6 | 2.0 | 1.4 | 2.7 | 2.2 | 1.7 | 1.1 | 1.8 | 1.4 | 1.3 | 2.3 | 1.1 | 1.6 | 2.01 |
| YNL284C | 1.1 | 0.7 | 0.3 | 1.4 | 1.8 | 1.0 | 2.8 | 1.6 | 0.9 | 1.9 | 1.3 | 0.7 | 1.0 | 1.5 | 0.8 | 2.4 | 1.0 | 1.5 | 1.26 |
| YDR268W | 0.8 | 1.5 | 0.4 | 1.2 | 2.2 | 1.0 | 2.4 | 1.0 | 1.0 | 2.0 | 1.1 | 0.7 | 1.0 | 1.4 | 1.0 | 1.6 | 1.1 | 1.2 | 0.47 |
| YMR193W | 1.4 | 1.2 | 0.5 | 1.9 | 2.2 | 1.7 | 2.7 | 1.7 | 0.8 | 1.1 | 1.6 | 0.6 | 0.9 | 1.5 | 0.8 | 3.7 | 1.1 | 1.7 | 0.93 |
| YOR374W | 1.3 | 4.5 | 7.3 | 3.9 | 0.9 | 1.4 | 1.9 | 0.9 | 2.3 | 5.9 | 2.6 | 1.5 | 1.7 | 1.1 | 2.5 | 2.5 | 1.2 | 2.4 | 1.24 |
| YER061C | 0.9 | 0.9 | 1.2 | 2.5 | 0.8 | 0.4 | 0.9 | 0.7 | 0.3 | 0.7 | 2.2 | 0.7 | 0.9 | 1.0 | 0.8 | 1.8 | 1.2 | 1.2 | 0.84 |
| YIL136W | 1.1 | 1.0 | 1.2 | 2.5 | 1.0 | 1.0 | 1.8 | 1.1 | 3.4 | 1.7 | 4.8 | 0.9 | 1.7 | 1.7 | 1.5 | 4.5 | 1.3 | 3.2 | 0.95 |
| YLL009C | 1.0 | 1.2 | 0.6 | 1.9 | 1.5 | 1.2 | 1.7 | 1.0 | 1.3 | 1.7 | 3.5 | 0.7 | 0.9 | 1.4 | 1.1 | 2.8 | 0.9 | 1.2 | 1.66 |
| YLR163C | 1.0 | 0.9 | 0.7 | 1.4 | 1.6 | 1.3 | 1.7 | 1.4 | 2.6 | 2.9 | 2.2 | 0.7 | 1.4 | 1.3 | 1.2 | 1.4 | 0.9 | 1.1 | 0.55 |
| YBR291C | 2.0 | 0.9 | 1.0 | 1.5 | 0.9 | 1.1 | 0.9 | 2.2 | 0.9 | 1.0 | 1.1 | 0.9 | 1.0 | 1.7 | 0.5 | 1.5 | 0.9 | 1.3 | 1.19 |
| YIL094C | 1.5 | 0.4 | 0.8 | 0.7 | 1.4 | 1.2 | 1.3 | 3.6 | 0.3 | 0.4 | 1.8 | 1.0 | 0.7 | 1.0 | 0.5 | 0.9 | 0.9 | 1.0 | 2.26 |
| YAL015C | 1.2 | 1.1 | 1.7 | 1.9 | 1.1 | 0.7 | 1.2 | 1.1 | 4.0 | 2.7 | 1.4 | 1.0 | 1.2 | 1.6 | 0.7 | 2.0 | 1.1 | 1.2 | 0.61 |
| YJR095W | 1.2 | 20.5 | 1.9 | 6.7 | 1.2 | 1.5 | 2.0 | 0.9 | 0.5 | 6.3 | 0.6 | 0.7 | 0.8 | 1.3 | 0.8 | 0.8 | 1.3 | 0.9 | 0.23 |
| YKL085W | 1.4 | 2.3 | 1.6 | 1.2 | 1.2 | 1.9 | 1.5 | 1.2 | 1.9 | 3.0 | 1.8 | 0.8 | 1.5 | 1.0 | 0.5 | 1.7 | 0.9 | 1.3 | 2.16 |
| YMR177W | 1.4 | 0.8 | 1.1 | 1.0 | 1.6 | 0.8 | 1.0 | 1.2 | 2.4 | 3.8 | 1.4 | 0.6 | 0.5 | 1.2 | 0.8 | 0.5 | 0.9 | 0.9 | 0.55 |
| YPL224C | 1.0 | 2.2 | 1.3 | 2.5 | 0.6 | 1.3 | 2.2 | 1.3 | 3.2 | 4.2 | 0.8 | 1.1 | 1.3 | 1.4 | 1.0 | 2.5 | 1.3 | 1.9 | 0.64 |
| YER014W | 1.0 | 0.9 | 0.9 | 0.6 | 0.8 | 4.0 | | 1.2 | 3.5 | 1.1 | 0.9 | 1.1 | 1.5 | 1.2 | 2.3 | 0.8 | 1.0 | 0.9 | 0.43 |
| YFR049W | 1.5 | 1.5 | 1.8 | 1.2 | 1.3 | | | 1.3 | 2.6 | 1.5 | 1.7 | 1.2 | 1.4 | 1.2 | 0.7 | 0.9 | 1.3 | 1.0 | 0.83 |
| YGR112W | 1.0 | 2.9 | 1.1 | 1.3 | 0.9 | 1.1 | 1.3 | 1.3 | 2.6 | 1.3 | 1.3 | 0.7 | 1.1 | 1.1 | 1.2 | 2.8 | 0.9 | 1.2 | 0.31 |
| YLL001W | 0.9 | 0.7 | 1.2 | 1.2 | 0.6 | 1.0 | 0.8 | 1.0 | 3.5 | 2.4 | 0.9 | 0.7 | 1.7 | 1.2 | 0.9 | 1.3 | 0.9 | 1.3 | 0.70 |
| YML042W | 0.9 | 1.4 | 1.5 | 2.2 | 1.0 | 0.9 | 0.8 | 1.6 | 3.8 | 2.4 | 2.5 | 1.0 | 1.2 | 1.4 | 1.7 | 4.8 | 1.0 | 0.9 | 0.29 |
| YNL213C | 1.7 | 11.6 | 0.9 | 1.5 | 1.3 | 1.2 | 1.4 | 1.8 | 3.0 | 1.5 | 1.5 | 0.8 | 0.9 | 1.8 | 1.0 | 1.7 | 0.9 | 1.2 | 0.70 |
| YOR386W | 1.0 | 0.8 | 1.3 | 1.5 | 1.2 | 3.3 | 1.8 | 1.0 | 4.1 | 3.0 | 1.4 | 1.2 | 2.4 | 0.9 | 1.3 | 1.2 | 2.1 | 1.7 | 0.46 |
| YBR037C | 0.8 | 0.8 | 1.3 | 2.4 | 0.7 | 1.2 | 1.2 | 1.0 | 3.0 | 1.9 | 1.5 | 0.7 | 1.2 | 1.2 | 1.0 | 2.7 | 1.6 | 1.3 | 0.60 |
| YCR024C | 1.1 | 1.0 | 1.5 | 0.8 | 0.8 | 2.9 | 1.0 | 1.2 | 2.5 | 1.7 | 1.4 | 0.8 | 0.9 | 1.3 | 1.0 | 1.2 | 0.9 | 1.0 | 0.32 |
| YDR194C | 0.7 | 0.9 | 0.5 | 1.0 | 1.0 | 1.0 | 1.3 | 1.4 | 2.8 | 2.2 | 1.2 | 0.6 | 1.2 | 0.8 | 1.1 | 0.8 | 0.6 | 0.8 | 2.19 |
| YER017C | 0.8 | 1.1 | 1.4 | 1.3 | 1.0 | 0.5 | 1.1 | 0.5 | 2.7 | 1.8 | 0.9 | 1.0 | 1.5 | 1.1 | 2.1 | 1.0 | 0.9 | 1.0 | 0.43 |
| YGL125W | 1.0 | 3.2 | 4.3 | 0.7 | 0.9 | | 1.7 | 0.8 | 2.1 | 2.1 | 1.6 | 1.0 | 1.4 | 1.0 | 1.9 | 1.2 | 1.2 | 1.1 | 0.27 |
| YGR029W | 1.1 | 1.4 | 1.0 | 1.0 | 0.8 | 0.6 | 1.6 | 1.7 | 2.7 | 1.1 | 1.2 | 0.6 | 1.0 | 2.0 | 0.4 | 1.1 | 1.1 | 1.3 | 0.71 |
| YHL038C | 1.0 | 1.2 | 1.4 | 0.8 | 1.3 | 0.8 | 1.2 | 1.1 | 4.0 | 1.7 | 0.8 | 1.6 | 1.3 | 0.6 | 0.8 | 0.5 | 0.9 | 0.6 | 1.95 |
| YKL192C | 1.1 | 1.0 | 3.7 | 1.1 | 1.1 | 1.8 | 1.1 | 0.9 | 3.5 | 1.6 | 1.1 | 1.2 | 1.7 | 1.5 | 1.2 | 2.1 | 1.0 | 0.8 | 1.57 |
| YKR052C | 0.9 | 1.1 | 2.6 | 2.6 | 1.0 | 0.5 | 1.9 | 1.1 | 1.9 | 3.6 | 1.3 | 0.9 | 1.2 | 1.1 | 0.5 | 2.5 | 0.8 | 1.5 | 1.15 |
| YML078W | 1.2 | 1.9 | 2.5 | 1.5 | 0.7 | 1.4 | 1.3 | 1.4 | 2.6 | 2.2 | 2.4 | 1.2 | 1.7 | 1.7 | 1.0 | 1.8 | 1.3 | 1.6 | 1.98 |
| YMR056C | 1.1 | 1.3 | 1.7 | 1.0 | 1.1 | 0.9 | 1.4 | 1.2 | 1.8 | 1.1 | 1.6 | 0.9 | 0.9 | 1.1 | 0.7 | 2.4 | 1.0 | 1.1 | 0.72 |
| YNL005C | 1.1 | 1.0 | 0.6 | 1.2 | 1.2 | 1.7 | 1.3 | 1.5 | 2.0 | 2.0 | 1.2 | 0.9 | 1.5 | 1.3 | 1.0 | 1.6 | 0.9 | 1.5 | 1.52 |
| YPR047W | 1.0 | 1.0 | 0.9 | 1.5 | 0.9 | 1.0 | 1.8 | 1.2 | 2.1 | 1.9 | 1.1 | 0.9 | 1.3 | 1.0 | 0.8 | 2.8 | 1.0 | 1.5 | 0.39 |
| YPR134W | 1.1 | 1.1 | 0.8 | 1.7 | 1.5 | 0.6 | 1.5 | 1.4 | 1.8 | 2.3 | 1.3 | 0.9 | 1.1 | 1.5 | 0.7 | 2.4 | 1.0 | 1.2 | 0.65 |
| YGL191W | 1.7 | 1.6 | 1.4 | 1.8 | 0.9 | 1.6 | 1.0 | 1.6 | 1.1 | 1.3 | 1.6 | 0.9 | 1.1 | 1.6 | 0.7 | 1.8 | 1.2 | 1.7 | 2.35 |
| YLR038C | 2.3 | 1.1 | 0.6 | 2.1 | 1.8 | 1.2 | 1.0 | 1.4 | 0.4 | 0.9 | 1.4 | 0.6 | 0.5 | 1.6 | 0.7 | 2.1 | 1.2 | 2.1 | 1.52 |
| YHR008C | 1.3 | 5.4 | 4.8 | 1.8 | 0.6 | 1.0 | 0.7 | 0.9 | 1.7 | 2.4 | 2.1 | 0.7 | 1.2 | 1.6 | 1.7 | 2.2 | 0.9 | 1.0 | 1.04 |
| YPR037C | 1.2 | 2.3 | 1.6 | 1.3 | 0.8 | 0.9 | 1.2 | 1.1 | 1.5 | 1.3 | 0.9 | 0.9 | 1.0 | 1.6 | 4.1 | 1.8 | 1.2 | 1.7 | 0.80 |
| YAL039C | 1.1 | 2.2 | 1.7 | 1.0 | 1.1 | 0.8 | 1.0 | 1.2 | 1.6 | 2.2 | 1.1 | 1.4 | 1.6 | 1.2 | 3.3 | 2.2 | 1.4 | 1.1 | 0.39 |
| YDL181W | 1.3 | 1.8 | 1.9 | 1.8 | 1.7 | 1.2 | 1.1 | 0.6 | 0.7 | 0.8 | 0.8 | 0.9 | 0.9 | 1.1 | 0.4 | 1.1 | 1.5 | 1.4 | 0.85 |
| YPL215W | 1.1 | 1.9 | 1.0 | 1.5 | 0.8 | 0.7 | 0.7 | 1.1 | 1.7 | 1.2 | 1.1 | 0.7 | 0.7 | 1.2 | 0.6 | 1.4 | 1.0 | 1.1 | 1.10 |
| YPL262W | 1.0 | 1.7 | 3.8 | 1.2 | 0.6 | 1.6 | 1.3 | 1.0 | 1.4 | 5.8 | 1.4 | 1.1 | 1.5 | 1.2 | 0.6 | 1.4 | 1.1 | 1.2 | 0.79 |
| YNL256W | 0.7 | 1.6 | 0.6 | 0.6 | 1.0 | 0.5 | 0.6 | 0.9 | 0.8 | 0.6 | 0.6 | 0.6 | 1.3 | 0.7 | 0.9 | 0.4 | 0.6 | 0.6 | 1.09 |
| YBL030C | 1.0 | 0.9 | 4.5 | 0.9 | 0.7 | 1.1 | 0.9 | 0.8 | 0.4 | 0.9 | 1.1 | 0.9 | 0.5 | 0.8 | 1.8 | 1.0 | 1.0 | 1.1 | 3.12 |
| YBR221C | 0.8 | 0.7 | 3.2 | 1.5 | 1.3 | 1.7 | 1.5 | 0.8 | 1.0 | 1.3 | 1.2 | 1.3 | 1.7 | 0.8 | 1.1 | 1.5 | 0.9 | 1.0 | 3.62 |
| YKL141W | 1.6 | 1.1 | 4.7 | 1.7 | 1.5 | 1.2 | 0.5 | 0.8 | 0.8 | 0.5 | 1.2 | 0.8 | 0.8 | 1.4 | 0.6 | 2.8 | 0.9 | 1.8 | 2.84 |
| YKR066C | 0.9 | 1.4 | 5.5 | 0.7 | 1.0 | 0.9 | 0.5 | 0.7 | 0.7 | 2.4 | 1.3 | 0.9 | 0.6 | 1.5 | 0.6 | 1.3 | 0.9 | 1.1 | 1.25 |
| YMR083W | 1.4 | 1.7 | 2.6 | 1.1 | 1.5 | 1.8 | 1.7 | 1.1 | 0.4 | 1.1 | 1.6 | 0.7 | 0.9 | 0.8 | 1.3 | 1.3 | 0.9 | 1.1 | 2.52 |
| YMR203W | 0.8 | 0.7 | 3.0 | 1.4 | 0.6 | 1.1 | 0.8 | 0.7 | 0.7 | 1.2 | 2.3 | 0.7 | 1.1 | 0.7 | 1.2 | 0.9 | 0.8 | 0.7 | 1.62 |
| YBL099W | 0.8 | 0.9 | 3.1 | 1.4 | 1.0 | 0.9 | 0.9 | 0.8 | 0.9 | 0.7 | 2.1 | 1.0 | 0.9 | 0.6 | 1.0 | 0.9 | 0.7 | 1.2 | 3.49 |
| YDR178W | 2.0 | 2.0 | 3.4 | 2.2 | 0.9 | 2.1 | 0.6 | 0.9 | 0.9 | 0.8 | 2.0 | 1.3 | 1.5 | 1.5 | 0.7 | 3.0 | 1.2 | 2.3 | 2.27 |
| YDR298C | 1.3 | 1.2 | 2.6 | 1.3 | 1.3 | 1.2 | 1.0 | 1.2 | 1.0 | 1.6 | 1.2 | 1.1 | 1.2 | 1.5 | 0.8 | 2.0 | 1.1 | 1.6 | 2.69 |
| YEL024W | 1.3 | 0.9 | 3.8 | 1.1 | 1.3 | 1.1 | 0.7 | 1.0 | 0.7 | 0.6 | 0.9 | 0.8 | 0.6 | 1.1 | 0.6 | 2.1 | 1.1 | 1.5 | 1.59 |
| YGR082W | 1.0 | 0.9 | 2.2 | 1.0 | 0.9 | 1.1 | 0.9 | 0.8 | 0.6 | 0.9 | 1.3 | 0.8 | 0.5 | 0.9 | 1.2 | 1.3 | 0.9 | 0.8 | 1.47 |
| YJL133W | 1.0 | 1.0 | 2.0 | 0.9 | 0.9 | 0.5 | 0.5 | 0.9 | 0.6 | 1.4 | 1.2 | 0.8 | 0.8 | 0.7 | 0.9 | 0.6 | 0.8 | 0.7 | 0.91 |
| YJR077C | 1.1 | 1.1 | 2.0 | 1.6 | 0.9 | 0.9 | 0.7 | 0.8 | 0.4 | 0.7 | 1.6 | 1.0 | 0.9 | 0.7 | 1.5 | 0.7 | 1.0 | 0.8 | 1.79 |
| YJR121W | 0.9 | 1.1 | 3.3 | 1.0 | 0.8 | 1.5 | 1.0 | 0.7 | 0.8 | 1.3 | 0.9 | 0.8 | 1.0 | 0.7 | 0.9 | 1.2 | 1.1 | 1.4 | 3.99 |
| YKL148C | 0.9 | 0.8 | 3.7 | 0.7 | 0.8 | 0.5 | 0.6 | 0.8 | 1.7 | 1.7 | 1.5 | 0.8 | 0.8 | 0.8 | 0.6 | 1.1 | 0.8 | 1.0 | 0.54 |
| YLL041C | 1.1 | 0.5 | 4.6 | 1.0 | 1.6 | 1.2 | 0.3 | 0.8 | 0.7 | 0.8 | 1.3 | 0.8 | 1.0 | 1.2 | 0.4 | 3.1 | 1.1 | 1.7 | 1.75 |
| YLR304C | 0.7 | 0.6 | 5.0 | 0.7 | 0.6 | 1.9 | 0.6 | 0.6 | 0.1 | 2.2 | 1.6 | 1.0 | 0.5 | 0.7 | 1.6 | 1.8 | 0.5 | 0.7 | 2.39 |
| YOR142W | 1.0 | 1.2 | 3.4 | 1.0 | 1.4 | 1.6 | 1.0 | 0.8 | 1.1 | 1.5 | 1.5 | 0.8 | 1.4 | 0.8 | 1.1 | 0.8 | 0.9 | 1.0 | 1.31 |
| YOR176W | 0.7 | 2.6 | 2.7 | 0.9 | 1.0 | 0.9 | 0.5 | 1.0 | 1.0 | 0.5 | 1.4 | 1.3 | 1.5 | 0.8 | 0.8 | 1.4 | 1.2 | 2.1 | 1.23 |
| YPL135W | 0.9 | 1.2 | 2.5 | 1.2 | 1.6 | 1.3 | 1.2 | 1.0 | 0.5 | 2.8 | 1.3 | 1.1 | 1.2 | 1.1 | 1.5 | 1.2 | 1.5 | 1.7 | 1.50 |
| YDR529C | 1.8 | 1.0 | 0.9 | 3.2 | 1.2 | 0.8 | 0.5 | 1.3 | 0.5 | 0.6 | 0.8 | 0.6 | 0.7 | 1.5 | 0.5 | 1.8 | 1.0 | 2.3 | 3.11 |
| YGL018C | 1.1 | 1.0 | 1.8 | 3.0 | 1.1 | 0.4 | 1.1 | 0.8 | 0.5 | 1.4 | 1.1 | 1.0 | 1.3 | 1.2 | 1.1 | 1.5 | 0.8 | 1.0 | 0.36 |
| YBR003W | 0.9 | 0.8 | 1.0 | 1.9 | 0.7 | 1.2 | 1.1 | 0.9 | 1.0 | 1.3 | 1.3 | 0.8 | 1.2 | 0.8 | 0.7 | 2.0 | 1.1 | 1.3 | 1.06 |
| YBR044C | 0.8 | 1.4 | 0.7 | 1.9 | 1.4 | 1.0 | 1.5 | 1.1 | 0.8 | 1.2 | 1.1 | 0.8 | 1.2 | 0.9 | 0.9 | 1.4 | 0.9 | 1.3 | 0.57 |
| YBR091C | 1.1 | 1.0 | 0.6 | 3.0 | 1.1 | 0.7 | 1.0 | 1.5 | 0.7 | 1.6 | 0.7 | 0.8 | 1.2 | 1.7 | 0.9 | 0.9 | 1.0 | 1.0 | 0.87 |
| YBR185C | 1.2 | 1.0 | 1.3 | 2.0 | 0.8 | 1.2 | 1.2 | 1.1 | 0.9 | 1.6 | 1.2 | 0.7 | 1.0 | 1.7 | 1.0 | 1.7 | 0.8 | 1.3 | 0.89 |
| YBR282W | 1.0 | 1.0 | 0.5 | 2.3 | 1.0 | 1.4 | 1.2 | 1.4 | 1.0 | 0.9 | 0.9 | 0.8 | 1.3 | 1.4 | 1.1 | 1.5 | 0.9 | 1.2 | 1.36 |
| YDR347W | 1.1 | 0.8 | 0.9 | 3.5 | 0.8 | 1.2 | 1.2 | 1.2 | 0.8 | 1.6 | 0.8 | 0.8 | 1.3 | 1.5 | 1.0 | 1.7 | 0.9 | 1.2 | 1.33 |
| YEL039C | 1.1 | 0.7 | 1.3 | 5.1 | 0.9 | 1.5 | 0.6 | 1.1 | 1.2 | 1.2 | 1.7 | 0.6 | 0.5 | 1.4 | 0.4 | 3.5 | 0.7 | 1.1 | 1.59 |
| YGR076C | 1.3 | 1.6 | 0.6 | 2.3 | 0.9 | 1.2 | 1.7 | 1.9 | 1.2 | 1.8 | 1.7 | 1.0 | 1.4 | 2.3 | 0.5 | 2.3 | 0.9 | 1.5 | 0.97 |
| YGR174C | 1.1 | 1.6 | 0.6 | 2.4 | 1.0 | 1.4 | 1.0 | 1.6 | 1.8 | 0.7 | 1.3 | 0.9 | 1.5 | 1.6 | 0.7 | 3.2 | 1.3 | 2.3 | 1.05 |
| YKL003C | 1.1 | 0.7 | 0.8 | 1.8 | 1.0 | 1.0 | 1.4 | 1.2 | 1.5 | 2.2 | 1.7 | 0.7 | 1.3 | 2.0 | 1.0 | 2.3 | 1.0 | 1.2 | 0.90 |
| YKL016C | 1.5 | 1.3 | 0.7 | 1.7 | 1.5 | 0.8 | 1.5 | 1.6 | 1.3 | 1.8 | 1.3 | 1.0 | 1.3 | 1.6 | 0.7 | 1.8 | 1.0 | 1.7 | 2.08 |
| YKL170W | 1.0 | 0.8 | 0.5 | 1.9 | 1.3 | 1.5 | 1.5 | 1.2 | 0.9 | 1.1 | 0.7 | 0.8 | 0.9 | 1.3 | 0.8 | 2.1 | 1.1 | 1.4 | 1.27 |
| YKL194C | 0.9 | 0.7 | 0.8 | 1.6 | 1.8 | 1.4 | 2.1 | 1.1 | 1.3 | 1.4 | 1.6 | 0.6 | 1.1 | 1.4 | 0.8 | 1.2 | 1.1 | 1.1 | 0.52 |
| YLR395C | 1.8 | 1.3 | 2.2 | 1.9 | 0.7 | 0.4 | | 1.0 | 1.0 | 0.9 | 1.5 | 0.6 | 0.5 | 1.7 | 0.4 | 1.2 | 1.2 | 2.3 | 1.76 |
| YML120C | 1.1 | 1.2 | 1.9 | 1.9 | 0.6 | 0.6 | 0.8 | 0.9 | 1.3 | 1.5 | 1.3 | 0.9 | 1.2 | 0.8 | 0.5 | 1.5 | 1.0 | 1.6 | 0.74 |
| YOR100C | 1.3 | 1.4 | 1.6 | 1.7 | 1.0 | 1.2 | 1.6 | 1.0 | 2.0 | 2.6 | 1.4 | 0.8 | 1.0 | 1.4 | 4.3 | 2.8 | 0.9 | 0.8 | 0.32 |
| YOR150W | 1.7 | 1.2 | 1.0 | 1.9 | 0.8 | 0.9 | 1.2 | 1.1 | 0.8 | 1.8 | 1.0 | 0.8 | 1.0 | 1.6 | 0.8 | 1.6 | 0.9 | 1.3 | 1.11 |
| YOR187W | 1.0 | 0.5 | 1.9 | 2.0 | 0.6 | 1.7 | 1.5 | 0.7 | 0.6 | 0.8 | 1.4 | 0.8 | 0.7 | 0.6 | 1.3 | 1.6 | 0.8 | 1.3 | 3.12 |
| YJL166W | 2.1 | 1.5 | 1.6 | 1.3 | 1.6 | 0.7 | 0.8 | 1.4 | 1.3 | 1.2 | 1.4 | 0.7 | 0.8 | 1.9 | 0.5 | 2.3 | 1.7 | 4.0 | 2.31 |
| YMR035W | 1.2 | 1.7 | 1.5 | 1.7 | 0.8 | 1.5 | 2.3 | 1.4 | 1.7 | 1.6 | 1.5 | 0.5 | 1.4 | 1.3 | 1.1 | 1.5 | 2.2 | 2.4 | 0.96 |
| YBL038W | 1.0 | 0.9 | 0.5 | 1.8 | 0.9 | 0.7 | 8.0 | 1.6 | 1.4 | 1.5 | 0.7 | 0.7 | 1.0 | 2.3 | 0.7 | 1.8 | 1.2 | 1.9 | 1.56 |
| YDR377W | 1.3 | 1.1 | 1.3 | 1.4 | 1.9 | 1.3 | 1.1 | 1.3 | 1.0 | 0.9 | 1.4 | 0.9 | 0.7 | 1.4 | 0.6 | 1.8 | 1.2 | 2.5 | 3.00 |
| YGL187C | 1.5 | 0.8 | 1.6 | 1.8 | 1.3 | 1.1 | 0.6 | 0.7 | 0.6 | 0.6 | 0.7 | 0.8 | 0.7 | 0.9 | 0.3 | 1.4 | 0.9 | 2.4 | 2.73 |
| YCR046C | 1.0 | 1.2 | 1.2 | 1.6 | 1.0 | 0.8 | 1.2 | 0.9 | 1.9 | 1.4 | 1.5 | 1.1 | 1.5 | 1.3 | 1.4 | 2.8 | 1.3 | 1.0 | 0.63 |
| YML129C | 1.1 | 0.9 | 0.9 | 1.2 | 1.7 | 1.7 | 1.8 | 1.2 | 1.4 | 2.2 | 1.2 | 0.8 | 1.0 | 1.4 | 1.1 | 2.9 | 1.3 | 1.3 | 1.04 |
| YOL096C | 1.1 | 1.0 | 1.2 | 1.6 | 1.2 | 1.3 | 1.7 | 1.0 | 1.3 | 1.4 | 1.2 | 1.2 | 1.4 | 1.4 | 1.0 | 2.3 | 1.2 | 1.2 | 0.64 |
| YBR122C | 0.9 | 0.9 | 0.3 | 1.1 | 1.5 | 1.1 | 2.0 | 1.3 | 1.1 | 1.4 | 1.2 | 0.7 | 0.8 | 1.2 | 0.7 | 1.9 | 0.9 | 1.5 | 1.53 |
| YBR251W | 1.0 | 0.9 | 1.0 | 1.2 | 1.3 | 2.0 | 1.4 | 1.3 | 0.5 | 1.1 | 1.5 | 1.1 | 1.0 | 1.6 | 0.8 | 3.0 | 0.9 | 1.4 | 1.13 |
| YCR083W | 1.1 | 2.8 | 1.5 | 1.8 | 1.6 | 2.4 | 1.8 | 1.5 | 1.8 | 1.8 | 1.8 | 1.1 | 1.8 | 2.4 | 1.0 | 3.1 | 1.3 | 1.3 | 0.98 |
| YDL067C | 1.8 | 1.0 | 1.6 | 1.3 | 2.0 | 0.9 | 0.8 | 1.1 | 0.9 | 0.9 | 1.4 | 1.0 | 0.6 | 1.4 | 0.7 | 1.8 | 1.2 | 1.7 | 1.85 |
| YDR079W | 1.3 | 1.1 | 0.8 | 1.1 | 1.2 | 1.7 | 1.3 | 2.6 | 0.9 | 0.8 | 1.3 | 1.1 | 1.3 | 1.5 | 0.6 | 2.6 | 1.1 | 1.3 | 1.06 |
| YGR062C | 0.9 | 1.0 | 0.7 | 1.4 | 0.7 | 0.9 | 1.3 | 0.8 | 1.0 | 1.4 | 1.6 | 1.0 | 1.5 | 1.2 | 0.5 | 1.8 | 0.9 | 1.1 | 0.54 |
| YJL096W | 1.0 | 1.0 | 0.8 | 1.3 | 1.9 | 1.0 | 1.7 | 1.3 | 1.0 | 1.4 | 1.2 | 1.0 | 0.7 | 2.1 | 1.0 | 2.2 | 1.1 | 1.2 | 1.21 |
| YJL180C | 0.9 | 1.6 | 1.1 | 1.3 | 1.5 | 1.3 | 1.6 | 1.0 | 1.4 | 1.4 | 1.4 | 0.8 | 1.2 | 1.4 | 0.6 | 2.0 | 0.9 | 1.4 | 1.08 |
| YLR295C | 1.4 | 1.1 | 1.1 | 1.7 | 2.2 | 1.1 | 1.1 | 1.2 | 1.4 | 1.2 | 1.3 | 0.5 | 0.8 | 1.2 | 0.5 | 2.4 | 0.9 | 1.5 | 1.07 |
| YMR023C | 1.4 | 1.3 | 0.5 | 1.9 | 1.7 | 1.1 | 1.9 | 1.4 | 1.5 | 2.6 | 0.8 | 0.7 | 1.1 | 1.6 | 0.7 | 2.1 | 1.1 | 1.4 | 0.48 |
| YMR267W | 0.8 | 1.8 | 0.7 | 0.9 | 1.0 | 1.4 | 1.2 | 1.5 | 0.4 | 0.8 | 1.0 | 0.8 | 0.7 | 1.0 | 0.9 | 2.6 | 0.9 | 1.3 | 0.94 |
| YNL073W | 0.8 | 1.4 | 0.9 | 1.7 | 0.6 | 0.5 | 1.1 | 1.0 | 0.9 | 1.8 | 1.1 | 0.7 | 1.2 | 0.7 | 0.7 | 1.8 | 0.8 | 1.0 | 0.52 |
| YOR316C | 0.7 | 2.5 | 1.5 | 1.5 | 1.0 | 0.9 | 1.4 | 0.8 | 1.6 | 2.0 | 2.4 | 1.1 | 1.6 | 1.0 | 1.5 | 2.2 | 0.8 | 1.0 | 0.93 |
| YPL040C | 1.1 | 1.0 | 0.8 | 1.7 | 0.8 | 1.1 | 0.8 | 1.1 | 0.2 | 1.2 | 0.9 | 0.9 | 1.0 | 1.1 | 1.1 | 2.7 | 0.8 | 1.1 | 0.33 |
| YPL134C | 1.5 | 1.0 | 1.6 | 1.6 | 1.4 | 1.2 | 1.2 | 1.3 | 0.6 | 0.8 | 1.7 | 0.8 | 1.0 | 1.4 | 1.1 | 3.3 | 1.3 | 1.3 | 0.70 |

TABLE 3
DNA repair protein genes

| | The level of expressed mRNA in the presence of chemical substance/The | | | | | | | | | | | | | | | | | | |
|---|---|---|---|---|---|---|---|---|---|---|---|---|---|---|---|---|---|---|---|
| | level of expressed mRNA in the absence of chemical substance | | | | | | | | | | | | | | | | | | |
| yeast genes | (1) | (2) | (3) | (4) | (5) | (6) | (7) | (8) | (9) | (10) | (11) | (12) | (13) | (14) | (15) | (16) | (17) | (18) | Intensity |
| YFL014W | 3.4 | 5.1 | 5.7 | 11.0 | 1.0 | 9.3 | 5.5 | 3.4 | 13.1 | 5.8 | 5.0 | 4.3 | 15.2 | 7.3 | 6.3 | 14.2 | 1.5 | 8.8 | 2.14 |
| YGL163C | 0.9 | 0.8 | 0.6 | 4.3 | 1.5 | 0.9 | 1.0 | 1.4 | 5.8 | 2.3 | 0.4 | 0.9 | 3.1 | 1.6 | 1.8 | 1.0 | 0.9 | 0.8 | 0.26 |
| YKL145W | 0.8 | 1.4 | 1.2 | 1.0 | 1.4 | 2.1 | 1.6 | 1.5 | 3.5 | 2.7 | 1.8 | 1.3 | 2.7 | 1.0 | 1.3 | 1.2 | 1.1 | 1.5 | 2.29 |
| YIL153W | 0.8 | 1.4 | 1.3 | 2.9 | 1.0 | 0.9 | 1.6 | 0.5 | 4.3 | 1.1 | 1.0 | 1.0 | 1.2 | 1.0 | 2.8 | 1.3 | 0.9 | 0.7 | 0.32 |
| YIR002C | 0.8 | 1.1 | 0.5 | 0.6 | 1.2 | 2.2 | 1.3 | 1.2 | 1.7 | 1.4 | 0.8 | 1.3 | 1.2 | 1.1 | 1.1 | 1.4 | 1.1 | 1.2 | 0.58 |
| YAL015C | 1.2 | 1.1 | 1.7 | 1.9 | 1.1 | 0.7 | 1.2 | 1.1 | 4.0 | 2.7 | 1.4 | 1.0 | 1.2 | 1.6 | 0.7 | 2.0 | 1.1 | 1.2 | 0.61 |
| YBR073W | 0.7 | 1.0 | 0.9 | 0.7 | 1.2 | 0.7 | 0.6 | 1.1 | 2.5 | 1.8 | 0.5 | 0.9 | 1.4 | 1.0 | 1.3 | 0.8 | 0.7 | 0.8 | 0.63 |
| YDL200C | 1.4 | 2.0 | 1.2 | 1.8 | 1.1 | 0.6 | 1.7 | 1.5 | 2.4 | 1.8 | 1.2 | 1.1 | 1.1 | 1.8 | 0.7 | 1.8 | 0.9 | 1.6 | 0.79 |
| YGL058W | 1.1 | 1.1 | 0.6 | 0.7 | 1.0 | 1.0 | 0.0 | 1.3 | 3.7 | 1.8 | 0.8 | 1.3 | 1.0 | 1.3 | 1.3 | 0.6 | 1.1 | 0.7 | 1.07 |
| YIL143C | 0.9 | 0.8 | 1.1 | 0.9 | 0.8 | 1.4 | 0.8 | 0.9 | 7.1 | 3.5 | 1.5 | 0.9 | 1.7 | 1.0 | 1.3 | 1.1 | 0.9 | 0.9 | 0.56 |
| YML032C | 0.8 | 0.9 | 1.1 | 0.7 | 1.0 | 0.9 | 0.8 | 0.9 | 3.0 | 1.1 | 1.0 | 0.9 | 1.8 | 1.0 | 1.7 | 1.2 | 1.0 | 1.0 | 0.66 |
| YNL250W | 0.9 | 2.2 | 0.8 | 1.2 | 0.8 | 0.8 | 1.2 | 1.3 | 4.2 | 2.3 | 1.0 | 1.4 | 1.3 | 1.6 | 1.3 | 1.4 | 1.0 | 1.0 | 0.31 |
| YOR386W | 1.0 | 0.8 | 1.3 | 1.5 | 1.2 | 3.3 | 1.8 | 1.0 | 4.1 | 3.0 | 1.4 | 1.2 | 2.4 | 0.9 | 1.3 | 1.2 | 2.1 | 1.7 | 0.46 |
| YBL019W | 0.9 | 9.1 | 0.7 | 1.2 | 1.0 | 1.0 | 1.2 | 1.2 | 2.6 | 1.4 | 1.0 | 0.8 | 0.9 | 1.1 | 0.6 | 1.9 | 0.8 | 1.1 | 0.38 |
| YDR369C | 1.1 | 1.1 | 2.4 | 1.1 | 1.0 | 0.6 | 0.4 | 1.0 | 2.4 | 1.8 | 1.1 | 0.9 | 0.1 | 0.7 | 1.2 | 0.6 | 0.8 | 0.7 | 1.88 |
| YEL037C | 0.9 | 2.0 | 0.9 | 1.0 | 0.5 | 0.6 | 0.8 | 0.8 | 2.6 | 1.2 | 1.0 | 1.1 | 2.0 | 1.0 | 1.6 | 0.5 | 1.0 | 0.8 | 0.82 |
| YER162C | 1.0 | 1.2 | 1.0 | 1.1 | 0.9 | 0.7 | 0.5 | 0.9 | 2.8 | 1.6 | 0.5 | 0.4 | 0.8 | 0.8 | 1.0 | 1.0 | 1.2 | 0.9 | 0.45 |
| YGR258C | 0.7 | 0.9 | 0.7 | 0.7 | 1.5 | 2.2 | 1.5 | 0.8 | 2.5 | 1.5 | 0.3 | 0.9 | 1.9 | 1.2 | 1.1 | 1.2 | 1.0 | 0.9 | 0.37 |
| YJR052W | 1.1 | 1.0 | 1.1 | 1.0 | 1.0 | 1.4 | 1.2 | 1.7 | 4.5 | 2.3 | 1.1 | 1.1 | 1.8 | 1.4 | 1.4 | 1.4 | 1.0 | 1.3 | 0.36 |
| YOR005C | 1.2 | 1.8 | 1.0 | 0.8 | 1.3 | 1.3 | 1.4 | 1.3 | 2.4 | 1.5 | 1.2 | 0.9 | 2.1 | 1.2 | 0.8 | 1.4 | 1.1 | 1.0 | 0.29 |
| YPL022W | 0.7 | 0.8 | 0.7 | 1.2 | 0.8 | 5.2 | 0.8 | 1.0 | 2.1 | 1.6 | 0.8 | 0.7 | 1.4 | 0.9 | 0.9 | 1.0 | 0.9 | 0.9 | 0.72 |
| YPL164C | 1.1 | 0.7 | 1.1 | 1.0 | 1.2 | 1.7 | 0.9 | 1.0 | 2.1 | 2.0 | 1.1 | 0.9 | 1.0 | 0.9 | 1.3 | 1.4 | 1.0 | 1.0 | 0.25 |
| YPL194W | 0.9 | 1.4 | 0.4 | 1.1 | 1.3 | 1.1 | 1.5 | 1.3 | 9.6 | 3.1 | 0.9 | 0.9 | 1.4 | 1.1 | 1.4 | 0.9 | 1.0 | 0.8 | 0.22 |
| YPR025C | 1.1 | 0.8 | 0.6 | 0.6 | 1.6 | 0.9 | 1.4 | 1.3 | 1.9 | 1.7 | 0.9 | 0.7 | 1.1 | 1.3 | 1.1 | 1.3 | 1.1 | 1.5 | 0.87 |
| YGR180C | 3.1 | 1.1 | 1.9 | 1.0 | 2.0 | 0.6 | 0.5 | 1.0 | 1.4 | 1.5 | 1.0 | 0.9 | 0.9 | 0.9 | 1.3 | 1.2 | 1.0 | 1.9 | 3.90 |
| YEL019C | 1.1 | 1.6 | 0.3 | 0.7 | 0.9 | 1.5 | 1.2 | 0.9 | 0.7 | 0.7 | 1.2 | 1.0 | 1.0 | 1.7 | 1.5 | 0.3 | 0.8 | 0.9 | 0.27 |
| YLR288C | 1.2 | 0.8 | 1.6 | 1.8 | 1.7 | 1.2 | 1.3 | 1.5 | 0.8 | 1.3 | 1.4 | 0.6 | 0.7 | 1.2 | 0.9 | 1.5 | 1.2 | 1.3 | 0.31 |
| YMR284W | 1.1 | 1.0 | 0.5 | 2.0 | 0.8 | 0.8 | 0.8 | 1.1 | 1.6 | 1.8 | 1.1 | 0.6 | 1.0 | 1.2 | 0.7 | 1.3 | 1.1 | 1.3 | 0.54 |
| YMR035W | 1.2 | 1.7 | 1.5 | 1.7 | 0.8 | 1.5 | 2.3 | 1.4 | 1.7 | 1.6 | 1.5 | 0.5 | 1.4 | 1.3 | 1.1 | 1.5 | 2.2 | 2.4 | 0.96 |
| YOL043C | 1.1 | 0.9 | 0.8 | 1.7 | 1.2 | 0.8 | 1.0 | 0.9 | 1.0 | 1.8 | 1.2 | 0.8 | 0.9 | 1.5 | 0.6 | 2.5 | 1.0 | 1.0 | 0.38 |
| YGR231C | 1.0 | 1.2 | 0.7 | 1.0 | 1.3 | 1.9 | 2.1 | 1.1 | 3.2 | 2.3 | 1.6 | 1.3 | 3.9 | 2.2 | 1.3 | 2.5 | 1.0 | 1.5 | 1.74 |
| YHR164C | 0.9 | 1.3 | 0.9 | 0.7 | 0.9 | 1.4 | 0.8 | 1.1 | 1.3 | 3.1 | 0.6 | 1.2 | 2.0 | 1.3 | 2.2 | 1.1 | 0.8 | 1.0 | 0.44 |
| YJR046W | 1.0 | 1.2 | 1.2 | 1.5 | 0.8 | 2.0 | 0.9 | 0.9 | 6.0 | 2.4 | 0.6 | 0.9 | 3.0 | 1.0 | 1.1 | 0.8 | 0.7 | 0.7 | 0.45 |
| YLR103C | 0.8 | 1.5 | 1.1 | 0.4 | 1.0 | 1.1 | 1.3 | 1.4 | 0.8 | 0.5 | 0.9 | 1.6 | 3.0 | 1.0 | 0.8 | 1.5 | 1.1 | 1.4 | 1.32 |
| YMR072W | 1.1 | 1.0 | 1.4 | 1.0 | 1.7 | 1.6 | 1.4 | 1.0 | 3.0 | 1.2 | 1.9 | 0.8 | 2.1 | 1.2 | 0.8 | 1.7 | 0.9 | 1.9 | 2.06 |
| YDR054C | 1.3 | 1.0 | 0.7 | 1.4 | 0.7 | 1.9 | 1.1 | 1.5 | 2.9 | 3.2 | 1.9 | 1.0 | 2.5 | 1.1 | 1.6 | 1.4 | 0.8 | 1.4 | 1.18 |
| YAR007C | 0.7 | 1.9 | 0.8 | 0.5 | 1.5 | 1.6 | 0.8 | 1.1 | 4.1 | 1.7 | 0.9 | 0.9 | 1.7 | 1.4 | 1.2 | 0.9 | 0.8 | 1.0 | 1.03 |
| YGL058W | 1.1 | 1.1 | 0.6 | 0.7 | 1.0 | 1.5 | 0.0 | 1.3 | 3.7 | 1.8 | 0.8 | 1.3 | 1.0 | 1.3 | 1.3 | 0.6 | 1.1 | 0.7 | 1.07 |
| YIL036W | 0.9 | 1.3 | 1.3 | 1.8 | 0.9 | 1.8 | 1.4 | 1.1 | 3.6 | 2.7 | 1.2 | 1.2 | 1.9 | 1.1 | 0.8 | 1.3 | 1.1 | 1.3 | 0.66 |
| YNL213C | 1.7 | 11.6 | 0.9 | 1.5 | 1.3 | 1.7 | 1.4 | 1.8 | 3.0 | 1.5 | 1.5 | 0.8 | 0.9 | 1.8 | 1.0 | 1.7 | 0.9 | 1.2 | 0.70 |
| YNL312W | 0.9 | 1.0 | 1.6 | 0.7 | 1.4 | 1.7 | 0.5 | 1.2 | 3.7 | 2.5 | 1.4 | 0.9 | 0.7 | 1.1 | 0.8 | 0.8 | 1.2 | 1.1 | 2.05 |
| YNL261W | 1.0 | 1.3 | 0.6 | 1.3 | 0.7 | 1.3 | 1.0 | 1.1 | 2.3 | 1.5 | 0.8 | 0.8 | 0.8 | 1.4 | 0.7 | 1.1 | 0.8 | 1.0 | 0.91 |
| YGR180C | 3.1 | 1.1 | 1.9 | 1.0 | 2.0 | 1.0 | 0.5 | 1.0 | 1.4 | 1.5 | 1.0 | 0.9 | 0.9 | 0.9 | 1.3 | 1.2 | 1.0 | 1.9 | 3.90 |
| YJL026W | 2.5 | 2.4 | 2.9 | 1.0 | 1.7 | 1.0 | 0.5 | 1.2 | 1.0 | 1.1 | 1.3 | 1.0 | 1.2 | 1.0 | 1.1 | 1.6 | 1.4 | 2.1 | 3.74 |
| YDL017W | 0.8 | 2.3 | 1.2 | 1.3 | 1.2 | 1.1 | 1.1 | 1.2 | 0.8 | 1.8 | 0.8 | 0.8 | 0.9 | 1.3 | 1.3 | 1.0 | 0.8 | 1.0 | 0.38 |
| YML058W | 1.9 | 1.2 | 5.8 | 1.9 | 0.6 | 1.3 | 0.6 | 0.7 | 1.1 | 1.2 | 3.3 | 0.9 | 1.4 | 1.1 | 2.5 | 1.3 | 1.3 | 1.5 | 2.14 |
| YLR233C | 1.1 | 0.9 | 2.0 | 2.0 | 1.2 | 0.8 | 0.8 | 1.1 | 0.6 | 0.5 | 0.8 | 0.9 | 1.0 | 1.1 | 1.1 | 1.1 | 1.0 | 1.2 | 0.43 |
| YMR284W | 1.1 | 1.0 | 0.5 | 2.0 | 0.8 | 1.2 | 0.8 | 1.1 | 1.6 | 1.8 | 1.1 | 0.6 | 1.0 | 1.2 | 0.7 | 1.3 | 1.1 | 1.3 | 0.54 |
| YGL163C | 0.9 | 0.8 | 0.6 | 4.3 | 1.5 | 0.9 | 1.0 | 1.4 | 5.8 | 2.3 | 0.4 | 0.9 | 3.1 | 1.6 | 1.8 | 1.0 | 0.9 | 0.8 | 0.26 |
| YGL127C | 1.0 | 1.9 | 1.3 | 1.7 | 1.3 | 0.6 | 1.0 | 0.9 | 1.2 | 1.5 | 1.0 | 1.4 | 1.7 | 1.0 | 1.5 | 1.3 | 1.0 | 1.2 | 0.54 |
| YMR072W | 1.1 | 1.0 | 1.4 | 1.0 | 1.7 | 2.4 | 1.4 | 1.0 | 3.0 | 1.2 | 1.9 | 0.8 | 2.1 | 1.2 | 0.8 | 1.7 | 0.9 | 1.9 | 2.06 |
| YGL249W | 1.4 | 0.8 | 0.5 | 0.8 | 1.1 | 2.5 | 1.2 | 0.9 | 0.3 | 2.4 | 0.6 | 0.8 | 0.8 | 1.9 | 0.7 | 1.4 | 1.0 | 1.0 | 0.20 |
| YBR272C | 0.8 | 1.8 | 1.3 | 1.0 | 1.1 | | 1.9 | 1.1 | 1.7 | 2.7 | 1.1 | 0.5 | 1.3 | 1.0 | 0.5 | 1.7 | 1.1 | 1.2 | 0.52 |
| YDL059C | 2.6 | 5.1 | 1.4 | 1.7 | 1.2 | 1.2 | 1.1 | 1.2 | 10.5 | 7.8 | 2.0 | 1.3 | 1.7 | 4.7 | 0.7 | 1.5 | 0.8 | 0.9 | 0.63 |
| YAR007C | 0.7 | 1.9 | 0.8 | 0.5 | 1.5 | 0.6 | 0.8 | 1.1 | 4.1 | 1.7 | 0.9 | 0.9 | 1.7 | 1.4 | 1.2 | 0.9 | 0.8 | 1.0 | 1.03 |
| YBR073W | 0.7 | 1.0 | 0.9 | 0.7 | 1.2 | 0.7 | 0.6 | 1.1 | 2.5 | 1.8 | 0.5 | 0.9 | 1.4 | 1.0 | 1.3 | 0.8 | 0.7 | 0.8 | 0.63 |
| YML032C | 0.8 | 0.9 | 1.1 | 0.7 | 1.0 | 0.9 | 0.8 | 0.9 | 3.0 | 1.1 | 1.0 | 0.9 | 1.8 | 1.0 | 1.7 | 1.2 | 1.0 | 1.0 | 0.66 |
| YNL250W | 0.9 | 2.2 | 0.8 | 1.2 | 0.8 | 0.8 | 1.2 | 1.3 | 4.2 | 2.3 | 1.0 | 1.4 | 1.3 | 1.6 | 1.3 | 1.4 | 1.0 | 1.0 | 0.31 |
| YCR014C | 0.8 | 1.0 | 0.8 | 0.6 | 1.2 | 1.1 | 1.1 | 1.0 | 2.4 | 1.0 | 0.8 | 0.7 | 1.2 | 1.0 | 0.7 | 1.5 | 1.0 | 0.9 | 0.34 |
| YDR369C | 1.1 | 1.1 | 2.4 | 1.1 | 1.0 | 0.6 | 0.4 | 1.0 | 2.4 | 1.8 | 1.1 | 0.9 | 0.1 | 0.7 | 1.2 | 0.6 | 0.8 | 0.7 | 1.88 |
| YIL072W | 0.8 | 3.1 | 1.1 | 2.6 | 1.5 | 1.2 | 1.7 | 1.4 | 2.9 | 2.1 | 1.8 | 1.1 | 1.4 | 2.0 | 1.4 | 1.4 | 1.0 | 1.1 | 0.23 |
| YOR005C | 1.2 | 1.8 | 1.0 | 0.8 | 1.3 | 1.3 | 1.4 | 1.3 | 2.4 | 1.5 | 1.2 | 0.9 | 2.1 | 1.2 | 0.8 | 1.4 | 1.1 | 1.0 | 0.29 |
| YPL164C | 1.1 | 0.7 | 1.1 | 1.0 | 1.2 | 1.7 | 0.9 | 1.0 | 2.1 | 2.0 | 1.1 | 0.9 | 1.0 | 0.9 | 1.3 | 1.4 | 1.0 | 1.0 | 0.25 |
| YPL194W | 0.9 | 1.4 | 0.4 | 1.1 | 1.3 | 1.1 | 1.5 | 1.3 | 9.6 | 3.1 | 0.9 | 0.9 | 1.4 | 1.1 | 1.4 | 0.9 | 1.0 | 0.8 | 0.22 |
| YGR180C | 3.1 | 1.1 | 1.9 | 1.0 | 2.0 | 0.6 | 0.5 | 1.0 | 1.4 | 1.5 | 1.0 | 0.9 | 0.9 | 0.9 | 1.3 | 1.2 | 1.0 | 1.9 | 3.90 |
| YEL019C | 1.1 | 1.6 | 0.3 | 0.7 | 0.9 | 1.5 | 1.2 | 0.9 | 0.7 | 0.7 | 1.2 | 1.0 | 1.0 | 1.7 | 1.5 | 0.3 | 0.8 | 0.9 | 0.27 |
| YML058W | 1.9 | 1.2 | 5.8 | 1.9 | 0.6 | 0.6 | 0.6 | 0.7 | 1.1 | 1.2 | 3.3 | 0.9 | 1.4 | 1.1 | 2.5 | 1.3 | 1.3 | 1.5 | 2.14 |
| YLR288C | 1.2 | 0.8 | 1.6 | 1.8 | 1.7 | 1.2 | 1.3 | 1.5 | 0.8 | 1.3 | 1.4 | 0.6 | 0.7 | 1.2 | 0.9 | 1.5 | 1.2 | 1.3 | 0.31 |
| YMR284W | 1.1 | 1.0 | 0.5 | 2.0 | 0.8 | 0.8 | 0.8 | 1.1 | 1.6 | 1.8 | 1.1 | 0.6 | 1.0 | 1.2 | 0.7 | 1.3 | 1.1 | 1.3 | 0.54 |
| YMR096W | 1.7 | 1.7 | 1.1 | 0.8 | 1.0 | 2.2 | 1.9 | 2.0 | 3.3 | 3.7 | 1.5 | 1.4 | 8.4 | 4.7 | 28.9 | 2.2 | 2.2 | 1.0 | 0.62 |
| YGL091C | 2.0 | 1.8 | 1.0 | 1.7 | 0.8 | 1.6 | 1.3 | 1.8 | 10.8 | 6.0 | 2.1 | 1.2 | 2.5 | 3.1 | 0.7 | 1.0 | 1.4 | 1.8 | 0.96 |

TABLE 4
Energy system protein genes

| | The level of expressed mRNA in the presence of chemical substance/The | | | | | | | | | | | | | | | | | | |
|---|---|---|---|---|---|---|---|---|---|---|---|---|---|---|---|---|---|---|---|
| | level of expressed mRNA in the absence of chemical substance | | | | | | | | | | | | | | | | | | |
| yeast genes | (1) | (2) | (3) | (4) | (5) | (6) | (7) | (8) | (9) | (10) | (11) | (12) | (13) | (14) | (15) | (16) | (17) | (18) | Intensity |
| YCR107W | 12.1 | 2.6 | 1.8 | 1.6 | 0.7 | 1.5 | 1.5 | 15.6 | 196.6 | 34.0 | 23.0 | 0.9 | 4.0 | 8.3 | 4.8 | 3.1 | 1.3 | 1.1 | 0.59 |
| YDL243C | 14.5 | 2.7 | 2.4 | 1.0 | 1.1 | 2.2 | 1.6 | 11.8 | 64.2 | 29.6 | 19.6 | 1.1 | 4.1 | 12.0 | 4.2 | 3.0 | 1.8 | 1.5 | 0.76 |
| YFL056C | 19.0 | 2.3 | 2.3 | 1.5 | 0.9 | 0.6 | 1.4 | 18.5 | 162.3 | 31.3 | 68.3 | 1.0 | 4.7 | 7.8 | 5.0 | 3.4 | 1.0 | 1.1 | 0.55 |
| YFL057C | 20.9 | 5.9 | 1.5 | 1.8 | 0.9 | 1.2 | 1.8 | 18.0 | 51.8 | 46.1 | 27.7 | 1.0 | 4.1 | 23.4 | 3.1 | 3.9 | 1.6 | 1.3 | 0.71 |
| YJR155W | 7.6 | 3.7 | 1.4 | 2.5 | 0.7 | 1.4 | 1.7 | 10.6 | 38.2 | 18.8 | 15.4 | 1.0 | 5.7 | 9.4 | 2.6 | 5.6 | 1.3 | 1.4 | 0.64 |
| YNL331C | 8.6 | 3.6 | 1.3 | 1.0 | 1.6 | 1.8 | 1.9 | 13.1 | 42.6 | 36.3 | 21.8 | 0.9 | 3.1 | 7.5 | 2.3 | 4.0 | 1.7 | 1.3 | 0.58 |
| YOL165C | 10.1 | 4.5 | 1.8 | 0.9 | 0.9 | 1.7 | 1.4 | 17.8 | 46.9 | 23.3 | 17.6 | 0.8 | 3.7 | 9.1 | 3.0 | 1.8 | 1.6 | 1.0 | 0.69 |
| YPL171C | 15.2 | 4.1 | 3.5 | 2.2 | 1.1 | 1.3 | 1.7 | 20.5 | 60.0 | 50.6 | 37.0 | 1.4 | 2.4 | 9.3 | 1.4 | 1.2 | 2.4 | 1.5 | 0.47 |
| YDL021W | 5.1 | 1.7 | 2.4 | 3.7 | 1.7 | 6.5 | 5.9 | 2.7 | 2.5 | 7.4 | 4.7 | 2.4 | 5.3 | 3.7 | 0.7 | 7.3 | 1.9 | 3.2 | 0.47 |
| YGR043C | 2.6 | 3.7 | 3.2 | 7.9 | 0.9 | 16.3 | 6.5 | 2.6 | 10.9 | 8.4 | 3.6 | 3.3 | 6.9 | 4.1 | 3.0 | 13.7 | 1.6 | 4.8 | 0.66 |
| YHR179W | 3.3 | 2.3 | 2.6 | 0.7 | 1.2 | 3.7 | 1.9 | 3.3 | 3.6 | 5.8 | 2.0 | 2.2 | 2.6 | 5.8 | 2.7 | 1.0 | 1.9 | 1.6 | 2.69 |
| YJR048W | 1.3 | 0.9 | 2.2 | 1.4 | 0.8 | 0.5 | 0.3 | 0.9 | 0.5 | 1.5 | 1.0 | 0.8 | 0.4 | 2.5 | 0.6 | 1.5 | 1.2 | 1.2 | 1.19 |
| YKR097W | 1.6 | 2.4 | 1.3 | 3.3 | 1.1 | 1.7 | 3.7 | 2.5 | 1.9 | 3.3 | 0.8 | 1.8 | 17.5 | 2.1 | 2.5 | 2.2 | 1.1 | 1.5 | 0.16 |
| YML087C | 1.8 | 1.6 | 0.8 | 2.5 | 0.9 | 0.9 | 1.1 | 1.3 | 1.9 | 4.9 | 1.6 | 0.8 | 1.5 | 2.1 | 0.3 | 2.0 | 0.7 | 0.8 | 0.52 |
| YPL088W | 3.3 | 1.3 | 0.6 | 2.5 | 1.5 | 7.1 | 3.7 | 1.9 | 0.6 | 2.8 | 1.1 | 3.6 | 4.8 | 1.6 | 3.2 | 4.3 | 9.1 | 8.3 | 0.69 |
| YDL174C | 0.9 | 1.8 | 0.9 | 1.3 | 0.5 | 1.7 | 3.6 | 0.8 | 0.5 | 1.7 | 0.9 | 2.8 | 5.9 | 1.1 | 0.6 | 5.1 | 2.2 | 4.0 | 0.63 |
| YCR012W | 1.2 | 1.5 | 5.1 | 1.2 | 1.1 | 2.5 | 1.4 | 0.8 | 1.4 | 1.5 | 1.8 | 1.2 | 4.1 | 0.9 | 1.4 | 1.4 | 0.8 | 1.3 | 4.48 |
| YFR053C | 1.8 | 1.4 | 3.0 | 2.2 | 0.9 | 3.2 | 2.1 | 2.8 | 0.6 | 1.1 | 1.5 | 1.7 | 3.0 | 0.6 | 0.6 | 3.0 | 1.4 | 2.2 | 4.17 |
| YGL062W | 0.6 | 1.3 | 1.1 | 0.6 | 0.9 | 1.0 | 1.3 | 0.8 | 1.6 | 2.5 | 0.7 | 1.6 | 4.5 | 1.2 | 2.7 | 1.5 | 1.1 | 0.9 | 0.77 |
| YGR192C | 1.4 | 1.0 | 3.8 | 1.0 | 1.1 | 1.7 | 1.7 | 1.1 | 0.9 | 1.3 | 2.3 | 2.2 | 3.4 | 1.0 | 1.9 | 1.1 | 1.1 | 1.3 | 7.49 |
| YGR244C | 1.0 | 1.2 | 1.6 | 1.6 | 0.8 | 2.6 | 3.9 | 1.8 | 1.4 | 1.1 | 1.8 | 1.9 | 2.6 | 1.2 | 2.0 | 2.6 | 1.9 | 3.1 | 1.12 |
| YGR254W | 1.2 | 1.3 | 3.8 | 1.3 | 1.2 | 1.9 | 1.5 | 1.4 | 0.8 | 1.2 | 1.3 | 1.7 | 3.1 | 0.6 | 2.4 | 1.4 | 1.3 | 1.2 | 7.01 |
| YIL160C | 0.8 | 2.4 | 2.3 | 3.5 | 1.0 | 1.6 | 1.1 | 1.2 | 3.2 | 3.8 | 2.4 | 1.0 | 2.3 | 1.5 | 5.6 | 8.8 | 2.4 | 3.0 | 0.27 |
| YJL052W | 1.6 | 0.9 | 4.0 | 1.8 | 0.7 | 2.4 | 2.1 | 1.5 | 1.6 | 2.0 | 4.3 | 2.4 | 5.6 | 1.3 | 2.3 | 2.3 | 1.2 | 1.9 | 6.19 |
| YJR009C | 1.1 | 1.7 | 5.6 | 1.0 | 1.2 | 1.6 | 1.6 | 1.1 | 1.1 | 1.3 | 1.1 | 1.5 | 2.7 | 1.0 | 2.1 | 1.5 | 1.1 | 1.3 | 5.86 |
| YLR345W | 1.4 | 1.4 | 1.4 | 1.2 | 1.2 | 1.6 | 1.2 | 1.4 | 4.5 | 4.0 | 2.5 | 1.6 | 3.5 | 1.5 | 1.0 | 1.2 | 1.2 | 1.4 | 2.65 |
| YMR118C | 1.3 | 2.9 | 1.4 | 1.6 | 1.2 | 2.0 | 1.0 | 1.0 | 9.3 | 1.4 | 0.7 | 0.8 | 4.2 | 0.9 | 0.9 | 8.1 | 0.8 | 1.0 | 0.38 |
| YNL071W | 0.9 | 0.9 | 1.7 | 1.5 | 0.6 | 1.3 | 1.4 | 0.9 | 1.1 | 1.2 | 1.6 | 1.3 | 2.5 | 0.8 | 1.1 | 1.1 | 1.0 | 1.2 | 1.61 |
| YNL241C | 1.3 | 2.5 | 4.3 | 1.0 | 0.8 | 0.9 | 3.2 | 0.9 | 3.4 | 7.4 | 3.0 | 2.0 | 4.9 | 1.1 | 7.0 | 2.8 | 1.0 | 1.0 | 0.68 |
| YPL240C | 0.7 | 1.4 | 2.4 | 0.9 | 1.3 | 1.2 | 1.4 | 1.2 | 3.5 | 2.6 | 1.0 | 0.8 | 2.9 | 0.7 | 1.2 | 0.7 | 0.8 | 1.0 | 4.83 |
| YAL060W | 1.1 | 1.8 | 3.2 | 2.7 | 1.2 | 4.2 | 3.3 | 0.9 | 0.6 | 2.5 | 2.4 | 0.8 | 2.2 | 0.9 | 0.9 | 3.2 | 1.1 | 1.8 | 2.39 |
| YCL040W | 0.9 | 5.6 | 10.1 | 2.0 | 0.5 | 3.5 | 2.9 | 0.7 | 0.9 | 3.0 | 8.2 | 2.3 | 5.6 | 0.7 | 3.4 | 3.1 | 1.4 | 1.7 | 1.98 |
| YDR001C | 1.0 | 2.2 | 2.6 | 1.0 | 1.1 | 2.2 | 1.5 | 0.9 | 2.2 | 3.0 | 0.7 | 1.1 | 2.8 | 1.0 | 4.4 | 1.9 | 1.1 | 1.6 | 0.75 |
| YDR231C | 1.1 | 1.6 | 1.0 | 1.5 | 1.1 | 2.2 | 1.7 | 1.3 | 1.5 | 1.4 | 1.3 | 1.1 | 2.4 | 1.5 | 1.0 | 3.0 | 1.2 | 1.3 | 0.92 |
| YER178W | 0.8 | 0.9 | 3.6 | 1.0 | 0.7 | 2.5 | 1.6 | 0.8 | 1.7 | 1.3 | 2.5 | 1.3 | 2.4 | 0.9 | 1.8 | 1.0 | 1.1 | 0.9 | 2.18 |
| YGR008C | 2.4 | 3.0 | 1.7 | 3.2 | 0.9 | 2.9 | 3.7 | 1.9 | 3.1 | 2.4 | 2.6 | 2.0 | 3.4 | 1.3 | 1.5 | 2.3 | 1.7 | 4.2 | 3.03 |
| YGR256W | 1.2 | 1.5 | 2.3 | 0.8 | 0.9 | 6.2 | 1.4 | 2.2 | 3.4 | 2.5 | 2.7 | 1.5 | 3.9 | 1.1 | 2.7 | 5.3 | 1.1 | 0.8 | 0.94 |
| YHL008C | 0.9 | 0.5 | 1.0 | 1.6 | 0.8 | 0.8 | 0.7 | 0.9 | 1.6 | 1.7 | 1.4 | 0.8 | 1.8 | 1.0 | 1.8 | 0.9 | 0.8 | 0.9 | 0.40 |
| YHR174W | 1.1 | 1.4 | 3.3 | 1.2 | 1.3 | 1.5 | 1.6 | 1.2 | 1.0 | 1.5 | 1.4 | 1.5 | 3.6 | 0.6 | 2.0 | 1.1 | 1.0 | 1.2 | 7.34 |
| YIL045W | 1.7 | 1.4 | 1.9 | 2.2 | 1.3 | 1.7 | 1.6 | 1.1 | 2.1 | 3.2 | 1.2 | 1.5 | 2.0 | 1.6 | 0.6 | 2.9 | 1.1 | 1.8 | 0.37 |
| YKL035W | 1.0 | 0.9 | 4.8 | 1.2 | 0.8 | 1.2 | 0.6 | 1.0 | 0.8 | 1.2 | 2.1 | 1.0 | 2.0 | 0.6 | 1.0 | 1.5 | 1.0 | 1.9 | 2.54 |
| YKL152C | 1.4 | 1.3 | 1.9 | 0.9 | 1.3 | 1.6 | 1.5 | 1.0 | 0.9 | 1.5 | 1.7 | 1.5 | 2.7 | 1.0 | 1.8 | 2.0 | 1.1 | 1.7 | 3.28 |
| YML054C | 1.5 | 1.8 | 1.3 | 3.4 | 1.3 | 1.8 | 1.2 | 1.5 | 4.1 | 1.8 | 1.4 | 1.8 | 2.8 | 2.1 | 1.1 | 7.8 | 1.1 | 1.6 | 0.25 |
| YML100W | 0.8 | 2.7 | 10.6 | 1.4 | 0.9 | 2.8 | 2.2 | 0.7 | 1.7 | 1.4 | 3.2 | 1.2 | 3.8 | 1.2 | 1.8 | 2.2 | 1.0 | 1.5 | 0.88 |
| YML125C | 0.8 | 0.9 | 0.7 | 0.7 | 0.9 | 1.3 | 1.0 | 1.3 | 2.6 | 1.6 | 1.0 | 1.0 | 2.2 | 1.6 | 1.9 | 0.7 | 1.2 | 0.9 | 2.21 |
| YMR089C | 1.0 | 1.1 | 0.9 | 0.9 | 0.8 | 1.2 | 1.2 | 1.3 | 5.6 | 2.5 | 1.3 | 0.8 | 2.3 | 0.9 | 1.8 | 1.1 | 0.9 | 1.1 | 0.69 |
| YMR105C | 2.0 | 3.0 | 5.0 | 4.2 | 0.9 | 2.8 | 2.8 | 1.1 | 0.6 | 2.9 | 3.0 | 1.7 | 3.3 | 1.0 | 0.9 | 2.0 | 1.6 | 2.6 | 1.21 |
| YNL237W | 1.3 | 1.3 | 1.5 | 1.0 | 1.5 | 4.6 | 4.9 | 1.1 | 0.9 | 2.1 | 1.5 | 1.8 | 3.2 | 1.3 | 1.6 | 4.4 | 1.3 | 1.1 | 0.21 |
| YOL126C | 1.1 | 0.8 | 1.0 | 1.6 | 0.7 | 1.5 | 1.4 | 0.9 | 0.8 | 2.4 | 1.8 | 1.1 | 2.6 | 1.2 | 1.4 | 2.5 | 1.6 | 2.4 | 0.59 |
| YOR347C | 0.9 | 0.9 | 2.1 | 2.2 | 0.7 | 0.9 | 0.9 | 0.8 | 0.5 | 0.9 | 1.4 | 1.2 | 1.8 | 0.9 | 2.0 | 1.4 | 1.7 | 1.3 | 1.31 |
| YPR026W | 0.9 | 1.2 | 5.0 | 1.3 | 0.8 | | 1.3 | 1.0 | 0.9 | 3.1 | 1.6 | 1.2 | 2.3 | 1.5 | 3.1 | 2.5 | 0.9 | 1.3 | 0.26 |
| YAL038W | 1.0 | 1.0 | 3.0 | 1.4 | 1.4 | 1.3 | 0.9 | 1.1 | 0.1 | 1.1 | 1.0 | 1.2 | 1.8 | 0.5 | 3.1 | 0.9 | 1.0 | 1.0 | 7.02 |
| YDR380W | 0.8 | 0.8 | 0.8 | 1.7 | 1.1 | 0.4 | 0.5 | 0.2 | 0.2 | 0.3 | 0.4 | 0.4 | 0.4 | 0.6 | 2.4 | 0.2 | 0.8 | 0.8 | 1.04 |
| YLR273C | 1.5 | 1.0 | 1.3 | 2.4 | | 1.2 | 1.2 | 1.3 | 2.8 | 2.4 | 1.4 | 0.9 | 1.7 | 1.3 | 2.6 | 1.5 | 1.0 | 1.0 | 0.20 |
| YGR207C | 1.5 | 1.1 | 1.0 | 1.1 | 1.5 | 2.8 | 1.7 | 1.9 | 2.1 | 2.6 | 1.4 | 1.1 | 1.0 | 2.2 | 0.6 | 1.8 | 1.2 | 1.8 | 1.49 |
| YNL037C | 1.4 | 1.8 | 1.7 | 1.1 | 1.7 | 2.8 | 2.8 | 1.0 | 0.6 | 3.2 | 1.4 | 1.3 | 1.9 | 1.3 | 0.9 | 1.4 | 0.9 | 1.3 | 2.05 |
| YOR136W | 1.0 | 0.9 | 3.5 | 1.0 | 1.5 | 3.9 | 3.2 | 1.1 | 0.3 | 3.4 | 1.4 | 1.2 | 1.5 | 0.9 | 1.2 | 1.3 | 0.8 | 1.3 | 3.20 |
| YPL271W | 1.2 | 3.2 | 4.1 | 1.2 | 1.4 | 3.3 | 1.2 | 0.9 | 0.6 | 1.2 | 1.7 | 0.8 | 1.2 | 1.3 | 0.8 | 1.4 | 1.2 | 1.3 | 1.34 |
| YAL054C | 1.2 | 1.1 | 1.4 | 1.6 | 1.1 | 2.2 | 1.1 | 1.1 | 8.9 | 4.1 | 1.3 | 1.0 | 1.8 | 1.3 | 0.9 | 2.5 | 1.5 | 1.4 | 0.24 |
| YEL011W | 2.0 | 1.6 | 1.7 | 6.3 | 1.1 | 2.8 | 2.7 | 0.9 | 2.5 | 1.6 | 3.7 | 1.5 | 1.5 | 1.5 | 0.4 | 2.4 | 1.4 | 2.5 | 0.89 |
| YFR015C | 1.2 | 0.8 | 3.2 | 2.1 | 1.2 | 3.0 | 7.3 | 0.7 | 0.3 | 1.8 | 3.9 | 0.4 | 2.5 | 3.1 | 0.4 | 2.4 | 1.3 | 1.2 | 0.66 |
| YGL253W | 0.8 | 1.0 | 3.2 | 0.7 | 1.3 | 2.4 | 1.7 | 1.1 | 0.3 | 1.4 | 1.0 | 1.2 | 1.5 | 0.5 | 0.8 | 0.4 | 0.7 | 0.8 | 4.63 |
| YIL111W | 1.9 | 1.0 | 1.8 | 2.7 | | 2.6 | 3.2 | 1.2 | 1.6 | 1.1 | 2.1 | 0.9 | 1.7 | 1.5 | 1.1 | 2.5 | 1.9 | 3.9 | 1.52 |
| YKL150W | 1.1 | 2.0 | 3.0 | 1.4 | 1.4 | 2.3 | 2.4 | 1.3 | 1.1 | 1.3 | 1.9 | 1.2 | 1.8 | 1.4 | 0.9 | 6.1 | 1.2 | 2.7 | 2.20 |
| YPR006C | 1.9 | 1.5 | 0.5 | 1.9 | 2.0 | 2.4 | 2.8 | 1.5 | 1.5 | 3.9 | 2.4 | 1.1 | 1.8 | 1.2 | 0.7 | 1.6 | 1.8 | 2.1 | 0.46 |
| YBR145W | 1.5 | 0.7 | 2.8 | 0.9 | 1.1 | 11.5 | 58.8 | 1.0 | 0.1 | 1.1 | 1.1 | 1.0 | 2.0 | 2.2 | 1.2 | 3.6 | 1.7 | 2.0 | 2.17 |
| YBR299W | 2.0 | 0.9 | 1.1 | 3.5 | 1.6 | 0.8 | 3.6 | 2.2 | 1.1 | 5.3 | 2.4 | 1.2 | 0.7 | 1.4 | 0.6 | 3.9 | 1.0 | 1.1 | 0.32 |
| YEL020C | 1.0 | 1.5 | 0.8 | 2.9 | 1.5 | 1.3 | 2.4 | 1.2 | 1.4 | 1.1 | 1.3 | 0.8 | 1.2 | 1.0 | 1.4 | 2.1 | 1.2 | 1.3 | 0.31 |
| YGL134W | 1.2 | 1.3 | 0.5 | 0.8 | 1.4 | 1.2 | 2.3 | 1.4 | 1.1 | 1.4 | 1.1 | 0.9 | 0.7 | 1.1 | 0.5 | 1.7 | 0.9 | 1.3 | 0.53 |
| YOL157C | 1.0 | 1.1 | 1.3 | 2.5 | 1.4 | 0.9 | 2.7 | 1.4 | 2.3 | 4.8 | 1.2 | 1.2 | 1.4 | 1.2 | 1.1 | 3.5 | 1.4 | 1.3 | 0.41 |
| YBR126C | 0.8 | 1.9 | 5.6 | 1.2 | 0.7 | 2.9 | 2.3 | 0.6 | 1.7 | | 1.1 | 1.3 | 2.1 | 0.7 | 1.0 | 1.7 | 1.5 | 1.3 | 1.96 |
| YCR005C | 1.2 | 1.9 | 2.0 | 1.2 | 0.9 | 1.6 | 4.4 | 1.2 | 1.5 | 1.5 | 2.1 | 0.5 | 0.7 | 0.8 | 0.7 | 0.7 | 1.6 | 1.7 | 2.38 |
| YIL172C | 1.1 | 1.1 | 1.6 | 1.7 | | 1.3 | 2.5 | 1.6 | 2.8 | 7.1 | 1.0 | 1.4 | 2.0 | 1.3 | 1.4 | 2.8 | 1.1 | 1.2 | 0.42 |
| YOR221C | 0.8 | 1.0 | 0.9 | 1.1 | 1.7 | 0.8 | 2.1 | 0.9 | 1.4 | 1.7 | 1.1 | 0.8 | 1.0 | 1.1 | 1.0 | 1.4 | 0.9 | 1.2 | 0.39 |
| YBR196C | 0.8 | 0.6 | 3.9 | 1.4 | 0.8 | 0.8 | 1.4 | 1.1 | 0.3 | 0.9 | 2.3 | 1.0 | 1.4 | 0.5 | 1.9 | 1.1 | 0.8 | 1.0 | 6.60 |
| YEL047C | 1.3 | 1.9 | 3.2 | 1.2 | 0.8 | 0.6 | 1.3 | 1.0 | 2.7 | 2.3 | 4.2 | 1.2 | 1.6 | 1.1 | 1.0 | 1.3 | 0.9 | 1.0 | 1.02 |
| YMR318C | 1.8 | 2.4 | 2.2 | 0.7 | 1.2 | 2.1 | 0.8 | 3.6 | 2.3 | 4.8 | 3.6 | 0.8 | 1.8 | 1.7 | 1.5 | 1.1 | 1.1 | 1.1 | 3.17 |
| YER061C | 0.9 | 0.9 | 1.2 | 2.5 | 0.8 | 0.4 | 0.9 | 0.7 | 0.3 | 0.7 | 2.2 | 0.7 | 0.9 | 1.0 | 0.8 | 1.8 | 1.2 | 1.2 | 0.84 |
| YJL045W | 1.8 | 2.2 | 1.6 | 5.3 | 0.7 | 0.6 | 0.7 | 0.9 | 9.7 | 1.6 | 2.3 | 1.1 | 1.6 | 1.2 | 3.4 | 2.4 | 0.9 | 0.9 | 0.42 |
| YLL009C | 1.0 | 1.2 | 0.6 | 1.9 | 1.5 | 1.2 | 1.7 | 1.0 | 1.3 | 1.7 | 3.5 | 0.7 | 0.9 | 1.4 | 1.1 | 2.8 | 0.9 | 1.2 | 1.66 |
| YPR160W | 1.4 | 3.8 | 3.6 | 3.3 | 0.7 | 4.5 | 1.8 | 0.9 | 0.9 | 1.3 | 4.4 | 2.1 | 2.2 | 1.1 | 1.4 | 5.3 | 1.0 | 2.9 | 1.42 |
| YDL085W | 1.2 | 1.9 | 1.2 | 2.0 | 1.2 | 1.2 | 1.4 | 1.0 | 7.8 | 3.4 | 2.7 | 0.8 | 1.7 | 1.2 | 0.7 | 4.5 | 0.9 | 0.9 | 0.23 |
| YJL221C | 1.1 | 1.0 | 1.1 | 1.3 | 0.9 | 6.6 | 2.5 | 1.8 | 2.7 | 4.4 | 0.8 | 1.1 | 1.4 | 1.1 | 1.1 | 3.3 | 1.1 | 1.4 | 0.41 |
| YKL085W | 1.5 | 2.3 | 1.6 | 1.2 | 1.2 | 1.9 | 1.5 | 1.2 | 1.9 | 3.0 | 1.8 | 0.8 | 1.5 | 1.0 | 0.5 | 1.7 | 0.9 | 1.3 | 2.16 |
| YLR174W | 1.2 | 1.5 | 1.7 | 2.1 | 0.9 | 0.9 | 1.6 | 1.1 | 2.5 | 8.3 | 1.3 | 1.3 | 1.8 | 1.7 | 0.8 | 4.6 | 0.9 | 1.2 | 0.41 |
| YNL009W | 1.1 | 1.9 | 2.0 | 1.4 | 1.4 | 1.1 | 1.2 | 1.1 | 1.4 | 3.3 | 1.3 | 0.9 | 1.3 | 1.3 | 2.7 | 3.3 | 1.2 | 2.3 | 0.45 |
| YNL117W | 0.9 | 4.7 | 1.7 | 0.8 | 0.8 | | 1.1 | 1.7 | 12.8 | 4.4 | 1.4 | 0.7 | 1.3 | 2.0 | 2.6 | 1.6 | 1.1 | 0.9 | 0.24 |
| YAL061W | 1.7 | 2.4 | 3.3 | 3.8 | 1.0 | 1.0 | 2.0 | 0.8 | 5.5 | 1.4 | 4.1 | 1.1 | 1.4 | 0.7 | 0.6 | 1.1 | 1.4 | 1.2 | 0.88 |
| YBR117C | 0.8 | 1.6 | 1.5 | 1.4 | 0.9 | 2.3 | 1.0 | 0.7 | 5.9 | 1.5 | 0.4 | 0.8 | 2.1 | 0.9 | 1.0 | 12.0 | 0.7 | 0.7 | 0.49 |
| YEL071W | 1.1 | 1.6 | 2.6 | 0.4 | 1.3 | 1.9 | 2.0 | 1.2 | 3.6 | 2.8 | 2.1 | 0.9 | 1.6 | 1.2 | 1.6 | 1.2 | 1.1 | 1.2 | 1.13 |
| YGR112W | 1.0 | 2.9 | 1.1 | 1.3 | 0.9 | 1.1 | 1.3 | 1.3 | 2.6 | 1.3 | 1.3 | 0.7 | 1.1 | 1.1 | 1.2 | 2.8 | 0.9 | 1.2 | 0.31 |
| YLR164W | 1.0 | 2.4 | 0.3 | 1.0 | 1.2 | 1.2 | 1.1 | 1.2 | 5.3 | 2.0 | 3.2 | 0.7 | 1.0 | 1.5 | 0.7 | 11.0 | 1.5 | 1.2 | 0.31 |
| YNR032W | 1.0 | 1.5 | 1.0 | 2.1 | 1.5 | 1.0 | 1.1 | 1.0 | 6.2 | 2.4 | 1.4 | 0.6 | 1.3 | 1.4 | 0.9 | 1.0 | 1.2 | 1.3 | 0.71 |
| YPL031C | 0.9 | 2.0 | 1.4 | 0.8 | 1.5 | 1.8 | 1.6 | 1.0 | 5.7 | 1.4 | 1.4 | 0.8 | 1.6 | 0.9 | 0.7 | 2.0 | 1.4 | 1.2 | 0.49 |
| YPR048W | 1.3 | 2.0 | 0.9 | 1.1 | 0.9 | 0.6 | 0.9 | 1.5 | 4.0 | 2.1 | 1.0 | 0.8 | 0.6 | 1.2 | 0.6 | 0.8 | 0.7 | 0.8 | 0.75 |
| YBL058W | 0.9 | 1.5 | 0.5 | 1.1 | 1.6 | 0.9 | 1.5 | 1.2 | 2.6 | 2.1 | 1.3 | 0.8 | 1.6 | 1.3 | 1.8 | 1.8 | 1.0 | 1.4 | 1.42 |
| YBR001C | 0.9 | 1.4 | 1.1 | 1.4 | 0.9 | 1.1 | 0.9 | 1.3 | 2.3 | 2.1 | 1.0 | 1.0 | 2.0 | 1.2 | 1.8 | 1.4 | 1.0 | 1.6 | 0.61 |
| YCR105W | 2.2 | 1.2 | 1.2 | 3.0 | 0.9 | 1.0 | 1.0 | 1.4 | 2.0 | 3.9 | 2.3 | 0.9 | 1.9 | 1.9 | 3.1 | 1.3 | 1.3 | 1.1 | 0.36 |
| YIR031C | 0.9 | 1.6 | 0.6 | 0.6 | 1.6 | 1.7 | 1.4 | 1.0 | 2.6 | 1.0 | 1.2 | 0.8 | 1.0 | 1.2 | 0.8 | 0.7 | 0.9 | 0.8 | 0.44 |
| YGL191W | 2.3 | 1.6 | 1.4 | 1.8 | 0.9 | 1.6 | 1.0 | 1.6 | 1.1 | 1.3 | 1.6 | 0.9 | 1.1 | 1.6 | 0.7 | 1.8 | 1.2 | 1.7 | 2.35 |
| YLR038C | 2.6 | 1.1 | 0.6 | 2.1 | 1.8 | 1.2 | 1.0 | 1.4 | 0.4 | 0.9 | 1.4 | 0.6 | 0.5 | 1.6 | 0.7 | 2.1 | 1.2 | 2.1 | 1.52 |
| YGR087C | 1.0 | 15.0 | 1.7 | 0.6 | 1.0 | 1.1 | 1.0 | 0.8 | 0.2 | 0.9 | 0.8 | 1.2 | 1.2 | 0.7 | 3.5 | 0.6 | 1.0 | 0.6 | 1.88 |
| YGL256W | 0.9 | 5.3 | 1.1 | 1.3 | 0.7 | 0.7 | 0.5 | 1.0 | 0.4 | 1.2 | 1.0 | 0.9 | 0.8 | 0.9 | 1.0 | 0.7 | 0.7 | 0.8 | 0.90 |
| YDL181W | 1.4 | 1.8 | 1.9 | 1.8 | 1.7 | 1.2 | 1.1 | 0.6 | 0.7 | 0.8 | 0.8 | 0.9 | 0.9 | 1.1 | 0.4 | 1.1 | 1.5 | 1.4 | 0.85 |
| YPL262W | 1.1 | 1.7 | 3.8 | 1.2 | 0.6 | 1.6 | 1.3 | 1.0 | 1.4 | 5.8 | 1.4 | 1.1 | 1.5 | 1.2 | 0.6 | 1.4 | 1.1 | 1.2 | 0.79 |
| YLR377C | 1.1 | 2.6 | 1.8 | 1.2 | 1.0 | 0.7 | 0.9 | 0.7 | 1.8 | 1.6 | 0.6 | 0.6 | 1.2 | 2.9 | 0.8 | 2.7 | 0.9 | 1.4 | 0.14 |
| YBR221C | 0.8 | 0.7 | 3.2 | 1.5 | 1.3 | 1.7 | 1.5 | 0.8 | 1.0 | 1.3 | 1.2 | 1.3 | 1.7 | 0.8 | 1.1 | 1.5 | 0.9 | 1.0 | 3.62 |
| YKL141W | 1.6 | 1.1 | 4.7 | 1.7 | 1.5 | 1.2 | 0.5 | 0.8 | 0.8 | 0.5 | 1.2 | 0.8 | 0.8 | 1.4 | 0.6 | 2.8 | 0.9 | 1.8 | 2.84 |
| YLR134W | 0.9 | 0.6 | 2.3 | 0.8 | 1.3 | 2.5 | 1.1 | 0.8 | 0.1 | 0.7 | 1.1 | 1.2 | 1.5 | 0.6 | 1.7 | 0.5 | 0.6 | 0.8 | 3.47 |
| YLR258W | 1.7 | 1.0 | 4.2 | 3.5 | 0.9 | 1.8 | 1.8 | 0.9 | 0.8 | 1.3 | 1.2 | 0.9 | 1.4 | 1.2 | 0.6 | 1.7 | 1.0 | 2.0 | 1.36 |
| YOR178C | 1.4 | 1.3 | 4.8 | 2.3 | 1.0 | 2.7 | 0.9 | 0.9 | 0.2 | 1.7 | 4.1 | 1.7 | 1.0 | 1.0 | 1.1 | 1.7 | 1.0 | 1.5 | 0.56 |
| YBL099W | 0.8 | 0.9 | 3.1 | 1.4 | 1.0 | 0.9 | 0.9 | 0.8 | 0.9 | 0.7 | 2.1 | 1.0 | 0.9 | 0.6 | 1.0 | 0.9 | 0.7 | 1.2 | 3.49 |
| YDR050C | 1.9 | 1.3 | 2.3 | 1.8 | 0.9 | 1.7 | 1.5 | 1.3 | 0.4 | 1.3 | 2.0 | 1.4 | 2.0 | 1.2 | 1.9 | 1.3 | 1.1 | 2.3 | 6.26 |
| YDR178W | 2.0 | 2.0 | 3.4 | 2.2 | 0.9 | 2.1 | 0.6 | 0.9 | 0.9 | 0.8 | 2.0 | 1.3 | 1.5 | 1.5 | 0.7 | 3.0 | 1.2 | 2.3 | 2.27 |
| YDR298C | 1.3 | 1.2 | 2.6 | 1.3 | 1.3 | 1.2 | 1.0 | 1.2 | 1.0 | 1.6 | 1.2 | 1.1 | 1.2 | 1.5 | 0.8 | 2.0 | 1.1 | 1.6 | 2.69 |
| YEL024W | 1.5 | 0.9 | 3.8 | 1.1 | 1.3 | 1.1 | 0.7 | 1.0 | 0.7 | 0.6 | 0.9 | 0.8 | 0.6 | 1.1 | 0.6 | 2.1 | 1.1 | 1.5 | 1.59 |
| YJL121C | 1.0 | 0.5 | 1.9 | 0.8 | 1.0 | 1.1 | 0.7 | 1.0 | 0.1 | 0.4 | 1.1 | 0.6 | 0.7 | 0.9 | 1.0 | 0.6 | 1.1 | 1.0 | 1.00 |
| YJR121W | 1.2 | 1.1 | 3.3 | 1.0 | 0.8 | 1.5 | 1.0 | 0.7 | 0.8 | 1.3 | 0.9 | 0.8 | 1.0 | 0.7 | 0.9 | 1.2 | 1.1 | 1.4 | 3.99 |
| YKL060C | 1.7 | 0.8 | 2.5 | 1.0 | 1.6 | 1.3 | 1.2 | 1.1 | 0.3 | 0.8 | 2.1 | 1.3 | 1.2 | 0.8 | 1.6 | 1.5 | 1.2 | 1.7 | 6.01 |
| YKL148C | 0.9 | 0.8 | 3.7 | 0.7 | 0.8 | 0.5 | 0.6 | 0.8 | 1.7 | 1.7 | 1.5 | 0.8 | 0.8 | 0.8 | 0.6 | 1.1 | 0.8 | 1.0 | 0.54 |
| YLL041C | 1.5 | 0.5 | 4.6 | 1.0 | 1.6 | 1.2 | 0.3 | 0.8 | 0.7 | 0.8 | 1.3 | 0.8 | 1.0 | 1.2 | 0.4 | 3.1 | 1.1 | 1.7 | 1.75 |
| YLR044C | 0.8 | 0.6 | 2.2 | 0.9 | 1.7 | 1.5 | 1.3 | 0.7 | 0.0 | 1.2 | 1.1 | 1.4 | 1.7 | 0.5 | 2.2 | 0.6 | 0.9 | 0.9 | 5.16 |
| YLR284C | 0.9 | 1.0 | 3.8 | 1.9 | 1.6 | 0.9 | 0.8 | 1.2 | 0.9 | 0.9 | 1.5 | 0.6 | 0.8 | 1.3 | 1.0 | 4.5 | 2.9 | 8.1 | 0.84 |
| YLR304C | 0.7 | 0.6 | 5.0 | 0.7 | 0.6 | 1.9 | 0.6 | 0.6 | 0.1 | 2.2 | 1.6 | 1.0 | 0.5 | 0.7 | 1.6 | 1.8 | 0.5 | 0.7 | 2.39 |
| YLR354C | 1.2 | 2.5 | 2.3 | 1.5 | 1.6 | 1.5 | 1.1 | 1.4 | 0.4 | 0.9 | 1.7 | 1.2 | 1.0 | 0.9 | 2.4 | 1.3 | 1.0 | 1.5 | 4.53 |
| YMR205C | 0.5 | 0.7 | 2.3 | 0.8 | 1.2 | 1.2 | 0.9 | 0.7 | 0.3 | 0.8 | 1.0 | 1.1 | 1.3 | 0.5 | 0.9 | 0.9 | 0.6 | 0.5 | 4.75 |
| YMR261C | 0.8 | 1.6 | 3.6 | 0.8 | 0.9 | 0.6 | 1.3 | 0.7 | 1.6 | 1.3 | 0.8 | 0.9 | 1.6 | 0.6 | 0.8 | 1.6 | 0.7 | 1.1 | 0.78 |
| YMR323W | 0.8 | 1.1 | 2.9 | 1.1 | 0.7 | 0.4 | 1.1 | 0.8 | 0.5 | 1.3 | 2.5 | 1.2 | 1.8 | 1.2 | 37.3 | 0.6 | 1.1 | 0.6 | 1.04 |
| YOL086C | 1.1 | 0.5 | 2.2 | 1.1 | 1.9 | 1.7 | 1.9 | 0.8 | 0.1 | 1.2 | 2.6 | 1.3 | 1.7 | 0.6 | 1.6 | 1.4 | 1.1 | 1.3 | 4.19 |
| YOR142W | 1.3 | 1.2 | 3.4 | 1.0 | 1.4 | 1.6 | 1.0 | 0.8 | 1.1 | 1.5 | 1.5 | 0.8 | 1.4 | 0.8 | 1.1 | 0.8 | 0.9 | 1.0 | 1.31 |
| YPL061W | 0.4 | 1.4 | 5.0 | 1.3 | 1.6 | 1.7 | 0.5 | 0.8 | 1.3 | 0.7 | 1.1 | 1.0 | 1.1 | 0.6 | 1.7 | 1.0 | 1.6 | 2.6 | 3.23 |
| YDL107W | 1.3 | 1.3 | 1.0 | 2.2 | 1.0 | 1.3 | 1.8 | 1.8 | 1.2 | 1.8 | 1.0 | 1.0 | 1.2 | 1.5 | 1.0 | 2.0 | 1.1 | 1.5 | 0.51 |
| YDR529C | 1.8 | 1.0 | 0.9 | 3.2 | 1.2 | 0.8 | 0.5 | 1.3 | 0.5 | 0.6 | 0.8 | 0.6 | 0.7 | 1.5 | 0.5 | 1.8 | 1.0 | 2.3 | 3.11 |
| YGL018C | 1.5 | 1.0 | 1.8 | 3.0 | 1.1 | 0.4 | 1.1 | 0.8 | 0.5 | 1.4 | 1.1 | 1.0 | 1.3 | 1.2 | 1.1 | 1.5 | 0.8 | 1.0 | 0.36 |
| YBR185C | 1.5 | 1.0 | 1.3 | 2.0 | 0.8 | 1.2 | 1.2 | 1.1 | 0.9 | 1.6 | 1.2 | 0.7 | 1.0 | 1.7 | 1.0 | 1.7 | 0.8 | 1.3 | 0.89 |
| YEL039C | 1.1 | 0.7 | 1.3 | 5.1 | 0.9 | 1.5 | 0.6 | 1.1 | 1.2 | 1.2 | 1.7 | 0.6 | 0.5 | 1.4 | 0.4 | 3.5 | 0.7 | 1.1 | 1.59 |
| YGR174C | 1.1 | 1.6 | 0.6 | 2.4 | 1.0 | 1.4 | 1.0 | 1.6 | 1.8 | 0.7 | 1.3 | 0.9 | 1.5 | 1.6 | 0.7 | 3.2 | 1.3 | 2.3 | 1.05 |
| YKL016C | 1.5 | 1.3 | 0.7 | 1.7 | 1.5 | 0.8 | 1.5 | 1.6 | 1.3 | 1.8 | 1.3 | 1.0 | 1.3 | 1.6 | 0.7 | 1.8 | 1.0 | 1.7 | 2.08 |
| YLR395C | 1.8 | 1.3 | 2.2 | 1.9 | 0.7 | 0.4 | | 1.0 | 1.0 | 0.9 | 1.5 | 0.6 | 0.5 | 1.7 | 0.4 | 1.2 | 1.2 | 2.3 | 1.76 |
| YML120C | 1.1 | 1.2 | 1.9 | 1.9 | 0.6 | 0.6 | 0.8 | 0.9 | 1.3 | 1.5 | 1.3 | 0.9 | 1.2 | 0.8 | 0.5 | 1.5 | 1.0 | 1.6 | 0.74 |
| YMR073C | 1.1 | 1.1 | 1.2 | 1.5 | 0.8 | 0.9 | 1.0 | 1.1 | 1.2 | 1.2 | 0.9 | 0.8 | 1.2 | 2.0 | 0.9 | 1.5 | 0.8 | 1.2 | 0.69 |
| YOR388C | 1.0 | 1.6 | 1.3 | 4.7 | 1.3 | 1.4 | 1.0 | 0.7 | 0.1 | 1.4 | 1.3 | 0.7 | 1.1 | 1.3 | 4.0 | 0.8 | 1.0 | 0.9 | 0.21 |
| YPL275W | 0.6 | 1.5 | 1.3 | 5.1 | 1.2 | 1.0 | 1.1 | 1.0 | 0.2 | 1.2 | 0.9 | 0.6 | 1.0 | 1.0 | 1.4 | 0.9 | 1.0 | 1.0 | 0.28 |
| YPL276W | 1.2 | 1.5 | 1.6 | 3.0 | 1.5 | 1.1 | 1.2 | 0.9 | −0.2 | 1.8 | −0.4 | 0.6 | 0.9 | 0.9 | 1.0 | 0.6 | 1.1 | 0.8 | 0.22 |
| YGL205W | 1.0 | 0.9 | 0.7 | 0.8 | 1.1 | 1.6 | 0.8 | 1.2 | 0.5 | 0.6 | 0.4 | 1.1 | 1.0 | 1.0 | 2.0 | 4.1 | 3.9 | 9.1 | 0.24 |
| YJL166W | 2.1 | 1.5 | 1.6 | 1.3 | 1.6 | 0.7 | 0.8 | 1.4 | 1.3 | 1.2 | 1.4 | 0.7 | 0.8 | 1.9 | 0.5 | 2.3 | 1.7 | 4.0 | 2.31 |
| YNL202W | 0.7 | 1.3 | 1.2 | 1.7 | 0.9 | 2.0 | 0.9 | 1.2 | 1.3 | 1.1 | 1.2 | 0.9 | 1.8 | 1.0 | 1.4 | 4.2 | 2.4 | 3.3 | 0.47 |
| YDR377W | 1.3 | 1.1 | 1.3 | 1.4 | 1.9 | 1.3 | 1.1 | 1.3 | 1.0 | 0.9 | 1.4 | 0.9 | 0.7 | 1.4 | 0.6 | 1.8 | 1.2 | 2.5 | 3.00 |
| YGL187C | 1.5 | 0.8 | 1.6 | 1.8 | 1.3 | 1.1 | 0.6 | 0.7 | 0.6 | 0.6 | 0.7 | 0.8 | 0.7 | 0.9 | 0.3 | 1.4 | 0.9 | 2.4 | 2.73 |
| YJL216C | 1.1 | 4.7 | 2.2 | 0.9 | 0.8 | | 1.5 | 1.9 | 1.5 | 1.3 | 1.1 | 1.0 | 1.1 | 1.1 | 1.2 | 1.5 | 1.2 | 2.0 | 0.25 |
| YKR009C | 1.0 | 1.2 | 1.5 | 0.9 | 1.0 | 1.8 | 1.0 | 0.9 | 2.4 | 2.1 | 1.2 | 1.0 | 1.8 | 1.0 | 1.0 | 4.6 | 1.9 | 2.5 | 0.27 |
| YCR046C | 1.0 | 1.2 | 1.2 | 1.6 | 1.0 | 0.8 | 1.2 | 0.9 | 1.9 | 1.4 | 1.5 | 1.1 | 1.5 | 1.3 | 1.4 | 2.8 | 1.3 | 1.0 | 0.63 |
| YML129C | 1.1 | 0.9 | 0.9 | 1.2 | 1.7 | 1.7 | 1.8 | 1.2 | 1.4 | 2.2 | 1.2 | 0.8 | 1.0 | 1.4 | 1.1 | 2.9 | 1.3 | 1.3 | 1.04 |
| YPR184W | 1.2 | 1.4 | 5.7 | 2.2 | 0.9 | 1.5 | 1.3 | 0.7 | 1.9 | 3.2 | 3.6 | 1.4 | 2.5 | 1.3 | 1.5 | 3.1 | 1.0 | 1.7 | 0.37 |
| YDL067C | 1.8 | 1.0 | 1.6 | 1.3 | 2.0 | 0.9 | 0.8 | 1.1 | 0.9 | 0.9 | 1.4 | 1.0 | 0.6 | 1.4 | 0.7 | 1.8 | 1.2 | 1.7 | 1.85 |
| YDL078C | 0.7 | 1.1 | 1.2 | 0.8 | 1.3 | 1.1 | 1.7 | 1.0 | 1.1 | 1.0 | 1.7 | 0.8 | 1.0 | 0.8 | 0.5 | 2.2 | 1.1 | 2.0 | 1.65 |
| YDR079W | 1.3 | 1.1 | 0.8 | 1.1 | 1.2 | 1.7 | 1.3 | 2.6 | 0.9 | 0.8 | 1.3 | 1.1 | 1.3 | 1.5 | 0.6 | 2.6 | 1.1 | 1.3 | 1.06 |
| YGR062C | 0.9 | 1.0 | 0.7 | 1.4 | 0.7 | 0.9 | 1.3 | 0.8 | 1.0 | 1.4 | 1.6 | 1.0 | 1.5 | 1.2 | 0.5 | 1.8 | 0.9 | 1.1 | 0.54 |
| YKR058W | 1.3 | 0.9 | 1.4 | 1.9 | 0.8 | 1.4 | 1.4 | 1.5 | 1.3 | 1.2 | 1.5 | 0.9 | 0.7 | 1.3 | 0.5 | 2.6 | 0.8 | 1.6 | 0.73 |
| YLR295C | 1.5 | 1.1 | 1.1 | 1.7 | 2.2 | 1.1 | 1.1 | 1.2 | 1.4 | 1.2 | 1.3 | 0.5 | 0.8 | 1.2 | 0.5 | 2.4 | 0.9 | 1.5 | 1.07 |
| YMR267W | 0.8 | 1.8 | 0.7 | 0.9 | 1.0 | 1.4 | 1.2 | 1.5 | 0.4 | 0.8 | 1.0 | 0.8 | 0.7 | 1.0 | 0.9 | 2.6 | 0.9 | 1.3 | 0.94 |

TABLE 5
Transport facilitation protein genes

| | The level of expressed mRNA in the presence of chemical substance/The | | | | | | | | | | | | | | | | | | |
|---|---|---|---|---|---|---|---|---|---|---|---|---|---|---|---|---|---|---|---|
| | level of expressed mRNA in the absence of chemical substance | | | | | | | | | | | | | | | | | | |
| yeast genes | (1) | (2) | (3) | (4) | (5) | (6) | (7) | (8) | (9) | (10) | (11) | (12) | (13) | (14) | (15) | (16) | (17) | (18) | Intensity |
| YBR008C | 3.0 | 2.4 | 4.9 | 0.7 | 0.9 | 1.0 | 0.9 | 2.8 | 54.6 | 21.1 | 9.4 | 0.8 | 2.0 | 4.1 | 0.8 | 3.1 | 2.0 | 1.2 | 0.37 |
| YBR296C | 7.0 | 4.3 | 2.2 | 2.5 | 1.4 | 1.4 | 0.2 | 1.1 | 0.4 | 1.8 | 1.5 | 1.6 | 2.0 | 2.3 | 1.7 | 0.3 | 3.8 | 4.0 | 0.54 |
| YDR406W | 0.8 | 1.2 | 3.1 | 1.1 | 1.0 | 5.9 | 1.8 | 0.6 | 1.1 | 1.7 | 1.4 | 3.9 | 7.7 | 0.5 | 0.8 | 5.5 | 2.7 | 5.6 | 0.53 |
| YOR153W | 1.6 | 0.9 | 5.1 | 1.1 | 1.0 | 7.4 | 2.6 | 0.5 | 3.2 | 1.2 | 1.0 | 4.0 | 11.8 | 0.3 | 3.6 | 1.6 | 2.5 | 3.1 | 1.91 |
| YGR281W | 1.2 | 1.0 | 1.3 | 2.2 | 0.6 | 1.9 | 0.9 | 0.7 | 2.7 | 1.4 | 0.7 | 2.4 | 12.9 | 0.9 | 2.1 | 1.1 | 2.8 | 3.1 | 1.04 |
| YHL047C | 0.6 | 4.4 | 1.0 | 1.0 | 1.2 | 8.4 | 16.8 | 1.5 | 1.2 | 11.9 | 1.0 | 2.6 | 3.8 | 0.5 | 1.1 | 2.3 | 1.2 | 1.2 | 0.74 |
| YBR294W | 6.3 | 14.4 | 0.9 | 2.6 | 1.4 | 1.2 | 1.1 | 1.0 | 5.2 | 5.2 | 1.2 | 1.0 | 3.1 | 1.3 | 1.0 | 0.9 | 1.0 | 0.9 | 0.18 |
| YGL006W | 2.5 | 1.3 | 1.3 | 2.6 | 1.0 | 1.0 | 1.3 | 0.9 | 3.0 | 1.2 | 0.8 | 0.9 | 4.5 | 0.9 | 1.1 | 1.7 | 2.4 | 4.1 | 1.37 |
| YGR197C | 1.1 | 2.4 | 2.5 | 1.0 | 0.7 | 0.5 | 1.3 | 0.8 | 16.7 | 3.2 | 1.1 | 1.2 | 2.4 | 1.5 | 1.0 | 1.5 | 1.7 | 1.9 | 0.37 |
| YJL034W | 0.7 | 1.7 | 3.0 | 0.8 | 1.2 | 1.1 | 1.3 | 1.5 | 2.8 | 1.1 | 1.3 | 1.6 | 6.1 | 0.6 | 7.9 | 0.6 | 1.1 | 1.0 | 4.58 |
| YNL055C | 1.1 | 1.8 | 5.5 | 2.0 | 0.9 | 2.6 | 1.2 | 0.7 | 1.1 | 1.0 | 1.9 | 1.7 | 2.4 | 0.9 | 1.1 | 2.6 | 1.2 | 1.7 | 4.10 |
| YBR052C | 1.6 | 1.6 | 1.5 | 2.6 | 1.4 | 2.1 | 3.0 | 1.3 | 2.3 | 2.5 | 1.7 | 1.3 | 2.1 | 1.5 | 0.7 | 3.1 | 1.8 | 3.1 | 2.77 |
| YBR207W | 0.8 | 1.7 | 0.8 | 2.0 | 1.7 | 1.6 | 4.3 | 0.9 | 1.7 | 2.2 | 1.1 | 0.9 | 2.2 | 0.9 | 1.4 | 1.4 | 1.2 | 1.4 | 1.39 |
| YBR293W | 1.9 | 3.1 | 1.0 | 1.8 | 0.8 | 0.9 | 0.9 | 0.9 | 5.5 | 2.4 | 1.1 | 1.0 | 3.0 | 1.4 | 3.1 | 1.6 | 1.0 | 0.9 | 0.94 |
| YDL198C | 1.4 | 1.0 | 2.1 | 1.4 | 0.7 | 0.8 | 1.0 | 1.1 | 1.9 | 2.7 | 2.5 | 1.2 | 2.0 | 1.7 | 1.2 | 1.4 | 1.5 | 1.1 | 1.19 |
| YDL245C | 1.0 | 2.4 | 1.6 | 2.2 | 1.3 | 1.1 | 1.2 | 1.2 | 0.4 | 2.8 | 1.5 | 1.0 | 2.0 | 1.2 | 1.8 | 1.5 | 1.0 | 1.1 | 0.28 |
| YDR497C | 0.7 | 0.5 | 0.6 | 1.3 | 0.7 | 0.6 | 0.5 | 0.6 | 0.4 | 0.8 | 0.7 | 0.9 | 2.7 | 0.6 | 10.1 | | 1.6 | 1.8 | 1.45 |
| YER053C | 1.6 | 1.8 | 1.9 | 1.7 | 0.6 | 2.8 | 2.8 | 1.3 | 1.3 | 3.9 | 2.4 | 1.7 | 4.1 | 1.3 | 1.1 | 2.8 | 1.2 | 2.3 | 1.83 |
| YFL041W | 0.7 | 1.6 | 1.4 | 1.2 | 1.2 | 2.0 | 3.4 | 1.0 | 0.8 | 5.1 | 0.9 | 1.1 | 1.8 | 1.0 | 0.9 | 1.4 | 1.1 | 0.9 | 0.89 |
| YGR055W | 2.5 | 5.7 | 12.0 | 0.7 | 1.3 | 1.1 | 0.6 | 1.1 | 5.0 | 2.4 | 1.5 | 0.9 | 1.9 | 1.2 | 2.1 | 0.6 | 0.7 | 0.7 | 1.42 |
| YJL219W | 1.2 | 2.5 | 3.1 | 1.6 | 1.2 | 1.5 | 0.9 | 1.5 | 4.0 | 2.3 | 2.3 | 1.1 | 4.0 | 1.1 | 2.6 | 1.4 | 1.6 | 1.2 | 0.91 |
| YJR106W | 0.9 | 3.8 | 1.5 | 1.1 | 0.9 | 1.2 | 1.0 | 0.9 | 2.0 | 1.6 | 1.6 | 0.9 | 3.5 | 0.9 | 1.2 | 1.9 | 1.2 | 1.3 | 0.29 |
| YKL146W | 0.8 | 1.3 | 1.3 | 0.9 | 1.0 | 1.3 | 0.9 | 0.8 | 4.6 | 2.8 | 0.9 | 1.1 | 2.7 | 1.0 | 1.5 | 1.3 | 1.0 | 0.9 | 0.42 |
| YLL028W | 0.6 | 0.9 | 4.6 | 0.5 | 0.9 | 1.7 | 1.0 | 0.7 | 1.2 | 2.3 | 0.7 | 1.3 | 4.1 | 0.7 | 3.1 | 0.6 | 1.2 | 1.2 | 1.02 |
| YLR348C | 1.1 | 4.5 | 1.2 | 1.2 | 0.9 | 1.9 | 0.9 | 0.9 | 2.1 | 1.3 | 1.3 | 0.9 | 1.9 | 1.0 | 2.4 | 1.1 | 1.1 | 1.0 | 0.64 |
| YOL119C | 2.6 | 4.8 | 1.5 | 2.3 | 1.5 | 0.9 | 2.2 | 2.0 | 5.0 | 7.4 | 3.4 | 0.9 | 1.6 | 2.4 | 4.7 | 2.8 | 1.4 | 1.0 | 0.37 |
| YOL163W | 1.5 | 3.2 | 2.1 | 3.3 | 0.9 | 0.5 | 0.9 | 1.1 | 7.5 | 6.2 | 1.6 | 1.0 | 2.4 | 1.2 | 1.0 | 1.4 | 0.8 | 0.8 | 0.30 |
| YOR035C | 1.2 | 1.2 | 0.3 | 1.2 | 1.0 | 1.9 | 1.3 | 1.0 | 1.3 | 1.7 | 1.0 | 1.1 | 2.5 | 0.9 | 3.8 | 1.8 | 1.6 | 2.1 | 0.52 |
| YOR130C | 0.8 | 1.7 | 1.4 | 1.0 | 1.8 | 1.6 | 1.3 | 0.8 | 2.0 | 1.7 | 0.6 | 0.9 | 2.1 | 1.3 | 1.0 | 0.8 | 1.5 | 1.1 | 0.51 |
| YOR273C | 0.7 | 1.0 | 1.7 | 1.0 | 1.1 | 0.4 | 0.4 | 0.6 | 0.6 | 0.5 | 1.5 | 0.8 | 2.6 | 0.5 | 0.8 | 0.6 | 3.2 | 3.1 | 1.20 |
| YOR332W | 1.1 | 1.0 | 1.0 | 1.0 | 0.9 | 1.8 | 0.9 | 1.5 | 1.8 | 1.1 | 1.5 | 1.0 | 2.2 | 1.1 | 1.2 | 1.2 | 1.3 | 1.7 | 3.58 |
| YCR098C | 1.6 | 3.0 | 1.7 | 2.0 | 1.4 | 1.6 | 1.2 | 1.0 | 1.4 | 2.4 | 1.3 | 1.3 | 1.4 | 1.1 | 11.0 | 0.8 | 1.7 | 1.6 | 0.22 |
| YGR138C | 1.1 | 1.6 | 1.0 | 1.2 | 0.8 | 0.8 | 0.7 | 1.0 | 0.5 | 1.0 | 0.7 | 0.6 | 0.6 | 0.9 | 4.5 | 0.7 | 0.7 | 0.7 | 1.24 |
| YBR295W | 1.4 | 1.5 | 3.3 | 1.1 | 0.8 | | 1.6 | 0.9 | 1.2 | 3.3 | 1.1 | 0.9 | 1.8 | 1.1 | 4.8 | 1.4 | 1.3 | 1.2 | 0.27 |
| YGL255W | 0.7 | 0.9 | 0.9 | 0.8 | 1.4 | 1.1 | 1.0 | 0.7 | 0.3 | 5.0 | 0.5 | 0.7 | 0.2 | 1.0 | 2.6 | 0.5 | 0.6 | 0.5 | 1.36 |
| YHL035C | 0.7 | 1.3 | 0.9 | 1.9 | 0.9 | 4.1 | 1.7 | 1.1 | 0.6 | 4.6 | 0.9 | 1.4 | 1.1 | 0.8 | 3.1 | 2.4 | 1.1 | 1.2 | 0.77 |
| YIL022W | 0.8 | 0.7 | 1.1 | 1.6 | 0.8 | 1.2 | 0.7 | 0.8 | 1.1 | 1.3 | 1.0 | 0.9 | 1.2 | 0.6 | 2.9 | 1.1 | 0.8 | 0.7 | 0.52 |
| YLR378C | 0.7 | 0.9 | 2.6 | 0.5 | 1.0 | 1.2 | 0.7 | 0.5 | 1.1 | 0.7 | 1.0 | 1.2 | 1.6 | 0.4 | 5.9 | 0.4 | 0.9 | 0.7 | 1.23 |
| YCL038C | 1.6 | 1.6 | 1.4 | 1.6 | 1.2 | 3.2 | 1.5 | 1.2 | 1.1 | 0.9 | 2.0 | 1.3 | 1.9 | 1.3 | 2.0 | 1.8 | 1.4 | 1.5 | 1.04 |
| YFL054C | 1.6 | 1.1 | 1.4 | 2.2 | 1.3 | 2.4 | 1.1 | 1.5 | 2.0 | 2.3 | 1.7 | 1.2 | 2.3 | 0.9 | 1.4 | 1.3 | 1.7 | 1.4 | 1.06 |
| YHL040C | 1.6 | 4.9 | 1.5 | 0.4 | 1.1 | 7.3 | 4.0 | 1.5 | 1.1 | 11.3 | 1.7 | 2.0 | 2.0 | 1.1 | 3.4 | 1.0 | 1.4 | 1.0 | 0.69 |
| YKR039W | 1.4 | 3.5 | 1.3 | 1.7 | 1.0 | 2.8 | 1.0 | 1.0 | 1.9 | 2.0 | 1.9 | 1.5 | 1.3 | 1.2 | 1.7 | 1.9 | 0.9 | 1.1 | 0.40 |
| YPL271W | 1.2 | 3.2 | 4.1 | 1.2 | 1.4 | 3.3 | 1.2 | 0.9 | 0.6 | 1.2 | 1.7 | 0.8 | 1.2 | 1.3 | 0.8 | 1.4 | 1.2 | 1.3 | 1.34 |
| YBR068C | 1.1 | 1.0 | 1.9 | 2.5 | 1.4 | 1.9 | | 1.0 | 0.8 | 2.5 | 0.8 | 0.8 | 2.2 | 1.8 | 1.6 | 5.5 | 1.1 | 1.3 | 1.99 |
| YCR037C | 0.7 | 1.0 | 0.8 | 0.8 | 1.3 | 1.8 | 1.0 | 0.7 | 0.6 | 0.6 | 0.8 | 0.7 | 1.3 | 0.8 | 1.0 | 1.1 | 1.4 | 0.9 | 0.74 |
| YDL128W | 0.8 | 1.1 | 2.1 | 1.0 | 1.0 | 2.3 | 0.9 | 0.8 | 0.5 | 0.7 | 1.2 | 0.9 | 1.6 | 0.7 | 1.1 | 1.0 | 0.9 | 1.0 | 2.80 |
| YDR270W | 0.7 | 1.5 | 1.4 | 1.3 | 1.0 | 2.2 | 1.7 | 1.2 | 2.9 | 3.6 | 1.0 | 1.0 | 1.8 | 1.0 | 1.4 | 1.1 | 0.9 | 0.7 | 0.38 |
| YEL065W | 0.3 | 3.8 | 1.2 | 0.4 | 1.4 | 2.3 | 4.3 | 0.9 | 0.1 | 4.9 | 0.6 | 1.7 | 0.8 | 0.4 | 2.2 | 2.6 | 0.7 | 0.6 | 2.10 |
| YGL008C | 0.6 | 0.7 | 3.1 | 0.9 | 0.8 | 1.9 | 1.4 | 0.7 | 0.0 | 0.4 | 0.7 | 2.4 | 1.9 | 1.2 | 1.0 | 2.1 | 1.1 | 1.0 | 4.08 |
| YGL104C | 0.8 | 2.0 | 2.1 | 1.8 | 1.0 | 2.1 | 1.2 | 0.8 | 4.6 | 2.1 | 1.8 | 1.1 | 2.0 | 1.0 | 0.9 | 1.7 | 1.3 | 1.3 | 0.58 |
| YGL167C | 0.9 | 0.7 | 1.6 | 1.3 | 1.2 | 2.0 | 1.2 | 0.8 | 1.3 | 1.1 | 0.8 | 1.1 | 1.5 | 0.8 | 1.3 | 1.3 | 1.4 | 1.4 | 1.41 |
| YGR065C | 0.8 | 1.5 | 2.6 | 1.1 | 1.2 | 2.4 | 2.4 | 1.0 | 1.2 | 2.6 | 1.1 | 0.5 | 0.8 | 1.3 | 1.2 | 1.1 | 0.7 | 0.7 | 0.68 |
| YHR092C | 2.3 | 1.0 | 7.5 | 2.5 | 1.4 | 1.7 | 0.5 | 1.5 | 0.2 | 0.9 | 1.5 | 1.0 | 0.5 | 1.6 | 0.2 | 1.2 | 0.9 | 1.3 | 6.09 |
| YIL088C | 0.9 | 2.1 | 2.1 | 1.3 | 1.6 | 2.2 | 1.7 | 0.8 | 1.0 | 1.7 | 1.8 | 0.9 | 1.5 | 1.2 | 1.2 | 1.8 | 1.2 | 1.4 | 1.89 |
| YNL259C | 1.6 | 3.5 | 1.1 | 1.2 | 1.1 | 3.9 | 3.8 | 1.5 | 1.6 | 2.1 | 1.6 | 0.7 | 1.1 | 1.4 | 0.7 | 1.7 | 2.6 | 2.4 | 1.22 |
| YOR270C | 0.7 | 0.5 | 2.0 | 0.8 | 0.8 | 1.8 | 1.0 | 0.6 | 0.4 | 0.9 | 1.5 | 0.7 | 1.3 | 0.7 | 1.1 | 0.8 | 0.9 | 0.9 | 3.49 |
| YPL265W | 0.7 | 0.4 | 2.3 | 1.1 | 1.0 | 2.3 | 8.9 | 2.3 | 0.2 | 1.2 | 1.8 | 0.9 | 1.1 | 1.1 | 1.2 | 2.0 | 0.5 | 0.4 | 1.42 |
| YPR124W | 0.2 | 0.4 | 0.3 | 1.4 | 2.0 | 2.0 | 2.9 | 2.7 | 0.2 | 1.5 | 2.0 | 0.8 | 0.6 | 0.4 | 1.1 | 2.4 | 1.0 | 1.3 | 2.57 |
| YER119C | 0.7 | 2.1 | 1.8 | 0.7 | 0.8 | 1.3 | 0.8 | 0.6 | 5.8 | 1.2 | 2.2 | 0.7 | 1.6 | 1.0 | 2.3 | 0.8 | 1.0 | 0.9 | 0.32 |
| YFL055W | 2.0 | 6.7 | 1.3 | 2.5 | 1.5 | 1.1 | 1.4 | 1.3 | 23.9 | 6.5 | 2.7 | 0.9 | 1.3 | 1.5 | 0.7 | 1.4 | 1.4 | 1.0 | 0.23 |
| YLL055W | 2.6 | 19.0 | 4.8 | 1.7 | 1.0 | 1.4 | 1.7 | 1.4 | 19.1 | 14.0 | 3.6 | 0.7 | 1.5 | 1.3 | 0.7 | 2.8 | 1.1 | 1.3 | 0.47 |
| YHL036W | 2.2 | 4.8 | 3.4 | 2.0 | 1.3 | 1.3 | 1.0 | 1.2 | 9.0 | 4.4 | 2.3 | 1.2 | 1.5 | 1.2 | 0.9 | 1.1 | 1.0 | 1.0 | 0.60 |
| YHR048W | 2.5 | 1.4 | 1.4 | 1.7 | 1.0 | 0.8 | 0.8 | 1.9 | 4.5 | 2.7 | 2.0 | 0.9 | 1.1 | 1.7 | 0.9 | 1.4 | 0.7 | 0.9 | 0.26 |
| YKL221W | 1.3 | 0.7 | 2.0 | 1.0 | 1.0 | 0.9 | 1.3 | 1.2 | 7.3 | 2.7 | 1.9 | 0.7 | 1.6 | 1.4 | 1.1 | 1.3 | 0.8 | 0.9 | 0.27 |
| YLR092W | 5.0 | 5.6 | 1.4 | 0.6 | 1.3 | 1.0 | 1.3 | 1.5 | 12.7 | 5.8 | 3.1 | 1.0 | 1.8 | 1.4 | 0.9 | 1.2 | 1.0 | 1.1 | 0.24 |
| YML116W | 4.1 | 1.3 | 1.5 | 1.4 | 1.2 | 0.9 | 1.4 | 2.2 | 1.4 | 3.1 | 4.3 | 0.5 | 0.8 | 2.0 | 1.9 | 1.0 | 1.0 | 1.0 | 0.94 |
| YBR291C | 2.0 | 0.9 | 1.0 | 1.5 | 0.9 | 1.1 | 0.9 | 2.2 | 0.9 | 1.0 | 1.1 | 0.9 | 1.0 | 1.7 | 0.5 | 1.5 | 0.9 | 1.3 | 1.19 |
| YCL069W | 0.9 | 4.5 | 0.9 | 0.8 | 0.9 | | 1.4 | 1.3 | 1.2 | 6.9 | 1.0 | 0.9 | 0.8 | 1.3 | 1.4 | 0.9 | 1.0 | 1.0 | 0.25 |
| YJR095W | 1.2 | 20.5 | 1.9 | 6.7 | 1.2 | 1.5 | 2.0 | 0.9 | 0.5 | 6.3 | 0.6 | 0.7 | 0.8 | 1.3 | 0.8 | 0.8 | 1.3 | 0.9 | 0.23 |
| YKL188C | 1.3 | 0.7 | 1.9 | 2.4 | 1.0 | 0.8 | 1.1 | 0.7 | 2.5 | 2.8 | 1.1 | 1.2 | 1.2 | 2.1 | 1.4 | 2.7 | 1.1 | 1.5 | 0.27 |
| YKL217W | 1.8 | 2.4 | 1.0 | 2.1 | 1.1 | 1.2 | 1.6 | 1.1 | 0.9 | 4.1 | 1.6 | 0.8 | 1.2 | 1.2 | 2.2 | 3.0 | 1.7 | 3.3 | 0.29 |
| YKR105C | 0.8 | 0.9 | 0.9 | 1.5 | 1.0 | 1.2 | 1.0 | 1.3 | 1.0 | 5.2 | 0.0 | 0.8 | 2.5 | 1.2 | 1.5 | 0.7 | 0.8 | 1.0 | 0.26 |
| YOL158C | 0.7 | 4.0 | 2.4 | 0.9 | 1.2 | 2.0 | 1.6 | 0.7 | 1.7 | 6.1 | 0.7 | 0.9 | 1.4 | 1.0 | 0.9 | 1.2 | 1.4 | 1.7 | 1.30 |
| YPL224C | 1.0 | 2.2 | 1.3 | 2.5 | 0.6 | 1.3 | 2.2 | 1.3 | 3.2 | 4.2 | 0.8 | 1.1 | 1.3 | 1.4 | 1.0 | 2.5 | 1.3 | 1.9 | 0.64 |
| YPR201W | 33.2 | 0.9 | 1.1 | 1.0 | 1.2 | 1.9 | 0.8 | 0.9 | 8.3 | 5.6 | 2.2 | 0.7 | 1.1 | 1.8 | 0.8 | 0.9 | 0.8 | 0.9 | 0.29 |
| YAL067C | 2.7 | 12.1 | 1.5 | 1.4 | 0.7 | 1.2 | 0.8 | 1.1 | 7.6 | 2.9 | 1.2 | 1.0 | 1.1 | 1.3 | 2.4 | 1.6 | 1.0 | 1.0 | 0.33 |
| YDL149W | 0.8 | 2.0 | 1.2 | 1.6 | 1.5 | 1.0 | 0.7 | 1.2 | 3.2 | 2.0 | 1.2 | 0.7 | 1.5 | 1.2 | 1.8 | 1.4 | 0.8 | 1.0 | 0.33 |
| YJL094C | 1.0 | 1.7 | 1.3 | 2.0 | 1.4 | 1.3 | 1.7 | 0.8 | 5.4 | 2.9 | 1.1 | 1.2 | 2.6 | 1.0 | 1.0 | 1.8 | 2.1 | 2.0 | 0.84 |
| YLL061W | 3.6 | 2.8 | 9.2 | 0.4 | 0.8 | 0.8 | 0.9 | 1.4 | 3.7 | 5.5 | 1.7 | 0.7 | 0.9 | 1.3 | 1.4 | 1.0 | 0.6 | 0.7 | 0.33 |
| YPL274W | 0.8 | 4.2 | 3.8 | 0.8 | 0.6 | 1.1 | 0.7 | 0.9 | 4.1 | 3.6 | 2.0 | 0.5 | 0.6 | 0.9 | 1.5 | 1.1 | 0.7 | 0.6 | 0.41 |
| YBR241C | 0.9 | 9.3 | 2.6 | 0.9 | 0.9 | 1.5 | 1.4 | 0.7 | 29.8 | 2.2 | 1.4 | 1.4 | 1.4 | 0.9 | 1.6 | 0.7 | 1.0 | 1.2 | 1.25 |
| YDL206W | 0.9 | 1.7 | 1.8 | 1.4 | 1.1 | 5.7 | 2.0 | 1.1 | 1.6 | 1.5 | 0.6 | 1.0 | 1.3 | 1.0 | 1.1 | 3.1 | 1.0 | 1.1 | 0.35 |
| YDR040C | 0.7 | 1.4 | 3.7 | 0.8 | 1.0 | 1.7 | 0.7 | 0.8 | 2.0 | 1.2 | 0.8 | 0.9 | 1.8 | 0.9 | 0.8 | 1.4 | 1.4 | 1.6 | 1.25 |
| YFR045W | 1.1 | 1.6 | 1.1 | 1.1 | 1.0 | 1.0 | 0.6 | 1.0 | 2.2 | 1.0 | 1.4 | 1.0 | 1.1 | 1.0 | 1.1 | 0.6 | 0.9 | 0.8 | 0.84 |
| YIL170W | 1.1 | 1.0 | 2.5 | 2.2 | 0.9 | 8.8 | 0.5 | 1.2 | 5.7 | 3.2 | 1.7 | 0.7 | 1.9 | 1.1 | 2.3 | 1.8 | 1.6 | 1.5 | 0.48 |
| YKL192C | 1.1 | 1.0 | 3.7 | 1.1 | 1.1 | 1.8 | 1.1 | 0.9 | 3.5 | 1.6 | 1.1 | 1.2 | 1.7 | 1.5 | 1.2 | 2.1 | 1.0 | 0.8 | 1.57 |
| YKL209C | 1.0 | 1.0 | 1.3 | 0.8 | 1.2 | 3.3 | 0.9 | 1.4 | 2.4 | 1.6 | 0.8 | 0.9 | 1.4 | 1.1 | 1.0 | 1.1 | 1.1 | 1.0 | 0.30 |
| YKR106W | 1.3 | 0.8 | 2.1 | 1.4 | 1.2 | 1.6 | 0.9 | 1.3 | 10.5 | 7.4 | 1.8 | 0.8 | 1.4 | 1.7 | | 2.8 | 0.9 | 0.8 | 0.16 |
| YMR056C | 1.1 | 1.3 | 1.7 | 1.0 | 1.1 | 0.9 | 1.4 | 1.2 | 1.8 | 1.1 | 1.6 | 0.9 | 0.9 | 1.1 | 0.7 | 2.4 | 1.0 | 1.1 | 0.72 |
| YPL147W | 1.0 | 0.8 | 2.3 | 1.2 | 1.2 | 1.1 | 1.0 | 0.9 | 2.4 | 2.4 | 1.3 | 0.9 | 1.5 | 1.0 | 2.4 | 2.5 | 1.5 | 3.6 | 0.33 |
| YIL166C | 1.9 | 12.5 | 2.1 | 1.2 | 1.0 | 1.9 | | 1.3 | 2.5 | 2.9 | 1.8 | 0.9 | 1.5 | 1.1 | 1.3 | 0.7 | 1.2 | 0.9 | 0.26 |
| YMR058W | 0.3 | 5.1 | 2.1 | 0.3 | 0.8 | 2.6 | 1.8 | 0.8 | 0.2 | 1.4 | 0.8 | 0.8 | 0.4 | 0.3 | 0.5 | 0.4 | 0.6 | 0.5 | 1.66 |
| YNL142W | 0.7 | 2.4 | 2.5 | 0.8 | 1.3 | 0.9 | 1.1 | 0.8 | 0.9 | 0.6 | 0.9 | 1.2 | 0.9 | 0.8 | 0.3 | 1.0 | 1.0 | 0.9 | 0.44 |
| YDL210W | 1.2 | 2.2 | 2.7 | 1.1 | 0.9 | 1.3 | 0.8 | 1.1 | 1.2 | 1.4 | 0.6 | 0.8 | 1.2 | 1.1 | 1.9 | 0.9 | 1.1 | 0.9 | 0.20 |
| YCL025C | 1.1 | 4.6 | 2.5 | 1.7 | 0.9 | 0.9 | 0.7 | 0.7 | 0.3 | 0.3 | 1.0 | 1.1 | 0.5 | 0.7 | 0.8 | 0.6 | 0.6 | 0.6 | 1.98 |
| YJL212C | 0.8 | 3.2 | 3.1 | 0.5 | 1.2 | 1.1 | 0.7 | 0.7 | 1.1 | 1.3 | 0.9 | 0.4 | 1.0 | 0.8 | 0.3 | 0.5 | 0.6 | 0.5 | 0.59 |
| YBR132C | 0.9 | 1.9 | 2.4 | 1.8 | 0.8 | 1.2 | 1.1 | 0.8 | 0.8 | 1.1 | 1.9 | 1.0 | 1.4 | 1.1 | 1.5 | 1.3 | 1.1 | 1.3 | 0.43 |
| YBL030C | 1.0 | 0.9 | 4.5 | 0.9 | 0.7 | 1.1 | 0.9 | 0.8 | 0.4 | 0.9 | 1.1 | 0.9 | 0.5 | 0.8 | 1.8 | 1.0 | 1.0 | 1.1 | 3.12 |
| YCR024C-A | 0.8 | 0.9 | 3.2 | | 0.9 | 1.2 | 0.8 | 0.8 | 0.2 | 0.7 | 1.5 | 1.0 | 0.5 | 0.8 | 1.3 | 1.5 | 0.9 | 1.4 | 4.02 |
| YDR342C | 2.8 | 1.1 | 12.2 | 5.7 | 1.6 | 1.1 | 0.8 | 1.2 | 0.2 | 2.2 | 2.9 | 1.0 | 0.6 | 0.9 | 0.5 | 2.4 | 1.0 | 2.2 | 5.23 |
| YDR343C | 1.2 | 1.0 | 20.6 | 4.6 | 1.3 | 1.3 | 0.7 | 1.2 | 0.3 | 2.1 | 2.3 | 1.0 | 0.8 | 0.8 | 0.5 | 2.8 | 1.1 | 2.3 | 5.81 |
| YEL027W | 1.2 | 0.6 | 3.4 | 1.1 | 1.0 | 1.1 | 0.8 | 0.9 | 0.9 | 1.1 | 0.9 | 0.9 | 0.7 | 1.4 | 1.5 | 1.6 | 1.0 | 1.4 | 4.75 |
| YHR094C | 0.7 | 1.2 | 5.3 | 1.6 | 1.1 | 1.6 | 0.8 | 1.2 | 0.3 | 1.2 | 0.6 | 0.9 | 0.7 | 0.6 | 2.7 | 0.9 | 1.4 | 1.4 | 4.82 |
| YHR175W | 0.8 | 2.0 | 2.4 | 1.5 | 0.9 | 1.0 | 1.6 | 0.9 | 0.6 | 2.0 | 1.1 | 1.4 | 1.2 | 0.7 | 1.3 | 2.3 | 1.3 | 1.2 | 1.01 |
| YIL056W | 1.1 | 0.9 | 6.2 | 1.4 | 1.2 | 1.0 | 0.6 | 0.7 | 1.2 | 1.6 | 0.7 | 1.0 | 1.5 | 1.0 | 0.5 | 0.9 | 1.5 | 1.2 | 0.71 |
| YMR203W | 0.8 | 0.7 | 3.0 | 1.4 | 0.6 | 1.1 | 0.8 | 0.7 | 0.7 | 1.2 | 2.3 | 0.7 | 1.1 | 0.7 | 1.2 | 0.9 | 0.8 | 0.7 | 1.62 |
| YBL099W | 0.8 | 0.9 | 3.1 | 1.4 | 1.0 | 0.9 | 0.9 | 0.8 | 0.9 | 0.7 | 2.1 | 1.0 | 0.9 | 0.6 | 1.0 | 0.9 | 0.7 | 1.2 | 3.49 |
| YBR127C | 0.8 | 0.7 | 2.2 | 1.1 | 1.4 | 1.1 | 0.9 | 1.1 | 0.6 | 1.3 | 1.1 | 0.9 | 0.9 | 0.6 | 1.3 | 1.4 | 0.8 | 1.0 | 4.59 |
| YDR038C | 0.6 | 1.3 | 3.1 | 0.9 | 0.9 | 1.8 | 0.7 | 0.9 | 1.4 | 0.9 | 0.9 | 0.7 | 2.0 | 0.8 | 0.8 | 1.4 | 1.3 | 1.5 | 1.32 |
| YDR039C | 0.6 | 1.6 | 3.2 | 0.9 | 1.3 | 1.1 | 0.8 | 0.6 | 1.8 | 1.0 | 0.9 | 0.8 | 1.8 | 0.9 | 1.2 | 1.4 | 1.1 | 1.6 | 1.39 |
| YDR298C | 1.3 | 1.2 | 2.6 | 1.3 | 1.3 | 1.2 | 1.0 | 1.2 | 1.0 | 1.6 | 1.2 | 1.1 | 1.2 | 1.5 | 0.8 | 2.0 | 1.1 | 1.6 | 2.69 |
| YDR345C | 0.8 | 0.9 | 5.6 | 2.6 | 1.3 | 1.2 | 1.1 | 1.2 | 0.2 | 1.1 | 1.5 | 1.4 | 1.2 | 0.8 | 0.8 | 1.0 | 1.3 | 1.8 | 5.65 |
| YEL063C | 0.7 | 0.8 | 2.4 | 1.3 | 1.2 | 1.3 | 0.7 | 0.6 | 0.8 | 1.7 | 0.8 | 1.1 | 1.1 | 0.8 | 1.3 | 1.0 | 0.7 | 0.7 | 1.12 |
| YFL011W | 1.2 | 0.7 | 3.7 | 3.3 | 1.3 | 1.0 | 0.8 | 0.8 | 0.3 | 1.6 | 2.1 | 0.9 | 0.8 | 0.8 | 1.7 | 1.2 | 0.8 | 1.0 | 1.25 |
| YGR082W | 1.0 | 0.9 | 2.2 | 1.0 | 0.9 | 1.1 | 0.9 | 0.8 | 0.6 | 0.9 | 1.3 | 0.8 | 0.5 | 0.9 | 1.2 | 1.3 | 0.9 | 0.8 | 1.47 |
| YGR191W | 0.8 | 0.7 | 1.6 | 1.5 | 0.8 | 1.1 | 0.7 | 0.7 | 0.1 | 0.7 | 1.6 | 0.7 | 1.0 | 0.7 | 1.5 | 0.7 | 0.8 | 0.8 | 1.58 |
| YGR260W | 0.6 | 0.7 | 3.4 | 0.8 | 0.8 | 0.9 | 0.9 | 0.5 | 0.3 | 0.4 | 1.3 | 0.6 | 0.5 | 0.7 | 1.8 | 1.0 | 0.9 | 0.9 | 1.71 |
| YHR026W | 0.9 | 1.0 | 1.9 | 1.0 | 1.1 | 1.3 | 0.9 | 1.6 | 1.1 | 0.9 | 1.4 | 0.7 | 0.7 | 1.3 | 2.4 | 1.2 | 0.9 | 1.1 | 3.40 |
| YJR077C | 1.1 | 1.1 | 2.0 | 1.6 | 0.9 | 0.9 | 0.7 | 0.8 | 0.4 | 0.7 | 1.6 | 1.0 | 0.9 | 0.7 | 1.5 | 0.7 | 1.0 | 0.8 | 1.79 |
| YJR121W | 0.9 | 1.1 | 3.3 | 1.0 | 0.8 | 1.5 | 1.0 | 0.7 | 0.8 | 1.3 | 0.9 | 0.8 | 1.0 | 0.7 | 0.9 | 1.2 | 1.1 | 1.4 | 3.99 |
| YLR081W | 1.3 | 0.8 | 2.8 | 3.6 | 1.0 | 0.9 | 0.7 | 0.9 | 0.1 | 2.2 | 2.6 | 1.0 | 0.6 | 1.0 | 0.7 | 1.2 | 0.7 | 0.9 | 1.46 |
| YMR011W | 1.3 | 1.0 | 9.4 | 5.5 | 0.9 | 0.6 | 0.8 | 1.1 | 0.0 | 0.7 | 1.5 | 0.8 | 0.4 | 0.6 | 0.6 | 1.2 | 1.3 | 1.6 | 4.95 |
| YOL156W | 1.1 | 0.7 | 2.5 | 1.1 | 1.2 | 1.2 | 1.0 | 1.0 | 2.3 | 2.1 | 1.1 | 0.9 | 1.7 | 0.9 | 1.9 | 0.9 | 0.9 | 0.9 | 0.53 |
| YPL036W | 0.5 | 0.8 | 2.8 | 0.9 | 1.3 | 0.7 | 0.3 | 0.5 | 0.1 | 0.3 | 0.8 | 0.5 | 0.3 | 0.6 | 1.0 | 0.7 | 0.4 | 0.5 | 3.66 |
| YGR096W | 1.5 | 1.0 | 1.1 | 4.6 | 1.2 | 1.7 | 0.9 | 1.0 | 0.8 | 0.8 | 0.8 | 0.8 | 1.1 | 1.2 | 0.9 | 0.8 | 0.8 | 1.0 | 0.49 |
| YIL006W | 1.0 | 1.1 | 1.4 | 2.9 | 0.8 | 0.3 | 0.8 | 0.6 | 0.9 | 1.1 | 1.0 | 0.9 | 0.8 | 1.0 | 1.5 | 1.7 | 0.8 | 0.9 | 0.28 |
| YKL016C | 1.5 | 1.3 | 0.7 | 1.7 | 1.5 | 0.8 | 1.5 | 1.6 | 1.3 | 1.8 | 1.3 | 1.0 | 1.3 | 1.6 | 0.7 | 1.8 | 1.0 | 1.7 | 2.08 |
| YKR067W | 0.8 | 2.6 | 1.5 | 2.5 | 1.3 | 1.8 | 2.0 | 0.8 | 1.6 | 2.3 | 1.3 | 1.0 | 1.3 | 1.3 | 1.3 | 2.0 | 0.8 | 1.0 | 0.58 |
| YMR162C | 0.8 | 1.1 | 1.1 | 2.0 | 0.9 | 1.0 | 0.8 | 0.8 | 0.7 | 1.0 | 1.7 | 0.7 | 0.9 | 1.0 | 1.0 | 0.8 | 0.8 | 1.1 | 0.32 |
| YOR348C | 1.1 | 0.7 | 1.2 | 2.0 | 0.9 | 0.7 | 0.8 | 0.8 | 0.5 | 0.4 | 1.1 | 0.7 | 1.1 | 1.2 | 1.6 | 1.7 | 0.9 | 0.9 | 0.18 |
| YPR192W | 0.9 | 1.1 | 0.9 | 5.0 | 1.2 | 0.9 | 0.9 | 0.6 | 0.7 | 1.3 | 0.7 | 0.5 | 1.1 | 0.7 | 1.7 | 2.6 | 1.6 | 0.9 | 0.28 |
| YPR194C | 2.2 | 1.4 | 0.5 | 2.9 | 1.3 | 1.5 | 0.7 | 1.0 | 0.5 | 0.6 | 0.7 | 0.7 | 1.0 | 1.0 | 0.4 | 0.8 | 1.0 | 0.9 | 0.27 |
| YDR086C | 1.3 | 1.0 | 1.2 | 1.4 | 1.0 | 0.8 | 2.0 | 1.9 | 0.6 | 0.8 | 1.1 | 0.7 | 0.7 | 1.5 | 2.1 | 1.3 | 1.4 | 1.9 | 2.60 |
| YGR224W | 0.9 | 1.2 | 1.2 | 1.2 | 1.4 | 0.8 | 0.4 | 0.6 | 0.9 | 0.9 | 0.8 | 0.6 | 0.8 | 0.9 | 1.1 | 1.0 | 2.8 | 1.3 | 0.27 |
| YDR387C | 0.8 | 0.9 | 1.2 | 2.2 | 0.9 | 1.2 | 1.3 | 0.7 | 1.4 | 2.0 | 2.0 | 0.9 | 1.3 | 0.9 | 1.4 | 2.7 | 1.2 | 1.1 | 0.86 |
| YFL050C | 0.7 | 1.0 | 1.1 | 2.2 | 1.0 | 0.9 | 0.8 | 0.9 | 0.5 | 1.6 | 1.2 | 0.9 | 1.2 | 1.0 | 1.4 | 2.3 | 0.8 | 1.0 | 0.36 |
| YBR298C | 1.1 | 1.0 | 1.6 | 2.3 | 1.4 | 0.4 | 1.7 | 0.5 | 0.3 | 1.1 | 1.5 | 1.1 | 0.8 | 0.9 | 0.2 | 2.1 | 0.9 | 0.8 | 0.88 |
| YLR295C | 1.4 | 1.1 | 1.1 | 1.7 | 2.2 | 1.1 | 1.1 | 1.2 | 1.4 | 1.2 | 1.3 | 0.5 | 0.8 | 1.2 | 0.5 | 2.4 | 0.9 | 1.5 | 1.07 |
| YNR072W | 1.1 | 1.5 | 0.5 | 0.7 | 1.0 | 0.9 | 1.5 | 0.9 | 0.7 | 1.8 | 1.4 | 1.0 | 1.5 | 1.0 | 1.8 | 2.1 | 1.3 | 1.0 | 0.26 |
| YOR316C | 0.7 | 2.5 | 1.5 | 1.5 | 1.0 | 0.9 | 1.4 | 0.8 | 1.6 | 2.0 | 2.4 | 1.1 | 1.6 | 1.0 | 1.5 | 2.2 | 0.8 | 1.0 | 0.93 |
| YOR328W | 1.0 | 1.0 | | 1.0 | 1.0 | 2.9 | | | | | 1.1 | 1.8 | | 0.8 | 3.3 | 2.1 | | | 0.19 |
| YPL134C | 1.5 | 1.0 | 1.6 | 1.6 | 1.4 | 1.2 | 1.2 | 1.3 | 0.6 | 0.8 | 1.7 | 0.8 | 1.0 | 1.4 | 1.1 | 3.3 | 1.3 | 1.3 | 0.70 |

TABLE 6
Stress protein genes

| | The level of expressed mRNA in the presence of chemical substance/ | | | | | | | | | | | | | | | | | | |
|---|---|---|---|---|---|---|---|---|---|---|---|---|---|---|---|---|---|---|---|
| | The level of expressed mRNA in the absence of chemical substance | | | | | | | | | | | | | | | | | | |
| yeast genes | (1) | (2) | (3) | (4) | (5) | (6) | (7) | (8) | (9) | (10) | (11) | (12) | (13) | (14) | (15) | (16) | (17) | (18) | Intensity |
| YBR072W | 18.5 | 17.4 | 18.4 | 10.5 | 1.2 | 32.2 | 6.6 | 2.1 | 29.4 | 43.4 | 6.3 | 16.3 | 38.5 | 5.8 | 4.9 | 23.1 | 1.8 | 2.2 | 0.59 |
| YFL014W | 3.4 | 5.1 | 5.7 | 11.0 | 1.0 | 9.3 | 5.5 | 3.4 | 13.1 | 5.8 | 5.0 | 4.3 | 15.2 | 7.3 | 6.3 | 14.2 | 1.5 | 8.8 | 2.14 |
| YLL060C | 12.5 | 4.2 | 2.3 | 2.6 | 0.9 | 1.4 | 2.0 | 5.8 | 13.0 | 23.2 | 14.1 | 1.1 | 1.8 | 10.6 | 1.5 | 3.6 | 1.9 | 2.3 | 0.57 |
| YAL005C | 0.6 | 1.6 | 7.0 | 0.9 | 1.2 | 1.6 | 1.2 | 0.9 | 3.9 | 1.8 | 1.0 | 1.2 | 5.0 | 0.4 | 1.6 | 0.7 | 0.8 | 0.5 | 5.23 |
| YBL075C | 1.3 | 4.3 | 3.1 | 1.6 | 1.1 | 0.8 | 1.3 | 0.8 | 60.3 | 6.7 | 1.6 | 1.1 | 8.0 | 1.1 | 2.6 | 2.2 | 1.2 | 0.7 | 1.17 |
| YBR169C | 1.0 | 2.1 | 3.9 | 1.9 | 0.8 | 4.9 | 2.5 | 0.9 | 7.0 | 4.3 | 2.2 | 1.7 | 7.2 | 1.1 | 1.6 | 2.4 | 1.3 | 1.6 | 1.10 |
| YCL035C | 2.0 | 2.3 | 1.5 | 1.7 | 1.5 | 5.1 | 2.7 | 1.4 | 1.9 | 2.3 | 2.1 | 1.5 | 2.8 | 2.5 | 1.9 | 5.5 | 2.6 | 4.2 | 1.74 |
| YCR060W | | 0.7 | 0.9 | | 0.8 | 3.3 | 2.4 | 1.2 | 0.7 | 1.1 | 1.2 | 1.7 | 8.6 | 1.7 | | 3.3 | 1.2 | 1.7 | 1.04 |
| YDR155C | 1.1 | 1.5 | 3.2 | 1.8 | 0.8 | 1.5 | 1.2 | 1.4 | 1.3 | 1.3 | 3.8 | 1.4 | 3.3 | 1.0 | 2.5 | 1.6 | 1.2 | 2.5 | 4.99 |
| YDR258C | 1.6 | 2.3 | 2.8 | 1.4 | 1.3 | 2.4 | 3.9 | 1.9 | 4.9 | 13.3 | 1.7 | 1.8 | 5.2 | 1.5 | 0.6 | 1.0 | 1.3 | 1.3 | 0.87 |
| YER103W | 0.7 | 2.1 | 5.2 | 2.1 | 1.1 | 1.7 | 1.0 | 0.9 | 20.3 | 9.0 | 1.8 | 1.9 | 9.0 | 0.9 | 3.2 | 1.2 | 1.4 | 0.6 | 2.10 |
| YKL210W | 0.5 | 1.7 | 2.7 | 0.7 | 0.8 | 1.4 | 1.0 | 0.9 | 3.5 | 2.9 | 1.4 | 1.3 | 2.9 | 0.8 | 1.0 | 0.9 | 0.7 | 1.1 | 2.29 |
| YLL024C | 0.5 | 1.6 | 5.3 | 0.8 | 0.7 | 1.0 | 1.1 | 0.7 | 2.8 | 2.4 | 1.6 | 1.0 | 4.8 | 0.4 | 1.3 | 0.5 | 0.5 | 0.6 | 5.12 |
| YLL026W | 1.8 | 2.9 | 7.7 | 1.7 | 0.9 | 1.9 | 2.9 | 1.4 | 11.4 | 6.6 | 1.7 | 1.9 | 8.1 | 0.8 | 0.9 | 1.6 | 1.7 | 2.5 | 2.56 |
| YNL160W | 2.1 | 3.7 | 10.6 | 2.0 | 1.3 | 5.8 | 2.4 | 1.2 | 3.5 | 2.7 | 5.4 | 1.4 | 6.0 | 1.3 | 0.7 | 4.0 | 1.0 | 1.0 | 1.77 |
| YNL241C | 1.3 | 2.5 | 4.3 | 1.0 | 0.8 | 0.9 | 3.2 | 0.9 | 3.4 | 7.4 | 3.0 | 2.0 | 4.9 | 1.1 | 7.0 | 2.8 | 1.0 | 1.0 | 0.68 |
| YOR027W | 0.7 | 2.4 | 4.4 | 0.8 | 1.0 | 1.6 | 1.3 | 0.9 | 3.5 | 4.0 | 1.3 | 1.3 | 5.1 | 0.8 | 1.4 | 0.6 | 0.9 | 0.8 | 1.52 |
| YPL240C | 0.7 | 1.4 | 2.4 | 0.9 | 1.3 | 1.2 | 1.4 | 1.2 | 3.5 | 2.6 | 1.0 | 0.8 | 2.9 | 0.7 | 1.2 | 0.7 | 0.8 | 1.0 | 4.83 |
| YDR293C | 0.9 | 0.9 | 1.2 | 1.4 | 0.6 | 0.9 | 0.9 | 0.6 | 1.2 | 1.2 | 1.1 | 0.9 | 2.3 | 0.8 | 1.7 | 1.6 | 1.4 | 1.3 | 1.12 |
| YDR436W | 0.9 | 0.9 | 1.1 | 1.4 | 0.7 | 1.1 | 1.2 | 0.9 | 3.5 | 1.1 | 2.0 | 0.9 | 2.7 | 1.2 | 0.8 | 1.4 | 0.9 | 1.2 | 0.53 |
| YDR519W | 1.3 | 3.2 | 1.7 | 1.6 | 0.7 | 1.3 | 1.9 | 1.4 | 0.5 | 1.2 | 1.2 | 1.7 | 2.5 | 1.3 | 2.0 | 1.5 | 1.8 | 2.4 | 2.01 |
| YEL030W | 0.7 | 0.9 | 1.9 | 1.1 | 0.4 | 0.6 | 0.9 | 0.7 | 2.3 | 2.5 | 1.7 | 0.9 | 2.7 | 0.7 | 0.8 | 0.8 | 0.9 | 0.6 | 1.28 |
| YER125W | 0.6 | 1.1 | 1.5 | 0.9 | 1.4 | 0.7 | 0.8 | 0.7 | 0.6 | 1.2 | 1.0 | 0.7 | 1.9 | 0.7 | 0.8 | 1.0 | 1.0 | 1.0 | 1.48 |
| YFL016C | 0.8 | 1.0 | 1.6 | 0.9 | 1.3 | 1.3 | 1.4 | 0.7 | 6.4 | 2.9 | 1.2 | 1.0 | 2.8 | 0.8 | 0.8 | 0.6 | 1.2 | 0.9 | 1.25 |
| YFR052W | 1.1 | 1.6 | 1.3 | 1.5 | 1.4 | 3.2 | 2.2 | 1.5 | 3.7 | 3.0 | 0.9 | 1.8 | 3.8 | 3.3 | 1.4 | 2.7 | 1.2 | 1.9 | 1.62 |
| YHR057C | 1.1 | 1.2 | 0.7 | 0.9 | 1.3 | 1.3 | 1.3 | 1.0 | 2.6 | 1.2 | 1.6 | 1.1 | 1.9 | 1.9 | 1.2 | 2.2 | 1.6 | 1.3 | 1.07 |
| YIR037W | 1.4 | 2.2 | 2.0 | 2.3 | 0.6 | 2.7 | 1.8 | 1.3 | 6.2 | 3.8 | 3.0 | 1.3 | 2.4 | 2.3 | 1.0 | 4.0 | 1.7 | 2.9 | 1.97 |
| YIR038C | 1.3 | 3.5 | 4.6 | 2.0 | 0.7 | 2.5 | 2.0 | 1.3 | 4.6 | 4.5 | 3.4 | 1.1 | 2.5 | 2.5 | 1.2 | 6.0 | 2.8 | 2.0 | 1.11 |
| YJR045C | 0.5 | 1.9 | 3.9 | | 0.5 | 1.2 | 1.0 | 0.8 | 3.2 | 3.0 | 1.4 | 1.0 | 2.3 | 0.5 | 2.3 | 0.8 | 0.7 | 0.9 | 3.78 |
| YLL039C | 1.1 | 1.0 | 1.7 | 1.2 | 0.9 | 1.5 | 1.1 | 1.1 | 3.4 | 1.5 | 1.1 | 1.0 | 2.2 | 1.4 | 3.0 | 1.1 | 1.6 | 2.1 | 3.60 |
| YLR259C | 0.7 | 1.0 | 3.1 | 1.6 | 1.1 | 0.9 | 1.8 | 0.8 | 1.8 | 2.6 | 3.0 | 0.7 | 2.5 | 0.8 | 2.1 | 1.3 | 0.8 | 1.0 | 2.09 |
| YML070W | 0.9 | 1.7 | 2.5 | 1.9 | 1.4 | 2.2 | 3.1 | 1.3 | 4.5 | 3.9 | 1.5 | 1.3 | 3.3 | 1.3 | 0.9 | 2.6 | 1.2 | 1.3 | 1.03 |
| YPL106C | 0.6 | 1.7 | 2.4 | 1.1 | 1.2 | 1.6 | 1.1 | 1.2 | 4.9 | 1.8 | 1.3 | 1.1 | 3.1 | 0.9 | 1.1 | 0.5 | 0.5 | 0.5 | 3.34 |
| YPR026W | 0.9 | 1.2 | 5.0 | 1.3 | 0.8 | | 1.3 | 1.0 | 0.9 | 3.1 | 1.6 | 1.2 | 2.3 | 1.5 | 3.1 | 2.5 | 0.9 | 1.3 | 0.26 |
| YDR077W | 1.2 | 1.0 | 3.5 | 2.1 | 0.8 | 1.0 | 0.7 | 0.8 | 0.7 | 0.7 | 6.8 | 0.8 | 1.0 | 0.3 | 6.1 | 1.0 | 1.4 | 1.1 | 4.77 |
| YKL163W | 1.1 | 1.4 | 3.4 | 1.9 | 0.6 | 1.1 | 0.9 | 0.8 | 0.7 | 1.6 | 1.3 | 0.9 | 1.5 | 0.5 | 16.8 | 3.0 | 0.8 | 1.0 | 2.01 |
| YLR109W | 0.8 | 2.9 | 6.0 | 0.9 | 1.3 | 2.1 | 2.6 | 0.9 | 2.4 | 2.7 | 1.5 | 1.6 | 3.2 | 2.3 | 3.9 | 1.8 | 1.1 | 1.2 | 3.86 |
| YOR208W | 1.3 | 1.0 | 1.2 | 2.9 | 1.4 | 1.8 | 1.1 | 1.0 | 1.5 | 2.0 | 1.2 | 1.2 | 1.5 | 1.3 | 4.4 | 1.9 | 1.6 | 1.6 | 0.56 |
| YDR098C | 0.8 | 1.4 | 1.0 | 0.8 | 1.5 | 1.1 | 1.5 | 1.2 | 3.7 | 1.5 | 1.4 | 1.3 | 1.2 | 1.5 | 2.2 | 0.8 | 1.0 | 1.2 | 1.59 |
| YHR030C | 2.1 | 0.8 | 0.6 | 2.9 | 1.8 | 0.9 | 1.8 | 1.5 | 0.6 | 2.5 | 1.5 | 0.7 | 1.0 | 1.1 | 3.4 | 1.4 | 2.0 | 1.8 | 1.05 |
| YJL159W | 1.0 | 1.3 | 2.8 | 1.1 | 1.0 | 1.8 | 0.9 | 0.8 | 0.5 | 1.2 | 1.2 | 1.0 | 1.7 | 0.6 | 5.3 | 1.0 | 1.4 | 0.9 | 2.45 |
| YMR173W | 1.5 | 3.6 | 3.0 | 0.9 | 1.1 | 5.0 | 2.4 | 2.3 | 2.7 | 8.2 | 3.2 | 1.4 | 2.0 | 2.3 | 2.5 | 1.2 | 1.9 | 1.8 | 1.37 |
| YCR021C | 5.2 | 1.4 | 4.2 | 8.6 | 0.9 | 2.2 | 0.9 | 0.8 | 2.2 | 1.0 | 0.6 | 0.5 | 0.9 | 1.6 | 0.5 | 0.6 | 0.7 | 0.8 | 1.36 |
| YDL022W | 0.9 | 2.6 | 6.9 | 1.5 | 0.7 | 2.1 | 1.5 | 0.8 | 1.2 | 1.8 | 2.8 | 1.2 | 1.5 | 0.8 | 0.9 | 1.1 | 1.0 | 1.4 | 1.79 |
| YDR033W | 1.1 | 1.3 | 4.6 | 1.5 | 1.1 | 6.7 | 0.8 | 1.0 | 0.3 | 1.0 | 0.7 | 1.0 | 0.7 | 1.0 | 0.8 | 1.0 | 0.7 | 1.0 | 5.78 |
| YFL020C | 1.0 | 1.1 | 1.9 | 1.2 | 1.1 | 2.0 | 1.4 | 0.7 | 1.3 | 1.4 | 1.2 | 1.3 | 1.4 | 1.0 | 1.4 | 0.9 | 1.5 | 0.9 | 0.70 |
| YGL073W | 1.0 | 2.1 | 0.9 | 1.0 | 1.7 | 1.9 | 1.3 | 0.8 | 1.9 | 2.4 | 0.8 | 0.8 | 1.5 | 1.2 | 0.9 | 1.1 | 2.5 | 1.2 | 0.66 |
| YIL033C | 0.8 | 1.3 | 3.3 | 1.3 | 0.7 | 2.0 | 1.1 | 0.7 | 2.4 | 1.3 | 1.8 | 1.1 | 2.5 | 0.7 | 1.0 | 2.0 | 1.0 | 1.2 | 1.18 |
| YMR021C | 1.0 | 0.7 | 0.9 | 1.1 | 1.6 | 2.2 | 2.5 | 1.3 | 0.6 | 1.3 | 0.9 | 0.8 | 1.4 | 1.3 | 1.0 | 2.0 | 3.3 | 3.4 | 1.14 |
| YBR126C | 0.8 | 1.9 | 5.6 | 1.2 | 0.7 | 2.9 | 2.3 | 0.6 | 1.7 | | 1.1 | 1.3 | 2.1 | 0.7 | 1.0 | 1.7 | 1.5 | 1.3 | 1.96 |
| YBR067C | 1.6 | 2.5 | 2.8 | 0.9 | 1.1 | 1.1 | 1.1 | 1.1 | 1.7 | 1.0 | 3.1 | 1.0 | 0.9 | 0.7 | 3.3 | 2.5 | 0.5 | 0.4 | 1.76 |
| YDR513W | 2.2 | 2.5 | 2.3 | 2.6 | 0.9 | 2.1 | 1.6 | 1.6 | 4.6 | 3.1 | 2.0 | 0.9 | 1.8 | 2.0 | 1.3 | 3.8 | 1.3 | 3.2 | 3.10 |
| YGR088W | 1.3 | 1.2 | 7.7 | 2.4 | 1.0 | 1.3 | 0.8 | 0.8 | 1.5 | 3.2 | 3.9 | 1.1 | 1.2 | 1.3 | 0.7 | 5.6 | 0.9 | 2.0 | 0.75 |
| YHR104W | 1.0 | 4.1 | 15.7 | 1.9 | 1.1 | 1.2 | 1.5 | 1.1 | 4.8 | 2.3 | 2.6 | 1.0 | 1.2 | 1.5 | 1.6 | 1.3 | 1.9 | 1.9 | 1.57 |
| YKL161C | 4.2 | 0.8 | 0.9 | 2.2 | 1.4 | 2.0 | 2.0 | 1.9 | 1.4 | 1.7 | 3.1 | 0.9 | 1.3 | 1.4 | 1.3 | 1.1 | 1.4 | 1.9 | 0.45 |
| YPL223C | 4.8 | 20.0 | 1.2 | 5.1 | 0.9 | 1.5 | 1.1 | 1.2 | 29.4 | 16.5 | 1.8 | 0.9 | 2.3 | 2.5 | 1.5 | 19.0 | 1.0 | 1.3 | 0.31 |
| YBR054W | 1.9 | 1.8 | 5.4 | 1.7 | 0.8 | 1.1 | 0.6 | 2.1 | 0.4 | 1.2 | 1.8 | 0.7 | 1.5 | 1.0 | 0.6 | 0.4 | 0.7 | 0.7 | 3.02 |
| YAL015C | 1.2 | 1.1 | 1.7 | 1.9 | 1.1 | 0.7 | 1.2 | 1.1 | 4.0 | 2.7 | 1.4 | 1.0 | 1.2 | 1.6 | 0.7 | 2.0 | 1.1 | 1.2 | 0.61 |
| YDL025C | 1.7 | 5.0 | 3.5 | 1.5 | 0.6 | 0.6 | 1.0 | 1.1 | 3.6 | 4.5 | 1.5 | 0.9 | 1.2 | 1.7 | 1.0 | 1.7 | 1.0 | 1.4 | 0.56 |
| YML007W | 0.9 | 0.8 | 0.9 | 1.3 | 1.0 | 0.5 | 1.1 | 1.9 | 2.1 | 2.5 | 1.3 | 1.1 | 1.7 | 1.2 | 1.0 | 1.4 | 1.0 | 1.4 | 1.54 |
| YER042W | 3.0 | 2.2 | 3.1 | 1.0 | 1.1 | 1.6 | 1.1 | 1.7 | 6.8 | 2.1 | 1.8 | 1.1 | 1.4 | 5.2 | 1.0 | 1.9 | 0.7 | 1.1 | 1.80 |
| YOL064C | 1.4 | 2.1 | 1.1 | 0.6 | 0.9 | 1.4 | 1.2 | 1.0 | 8.7 | 2.7 | 1.0 | 0.9 | 2.0 | 0.9 | 1.0 | 1.7 | 1.2 | 1.3 | 1.32 |
| YGL181W | 0.9 | 0.9 | 1.2 | 1.1 | 1.1 | 1.9 | 1.3 | 1.2 | 2.7 | 2.3 | 1.2 | 1.0 | 2.8 | 1.0 | 1.5 | 1.3 | 1.0 | 1.3 | 0.98 |
| YJL128C | 0.8 | 1.4 | 0.4 | 0.8 | 1.2 | 1.3 | 1.8 | 0.9 | 2.0 | 1.7 | 0.6 | 1.0 | 1.7 | 0.8 | 1.2 | 0.9 | 1.0 | 1.0 | 0.42 |
| YJL165C | 1.0 | 0.8 | 1.3 | 1.6 | 1.1 | 0.5 | 0.8 | 0.7 | 2.8 | 1.0 | 1.7 | 0.9 | 1.5 | 0.9 | 1.0 | 1.5 | 1.3 | 1.8 | 0.87 |
| YJR090C | 0.8 | 1.0 | 0.6 | 0.8 | 1.2 | 1.2 | 0.8 | 0.8 | 1.9 | 1.2 | 0.4 | 0.8 | 2.2 | 1.0 | 1.6 | 0.8 | 1.1 | 1.0 | 0.60 |
| YMR186W | 0.6 | 1.4 | 2.0 | 0.9 | 1.3 | 1.3 | 1.2 | 0.8 | 2.6 | 1.3 | 1.1 | 1.0 | 2.4 | 0.6 | 1.3 | 0.6 | 0.7 | 1.1 | 6.64 |
| YNL064C | 0.9 | 1.8 | 1.8 | 1.1 | 0.9 | 0.8 | 0.6 | 1.4 | 2.4 | 1.8 | 0.9 | 0.8 | 1.1 | 0.8 | 0.7 | 0.3 | 0.7 | 0.7 | 3.79 |
| YOR008C | 0.7 | 0.8 | 1.4 | 1.1 | 1.4 | 1.1 | 0.8 | 0.8 | 1.9 | 1.0 | 1.0 | 0.7 | 0.9 | 0.9 | 0.9 | 1.2 | 0.8 | 1.3 | 1.75 |
| YPL194W | 0.9 | 1.4 | 0.4 | 1.1 | 1.3 | 1.1 | 1.5 | 1.3 | 9.6 | 3.1 | 0.9 | 0.9 | 1.4 | 1.1 | 1.4 | 0.9 | 1.0 | 0.8 | 0.22 |
| YBL093C | 1.0 | 2.0 | 0.4 | 1.4 | 1.0 | 0.8 | 1.3 | 1.6 | 0.7 | 1.1 | 1.3 | 1.1 | 2.1 | 1.0 | 0.5 | 0.9 | 1.4 | 1.3 | 1.47 |
| YKR053C | 0.9 | 2.0 | 1.0 | 1.2 | 1.0 | 1.1 | 1.7 | 0.9 | 0.8 | 1.8 | 0.5 | 0.7 | 0.7 | 1.3 | 0.3 | 0.9 | 2.8 | 2.4 | 0.35 |
| YJL158C | 0.9 | 0.6 | 2.8 | 1.5 | 1.4 | 0.5 | 0.4 | 0.7 | 0.2 | 0.5 | 1.9 | 1.1 | 0.4 | 0.5 | 1.7 | 0.6 | 1.4 | 1.4 | 3.48 |
| YER057C | 1.2 | 2.0 | 3.0 | 1.8 | 0.8 | 1.6 | 1.4 | 1.0 | 0.6 | 1.4 | 1.8 | 1.5 | 1.0 | 1.4 | 1.7 | 2.8 | 1.1 | 1.8 | 2.03 |
| YHL046C | 1.1 | 1.7 | 2.1 | 1.3 | 0.9 | 0.8 | 1.3 | 1.0 | 1.0 | 2.1 | 0.4 | 1.1 | 1.4 | 1.4 | 1.0 | 1.4 | 0.9 | 0.9 | 0.55 |
| YIL011W | 0.7 | 0.5 | 3.9 | 0.6 | 1.1 | 0.7 | 0.6 | 0.7 | 0.0 | 0.5 | 2.5 | 0.6 | 0.5 | 0.5 | 0.9 | 1.0 | 0.8 | 0.8 | 0.57 |
| YKL164C | 0.6 | 1.1 | 2.5 | 0.9 | 1.1 | 1.4 | 0.8 | 0.8 | 0.4 | 0.7 | 1.0 | 0.8 | 0.9 | 0.3 | 1.2 | 1.1 | 0.8 | 0.8 | 2.75 |
| YNR076W | 1.3 | 1.3 | 2.5 | 1.0 | 1.2 | 1.2 | 1.2 | 1.0 | 1.3 | 2.5 | 1.4 | 0.7 | 0.9 | 0.8 | 1.1 | 0.8 | 1.5 | 0.8 | 0.42 |
| YOL161C | 1.2 | 1.1 | 2.4 | 1.4 | 0.9 | 1.0 | 0.9 | 0.8 | 0.7 | 2.0 | 1.5 | 1.3 | 1.2 | 0.9 | 1.1 | 1.6 | 1.1 | 1.0 | 0.89 |
| YOR009W | 0.9 | 0.8 | 3.2 | 0.7 | 0.8 | 0.9 | 0.6 | 0.7 | 0.4 | 0.5 | 1.4 | 0.4 | 0.4 | 0.7 | 0.6 | 0.8 | 0.9 | 0.8 | 0.36 |
| YOR010C | 0.7 | 0.7 | 3.2 | 0.6 | 1.2 | 0.5 | 0.4 | 0.5 | 0.6 | 0.7 | 1.2 | 0.5 | 0.5 | 0.4 | 0.6 | 1.1 | 1.4 | 0.6 | 0.61 |
| YPL059W | 1.2 | 0.9 | 2.6 | 1.3 | 1.2 | 1.3 | 0.7 | 1.4 | 2.0 | 1.3 | 1.6 | 0.8 | 0.7 | 1.3 | 1.0 | 2.1 | 1.8 | 1.3 | 1.35 |
| YBR044C | 0.8 | 1.4 | 0.7 | 1.9 | 1.4 | 1.0 | 1.5 | 1.1 | 0.8 | 1.2 | 1.1 | 0.8 | 1.2 | 0.9 | 0.9 | 1.4 | 0.9 | 1.3 | 0.57 |
| YEL039C | 1.1 | 0.7 | 1.3 | 5.1 | 0.9 | 1.5 | 0.6 | 1.1 | 1.2 | 1.2 | 1.7 | 0.6 | 0.5 | 1.4 | 0.4 | 3.5 | 0.7 | 1.1 | 1.59 |
| YGL115W | 0.9 | 0.6 | 1.2 | 2.0 | 1.2 | 1.2 | 1.4 | 1.0 | 1.2 | 1.2 | 1.0 | 0.9 | 0.7 | 1.2 | 0.7 | 1.7 | 0.8 | 1.2 | 2.46 |
| YLR006C | 0.7 | 0.7 | 0.9 | 6.2 | 1.6 | 0.9 | 1.1 | 0.9 | 1.1 | 1.1 | 1.5 | 0.7 | 0.8 | 1.0 | 0.9 | 0.8 | 1.6 | 1.0 | 0.40 |
| YPR005C | 1.3 | 0.8 | 1.5 | 3.8 | 1.0 | 0.8 | 1.2 | 1.2 | 2.8 | 2.0 | 0.4 | 0.6 | 0.7 | 1.3 | 2.7 | 0.8 | 1.0 | 1.1 | 0.24 |
| YHL028W | 0.7 | 0.8 | 1.4 | 0.5 | 1.1 | 1.8 | 2.1 | 0.4 | 0.2 | 0.4 | 0.7 | 1.5 | 2.1 | 1.3 | 0.6 | 2.6 | 1.6 | 2.5 | 0.62 |
| YCR083W | 1.1 | 2.8 | 1.5 | 1.8 | 1.6 | 2.4 | 1.8 | 1.5 | 1.8 | 1.8 | 1.8 | 1.1 | 1.8 | 2.4 | 1.0 | 3.1 | 1.3 | 1.3 | 0.98 |
| YPL140C | 0.9 | 1.3 | 0.9 | 1.0 | 0.8 | 0.3 | 1.4 | 1.1 | 0.9 | 0.9 | 0.8 | 0.8 | 0.9 | 1.1 | 0.7 | 1.8 | 0.9 | 1.4 | 0.46 |

TABLE 7
Metabolism protein genes

| | The level of expressed mRNA in the presence of chemical substance/ | | | | | | | | | | | | | | | | | | |
|---|---|---|---|---|---|---|---|---|---|---|---|---|---|---|---|---|---|---|---|
| | The level of expressed mRNA in the absence of chemical substance | | | | | | | | | | | | | | | | | | |
| | | | | | | | | | | | | | | | | | | | Inten- |
| yeast genes | (1) | (2) | (3) | (4) | (5) | (6) | (7) | (8) | (9) | (10) | (11) | (12) | (13) | (14) | (15) | (16) | (17) | (18) | sity |
| YCR107W | 12.0 | 2.6 | 1.8 | 1.6 | 0.7 | 1.5 | 1.5 | 15.6 | 196.6 | 34.0 | 23.0 | 0.9 | 4.0 | 8.3 | 4.8 | 3.1 | 1.3 | 1.1 | 0.59 |
| YDL243C | 14.4 | 2.7 | 2.4 | 1.0 | 1.1 | 2.2 | 1.6 | 11.8 | 64.2 | 29.6 | 19.6 | 1.1 | 4.1 | 12.0 | 4.2 | 3.0 | 1.8 | 1.5 | 0.76 |
| YFL014W | 3.4 | 7.2 | 5.7 | 11.0 | 1.0 | 9.3 | 5.5 | 3.4 | 13.1 | 5.8 | 5.0 | 4.3 | 15.2 | 7.3 | 6.3 | 14.2 | 1.5 | 8.8 | 2.14 |
| YFL056C | 19.0 | 2.3 | 2.3 | 1.5 | 0.9 | 0.6 | 1.4 | 18.5 | 162.3 | 31.3 | 68.3 | 1.0 | 4.7 | 7.8 | 5.0 | 3.4 | 1.0 | 1.1 | 0.55 |
| YFL057C | 20.9 | 5.8 | 1.5 | 1.8 | 0.9 | 1.2 | 1.8 | 18.0 | 51.8 | 46.1 | 27.7 | 1.0 | 4.1 | 23.4 | 3.1 | 3.9 | 1.6 | 1.3 | 0.71 |
| YJR155W | 10.6 | 3.7 | 1.4 | 2.5 | 0.7 | 1.4 | 1.7 | 10.6 | 38.2 | 18.8 | 15.4 | 1.0 | 5.7 | 9.4 | 2.6 | 5.6 | 1.3 | 1.4 | 0.64 |
| YNL331C | 8.6 | 3.6 | 1.3 | 1.0 | 1.6 | 1.8 | 1.9 | 13.1 | 42.6 | 36.3 | 21.8 | 0.9 | 3.1 | 7.5 | 2.3 | 4.0 | 1.7 | 1.3 | 0.58 |
| YNL332W | 1.1 | 1.5 | 1.1 | 2.0 | 1.6 | 1.1 | 2.1 | 1.5 | 3.3 | 2.1 | 2.6 | 1.2 | 2.1 | 3.0 | 5.4 | 2.2 | 1.0 | 1.0 | 0.24 |
| YOL165C | 10.1 | 4.5 | 1.8 | 0.9 | 0.9 | 1.7 | 1.4 | 17.8 | 46.9 | 23.3 | 17.6 | 0.8 | 3.7 | 9.1 | 3.0 | 1.8 | 1.6 | 1.0 | 0.69 |
| YPR167C | 5.3 | 5.8 | 1.9 | 0.8 | 1.4 | 2.6 | 1.2 | 5.5 | 76.6 | 9.0 | 1.9 | 1.3 | 1.9 | 4.2 | 0.9 | 0.9 | 1.7 | 1.4 | 0.54 |
| YBR256C | 3.0 | 1.4 | 0.6 | 1.3 | 1.6 | 2.0 | 2.1 | 2.3 | 18.1 | 6.8 | 3.2 | 1.7 | 3.5 | 5.6 | 1.6 | 3.0 | 2.6 | 3.3 | 1.97 |
| YBR296C | 7.0 | 11.2 | 2.2 | 2.5 | 1.4 | 1.4 | 0.2 | 1.1 | 0.4 | 1.8 | 1.5 | 1.6 | 2.0 | 2.3 | 1.7 | 0.3 | 3.8 | 4.0 | 0.54 |
| YDL021W | 4.8 | 1.7 | 2.4 | 3.7 | 1.7 | 6.5 | 5.9 | 2.7 | 2.5 | 7.4 | 4.7 | 2.4 | 5.3 | 3.7 | 0.7 | 7.3 | 1.9 | 3.2 | 0.47 |
| YFL061W | 1.5 | 1.4 | 2.1 | 3.0 | 1.0 | 0.5 | 1.2 | 1.2 | 3.2 | 9.9 | 1.1 | 0.9 | 1.2 | 6.0 | 2.1 | 1.0 | 1.2 | 0.9 | 0.31 |
| YGR043C | 2.5 | 4.7 | 3.2 | 7.9 | 0.9 | 16.3 | 6.5 | 2.6 | 10.9 | 8.4 | 3.6 | 3.3 | 6.9 | 4.1 | 3.0 | 13.7 | 1.6 | 4.8 | 0.66 |
| YHR112C | 1.8 | 3.1 | 0.7 | 1.4 | 1.3 | 2.9 | 3.4 | 1.7 | 10.5 | 4.4 | 1.5 | 1.9 | 3.9 | 2.4 | 2.0 | 2.7 | 1.6 | 1.8 | 0.55 |
| YIR030C | 1.4 | 1.0 | 0.9 | 1.3 | 0.9 | 0.5 | 1.2 | 1.4 | 5.4 | 2.1 | 1.4 | 0.9 | 1.3 | 2.9 | 0.7 | 0.8 | 0.9 | 1.0 | 0.42 |
| YJR010W | 9.1 | 7.3 | 4.9 | 0.8 | 1.1 | 2.5 | 1.4 | 3.1 | 30.0 | 11.1 | 2.9 | 1.4 | 3.2 | 5.4 | 1.7 | 1.7 | 1.2 | 1.0 | 0.56 |
| YKL001C | 6.8 | 21.4 | 5.6 | 1.8 | 0.8 | 0.9 | 1.1 | 3.4 | 10.3 | 3.4 | 2.6 | 2.1 | 1.6 | 6.2 | 1.1 | 1.8 | 2.7 | 1.7 | 0.91 |
| YKR097W | 1.3 | 2.4 | 1.3 | 3.3 | 1.1 | 1.7 | 3.7 | 2.5 | 1.9 | 3.3 | 0.8 | 1.8 | 17.5 | 2.1 | 2.5 | 2.2 | 1.1 | 1.5 | 0.16 |
| YLR303W | 7.3 | 9.8 | 12.1 | 1.1 | 1.1 | 3.0 | 3.6 | 5.3 | 18.6 | 5.6 | 9.8 | 4.3 | 9.9 | 3.8 | 5.8 | 3.4 | 1.5 | 1.4 | 1.42 |
| YNL274C | 1.4 | 18.7 | 2.6 | 1.9 | 0.7 | 4.2 | 2.0 | 2.3 | 6.3 | 5.3 | 4.0 | 1.1 | 2.3 | 3.3 | 2.5 | 6.4 | 1.4 | 2.6 | 0.85 |
| YOL151W | 6.7 | 3.8 | 9.1 | 1.3 | 1.3 | 8.4 | 4.4 | 6.7 | 22.8 | 17.0 | 9.0 | 4.6 | 16.9 | 4.0 | 2.8 | 3.7 | 4.0 | 5.6 | 1.12 |
| YOR226C | 1.3 | 2.2 | 1.7 | 1.1 | 1.3 | 1.2 | 1.2 | 1.2 | 1.3 | 5.9 | 1.5 | 0.6 | 0.9 | 2.7 | 1.0 | 0.7 | 1.0 | 0.7 | 0.58 |
| YJL153C | 1.3 | 1.7 | 1.7 | 17.6 | 0.7 | 0.8 | 2.3 | 1.3 | 3.9 | 2.5 | 0.7 | 10.2 | 38.0 | 1.4 | 37.0 | 1.0 | 6.5 | 1.8 | 0.27 |
| YOR153W | 1.6 | 0.9 | 5.1 | 1.1 | 1.0 | 7.4 | 2.6 | 0.5 | 3.2 | 1.2 | 1.0 | 4.0 | 11.8 | 0.3 | 3.6 | 1.6 | 2.5 | 3.1 | 1.91 |
| YPL088W | 3.3 | 1.3 | 0.6 | 2.5 | 1.5 | 7.1 | 3.7 | 1.9 | 0.6 | 2.8 | 1.1 | 3.6 | 4.8 | 1.6 | 3.2 | 4.3 | 9.1 | 8.3 | 0.69 |
| YDL124W | 2.1 | 8.5 | 5.7 | 1.7 | 0.7 | 3.4 | 2.9 | 2.1 | 3.3 | 5.3 | 3.4 | 2.4 | 6.0 | 1.6 | 3.4 | 3.6 | 2.2 | 2.6 | 2.43 |
| YDL174C | 0.9 | 1.8 | 0.9 | 1.3 | 0.5 | 1.7 | 3.6 | 0.8 | 0.5 | 1.7 | 0.9 | 2.8 | 5.9 | 1.1 | 0.6 | 5.1 | 2.2 | 4.0 | 0.63 |
| YGL156W | 1.3 | 2.5 | 1.8 | 2.2 | 1.4 | 2.1 | 2.1 | 1.4 | 5.6 | 2.4 | 2.1 | 2.6 | 6.0 | 1.6 | 3.0 | 4.1 | 1.4 | 1.9 | 0.29 |
| YGR157W | 1.0 | 0.9 | 5.2 | 0.8 | 1.2 | 1.0 | 0.8 | 0.7 | 3.3 | 2.0 | 1.0 | 1.8 | 4.8 | 0.7 | 4.6 | 1.0 | 0.8 | 1.3 | 2.10 |
| YHR043C | 1.3 | 1.8 | 1.2 | 1.7 | 0.9 | 2.2 | 2.9 | 3.0 | 1.2 | 1.0 | 1.6 | 2.2 | 2.7 | 1.3 | 2.1 | 2.2 | 1.4 | 1.7 | 1.38 |
| YHR044C | 1.3 | 1.3 | 1.7 | 1.5 | 1.2 | 3.3 | 2.6 | 2.0 | 1.1 | 1.1 | 1.7 | 2.3 | 4.3 | 1.3 | 1.5 | 2.4 | 1.3 | 1.4 | 1.12 |
| YJR073C | 1.1 | 2.4 | 2.7 | 2.7 | 0.6 | 2.2 | 2.9 | 1.0 | 6.8 | 2.8 | 2.8 | 4.1 | 10.0 | 2.3 | 28.9 | 3.7 | 2.9 | 2.6 | 0.99 |
| YOR303W | 1.1 | 2.2 | 0.5 | 1.0 | 1.2 | 1.8 | 1.2 | 1.6 | 0.9 | 5.2 | 0.9 | 1.9 | 3.3 | 1.6 | 4.1 | 1.2 | 1.2 | 0.8 | 1.47 |
| YBR294W | 6.3 | 12.0 | 0.9 | 2.6 | 1.4 | 1.2 | 1.1 | 1.0 | 5.2 | 5.2 | 1.2 | 1.0 | 3.1 | 1.3 | 1.0 | 0.9 | 1.0 | 0.9 | 0.18 |
| YCL043C | 0.6 | 3.7 | 2.5 | 0.7 | 0.9 | 1.3 | 1.0 | 0.8 | 1.6 | 1.2 | 1.7 | 1.5 | 4.1 | 0.6 | 5.7 | 0.8 | 0.9 | 1.0 | 2.37 |
| YCL050C | 1.0 | 1.1 | 1.9 | 0.7 | 1.4 | 1.2 | 1.2 | 1.2 | 3.0 | 2.9 | 1.3 | 1.2 | 2.8 | 1.2 | 0.8 | 0.7 | 1.0 | 1.0 | 2.20 |
| YCR012W | 1.1 | 1.5 | 5.1 | 1.2 | 1.1 | 2.5 | 1.4 | 0.8 | 1.4 | 1.5 | 1.8 | 1.2 | 4.1 | 0.9 | 1.4 | 1.4 | 0.8 | 1.3 | 4.48 |
| YDR158W | 1.1 | 0.5 | 1.8 | 0.8 | 1.3 | 1.0 | 0.8 | 1.2 | 1.1 | 1.7 | 1.6 | 1.0 | 2.6 | 0.9 | 1.0 | 0.6 | 1.0 | 0.9 | 3.54 |
| YDR204W | 0.9 | 2.3 | 3.0 | 1.4 | 1.0 | 2.2 | 2.2 | 1.1 | 5.7 | 3.7 | 2.2 | 1.2 | 2.9 | 1.1 | 0.6 | 2.8 | 1.1 | 1.7 | 0.62 |
| YDR313C | 1.2 | 1.2 | 1.2 | 1.4 | 1.3 | 1.4 | 1.5 | 1.2 | 4.7 | 2.6 | 1.5 | 1.2 | 2.3 | 1.3 | 0.8 | 2.9 | 1.1 | 1.3 | 0.84 |
| YDR368W | 2.0 | 2.2 | 2.2 | 1.8 | 1.0 | 3.0 | 2.1 | 1.7 | 3.8 | 3.8 | 1.8 | 1.4 | 2.6 | 2.0 | 1.2 | 2.3 | 1.1 | 2.0 | 1.70 |
| YER091C | 3.6 | 3.9 | 30.7 | 1.3 | 0.9 | 2.3 | 2.1 | 0.9 | 5.8 | 1.4 | 3.2 | 2.0 | 5.4 | 1.2 | 10.2 | 2.7 | 1.2 | 1.0 | 1.23 |
| YFR053C | 1.7 | 1.4 | 3.0 | 2.2 | 0.9 | 3.2 | 2.1 | 2.8 | 0.6 | 1.1 | 1.5 | 1.7 | 3.0 | 0.6 | 0.6 | 3.0 | 1.4 | 2.2 | 4.17 |
| YGL062W | 0.6 | 1.3 | 1.1 | 0.6 | 0.9 | 1.0 | 1.3 | 0.8 | 1.6 | 2.5 | 0.7 | 1.6 | 4.5 | 1.2 | 2.7 | 1.5 | 1.1 | 0.9 | 0.77 |
| YGL157W | 0.7 | 0.9 | 1.8 | 0.9 | 1.5 | 5.4 | 3.3 | 1.4 | 0.6 | 1.8 | 1.4 | 1.5 | 4.8 | 1.2 | 5.1 | 1.7 | 1.8 | 2.3 | 1.69 |
| YGL184C | 4.6 | 15.9 | 57.4 | 1.3 | 1.1 | 1.0 | 1.2 | 1.9 | 37.9 | 66.8 | 1.7 | 2.6 | 7.4 | 1.4 | 0.4 | 1.5 | 1.0 | 0.9 | 0.16 |
| YGR032W | 1.0 | 1.4 | 1.0 | 1.3 | 0.7 | 1.9 | 0.8 | 0.6 | 0.5 | 0.7 | 1.5 | 1.5 | 3.9 | 1.1 | 5.4 | 1.5 | 3.2 | 2.8 | 1.18 |
| YGR124W | 0.5 | 0.6 | 1.7 | 0.7 | 1.2 | 1.1 | 0.7 | 1.0 | 2.2 | 1.3 | 1.1 | 1.1 | 2.3 | 0.5 | 1.5 | 0.3 | 0.7 | 0.6 | 3.56 |
| YGR192C | 1.4 | 1.0 | 3.8 | 1.0 | 1.1 | 1.7 | 1.7 | 1.1 | 0.9 | 1.3 | 2.3 | 2.2 | 3.4 | 1.0 | 1.9 | 1.1 | 1.1 | 1.3 | 7.49 |
| YGR244C | 1.0 | 1.2 | 1.6 | 1.6 | 0.8 | 2.6 | 3.9 | 1.8 | 1.4 | 1.1 | 1.8 | 1.9 | 2.6 | 1.2 | 2.0 | 2.6 | 1.9 | 3.1 | 1.12 |
| YGR254W | 1.2 | 1.3 | 3.8 | 1.3 | 1.2 | 1.9 | 1.5 | 1.4 | 0.8 | 1.2 | 1.3 | 1.7 | 3.1 | 0.6 | 2.4 | 1.4 | 1.3 | 1.2 | 7.01 |
| YHR018C | 1.0 | 1.0 | 1.5 | 1.4 | 1.7 | 1.1 | 0.8 | 1.0 | 1.4 | 4.4 | 1.4 | 1.4 | 3.3 | 1.0 | 4.4 | 1.0 | 1.9 | 1.2 | 1.21 |
| YIL160C | 0.8 | 2.4 | 2.3 | 3.5 | 1.0 | 1.6 | 1.1 | 1.2 | 3.2 | 3.8 | 2.4 | 1.0 | 2.3 | 1.5 | 5.6 | 8.8 | 2.4 | 3.0 | 0.27 |
| YIR017C | 3.5 | 8.6 | 2.5 | 1.9 | 0.8 | 1.9 | 1.5 | 1.2 | 5.2 | 3.4 | 4.2 | 1.2 | 3.4 | 1.9 | 2.3 | 3.0 | 1.5 | 1.3 | 0.36 |
| YJL052W | 1.5 | 0.9 | 4.0 | 1.8 | 0.7 | 2.4 | 2.1 | 1.5 | 1.6 | 2.0 | 4.3 | 2.4 | 5.6 | 1.3 | 2.3 | 2.3 | 1.2 | 1.9 | 6.19 |
| YJR009C | 1.1 | 1.7 | 5.6 | 1.0 | 1.2 | 1.6 | 1.6 | 1.1 | 1.1 | 1.3 | 1.1 | 1.5 | 2.7 | 1.0 | 2.1 | 1.5 | 1.1 | 1.3 | 5.86 |
| YJR130C | 1.1 | 0.6 | 0.8 | 0.9 | 1.4 | 2.0 | 1.3 | 1.3 | 5.2 | 3.8 | 1.7 | 1.0 | 2.9 | 1.1 | 0.9 | 1.5 | 1.1 | 1.3 | 0.62 |
| YJR149W | 1.0 | 1.8 | 1.4 | 2.3 | 0.8 | 1.0 | 1.7 | 1.0 | 1.4 | 1.0 | 0.7 | 1.0 | 3.5 | 1.4 | 1.4 | 2.4 | 1.1 | 1.2 | 0.34 |
| YKL218C | 2.1 | 1.0 | 2.2 | 1.8 | 1.2 | 3.6 | 1.6 | 1.7 | 3.4 | 2.7 | 1.7 | 1.2 | 5.1 | 1.4 | 0.8 | 3.1 | 2.5 | 2.6 | 0.60 |
| YLR027C | 1.0 | 1.5 | 2.3 | 0.8 | 0.9 | 2.0 | 1.4 | 0.7 | 1.6 | 2.7 | 1.6 | 1.3 | 2.5 | 1.0 | 1.4 | 0.9 | 1.3 | 1.3 | 1.39 |
| YLR133W | 0.9 | 1.6 | 1.4 | 0.7 | 1.0 | 2.2 | 1.3 | 0.7 | 6.1 | 2.3 | 0.6 | 1.5 | 3.3 | 1.1 | 1.9 | 1.0 | 1.1 | 1.3 | 0.36 |
| YLR155C | 1.4 | 1.1 | 2.4 | 1.8 | 1.7 | 1.6 | 2.0 | 1.0 | 3.0 | 1.8 | 2.0 | 1.1 | 3.3 | 1.1 | 1.2 | 2.2 | 1.4 | 2.2 | 1.63 |
| YLR158C | 1.4 | 1.2 | 2.1 | 1.7 | 1.7 | 1.5 | 2.0 | 1.1 | 2.7 | s | 2.4 | 1.1 | 3.2 | 1.2 | 1.1 | 2.1 | 1.4 | 2.0 | 1.57 |
| YLR195C | 0.8 | 0.9 | 1.0 | 0.8 | 1.0 | 0.8 | 1.1 | 1.0 | 0.6 | 0.7 | 0.8 | 1.3 | 2.3 | 0.7 | 0.9 | 0.8 | 0.9 | 1.0 | 1.55 |
| YLR345W | 1.4 | 1.4 | 1.4 | 1.2 | 1.2 | 1.6 | 1.2 | 1.4 | 4.5 | 4.0 | 2.5 | 1.6 | 3.5 | 1.5 | 1.0 | 1.2 | 1.2 | 1.4 | 2.65 |
| YML004C | 0.8 | 1.6 | 2.5 | 1.0 | 0.7 | 1.7 | 2.1 | 1.9 | 3.9 | 2.2 | 2.2 | 1.1 | 2.4 | 1.4 | 2.1 | 4.5 | 1.5 | 2.4 | 2.41 |
| YML131W | 11.2 | 4.3 | 7.7 | 1.9 | 0.7 | 2.0 | 3.0 | 6.1 | 11.1 | 17.7 | 14.4 | 1.1 | 2.4 | 2.7 | 2.2 | 1.6 | 2.5 | 3.0 | 1.15 |
| YNL071W | 0.9 | 0.9 | 1.7 | 1.5 | 0.6 | 1.3 | 1.4 | 0.9 | 1.1 | 1.2 | 1.6 | 1.3 | 2.5 | 0.8 | 1.1 | 1.1 | 1.0 | 1.2 | 1.61 |
| YNL241C | 1.3 | 2.5 | 4.3 | 1.0 | 0.8 | 0.9 | 3.2 | 0.9 | 3.4 | 7.4 | 3.0 | 2.0 | 4.9 | 1.1 | 7.0 | 2.8 | 1.0 | 1.0 | 0.68 |
| YOR120W | 1.8 | 5.4 | 2.8 | 3.2 | 1.4 | 3.4 | 2.7 | 2.0 | 3.3 | 3.0 | 3.0 | 1.2 | 3.6 | 2.4 | 1.5 | 3.9 | 1.3 | 2.6 | 1.06 |
| YAL023C | 0.4 | 1.5 | 1.0 | 0.7 | 1.0 | 0.6 | 0.8 | 0.6 | 0.3 | 0.4 | 1.7 | 1.2 | 2.3 | 0.4 | 2.5 | 0.4 | 1.0 | 0.7 | 1.49 |
| YAL060W | 1.1 | 1.8 | 3.2 | 2.7 | 1.2 | 4.2 | 3.3 | 0.9 | 0.6 | 2.5 | 2.4 | 0.8 | 2.2 | 0.9 | 0.9 | 3.2 | 1.1 | 1.8 | 2.39 |
| YAL062W | 0.8 | 1.5 | 1.4 | 1.1 | 1.0 | 1.2 | 1.1 | 0.8 | 1.4 | 0.7 | 1.2 | 1.0 | 2.1 | 0.8 | 1.1 | 1.3 | 1.0 | 0.8 | 0.86 |
| YBR006W | 1.3 | 1.6 | 2.6 | 1.5 | 1.3 | 3.1 | 3.3 | 1.0 | 4.5 | 2.7 | 1.9 | 1.1 | 2.2 | 1.1 | 1.1 | 3.0 | 1.2 | 1.8 | 0.62 |
| YBR056W | 1.4 | 1.7 | 1.4 | 2.3 | 1.4 | 2.3 | 5.5 | 1.4 | 3.0 | 3.3 | 1.8 | 1.4 | 3.0 | 1.0 | 0.7 | 2.8 | 2.4 | 3.4 | 1.13 |
| YBR149W | 1.1 | 2.0 | 2.7 | 1.7 | 1.4 | 2.8 | 3.1 | 1.5 | 1.7 | 1.9 | 1.9 | 1.4 | 2.2 | 1.2 | 0.7 | 2.7 | 2.1 | 3.4 | 2.72 |
| YBR284W | 0.9 | 1.5 | 2.8 | 4.8 | 1.3 | 1.9 | 1.0 | 0.9 | 6.5 | 3.2 | 1.4 | 1.2 | 2.8 | 1.6 | 0.8 | 0.8 | 1.1 | 1.0 | 0.25 |
| YCL018W | 1.2 | 2.7 | 2.4 | 1.4 | 0.8 | 2.3 | 2.3 | 1.3 | 2.1 | 4.3 | 3.7 | 1.6 | 3.2 | 1.0 | 0.6 | 2.3 | 1.3 | 1.0 | 0.99 |
| YCL040W | 0.9 | 7.1 | 10.1 | 2.0 | 0.5 | 3.5 | 2.9 | 0.7 | 0.9 | 3.0 | 8.2 | 2.3 | 5.6 | 0.7 | 3.4 | 3.1 | 1.4 | 1.7 | 1.98 |
| YDL010W | 1.7 | 0.8 | 0.6 | 0.7 | 1.2 | 1.1 | 1.9 | 1.1 | 2.2 | 1.3 | 1.3 | 0.9 | 2.0 | 2.2 | 1.4 | 1.4 | 1.5 | 2.4 | 0.93 |
| YDL024C | 1.6 | 1.5 | 1.2 | 2.2 | 1.0 | 1.7 | 0.9 | 1.7 | 4.2 | 3.4 | 1.2 | 1.7 | 2.6 | 1.9 | 2.1 | 2.8 | 0.9 | 1.1 | 0.40 |
| YDL095W | 0.5 | 1.3 | 1.0 | 0.8 | 0.8 | 1.0 | 0.8 | 0.7 | 1.2 | 0.7 | 1.0 | 1.1 | 2.2 | 0.5 | 2.0 | 0.5 | 0.7 | 0.9 | 1.57 |
| YDL245C | 1.0 | 2.4 | 1.6 | 2.2 | 1.3 | 1.1 | 1.2 | 1.2 | 0.4 | 2.8 | 1.5 | 1.0 | 2.0 | 1.2 | 1.8 | 1.5 | 1.0 | 1.1 | 0.28 |
| YDL246C | 1.2 | 1.5 | 1.3 | 1.1 | 1.1 | 1.4 | 2.1 | 2.4 | 4.4 | 3.6 | 2.6 | 1.2 | 2.9 | 1.7 | 2.6 | 1.7 | 1.8 | 1.8 | 0.43 |
| YDR001C | 0.8 | 2.2 | 2.6 | 1.0 | 1.1 | 2.2 | 1.5 | 0.9 | 2.2 | 3.0 | 0.7 | 1.1 | 2.8 | 1.0 | 4.4 | 1.9 | 1.1 | 1.6 | 0.75 |
| YDR058C | 1.2 | 2.2 | 0.8 | 2.7 | 0.7 | 1.0 | 1.4 | 1.9 | 2.4 | 1.7 | 1.7 | 1.2 | 2.5 | 1.8 | 1.2 | 1.9 | 1.1 | 1.6 | 0.56 |
| YDR072C | 1.3 | 1.3 | 1.7 | 1.2 | 0.9 | 1.5 | 0.9 | 0.7 | 0.6 | 0.7 | 0.7 | 0.9 | 1.9 | 0.6 | 0.4 | 0.7 | 1.9 | 1.9 | 2.81 |
| YDR127W | 0.8 | 0.9 | 1.5 | 0.7 | 1.2 | 0.8 | 0.9 | 0.7 | 0.6 | 1.3 | 1.3 | 0.8 | 1.7 | 1.0 | 1.3 | 0.6 | 0.9 | 0.7 | 0.78 |
| YDR261C | 1.2 | 1.2 | 1.9 | 0.7 | 1.3 | 1.4 | 0.8 | 0.9 | 1.7 | 1.0 | 1.2 | 1.0 | 1.9 | 0.9 | 2.3 | 0.6 | 0.9 | 0.8 | 0.68 |
| YDR272W | 1.0 | 4.0 | 0.8 | 1.8 | 0.7 | 2.3 | 1.7 | 2.1 | 2.5 | 1.9 | 2.1 | 1.2 | 2.4 | 1.4 | 1.1 | 2.1 | 1.4 | 2.3 | 1.51 |
| YDR497C | 0.7 | 0.5 | 0.6 | 1.3 | 0.7 | 0.6 | 0.5 | 0.6 | 0.4 | 0.8 | 0.7 | 0.9 | 2.7 | 0.6 | 10.1 | | 1.6 | 1.8 | 1.45 |
| YDR516C | 1.2 | 1.5 | 7.8 | 2.1 | 1.3 | 1.8 | 3.9 | 1.3 | 1.8 | 5.2 | 2.8 | 1.1 | 2.5 | 0.7 | 0.4 | 1.7 | 1.1 | 1.2 | 1.66 |
| YER053C | 1.6 | 1.8 | 1.9 | 1.7 | 0.6 | 2.8 | 2.8 | 1.3 | 1.3 | 3.9 | 2.4 | 1.7 | 4.1 | 1.3 | 1.1 | 2.8 | 1.2 | 2.3 | 1.83 |
| YER096W | 1.1 | 2.3 | 2.0 | 2.0 | 1.3 | 1.2 | 0.9 | 1.0 | 7.5 | 3.5 | 1.1 | 0.9 | 2.3 | 3.1 | 1.4 | 2.0 | 1.0 | 1.2 | 0.22 |
| YER178W | 0.8 | 0.9 | 3.6 | 1.0 | 0.7 | 2.5 | 1.6 | 0.8 | 1.7 | 1.3 | 2.5 | 1.3 | 2.4 | 0.9 | 1.8 | 1.0 | 1.1 | 0.9 | 2.18 |
| YFL030W | 1.6 | 6.0 | 3.1 | 2.0 | 1.1 | 1.3 | 0.6 | 1.6 | 24.5 | 4.9 | 5.4 | 1.2 | 2.2 | 0.8 | 1.1 | 0.6 | 1.0 | 0.8 | 0.50 |
| YFL031W | 0.5 | 1.3 | 2.1 | 0.7 | 1.0 | 0.3 | 0.9 | 0.7 | 1.0 | 0.7 | 1.2 | 1.0 | 2.6 | 0.4 | 2.7 | 0.9 | 1.2 | 1.1 | 3.16 |
| YFR047C | 1.3 | 4.0 | 2.7 | 2.6 | 1.2 | 2.0 | 2.0 | 1.0 | 3.0 | 1.5 | 2.3 | 1.7 | 2.7 | 1.9 | 0.6 | 3.6 | 1.5 | 1.6 | 1.57 |
| YGL248W | 1.3 | 1.3 | 0.9 | 0.7 | 1.3 | 2.7 | 1.6 | 0.8 | 0.2 | 2.9 | 1.0 | 0.8 | 2.0 | 1.3 | 1.8 | 2.9 | 1.1 | 1.2 | 0.22 |
| YGR037C | 1.0 | 1.2 | 2.2 | 0.8 | 1.0 | 1.1 | 1.4 | 1.4 | 1.7 | 1.4 | 1.4 | 1.5 | 2.7 | 1.5 | 1.5 | 2.6 | 1.4 | 2.0 | 3.40 |
| YGR055W | 2.5 | 5.7 | 12.0 | 0.7 | 1.3 | 1.1 | 0.6 | 1.1 | 5.0 | 2.4 | 1.5 | 0.9 | 1.9 | 1.2 | 2.1 | 0.6 | 0.7 | 0.7 | 1.42 |
| YGR194C | 0.9 | 1.0 | 1.3 | 2.1 | 0.8 | 1.9 | 2.2 | 1.2 | 2.4 | 2.1 | 0.7 | 0.8 | 2.2 | 1.0 | 0.6 | 2.6 | 1.1 | 2.0 | 0.63 |
| YGR256W | 1.2 | 1.5 | 2.3 | 0.8 | 0.9 | 6.2 | 1.4 | 2.2 | 3.4 | 2.5 | 2.7 | 1.5 | 3.9 | 1.1 | 2.7 | 5.3 | 1.1 | 0.8 | 0.94 |
| YHR111W | 1.4 | 1.3 | 0.8 | 1.9 | 1.5 | 1.3 | 1.6 | 1.3 | 4.5 | 4.2 | 1.9 | 0.9 | 2.2 | 1.7 | 1.1 | 1.3 | 1.1 | 1.2 | 0.44 |
| YHR174W | 1.1 | 1.4 | 3.3 | 1.2 | 1.3 | 1.5 | 1.6 | 1.2 | 1.0 | 1.5 | 1.4 | 1.5 | 3.6 | 0.6 | 2.0 | 1.1 | 1.0 | 1.2 | 7.34 |
| YHR176W | 1.8 | 2.0 | 1.1 | 1.4 | 1.0 | 2.3 | 1.4 | 1.3 | 6.3 | 6.4 | 1.2 | 1.2 | 1.9 | 1.3 | 1.5 | 1.8 | 1.4 | 1.6 | 0.27 |
| YIL045W | 1.7 | 1.4 | 1.9 | 2.2 | 1.3 | 1.7 | 1.6 | 1.1 | 2.1 | 3.2 | 1.2 | 1.5 | 2.0 | 1.6 | 0.6 | 2.9 | 1.1 | 1.8 | 0.37 |
| YIL107C | 1.5 | 0.8 | 1.1 | 1.5 | 0.8 | 1.0 | 1.6 | 1.3 | 3.5 | 3.5 | 0.9 | 1.1 | 1.9 | 1.6 | 1.0 | 2.7 | 1.2 | 2.0 | 0.58 |
| YIL155C | 1.0 | 0.7 | 1.4 | 3.7 | 1.3 | 1.4 | 2.2 | 1.0 | 2.5 | 2.9 | 1.3 | 1.4 | 2.0 | 1.3 | 1.4 | 3.8 | 1.1 | 1.4 | 0.51 |
| YIR034C | 1.2 | 1.1 | 2.5 | 0.6 | 1.1 | 1.3 | 0.8 | 1.7 | 3.2 | 3.5 | 3.0 | 1.4 | 2.6 | 1.2 | 1.9 | 1.3 | 1.0 | 1.0 | 0.92 |
| YIR036C | 1.0 | 1.7 | 2.6 | 1.8 | 0.9 | 0.7 | 1.0 | 1.0 | 3.3 | 1.9 | 2.8 | 1.2 | 1.9 | 1.2 | 1.1 | 4.0 | 1.5 | 1.3 | 0.64 |
| YJL031C | 1.6 | 1.2 | 0.5 | 1.3 | 1.7 | 1.6 | 2.3 | 1.8 | 4.7 | 3.6 | 1.9 | 1.3 | 2.6 | 3.0 | 2.0 | 1.9 | 1.2 | 1.9 | 1.02 |
| YJL068C | 1.2 | 2.7 | 3.4 | 1.6 | 0.7 | 1.1 | 1.2 | 1.1 | 6.5 | 3.1 | 3.2 | 1.7 | 2.7 | 1.5 | 0.5 | 2.6 | 1.4 | 1.5 | 0.95 |
| YJL099W | 1.2 | 1.2 | 0.3 | 1.3 | 1.9 | 2.0 | 1.8 | 1.0 | 3.1 | 3.1 | 0.9 | 1.0 | 2.4 | 1.8 | 1.4 | 1.7 | 1.2 | 1.6 | 0.55 |
| YJL172W | 0.8 | 2.1 | 1.8 | 1.5 | 0.9 | 3.8 | 1.0 | 0.7 | 0.7 | 1.9 | 1.4 | 1.1 | 3.1 | 0.9 | 1.2 | 0.5 | 1.8 | 1.8 | 0.62 |
| YJL219W | 1.2 | 2.5 | 3.1 | 1.6 | 1.2 | 1.5 | 0.9 | 1.5 | 4.0 | 2.3 | 2.3 | 1.1 | 4.0 | 1.1 | 2.6 | 1.4 | 1.6 | 1.2 | 0.91 |
| YJR137C | 2.7 | 2.9 | 4.6 | 0.8 | 0.6 | 1.5 | 0.8 | 1.6 | 9.0 | 4.8 | 2.0 | 1.4 | 2.1 | 1.2 | 1.0 | 1.3 | 0.9 | 0.8 | 0.46 |
| YKL035W | 1.0 | 0.9 | 4.8 | 1.2 | 0.8 | 1.2 | 0.6 | 1.0 | 0.8 | 1.2 | 2.1 | 1.0 | 2.0 | 0.6 | 1.0 | 1.5 | 1.0 | 1.9 | 2.54 |
| YKL091C | 1.2 | 1.3 | 1.4 | 2.6 | 1.0 | 2.2 | 1.9 | 1.3 | 2.3 | 3.2 | 1.7 | 1.3 | 2.0 | 1.5 | 1.1 | 4.6 | 1.4 | 2.0 | 0.83 |
| YKL104C | 1.0 | 1.0 | 1.6 | 1.2 | 1.4 | 0.9 | 2.3 | 1.1 | 1.4 | 1.8 | 2.2 | 1.6 | 2.2 | 0.8 | 3.3 | 0.6 | 1.0 | 1.1 | 1.25 |
| YKL152C | 1.3 | 1.3 | 1.9 | 0.9 | 1.3 | 1.6 | 1.5 | 1.0 | 0.9 | 1.5 | 1.7 | 1.5 | 2.7 | 1.0 | 1.8 | 2.0 | 1.1 | 1.7 | 3.28 |
| YKL213C | 0.9 | 0.8 | 1.0 | 1.3 | 0.7 | 1.0 | 1.0 | 1.1 | 4.8 | 2.1 | 1.3 | 0.9 | 2.1 | 0.9 | 1.1 | 1.5 | 1.4 | 1.6 | 0.77 |
| YKL215C | 0.6 | 1.1 | 1.3 | 0.8 | 1.0 | 2.1 | 1.1 | 1.1 | 1.3 | 1.9 | 0.5 | 1.0 | 2.6 | 1.0 | 0.9 | 0.8 | 0.8 | 0.9 | 0.38 |
| YLL058W | 1.0 | 3.8 | 1.4 | 0.9 | 0.8 | 1.4 | 1.5 | 0.9 | 3.5 | 3.6 | 2.0 | 1.0 | 2.3 | 0.8 | 1.9 | 1.1 | 1.4 | 1.3 | 0.39 |
| YLR299W | 1.2 | 1.9 | 1.5 | 1.3 | 0.6 | 0.9 | 0.8 | 1.0 | 4.9 | 2.3 | 1.7 | 1.0 | 2.9 | 1.3 | 1.5 | 0.9 | 1.3 | 1.4 | 0.57 |
| YLR348C | 1.1 | 4.5 | 1.2 | 1.2 | 0.9 | 1.9 | 0.9 | 0.9 | 2.1 | 1.3 | 1.3 | 0.9 | 1.9 | 1.0 | 2.4 | 1.1 | 1.1 | 1.0 | 0.64 |
| YML054C | 1.5 | 1.8 | 1.3 | 3.4 | 1.3 | 1.8 | 1.2 | 1.5 | 4.1 | 1.8 | 1.4 | 1.8 | 2.8 | 2.1 | 1.1 | 7.8 | 1.1 | 1.6 | 0.25 |
| YML070W | 0.9 | 1.7 | 2.5 | 1.9 | 1.4 | 2.2 | 3.1 | 1.3 | 4.5 | 3.9 | 1.5 | 1.3 | 3.3 | 1.3 | 0.9 | 2.6 | 1.2 | 1.3 | 1.03 |
| YML100W | 0.8 | 2.7 | 10.6 | 1.4 | 0.9 | 2.8 | 2.2 | 0.7 | 1.7 | 1.4 | 3.2 | 1.2 | 3.8 | 1.2 | 1.8 | 2.2 | 1.0 | 1.5 | 0.88 |
| YMR008C | 0.7 | 0.9 | 1.4 | 1.0 | 1.1 | 1.7 | 0.9 | 0.5 | 0.3 | 0.6 | 1.3 | 1.1 | 3.7 | 0.7 | 2.0 | 1.2 | 3.0 | 1.9 | 1.59 |
| YMR020W | 1.3 | 1.0 | 0.9 | 1.7 | 1.4 | 1.6 | 2.7 | 1.2 | 2.3 | 2.9 | 1.5 | 1.1 | 3.0 | 1.1 | 1.8 | 2.8 | 2.3 | 2.2 | 0.81 |
| YMR105C | 1.9 | 3.0 | 5.0 | 4.2 | 0.9 | 2.8 | 2.8 | 1.1 | 0.6 | 2.9 | 3.0 | 1.7 | 3.3 | 1.0 | 0.9 | 2.0 | 1.6 | 2.6 | 1.21 |
| YMR271C | 1.6 | 1.6 | 1.3 | 2.0 | 1.1 | 4.2 | 3.4 | 1.8 | 8.0 | 4.4 | 1.7 | 1.3 | 3.6 | 1.8 | 0.6 | 5.7 | 1.3 | 2.2 | 0.70 |
| YNL012W | 1.3 | 1.3 | 1.8 | 0.8 | 1.3 | 1.7 | 2.0 | 1.8 | 3.3 | 3.2 | 1.6 | 1.3 | 2.9 | 1.0 | 1.6 | 1.2 | 1.3 | 1.5 | 0.24 |
| YNL045W | 0.8 | 1.3 | 2.0 | 1.3 | 1.1 | 2.2 | 1.8 | 0.7 | 1.3 | 1.1 | 2.1 | 0.9 | 2.6 | 0.8 | 0.8 | 2.4 | 1.1 | 1.1 | 0.80 |
| YNL104C | 1.1 | 1.3 | 3.0 | 0.9 | 0.8 | 0.8 | 0.6 | 0.9 | 1.1 | 2.0 | 2.1 | 1.1 | 2.1 | 0.9 | 2.5 | 1.5 | 0.9 | 0.9 | 1.69 |
| YNL231C | 1.5 | 0.8 | 0.8 | 0.5 | 2.1 | 1.8 | 2.5 | 1.5 | 2.2 | 1.3 | 1.3 | 1.8 | 2.9 | 0.9 | 0.9 | 0.7 | 3.1 | 4.0 | 2.13 |
| YNR019W | 1.0 | 1.3 | 1.6 | 1.2 | 0.8 | 1.3 | 0.8 | 0.7 | 1.8 | 1.9 | 0.7 | 1.0 | 2.1 | 1.0 | 1.6 | 0.8 | 2.4 | 2.0 | 0.37 |
| YNR033W | 0.6 | 0.8 | 0.8 | 1.0 | 1.3 | 1.6 | 1.2 | 1.1 | 3.6 | 4.2 | 0.4 | 1.1 | 3.8 | 0.9 | 1.3 | 2.2 | 0.9 | 1.1 | 0.72 |
| YNR059W | 1.1 | 0.9 | 0.2 | 1.4 | 1.4 | 0.8 | 1.6 | 1.3 | 1.8 | 1.4 | 1.0 | 0.8 | 2.4 | 1.0 | 1.0 | 2.0 | 2.6 | 2.2 | 0.45 |
| YOL126C | 1.0 | 0.8 | 1.0 | 1.6 | 0.7 | 1.5 | 1.4 | 0.9 | 0.8 | 2.4 | 1.8 | 1.1 | 2.6 | 1.2 | 1.4 | 2.5 | 1.6 | 2.4 | 0.59 |
| YOL153C | 1.0 | 1.2 | 2.1 | 3.9 | 1.2 | 1.9 | 3.3 | 1.0 | 2.5 | 3.2 | 2.4 | 1.9 | 2.9 | 1.5 | 1.2 | 5.4 | 1.1 | 1.7 | 0.37 |
| YOR099W | 0.8 | 1.3 | 2.1 | 1.3 | 1.5 | 0.9 | 1.0 | 0.9 | 0.6 | 0.7 | 1.2 | 1.1 | 1.6 | 0.7 | 1.5 | 1.1 | 1.2 | 2.3 | 3.94 |
| YOR130C | 0.8 | 1.7 | 1.4 | 1.0 | 1.8 | 1.6 | 1.3 | 0.8 | 2.0 | 1.7 | 0.6 | 0.9 | 2.1 | 1.3 | 1.0 | 0.8 | 1.5 | 1.1 | 0.51 |
| YOR336W | 0.7 | 1.4 | 0.7 | 1.0 | 1.3 | 1.7 | 1.2 | 0.8 | 1.7 | 1.5 | 1.4 | 1.0 | 2.1 | 1.1 | 0.6 | 0.9 | 1.0 | 1.0 | 0.44 |
| YOR347C | 0.9 | 0.9 | 2.1 | 2.2 | 0.7 | 0.9 | 0.9 | 0.8 | 0.5 | 0.9 | 1.4 | 1.2 | 1.8 | 0.9 | 2.0 | 1.4 | 1.7 | 1.3 | 1.31 |
| YPL017C | 1.0 | 2.5 | 0.8 | 1.0 | 1.3 | 1.5 | 1.4 | 1.3 | 3.0 | 6.7 | 0.4 | 1.0 | 1.9 | 1.6 | 1.0 | 1.3 | 1.3 | 0.8 | 0.19 |
| YPR026W | 0.9 | 1.2 | 5.0 | 1.3 | 0.8 | | 1.3 | 1.0 | 0.9 | 3.1 | 1.6 | 1.2 | 2.3 | 1.5 | 3.1 | 2.5 | 0.9 | 1.3 | 0.26 |
| YAL012W | 1.1 | 6.5 | 10.4 | 0.8 | 0.6 | 1.6 | 0.6 | 0.7 | 4.7 | 3.9 | 4.0 | 1.1 | 1.1 | 0.9 | 3.1 | 1.0 | 0.9 | 0.8 | 0.96 |
| YBR029C | 0.7 | 0.2 | 1.8 | 1.0 | 1.4 | 0.8 | 0.4 | 0.7 | 2.0 | 1.1 | 0.7 | 1.3 | 1.7 | 0.9 | 3.5 | 0.9 | 0.6 | 0.8 | 1.56 |
| YBR222C | 0.6 | 1.2 | 1.3 | 0.8 | 0.7 | 1.3 | 1.0 | 0.6 | 0.7 | 1.0 | 1.3 | 0.9 | 1.6 | 0.5 | 4.3 | 2.1 | 0.8 | 0.9 | 0.52 |
| YCL009C | 0.6 | 0.9 | 2.0 | 0.5 | 0.7 | 1.8 | 1.3 | 1.1 | 0.9 | 1.6 | 1.5 | 1.7 | 1.6 | 0.7 | 6.5 | 0.9 | 1.1 | 0.8 | 0.68 |
| YCL064C | 0.6 | 0.6 | 0.8 | 0.8 | 1.4 | 3.3 | 13.4 | 2.4 | 1.7 | 5.2 | 1.8 | 0.7 | 1.6 | 0.8 | 21.4 | 1.0 | 0.3 | 0.3 | 1.42 |
| YCR098C | 1.6 | 3.0 | 1.7 | 2.0 | 1.4 | 1.6 | 1.2 | 1.0 | 1.4 | 2.4 | 1.3 | 1.3 | 1.4 | 1.1 | 11.0 | 0.8 | 1.7 | 1.6 | 0.22 |
| YDR502C | 1.2 | 2.8 | 2.4 | 1.2 | 1.5 | 1.5 | 0.9 | 0.9 | 1.6 | 0.9 | 1.3 | 1.3 | 1.2 | 0.8 | 8.2 | 1.0 | 0.9 | 0.9 | 2.75 |
| YER026C | 0.8 | 0.6 | 3.3 | 1.0 | 1.2 | 2.5 | 1.6 | 0.8 | 0.9 | 1.5 | 0.9 | 1.7 | 1.9 | 1.1 | 4.0 | 1.4 | 1.7 | 3.2 | 4.48 |
| YHR137W | 0.7 | 1.0 | 2.4 | 1.7 | 1.0 | 0.7 | 0.4 | 0.4 | 0.2 | 0.9 | 0.6 | 0.6 | 0.6 | 0.8 | 2.8 | 0.4 | 0.8 | 1.3 | 1.40 |
| YMR189W | 0.7 | 0.8 | 1.9 | 2.8 | 0.4 | 1.9 | 0.8 | 0.4 | 0.7 | 0.9 | 8.2 | 0.6 | 0.9 | 1.6 | 5.6 | 0.9 | 1.0 | 0.8 | 0.88 |
| YNL106C | 1.0 | 1.4 | 0.8 | 1.5 | 0.8 | 1.0 | 0.9 | 0.8 | 0.7 | 1.4 | 0.9 | 0.8 | 0.9 | 1.1 | 3.3 | 1.2 | 1.1 | 0.9 | 0.37 |
| YNL169C | 0.7 | 1.4 | 1.4 | 1.2 | 0.7 | 1.4 | 0.7 | 0.8 | 1.1 | 1.1 | 0.8 | 1.0 | 1.7 | 0.9 | 3.4 | 0.8 | 1.1 | 1.3 | 1.05 |
| YNL322C | 0.8 | 0.9 | 1.9 | 0.8 | 1.4 | 1.1 | 0.9 | 0.7 | 0.7 | 0.8 | 0.6 | 0.7 | 0.8 | 1.2 | 3.7 | 0.9 | 0.9 | 1.0 | 0.75 |
| YAL038W | 1.0 | 1.0 | 3.0 | 1.4 | 1.4 | 1.3 | 0.9 | 1.1 | 0.1 | 1.1 | 1.0 | 1.2 | 1.8 | 0.5 | 3.1 | 0.9 | 1.0 | 1.0 | 7.02 |
| YBR023C | 0.5 | 0.8 | 1.3 | 1.1 | 0.8 | 0.5 | 0.7 | 0.5 | 0.3 | 0.4 | 0.9 | 0.8 | 0.5 | 0.8 | 2.8 | 0.7 | 0.7 | 0.9 | 0.93 |
| YCR048W | 0.5 | 1.1 | 1.0 | 0.6 | 0.9 | 0.2 | 0.9 | 0.8 | 0.8 | 0.5 | 1.2 | 0.9 | 1.3 | 0.9 | 3.1 | 1.1 | 1.2 | 0.8 | 0.29 |
| YDR380W | 0.8 | 0.8 | 0.8 | 1.7 | 1.1 | 0.4 | 0.5 | 0.2 | 0.2 | 0.3 | 0.4 | 0.4 | 0.4 | 0.6 | 2.4 | 0.2 | 0.8 | 0.8 | 1.04 |
| YER069W | 1.3 | 0.8 | 3.3 | 1.4 | 1.5 | 1.3 | 1.0 | 1.5 | 0.8 | 10.4 | 1.5 | 0.8 | 1.4 | 1.3 | 2.4 | 1.1 | 1.1 | 0.9 | 0.25 |
| YGL022W | 0.5 | 0.8 | 1.4 | 0.3 | 1.0 | 0.9 | 0.7 | 0.4 | 0.9 | 0.4 | 0.8 | 0.7 | 1.0 | 0.5 | 4.0 | 0.3 | 0.9 | 0.7 | 0.91 |
| YGL126W | 0.6 | 1.3 | 1.4 | 0.8 | 1.1 | 1.6 | | 0.7 | 1.6 | 0.7 | 1.0 | 0.6 | | | 2.8 | | 0.9 | 0.9 | 0.34 |
| YGL209W | 1.2 | 1.6 | 1.1 | 2.6 | 0.7 | 1.0 | 1.0 | 1.1 | 0.6 | 0.4 | 0.8 | 1.3 | 1.9 | 1.6 | 3.2 | 2.8 | 1.1 | 1.5 | 0.63 |
| YGR282C | 1.3 | 0.7 | 4.6 | 1.9 | 1.0 | 2.2 | 1.1 | 1.0 | 0.8 | 0.8 | 1.5 | 1.3 | 1.2 | 1.0 | 2.9 | 1.4 | 1.1 | 1.5 | 5.04 |
| YIL154C | 0.8 | 1.3 | 1.9 | 0.8 | 0.9 | 1.1 | 1.3 | 0.6 | 2.4 | 1.0 | 1.2 | 1.1 | 1.7 | 0.9 | 4.3 | 1.2 | 1.2 | 1.0 | 0.39 |
| YJL088W | 0.9 | 0.8 | 2.2 | 0.5 | 0.9 | 2.8 | | 1.3 | 3.2 | 4.1 | 0.9 | 0.7 | 1.0 | 2.0 | 5.7 | 0.7 | 1.4 | 0.9 | 0.27 |
| YJR148W | 0.7 | 1.4 | 2.0 | 3.5 | 0.6 | 2.0 | 2.1 | 0.6 | 0.7 | 3.0 | 0.6 | 0.8 | 1.1 | 1.3 | 3.2 | 1.3 | 1.3 | 1.7 | 1.50 |
| YLR180W | 1.3 | 1.4 | 2.4 | 1.0 | 0.8 | 1.2 | 0.6 | 0.9 | 0.8 | 0.9 | 1.7 | 0.6 | 0.6 | 0.6 | 2.5 | 0.5 | 0.8 | 0.7 | 3.23 |
| YLR273C | 1.4 | 1.0 | 1.3 | 2.4 | | 1.2 | 1.2 | 1.3 | 2.8 | 2.4 | 1.4 | 0.9 | 1.7 | 1.3 | 2.6 | 1.5 | 1.0 | 1.0 | 0.20 |
| YLR300W | 0.9 | 0.8 | 0.8 | 1.0 | 0.8 | 1.1 | 0.5 | 0.8 | 0.0 | 0.5 | 1.0 | 0.5 | 0.2 | 0.6 | 2.7 | 0.3 | 0.7 | 0.5 | 5.14 |
| YLR307W | 0.9 | 0.4 | 0.7 | 3.3 | 1.4 | 1.2 | 1.5 | 1.2 | 0.6 | 0.7 | 0.8 | 0.7 | 1.0 | 1.9 | 3.9 | 0.9 | 1.1 | 1.0 | 0.16 |
| YMR296C | 0.6 | 0.7 | 1.2 | 2.6 | 0.4 | 0.4 | 0.6 | 0.5 | 0.4 | 0.6 | 0.8 | 0.8 | 0.9 | 0.7 | 6.7 | 0.5 | 0.8 | 0.7 | 0.52 |
| YOL058W | 0.8 | 0.5 | 1.7 | 0.9 | 1.2 | 0.8 | 0.4 | 1.6 | 0.1 | 15.7 | 2.3 | 1.3 | 1.2 | 1.5 | 2.7 | 0.9 | 1.2 | 0.8 | 0.82 |
| YBR183W | 1.8 | 1.5 | 2.0 | 2.9 | 0.9 | 2.5 | 1.7 | 1.0 | 0.4 | 2.0 | 1.9 | 1.2 | 1.4 | 1.3 | 1.1 | 2.1 | 1.4 | 2.4 | 1.47 |
| YDR019C | 1.4 | 0.5 | 1.7 | 4.0 | 0.9 | 4.8 | 1.4 | 0.7 | 0.4 | 0.7 | 1.8 | 0.7 | 1.0 | 1.6 | 1.8 | 1.8 | 1.2 | 1.6 | 2.48 |
| YIL167W | 3.3 | 1.6 | 2.0 | 1.0 | 1.0 | 7.7 | 1.2 | 1.9 | 6.9 | 12.7 | 1.9 | 0.7 | 1.2 | 1.4 | 0.6 | 2.3 | 0.7 | 0.8 | 2.11 |
| YKR039W | 1.4 | 3.5 | 1.3 | 1.7 | 1.0 | 2.8 | 1.0 | 1.0 | 1.9 | 2.0 | 1.9 | 1.5 | 1.3 | 1.2 | 1.7 | 1.9 | 0.9 | 1.1 | 0.40 |
| YNL037C | 1.4 | 1.8 | 1.7 | 1.1 | 1.7 | 2.8 | 2.8 | 1.0 | 0.6 | 3.2 | 1.4 | 1.3 | 1.9 | 1.3 | 0.9 | 1.4 | 0.9 | 1.3 | 2.05 |
| YNR002C | 1.3 | 1.6 | 1.0 | 2.0 | 1.2 | 2.3 | 1.2 | 1.0 | 3.9 | 1.6 | 1.6 | 1.2 | 2.1 | 1.2 | 1.0 | 2.4 | 1.5 | 2.1 | 0.29 |
| YOL143C | 1.0 | 1.4 | 1.2 | 1.2 | 0.9 | 3.5 | 1.3 | 1.2 | 0.8 | 1.0 | 2.0 | 1.2 | 1.2 | 0.7 | 1.0 | 2.2 | 1.8 | 1.4 | 2.30 |
| YOR136W | 1.0 | 0.9 | 3.5 | 1.0 | 1.5 | 3.9 | 3.2 | 1.1 | 0.3 | 3.4 | 1.4 | 1.2 | 1.5 | 0.9 | 1.2 | 1.3 | 0.8 | 1.3 | 3.20 |
| YAL044C | 1.5 | 1.1 | 1.0 | 3.0 | 0.8 | 2.2 | 1.0 | 0.8 | 0.4 | 0.6 | 1.4 | 0.6 | 1.1 | 1.4 | 0.7 | 1.0 | 1.4 | 1.8 | 4.07 |
| YAL054C | 1.2 | 1.1 | 1.4 | 1.6 | 1.1 | 2.2 | 1.1 | 1.1 | 8.9 | 4.1 | 1.3 | 1.0 | 1.8 | 1.3 | 0.9 | 2.5 | 1.5 | 1.4 | 0.24 |
| YAR071W | 1.6 | 0.8 | 1.0 | 0.7 | 1.6 | 2.0 | 0.3 | 1.4 | 0.3 | 0.3 | 1.1 | 2.0 | 0.3 | 1.1 | 1.1 | 0.3 | 0.7 | 1.6 | 3.21 |
| YBL001C | 1.1 | 1.5 | 1.0 | 1.6 | 1.0 | 2.4 | 1.3 | 1.0 | 1.5 | 0.9 | 2.3 | 0.7 | 1.0 | 1.5 | 1.0 | 1.7 | 1.6 | 1.8 | 2.38 |
| YBR014C | 1.1 | 1.2 | 0.8 | 0.9 | 1.4 | 2.0 | 1.4 | 1.2 | 1.3 | 0.8 | 1.8 | 0.6 | 0.9 | 1.2 | 1.2 | 1.5 | 1.0 | 1.6 | 1.29 |
| YBR035C | 1.1 | 1.0 | 1.7 | 1.1 | 1.3 | 2.3 | 1.5 | 1.5 | 2.2 | 1.5 | 2.0 | 1.2 | 1.8 | 1.3 | 0.7 | 2.1 | 1.5 | 1.9 | 1.89 |
| YBR068C | 1.1 | 1.0 | 1.9 | 2.5 | 1.4 | 1.9 | | 1.0 | 0.8 | 2.5 | 0.8 | 0.8 | 2.2 | 1.8 | 1.6 | 5.5 | 1.1 | 1.3 | 1.99 |
| YBR111C | 1.0 | 0.9 | 1.0 | 1.7 | 1.3 | 3.0 | 2.2 | 1.7 | 1.9 | 1.9 | 1.5 | 1.2 | 1.6 | 1.6 | 0.7 | 3.5 | 1.4 | 2.4 | 3.20 |
| YCR037C | 0.7 | 1.0 | 0.8 | 0.8 | 1.3 | 1.8 | 1.0 | 0.7 | 0.6 | 0.6 | 0.8 | 0.7 | 1.3 | 0.8 | 1.0 | 1.1 | 1.4 | 0.9 | 0.74 |
| YDL022W | 0.9 | 2.6 | 6.9 | 1.5 | 0.7 | 2.1 | 1.5 | 0.8 | 1.2 | 1.8 | 2.8 | 1.2 | 1.5 | 0.8 | 0.9 | 1.1 | 1.0 | 1.4 | 1.79 |
| YDR009W | 1.4 | 0.8 | 1.5 | 1.0 | 1.2 | 2.4 | 1.4 | 1.2 | 1.3 | 2.0 | 0.1 | 1.0 | 1.2 | 1.5 | 3.2 | 1.0 | 1.2 | 1.0 | 0.21 |
| YDR410C | 0.8 | 2.0 | 1.3 | 0.9 | 1.1 | 2.5 | 1.4 | 0.9 | 1.2 | 1.5 | 1.6 | 0.9 | 1.2 | 1.3 | 1.2 | 0.8 | 1.0 | 1.0 | 1.33 |
| YDR487C | 2.2 | 0.9 | 1.3 | 1.1 | 1.3 | 2.3 | 1.3 | 1.8 | 2.7 | 2.3 | 2.1 | 1.3 | 1.5 | 2.5 | 1.0 | 1.5 | 1.5 | 1.9 | 2.61 |
| YEL011W | 2.0 | 1.6 | 1.7 | 6.3 | 1.1 | 2.8 | 2.7 | 0.9 | 2.5 | 1.6 | 3.7 | 1.5 | 1.5 | 1.5 | 0.4 | 2.4 | 1.4 | 2.5 | 0.89 |
| YFR015C | 1.2 | 0.8 | 3.2 | 2.1 | 1.2 | 3.0 | 7.3 | 0.7 | 0.3 | 1.8 | 3.9 | 0.4 | 2.5 | 3.1 | 0.4 | 2.4 | 1.3 | 1.2 | 0.66 |
| YGL001C | 1.0 | 1.0 | 1.4 | 1.1 | 1.5 | 2.8 | 1.4 | 1.1 | 0.8 | 0.8 | 1.6 | 0.9 | 1.0 | 1.1 | 0.9 | 2.2 | 1.9 | 2.1 | 2.53 |
| YGL104C | 0.8 | 2.0 | 2.1 | 1.8 | 1.0 | 2.1 | 1.2 | 0.8 | 4.6 | 2.1 | 1.8 | 1.1 | 2.0 | 1.0 | 0.9 | 1.7 | 1.3 | 1.3 | 0.58 |
| YGL154C | 1.3 | 1.4 | 1.5 | 0.9 | 1.0 | 1.7 | 0.7 | 1.2 | 2.0 | 1.4 | 0.9 | 0.9 | 2.0 | 1.1 | 0.8 | 0.7 | 1.0 | 1.1 | 0.57 |
| YGL253W | 0.8 | 1.0 | 3.2 | 0.7 | 1.3 | 2.4 | 1.7 | 1.1 | 0.3 | 1.4 | 1.0 | 1.2 | 1.5 | 0.5 | 0.8 | 0.4 | 0.7 | 0.8 | 4.63 |
| YGR060W | 0.7 | 0.6 | 0.9 | 0.7 | 1.9 | 2.5 | 0.5 | 1.0 | 0.5 | 0.4 | 0.8 | 1.0 | 0.6 | 0.8 | 1.4 | 2.1 | 1.3 | 1.6 | 4.19 |
| YHR037W | 0.7 | 0.9 | 1.3 | 1.6 | 0.9 | 1.8 | 1.2 | 1.0 | 0.9 | 2.2 | 1.7 | 1.1 | 1.1 | 1.1 | 1.1 | 2.0 | 1.0 | 1.4 | 1.15 |
| YHR092C | 2.3 | 1.0 | 7.5 | 2.5 | 1.4 | 1.7 | 0.5 | 1.5 | 0.2 | 0.9 | 1.5 | 1.0 | 0.5 | 1.6 | 0.2 | 1.2 | 0.9 | 1.3 | 6.09 |
| YHR190W | 0.8 | 3.9 | 1.2 | 1.2 | 1.8 | 1.7 | 1.9 | 1.2 | 2.4 | 1.9 | 1.3 | 1.1 | 1.7 | 1.3 | 1.3 | 1.8 | 1.3 | 1.8 | 2.15 |
| YIL033C | 0.8 | 1.3 | 3.3 | 1.3 | 0.7 | 2.0 | 1.1 | 0.7 | 2.4 | 1.3 | 1.8 | 1.1 | 2.5 | 0.7 | 1.0 | 2.0 | 1.0 | 1.2 | 1.18 |
| YIR035C | 1.4 | 1.8 | 0.9 | 1.6 | 0.7 | 2.1 | 1.3 | 1.3 | 0.7 | 1.2 | 1.3 | 0.9 | 1.1 | 1.1 | 1.4 | 1.0 | 1.1 | 0.9 | 1.51 |
| YJL132W | 1.0 | 0.7 | 0.8 | 1.6 | 1.3 | 2.0 | 1.7 | 1.0 | 1.6 | 1.4 | 1.4 | 1.0 | 1.3 | 1.4 | 1.1 | 2.1 | 1.3 | 1.5 | 0.37 |
| YJL196C | 0.9 | 0.9 | 2.5 | 1.3 | 1.1 | 2.0 | 0.7 | 0.7 | 1.1 | 0.9 | 2.3 | 1.1 | 0.6 | 0.8 | 0.6 | 1.7 | 0.9 | 1.9 | 2.85 |
| YJR142W | 1.2 | 0.6 | 1.1 | 1.1 | 1.3 | 2.4 | 1.7 | 1.0 | 0.6 | 0.8 | 0.9 | 0.8 | 1.6 | 1.3 | 0.6 | 2.0 | 1.2 | 1.4 | 0.94 |
| YKL067W | 1.1 | 1.9 | 2.1 | 1.4 | 1.9 | 2.2 | 2.6 | 1.2 | 0.9 | 1.6 | 1.8 | 0.9 | 1.7 | 1.2 | 1.1 | 2.4 | 1.4 | 3.2 | 3.29 |
| YLR142W | 4.4 | 2.7 | 1.1 | 6.1 | 1.3 | 3.1 | 1.2 | 2.1 | 3.1 | 4.4 | 3.8 | 0.8 | 1.2 | 2.1 | 1.6 | 3.4 | 1.9 | 3.2 | 0.28 |
| YML110C | 1.1 | 0.8 | 1.9 | 1.6 | 0.8 | 2.6 | 2.0 | 1.4 | 2.7 | 2.2 | 1.7 | 1.1 | 1.8 | 1.4 | 1.3 | 2.3 | 1.1 | 1.6 | 2.01 |
| YMR272C | 0.7 | 1.0 | 1.6 | 0.7 | 1.1 | 2.6 | 1.0 | 0.8 | 0.4 | 0.6 | 0.8 | 1.2 | 1.2 | 0.8 | 1.1 | 1.5 | 1.3 | 1.4 | 1.66 |
| YNL130C | 0.6 | 0.7 | 2.4 | 0.7 | 0.8 | 2.4 | 1.3 | 0.8 | 1.7 | 2.4 | 1.5 | 0.9 | 1.9 | 1.0 | 2.5 | 0.7 | 0.7 | 1.2 | 1.18 |
| YPR006C | 1.9 | 1.5 | 0.5 | 1.9 | 2.0 | 2.4 | 2.8 | 1.5 | 1.5 | 3.9 | 2.4 | 1.1 | 1.8 | 1.2 | 0.7 | 1.6 | 1.8 | 2.1 | 0.46 |
| YBR050C | 2.4 | 1.9 | 1.9 | 3.1 | 1.0 | 4.1 | 2.6 | 1.7 | 5.8 | 3.2 | 1.1 | 1.0 | 1.3 | 1.3 | 0.5 | 1.7 | 1.5 | 1.8 | 0.41 |
| YBR145W | 1.5 | 0.7 | 2.8 | 0.9 | 1.1 | 11.5 | 58.8 | 1.0 | 0.1 | 1.1 | 1.1 | 1.0 | 2.0 | 2.2 | 1.2 | 3.6 | 1.7 | 2.0 | 2.17 |
| YBR299W | 1.8 | 0.9 | 1.1 | 3.5 | 1.6 | 0.8 | 3.6 | 2.2 | 1.1 | 5.3 | 2.4 | 1.2 | 0.7 | 1.4 | 0.6 | 3.9 | 1.0 | 1.1 | 0.32 |
| YEL020C | 1.0 | 1.5 | 0.8 | 2.9 | 1.5 | 1.3 | 2.4 | 1.2 | 1.4 | 1.1 | 1.3 | 0.8 | 1.2 | 1.0 | 1.4 | 2.1 | 1.2 | 1.3 | 0.31 |
| YGL039W | 0.8 | 1.4 | 2.1 | 1.3 | 1.3 | 1.7 | 4.2 | 1.3 | 0.7 | 2.8 | 1.1 | 1.3 | 1.5 | 0.9 | 1.5 | 1.5 | 0.6 | 1.0 | 1.09 |
| YGL134W | 0.9 | 1.3 | 0.5 | 0.8 | 1.4 | 1.2 | 2.3 | 1.4 | 1.1 | 1.4 | 1.1 | 0.9 | 0.7 | 1.1 | 0.5 | 1.7 | 0.9 | 1.3 | 0.53 |
| YJR159W | 1.4 | 2.3 | 1.3 | 1.7 | 0.9 | 1.4 | 2.3 | 2.7 | 5.2 | 2.8 | 2.2 | 1.5 | 2.1 | 2.0 | 2.3 | 5.2 | 1.7 | 2.3 | 0.30 |
| YOL157C | 1.0 | 1.1 | 1.3 | 2.5 | 1.4 | 0.9 | 2.7 | 1.4 | 2.3 | 4.8 | 1.2 | 1.2 | 1.4 | 1.2 | 1.1 | 3.5 | 1.4 | 1.3 | 0.41 |
| YOR344C | 1.1 | 1.1 | 0.4 | 0.7 | 1.9 | 2.0 | 2.4 | 1.4 | 0.3 | 0.9 | 0.6 | 1.3 | 1.8 | 0.6 | 0.7 | 0.6 | 1.3 | 1.3 | 2.11 |
| YPL265W | 0.7 | 0.4 | 2.3 | 1.1 | 1.0 | 2.3 | 8.9 | 2.3 | 0.2 | 1.2 | 1.8 | 0.9 | 1.1 | 1.1 | 1.2 | 2.0 | 0.5 | 0.4 | 1.42 |
| YBR126C | 0.8 | 1.9 | 5.6 | 1.2 | 0.7 | 2.9 | 2.3 | 0.6 | 1.7 | | 1.1 | 1.3 | 2.1 | 0.7 | 1.0 | 1.7 | 1.5 | 1.3 | 1.96 |
| YCR005C | 1.1 | 1.9 | 2.0 | 1.2 | 0.9 | 1.6 | 4.4 | 1.2 | 1.5 | 1.5 | 2.1 | 0.5 | 0.7 | 0.8 | 0.7 | 0.7 | 1.6 | 1.7 | 2.38 |
| YDR452W | 1.1 | 1.1 | 1.3 | 0.8 | 1.2 | 2.0 | 1.9 | 1.0 | 1.4 | 1.3 | 1.5 | 0.9 | 1.4 | 1.2 | 1.3 | 1.3 | 1.9 | 2.2 | 1.54 |
| YGR019W | 1.2 | 0.8 | 1.4 | 1.7 | 0.8 | 2.4 | 2.4 | 1.2 | 2.9 | | 1.1 | 1.2 | 2.1 | 1.3 | 2.2 | 2.4 | 1.3 | 2.0 | 0.79 |
| YGR255C | 0.9 | 1.4 | 1.4 | 1.5 | 1.3 | 3.2 | 1.8 | 1.0 | 2.5 | 1.8 | 2.0 | 1.3 | 2.5 | 0.9 | 1.0 | 1.1 | 1.1 | 1.1 | 0.81 |
| YIL098C | 1.4 | 1.6 | 0.5 | 2.0 | 1.7 | 1.7 | 2.2 | 1.6 | 1.9 | 1.9 | 1.4 | 0.8 | 1.3 | 1.9 | 0.7 | 2.0 | 1.2 | 1.6 | 0.75 |
| YIL172C | 1.1 | 1.1 | 1.6 | 1.7 | | 1.3 | 2.5 | 1.6 | 2.8 | 7.1 | 1.0 | 1.4 | 2.0 | 1.3 | 1.4 | 2.8 | 1.1 | 1.2 | 0.42 |
| YLR100W | 0.8 | 1.4 | 1.3 | 1.9 | 1.8 | 1.8 | 2.3 | 1.0 | 0.9 | 1.0 | 0.9 | 1.1 | 1.6 | 1.1 | 0.6 | 1.9 | 1.5 | 2.1 | 1.88 |
| YOR221C | 0.8 | 1.0 | 0.9 | 1.1 | 1.7 | 0.8 | 2.1 | 0.9 | 1.4 | 1.7 | 1.1 | 0.8 | 1.0 | 1.1 | 1.0 | 1.4 | 0.9 | 1.2 | 0.39 |
| YPL123C | 1.2 | 0.8 | 1.8 | 3.0 | 0.8 | 1.9 | 2.7 | 1.2 | 4.2 | 2.3 | 1.9 | 1.0 | 2.2 | 1.6 | 1.4 | 2.6 | 1.6 | 3.2 | 0.71 |
| YBR093C | 2.9 | 1.2 | 1.7 | 1.3 | 1.1 | 0.6 | 0.3 | 1.7 | 0.1 | 0.5 | 3.1 | 2.0 | 0.2 | 1.3 | 2.7 | 0.4 | 0.8 | 1.9 | 3.33 |
| YBR196C | 0.8 | 0.6 | 3.9 | 1.4 | 0.8 | 0.8 | 1.4 | 1.1 | 0.3 | 0.9 | 2.3 | 1.0 | 1.4 | 0.5 | 1.9 | 1.1 | 0.8 | 1.0 | 6.60 |
| YER023W | 0.8 | 0.6 | 1.2 | 1.2 | 0.8 | 1.3 | 0.7 | 0.9 | 1.1 | 0.6 | 2.1 | 0.8 | 1.2 | 0.7 | 1.2 | 1.6 | 1.5 | 1.4 | 1.77 |
| YFL055W | 2.0 | 6.7 | 1.3 | 2.5 | 1.5 | 1.1 | 1.4 | 1.3 | 23.9 | 6.5 | 2.7 | 0.9 | 1.3 | 1.5 | 0.7 | 1.4 | 1.4 | 1.0 | 0.23 |
| YIL124W | 0.9 | 0.8 | 2.8 | 1.6 | 0.9 | 1.2 | 1.3 | 0.9 | 1.0 | 0.7 | 2.8 | 1.1 | 0.9 | 1.1 | 0.6 | 3.1 | 1.2 | 2.1 | 2.39 |
| YMR318C | 1.4 | 2.4 | 2.2 | 0.7 | 1.2 | 2.1 | 0.8 | 3.6 | 2.3 | 4.8 | 3.6 | 0.8 | 1.8 | 1.7 | 1.5 | 1.1 | 1.1 | 1.1 | 3.17 |
| YBR067C | 1.6 | 2.5 | 2.8 | 0.9 | 1.1 | 1.1 | 1.1 | 1.1 | 1.7 | 1.0 | 3.1 | 1.0 | 0.9 | 0.7 | 3.3 | 2.5 | 0.5 | 0.4 | 1.76 |
| YBR115C | 0.6 | 0.7 | 1.4 | 0.4 | 0.7 | 0.9 | 1.1 | 1.3 | 0.8 | 1.6 | 1.8 | 0.9 | 2.3 | 0.7 | 1.1 | 0.5 | 0.6 | 0.6 | 0.64 |
| YDL131W | 1.1 | 0.9 | 1.8 | 1.0 | 1.0 | 0.9 | 0.7 | 2.2 | 0.8 | 2.7 | 2.2 | 1.2 | 1.0 | 0.9 | 1.1 | 0.9 | 0.7 | 0.6 | 1.81 |
| YDL168W | 2.3 | 2.0 | 2.1 | 0.9 | 1.2 | 1.4 | 1.1 | 1.7 | 8.2 | 4.7 | 1.9 | 0.6 | 1.3 | 1.6 | 1.2 | 1.1 | 0.9 | 0.8 | 1.08 |
| YDR216W | 1.3 | 1.1 | 2.0 | 2.6 | 0.8 | 1.3 | 1.0 | 1.0 | 2.5 | 2.7 | 2.1 | 1.0 | 2.4 | 0.9 | 2.5 | 1.6 | 1.0 | 1.8 | 0.44 |
| YDR253C | 5.7 | 6.4 | 1.4 | 3.2 | 0.9 | 0.8 | 1.0 | 1.7 | 14.8 | 6.3 | 2.3 | 1.0 | 2.2 | 3.6 | 1.2 | 1.6 | 1.0 | 1.0 | 0.38 |
| YDR513W | 2.2 | 2.5 | 2.3 | 2.6 | 0.9 | 2.1 | 1.6 | 1.6 | 4.6 | 3.1 | 2.0 | 0.9 | 1.8 | 2.0 | 1.3 | 3.8 | 1.3 | 3.2 | 3.10 |
| YER061C | 0.9 | 0.9 | 1.2 | 2.5 | 0.8 | 0.4 | 0.9 | 0.7 | 0.3 | 0.7 | 2.2 | 0.7 | 0.9 | 1.0 | 0.8 | 1.8 | 1.2 | 1.2 | 0.84 |
| YFL052W | 1.3 | 0.8 | 0.7 | 2.3 | 1.5 | 2.1 | 1.4 | 1.3 | 0.6 | 1.2 | 2.3 | 0.8 | 1.0 | 1.6 | 0.4 | 1.0 | 1.1 | 0.9 | 0.32 |
| YFL058W | 1.2 | 1.7 | 1.1 | 1.4 | 1.1 | 2.0 | 0.9 | 1.7 | 3.2 | 2.4 | 2.4 | 1.2 | 1.2 | 1.0 | 1.7 | 0.7 | 1.1 | 0.9 | 0.40 |
| YFR030W | 3.1 | 5.5 | 8.5 | 0.5 | 0.8 | 1.8 | 1.1 | 1.7 | 24.1 | 9.0 | 2.4 | 2.4 | 4.1 | 1.3 | 1.7 | 1.6 | 1.3 | 0.9 | 0.30 |
| YGL202W | 0.8 | 0.7 | 2.2 | 0.7 | 0.7 | 0.7 | 0.6 | 1.0 | 1.3 | 1.7 | 2.4 | 0.9 | 1.1 | 0.8 | 2.2 | 0.6 | 1.3 | 0.9 | 1.70 |
| YGR070W | 1.1 | 1.0 | 2.0 | 1.5 | 1.4 | | 1.4 | 1.8 | 1.1 | 1.5 | 1.8 | 0.6 | 1.4 | 1.0 | 2.6 | 2.3 | 1.2 | 1.3 | 0.40 |
| YHL036W | 2.2 | 4.8 | 3.4 | 2.0 | 1.3 | 1.3 | 1.0 | 1.2 | 9.0 | 4.4 | 2.3 | 1.2 | 1.5 | 1.2 | 0.9 | 1.1 | 1.0 | 1.0 | 0.60 |
| YHR104W | 1.0 | 4.1 | 15.7 | 1.9 | 1.1 | 1.2 | 1.5 | 1.1 | 4.8 | 2.3 | 2.6 | 1.0 | 1.2 | 1.5 | 1.6 | 1.3 | 1.9 | 1.9 | 1.57 |
| YJL045W | 1.8 | 2.2 | 1.6 | 5.3 | 0.7 | 0.6 | 0.7 | 0.9 | 9.7 | 1.6 | 2.3 | 1.1 | 1.6 | 1.2 | 3.4 | 2.4 | 0.9 | 0.9 | 0.42 |
| YJL060W | 1.8 | 2.6 | 9.6 | 1.0 | 1.4 | 1.3 | 1.3 | 0.8 | 5.3 | 2.8 | 1.6 | 1.5 | 2.1 | 1.7 | 1.4 | 1.5 | 1.2 | 1.1 | 0.95 |
| YJL155C | 2.1 | 5.0 | 0.8 | 2.4 | 0.8 | 0.8 | 2.0 | 1.5 | 3.8 | 2.8 | 2.4 | 1.0 | 6.6 | 1.2 | 1.2 | 2.3 | 1.8 | 3.1 | 0.60 |
| YJR109C | 0.8 | 0.7 | 1.4 | 0.9 | 0.7 | 0.9 | 0.8 | 1.0 | 1.8 | 4.3 | 1.5 | 1.4 | 3.1 | 0.9 | 1.3 | 0.5 | 1.1 | 0.6 | 0.84 |
| YJR156C | 1.6 | 3.2 | 2.4 | 1.8 | 0.9 | 0.9 | 1.3 | 1.5 | 3.4 | 2.8 | 3.0 | 1.1 | 1.6 | 2.4 | 3.0 | 2.3 | 0.8 | 1.1 | 0.24 |
| YLR092W | 5.0 | 7.5 | 1.4 | 0.6 | 1.3 | 1.0 | 1.3 | 1.5 | 12.7 | 5.8 | 3.1 | 1.0 | 1.8 | 1.4 | 0.9 | 1.2 | 1.0 | 1.1 | 0.24 |
| YMR081C | 3.5 | 1.4 | 2.0 | 5.8 | 1.7 | 1.1 | 0.9 | 1.7 | 0.3 | 2.2 | 2.5 | 1.1 | 0.7 | 1.8 | 0.2 | 3.0 | 1.9 | 3.0 | 0.62 |
| YMR250W | 0.9 | 5.1 | 5.6 | 1.3 | 0.6 | 4.5 | 3.0 | 0.9 | 6.1 | 2.4 | 4.3 | 1.2 | 2.6 | 1.1 | 2.0 | 5.0 | 1.2 | 3.0 | 0.84 |
| YNL277W | 5.7 | 10.3 | 5.3 | 0.6 | 1.1 | 1.7 | 1.4 | 2.2 | 55.0 | 10.9 | 3.2 | 1.3 | 1.8 | 1.5 | 0.6 | 1.3 | 0.9 | 0.7 | 0.27 |
| YOR184W | 1.1 | 0.9 | 2.1 | 2.1 | 1.0 | 0.6 | 1.2 | 0.7 | 0.9 | 0.7 | 2.7 | 0.7 | 0.6 | 0.7 | 1.0 | 0.9 | 1.2 | 1.2 | 3.19 |
| YPR160W | 1.4 | 3.8 | 3.6 | 3.3 | 0.7 | 4.5 | 1.8 | 0.9 | 0.9 | 1.3 | 4.4 | 2.1 | 2.2 | 1.1 | 1.4 | 5.3 | 1.0 | 2.9 | 1.42 |
| YDL182W | 0.8 | 0.7 | 1.3 | 0.8 | 1.3 | 1.0 | 1.0 | 2.9 | 0.8 | 2.4 | 2.0 | 1.4 | 1.5 | 0.8 | 0.7 | 1.1 | 0.6 | 0.6 | 2.31 |
| YBR291C | 2.0 | 0.9 | 1.0 | 1.5 | 0.9 | 1.1 | 0.9 | 2.2 | 0.9 | 1.0 | 1.1 | 0.9 | 1.0 | 1.7 | 0.5 | 1.5 | 0.9 | 1.3 | 1.19 |
| YIL094C | 1.5 | 0.4 | 0.8 | 0.7 | 1.4 | 1.2 | 1.3 | 3.6 | 0.3 | 0.4 | 1.8 | 1.0 | 0.7 | 1.0 | 0.5 | 0.9 | 0.9 | 1.0 | 2.26 |
| YNR050C | 1.7 | 0.5 | 1.2 | 0.6 | 1.3 | 0.9 | 1.0 | 2.2 | 0.4 | 0.7 | 1.5 | 1.0 | 1.1 | 0.6 | 0.3 | 1.4 | 0.6 | 0.7 | 2.13 |
| YDL244W | 1.1 | 3.5 | 1.8 | 1.6 | 0.9 | 1.3 | 1.7 | 1.8 | 2.5 | 3.7 | 1.2 | 1.2 | 2.0 | 2.0 | 2.9 | 1.5 | 1.1 | 1.0 | 0.25 |
| YDR054C | 1.3 | 1.0 | 0.7 | 1.4 | 0.7 | 1.0 | 1.1 | 1.5 | 2.9 | 3.2 | 1.9 | 1.0 | 2.5 | 1.1 | 1.6 | 1.4 | 0.8 | 1.4 | 1.18 |
| YDR353W | 0.9 | 1.6 | 3.4 | 1.6 | 0.6 | 0.9 | 1.0 | 1.1 | 2.0 | 5.1 | 1.4 | 0.8 | 0.7 | 1.6 | 1.3 | 1.0 | 0.9 | 1.3 | 3.20 |
| YEL070W | 1.4 | 0.9 | 1.6 | 3.4 | 1.3 | 1.2 | 1.3 | 1.2 | 1.9 | 13.0 | 1.1 | 1.1 | 1.1 | 2.1 | 3.2 | 1.9 | 0.9 | 1.0 | 0.27 |
| YIL168W | 2.8 | 1.4 | 1.0 | 1.2 | 1.4 | 0.7 | 1.3 | 1.6 | 7.8 | 19.3 | 1.9 | 0.8 | 0.9 | 1.5 | 1.2 | 1.2 | 1.0 | 1.1 | 0.39 |
| YJL221C | 1.1 | 1.0 | 1.1 | 1.3 | 0.9 | 6.6 | 2.5 | 1.8 | 2.7 | 4.4 | 0.8 | 1.1 | 1.4 | 1.1 | 1.1 | 3.3 | 1.1 | 1.4 | 0.41 |
| YJR095W | 1.2 | 20.5 | 1.9 | 6.7 | 1.2 | 1.5 | 2.0 | 0.9 | 0.5 | 6.3 | 0.6 | 0.7 | 0.8 | 1.3 | 0.8 | 0.8 | 1.3 | 0.9 | 0.23 |
| YKL085W | 1.4 | 2.3 | 1.6 | 1.2 | 1.2 | 1.9 | 1.5 | 1.2 | 1.9 | 3.0 | 1.8 | 0.8 | 1.5 | 1.0 | 0.5 | 1.7 | 0.9 | 1.3 | 2.16 |
| YKL188C | 1.3 | 0.7 | 1.9 | 2.4 | 1.0 | 0.8 | 1.1 | 0.7 | 2.5 | 2.8 | 1.1 | 1.2 | 1.2 | 2.1 | 1.4 | 2.7 | 1.1 | 1.5 | 0.27 |
| YKL217W | 1.8 | 2.4 | 1.0 | 2.1 | 1.1 | 1.2 | 1.6 | 1.1 | 0.9 | 4.1 | 1.6 | 0.8 | 1.2 | 1.2 | 2.2 | 3.0 | 1.7 | 3.3 | 0.29 |
| YKR061W | 2.0 | 0.9 | 0.5 | 1.9 | 1.2 | 1.2 | 1.4 | 1.5 | 1.5 | 2.6 | 1.1 | 0.9 | 0.9 | 1.6 | 0.9 | 0.8 | 1.5 | 1.6 | 0.70 |
| YLR174W | 1.2 | 1.5 | 1.7 | 2.1 | 0.9 | 0.9 | 1.6 | 1.1 | 2.5 | 8.3 | 1.3 | 1.3 | 1.8 | 1.7 | 0.8 | 4.6 | 0.9 | 1.2 | 0.41 |
| YLR260W | 1.1 | 1.5 | 1.5 | 2.4 | 1.2 | 1.0 | 1.2 | 1.2 | 2.6 | 3.2 | 0.6 | 0.8 | 1.9 | 1.2 | 1.0 | 1.0 | 1.1 | 1.2 | 0.44 |
| YNL009W | 1.1 | 1.9 | 2.0 | 1.4 | 1.4 | 1.1 | 1.2 | 1.1 | 1.4 | 3.3 | 1.3 | 0.9 | 1.3 | 1.3 | 2.7 | 3.3 | 1.2 | 2.3 | 0.45 |
| YNL117W | 0.9 | 4.7 | 1.7 | 0.8 | 0.8 | | 1.1 | 1.7 | 12.8 | 4.4 | 1.4 | 0.7 | 1.3 | 2.0 | 2.6 | 1.6 | 1.1 | 0.9 | 0.24 |
| YNL183C | 0.9 | 3.5 | 6.4 | 1.3 | 0.7 | 1.2 | 1.3 | 0.9 | 4.8 | 4.3 | 1.3 | 1.1 | 2.3 | 1.1 | 1.1 | 1.2 | 0.8 | 1.0 | 0.47 |
| YNR073C | 1.1 | 1.4 | 0.8 | 2.7 | 1.4 | 1.7 | 2.2 | 1.5 | 2.5 | 16.8 | 2.2 | 1.1 | 1.2 | 2.3 | 1.7 | 1.6 | 1.0 | 0.9 | 0.18 |
| YPL161C | 1.0 | 1.7 | 1.1 | 1.1 | 0.8 | 0.7 | 1.3 | 1.8 | 1.2 | 2.7 | 1.0 | 1.0 | 1.2 | 1.1 | 0.8 | 1.4 | 1.3 | 1.3 | 0.41 |
| YAL061W | 1.7 | 2.4 | 3.3 | 3.8 | 1.0 | 1.0 | 2.0 | 0.8 | 5.5 | 1.4 | 4.1 | 1.1 | 1.4 | 0.7 | 0.6 | 1.1 | 1.4 | 1.2 | 0.88 |
| YAL067C | 2.7 | 7.5 | 1.5 | 1.4 | 0.7 | 1.2 | 0.8 | 1.1 | 7.6 | 2.9 | 1.2 | 1.0 | 1.1 | 1.3 | 2.4 | 1.6 | 1.0 | 1.0 | 0.33 |
| YBL033C | 1.3 | 1.9 | 2.4 | 1.0 | 1.1 | 0.4 | 0.9 | 1.4 | 6.6 | 4.2 | 1.1 | 0.9 | 1.8 | 1.4 | 1.0 | 1.2 | 1.2 | 1.0 | 0.53 |
| YBL086C | 0.8 | 0.5 | 1.0 | 0.6 | 1.3 | 0.7 | 1.0 | 1.1 | 3.3 | 1.4 | 1.3 | 0.5 | 0.9 | 0.8 | 0.5 | 1.4 | 0.9 | 1.0 | 0.43 |
| YBR117C | 0.8 | 1.6 | 1.5 | 1.4 | 0.9 | 2.3 | 1.0 | 0.7 | 5.9 | 1.5 | 0.4 | 0.8 | 2.1 | 0.9 | 1.0 | 12.0 | 0.7 | 0.7 | 0.49 |
| YBR213W | 1.0 | 1.6 | 1.0 | 0.5 | 1.1 | 2.4 | 1.3 | 1.3 | 11.4 | 2.3 | 0.5 | 1.2 | 1.4 | 1.3 | 1.7 | 0.8 | 0.9 | 0.9 | 0.23 |
| YCR036W | 1.3 | 1.2 | 1.3 | 1.3 | 1.5 | 1.4 | 1.5 | 1.1 | 2.6 | 1.5 | 1.0 | 1.3 | 1.8 | 1.5 | 1.7 | 1.6 | 1.6 | 1.7 | 1.35 |
| YDL132W | 0.8 | 1.2 | 1.1 | 0.7 | 1.3 | 0.9 | 1.4 | 1.0 | 3.8 | 2.4 | 1.3 | 0.7 | 2.0 | 0.9 | 1.7 | 1.0 | 0.9 | 1.1 | 0.74 |
| YER014W | 1.0 | 0.9 | 0.9 | 0.6 | 0.8 | 4.0 | | 1.2 | 3.5 | 1.1 | 0.9 | 1.1 | 1.5 | 1.2 | 2.3 | 0.8 | 1.0 | 0.9 | 0.43 |
| YER042W | 3.0 | 2.2 | 3.1 | 1.0 | 1.1 | 1.6 | 1.1 | 1.7 | 6.8 | 2.1 | 1.8 | 1.1 | 1.4 | 5.2 | 1.0 | 1.9 | 0.7 | 1.1 | 1.80 |
| YER090W | 1.0 | 1.1 | 2.0 | 0.7 | 1.3 | 0.7 | 0.9 | 1.4 | 3.7 | 2.5 | 1.8 | 1.3 | 1.2 | 1.2 | 1.5 | 0.8 | 1.1 | 0.9 | 0.97 |
| YGL026C | 0.7 | 9.3 | 1.9 | 0.6 | 1.2 | 1.7 | 0.9 | 0.8 | 3.7 | 1.5 | 3.4 | 1.3 | 1.8 | 0.9 | 2.4 | 0.7 | 1.5 | 0.8 | 0.87 |
| YGL252C | 1.1 | 2.4 | 1.4 | 0.7 | 1.3 | 0.8 | 1.4 | 1.2 | 2.7 | 1.7 | 0.9 | 0.9 | 1.2 | 1.0 | 1.3 | 1.2 | 1.0 | 1.3 | 0.91 |
| YGL254W | 1.2 | 1.1 | 0.8 | 0.6 | 1.1 | 0.7 | 1.3 | 1.4 | 3.1 | 1.8 | 1.1 | 1.2 | 2.4 | 1.3 | 1.1 | 1.4 | 0.8 | 1.2 | 0.53 |
| YGR276C | 0.9 | 0.8 | 0.3 | 1.3 | 1.5 | 0.7 | 1.9 | 1.5 | 4.5 | 2.2 | 0.9 | 1.0 | 1.8 | 1.0 | 0.8 | 1.3 | 0.9 | 1.1 | 0.97 |
| YHR106W | 0.9 | 1.7 | 1.7 | 1.5 | 1.7 | 1.3 | 1.2 | 1.0 | 2.8 | 2.5 | 1.4 | 0.7 | 1.0 | 1.2 | 0.9 | 2.4 | 1.3 | 1.1 | 1.73 |
| YIL046W | 1.5 | 2.7 | 1.6 | 0.7 | 1.4 | 0.9 | 1.0 | 1.0 | 10.7 | 2.5 | 1.2 | 1.0 | 1.7 | 1.1 | 1.3 | 1.8 | 9.0 | 5.8 | 0.68 |
| YJL071W | 1.0 | 1.5 | 1.3 | 1.6 | 1.4 | 1.0 | 1.3 | 1.1 | 2.8 | 2.2 | 1.3 | 1.1 | 1.0 | 1.0 | 1.5 | 1.2 | 0.9 | 0.9 | 0.41 |
| YJR139C | 1.4 | 2.6 | 2.9 | 1.1 | 1.2 | 1.8 | 1.3 | 1.1 | 4.1 | 1.9 | 1.5 | 1.0 | 1.5 | 1.4 | 0.7 | 1.2 | 1.0 | 1.3 | 4.47 |
| YKR069W | 3.4 | 6.3 | 5.0 | 3.1 | 0.7 | 0.5 | 1.0 | 1.2 | 10.9 | 9.0 | 2.0 | 1.4 | 1.4 | 1.7 | 1.1 | 1.8 | 1.0 | 1.0 | 0.25 |
| YLL061W | 3.6 | 2.8 | 9.2 | 0.4 | 0.8 | 0.8 | 0.9 | 1.4 | 3.7 | 5.5 | 1.7 | 0.7 | 0.9 | 1.3 | 1.4 | 1.0 | 0.6 | 0.7 | 0.33 |
| YLR070C | 1.0 | 1.3 | 1.4 | 1.1 | 0.7 | 1.1 | 1.3 | 1.0 | 3.5 | 1.6 | 0.4 | 0.7 | 2.0 | 1.3 | 0.5 | 2.1 | 1.0 | 0.9 | 0.31 |
| YLR099C | 2.0 | 2.2 | 1.4 | 0.8 | 0.9 | 0.4 | 0.2 | 1.2 | 3.6 | 1.1 | 1.2 | 0.9 | 0.8 | 0.7 | 0.9 | 0.7 | 0.7 | 0.5 | 0.92 |
| YLR157C | 1.1 | 1.1 | 3.4 | 1.1 | 1.3 | 1.9 | 1.9 | 1.5 | 5.1 | 1.8 | 1.9 | 1.2 | 1.8 | 1.1 | 1.1 | 2.1 | 1.6 | 2.1 | 1.70 |
| YLR160C | 1.2 | 1.4 | 3.9 | 1.1 | 1.3 | 1.8 | 1.9 | 2.0 | 4.8 | 1.8 | 1.6 | 1.2 | 2.0 | 1.2 | 1.0 | 2.0 | 1.6 | 2.2 | 1.59 |
| YLR164W | 1.0 | 2.4 | 0.3 | 1.0 | 1.2 | 1.2 | 1.1 | 1.2 | 5.3 | 2.0 | 3.2 | 0.7 | 1.0 | 1.5 | 0.7 | 11.0 | 1.5 | 1.2 | 0.31 |
| YML042W | 0.9 | 1.4 | 1.5 | 2.2 | 1.0 | 0.9 | 0.8 | 1.6 | 3.8 | 2.4 | 2.5 | 1.0 | 1.2 | 1.4 | 1.7 | 4.8 | 1.0 | 0.9 | 0.29 |
| YOL049W | 0.9 | 1.1 | 1.3 | 0.9 | 1.4 | 0.6 | 1.1 | 1.0 | 3.0 | 1.9 | 1.8 | 0.8 | 0.9 | 0.9 | 0.7 | 1.3 | 1.0 | 1.3 | 1.61 |
| YOL064C | 1.4 | 2.1 | 1.1 | 0.6 | 0.9 | 1.4 | 1.2 | 1.0 | 8.7 | 2.7 | 1.0 | 0.9 | 2.0 | 0.9 | 1.0 | 1.7 | 1.2 | 1.3 | 1.32 |
| YOR377W | 0.6 | 0.6 | 0.6 | 1.0 | 1.4 | 1.6 | 1.6 | 1.0 | 3.8 | 1.1 | 1.2 | 0.9 | 1.3 | 0.9 | 1.2 | 1.3 | 1.1 | 1.1 | 0.46 |
| YPL031C | 0.9 | 2.0 | 1.4 | 0.8 | 1.5 | 1.8 | 1.6 | 1.0 | 5.7 | 1.4 | 1.4 | 0.8 | 1.6 | 0.9 | 0.7 | 2.0 | 1.4 | 1.2 | 0.49 |
| YPL113C | 1.4 | 1.7 | 0.4 | 2.4 | 1.3 | 1.0 | 1.9 | 1.4 | 5.5 | 2.0 | 1.5 | 1.1 | 1.7 | 1.6 | 0.9 | 2.5 | 1.2 | 1.7 | 0.28 |
| YPL274W | 0.8 | 4.2 | 3.8 | 0.8 | 0.6 | 1.1 | 0.7 | 0.9 | 4.1 | 3.6 | 2.0 | 0.5 | 0.6 | 0.9 | 1.5 | 1.1 | 0.7 | 0.6 | 0.41 |
| YPR048W | 1.3 | 2.0 | 0.9 | 1.1 | 0.9 | 0.6 | 0.9 | 1.5 | 4.0 | 2.1 | 1.0 | 0.8 | 0.6 | 1.2 | 0.6 | 0.8 | 0.7 | 0.8 | 0.75 |
| YBR001C | 0.9 | 1.4 | 1.1 | 1.4 | 0.9 | 1.1 | 0.9 | 1.3 | 2.3 | 2.1 | 1.0 | 1.0 | 2.0 | 1.2 | 1.8 | 1.4 | 1.0 | 1.6 | 0.61 |
| YBR018C | 0.9 | 1.1 | 1.9 | 2.9 | 0.9 | 0.7 | 1.4 | 0.6 | 1.3 | 1.7 | 5.8 | 1.0 | 1.0 | 2.4 | 1.3 | 6.2 | 0.8 | 0.9 | 0.20 |
| YBR204C | 1.0 | 1.0 | 1.5 | 1.8 | 1.1 | 0.9 | 1.7 | 1.1 | 1.8 | 1.9 | 2.2 | 1.0 | 1.7 | 1.4 | 1.2 | 2.3 | 1.1 | 1.5 | 0.84 |
| YBR241C | 0.9 | 9.3 | 2.6 | 0.9 | 0.9 | 1.5 | 1.4 | 0.7 | 29.8 | 2.2 | 1.4 | 1.4 | 1.4 | 0.9 | 1.6 | 0.7 | 1.0 | 1.2 | 1.25 |
| YCR105W | 2.2 | 1.2 | 1.2 | 3.0 | 0.9 | 1.0 | 1.0 | 1.4 | 2.0 | 3.9 | 2.3 | 0.9 | 1.9 | 1.9 | 3.1 | 1.3 | 1.3 | 1.1 | 0.36 |
| YDR287W | 1.1 | 8.4 | 1.9 | 1.2 | 1.2 | 1.6 | 1.2 | 1.2 | 2.5 | 1.8 | 1.5 | 1.1 | 1.5 | 1.2 | 1.6 | 2.7 | 1.5 | 1.3 | 0.52 |
| YDR294C | 0.7 | 1.1 | 1.9 | 1.2 | 0.9 | 0.7 | 1.0 | 1.0 | 2.0 | 2.9 | 1.2 | 1.1 | 1.6 | 1.0 | 2.0 | 1.7 | 0.9 | 1.1 | 1.02 |
| YER052C | 1.1 | 1.0 | 3.6 | 1.1 | 0.6 | 0.6 | 0.5 | 1.0 | 2.7 | 2.4 | 1.1 | 0.9 | 2.1 | 0.7 | 1.2 | 0.2 | 0.8 | 0.4 | 1.54 |
| YFL021W | 0.8 | 1.4 | 2.4 | 1.7 | 1.1 | 0.8 | 0.8 | 0.6 | 2.7 | 1.2 | 1.3 | 0.9 | 1.5 | 1.1 | 1.3 | 1.0 | 0.8 | 0.5 | 0.40 |
| YGL040C | 0.7 | 1.2 | 1.4 | 0.9 | 1.3 | 2.0 | 1.1 | 1.0 | 2.1 | 1.9 | 0.9 | 0.6 | 1.6 | 1.3 | 0.7 | 1.5 | 0.7 | 1.0 | 1.77 |
| YGL125W | 1.0 | 3.2 | 4.3 | 0.7 | 0.9 | | 1.7 | 0.8 | 2.1 | 2.1 | 1.6 | 1.0 | 1.4 | 1.0 | 1.9 | 1.2 | 1.2 | 1.1 | 0.27 |
| YGR007W | 1.3 | 1.7 | 0.7 | 1.6 | 0.6 | 0.8 | 1.2 | 1.1 | 2.2 | 2.3 | 0.9 | 0.8 | 1.4 | 1.8 | 1.9 | 1.3 | 1.1 | 1.4 | 0.74 |
| YGR155W | 1.3 | 4.4 | 1.2 | 0.6 | 1.3 | 1.6 | 1.1 | 1.4 | 2.4 | 1.7 | 1.3 | 1.0 | 1.4 | 0.7 | 0.5 | 1.0 | 0.5 | 0.6 | 4.15 |
| YIL099W | 0.9 | 1.6 | 1.5 | 0.4 | 0.8 | | 101.4 | 1.0 | 2.2 | 1.8 | 0.9 | 0.9 | 1.4 | 1.6 | 5.5 | 1.2 | 1.0 | 1.1 | 0.18 |
| YIL170W | 1.1 | 1.0 | 2.5 | 2.2 | 0.9 | 8.8 | 0.5 | 1.2 | 5.7 | 3.2 | 1.7 | 0.7 | 1.9 | 1.1 | 2.3 | 1.8 | 1.6 | 1.5 | 0.48 |
| YIR031C | 0.8 | 1.6 | 0.6 | 0.6 | 1.6 | 1.7 | 1.4 | 1.0 | 2.6 | 1.0 | 1.2 | 0.8 | 1.0 | 1.2 | 0.8 | 0.7 | 0.9 | 0.8 | 0.44 |
| YIR032C | 1.1 | 1.9 | 1.3 | 2.6 | 1.0 | 0.6 | 1.0 | 1.2 | 2.6 | 2.3 | 1.4 | 1.0 | 1.4 | 1.6 | 0.6 | 1.6 | 0.9 | 1.1 | 0.40 |
| YJL128C | 0.8 | 1.4 | 0.4 | 0.8 | 1.2 | 1.3 | 1.8 | 0.9 | 2.0 | 1.7 | 0.6 | 1.0 | 1.7 | 0.8 | 1.2 | 0.9 | 1.0 | 1.0 | 0.42 |
| YJR090C | 0.8 | 1.0 | 0.6 | 0.8 | 1.2 | 1.2 | 0.8 | 0.8 | 1.9 | 1.2 | 0.4 | 0.8 | 2.2 | 1.0 | 1.6 | 0.8 | 1.1 | 1.0 | 0.60 |
| YJR103W | 1.1 | 3.8 | 1.2 | 1.6 | 0.7 | 0.6 | 0.6 | 0.8 | 1.8 | 3.0 | 1.1 | 0.6 | 0.7 | 0.7 | 3.2 | 2.3 | 0.9 | 1.0 | 0.74 |
| YJR153W | 1.1 | 1.7 | 1.1 | 0.8 | 0.9 | 0.4 | 1.3 | 0.9 | 2.4 | 1.6 | 2.0 | 0.7 | 1.3 | 1.7 | 2.9 | 1.6 | 0.8 | 1.0 | 0.18 |
| YKL192C | 1.1 | 1.0 | 3.7 | 1.1 | 1.1 | 1.8 | 1.1 | 0.9 | 3.5 | 1.6 | 1.1 | 1.2 | 1.7 | 1.5 | 1.2 | 2.1 | 1.0 | 0.8 | 1.57 |
| YLR025W | 1.2 | 5.1 | 0.7 | 1.1 | 1.9 | 1.5 | 1.7 | 1.5 | 2.4 | 1.5 | 1.1 | 0.9 | 1.0 | 1.7 | 1.5 | 2.2 | 1.2 | 1.3 | 1.15 |
| YML051W | 1.0 | 1.4 | 1.8 | 0.9 | 1.0 | 0.7 | 0.9 | 0.9 | 2.0 | 1.5 | 1.4 | 0.7 | 1.2 | 1.1 | 1.4 | 1.1 | 0.7 | 1.0 | 0.57 |
| YML099C | 0.6 | 1.0 | 1.1 | 1.3 | 1.0 | 0.5 | 1.0 | 0.7 | 2.2 | 1.7 | 1.6 | 1.1 | 1.3 | 0.9 | 1.0 | 1.6 | 0.7 | 0.8 | 0.48 |
| YMR056C | 1.1 | 1.3 | 1.7 | 1.0 | 1.1 | 0.9 | 1.4 | 1.2 | 1.8 | 1.1 | 1.6 | 0.9 | 0.9 | 1.1 | 0.7 | 2.4 | 1.0 | 1.1 | 0.72 |
| YNL257C | 0.7 | 1.0 | 0.9 | 0.8 | 0.9 | 2.0 | 1.1 | 1.1 | 2.1 | 1.4 | 1.0 | 1.1 | 1.7 | 0.8 | 1.0 | 1.4 | 0.9 | 1.3 | 0.63 |
| YNL264C | 1.0 | 2.4 | 1.3 | 1.0 | 0.8 | 1.4 | 1.5 | 1.1 | 3.3 | 1.3 | 1.0 | 1.1 | 1.5 | 1.2 | 1.1 | 1.3 | 0.7 | 1.1 | 0.48 |
| YNR071C | 1.4 | 1.0 | 0.9 | 3.2 | 1.1 | 0.9 | 1.4 | 1.2 | 3.1 | 7.0 | 1.3 | 0.6 | 1.7 | 1.1 | 1.8 | 1.0 | 1.0 | 0.9 | 0.16 |
| YOL065C | 1.0 | 1.7 | 1.6 | 1.3 | 0.8 | 1.3 | 1.9 | 1.0 | 2.1 | 1.8 | 1.0 | 0.8 | 1.3 | 1.1 | 0.7 | 2.6 | 1.1 | 1.5 | 0.43 |
| YOL067C | 0.8 | 0.9 | 0.9 | 1.4 | 0.7 | 0.3 | 0.7 | 1.0 | 2.1 | 1.3 | 1.3 | 0.7 | 1.7 | 0.7 | 1.2 | 1.1 | 1.4 | 0.9 | 0.55 |
| YPL147W | 1.0 | 0.8 | 2.3 | 1.2 | 1.2 | 1.1 | 1.0 | 0.9 | 2.4 | 2.4 | 1.3 | 0.9 | 1.5 | 1.0 | 2.4 | 2.5 | 1.5 | 3.6 | 0.33 |
| YDR043C | 2.8 | 1.1 | 1.2 | 6.9 | 1.7 | 1.4 | 1.3 | 1.2 | 1.0 | 2.7 | 1.3 | 0.9 | 0.7 | 1.8 | 1.2 | 1.4 | 1.9 | 1.2 | 0.66 |
| YGR180C | 3.1 | 1.1 | 1.9 | 1.0 | 2.0 | 0.6 | 0.5 | 1.0 | 1.4 | 1.5 | 1.0 | 0.9 | 0.9 | 0.9 | 1.3 | 1.2 | 1.0 | 1.9 | 3.90 |
| YJL026W | 2.5 | 2.4 | 2.9 | 1.0 | 1.7 | 1.4 | 0.5 | 1.2 | 1.0 | 1.1 | 1.3 | 1.0 | 1.2 | 1.0 | 1.1 | 1.6 | 1.4 | 2.1 | 3.74 |
| YGR087C | 1.0 | 15.0 | 1.7 | 0.6 | 1.0 | 1.1 | 1.0 | 0.8 | 0.2 | 0.9 | 0.8 | 1.2 | 1.2 | 0.7 | 3.5 | 0.6 | 1.0 | 0.6 | 1.88 |
| YGL256W | 0.9 | 5.3 | 1.1 | 1.3 | 0.7 | 0.7 | 0.5 | 1.0 | 0.4 | 1.2 | 1.0 | 0.9 | 0.8 | 0.9 | 1.0 | 0.7 | 0.7 | 0.8 | 0.90 |
| YAL039C | 1.1 | 2.2 | 1.7 | 1.0 | 1.1 | 0.8 | 1.0 | 1.2 | 1.6 | 2.2 | 1.1 | 1.4 | 1.6 | 1.2 | 3.3 | 2.2 | 1.4 | 1.1 | 0.39 |
| YJR107W | 1.0 | 3.1 | 1.5 | 1.8 | 0.7 | 1.5 | 1.3 | 1.3 | 1.4 | 0.9 | 1.3 | 1.1 | 1.7 | 1.4 | 1.1 | 1.1 | 1.4 | 1.3 | 0.41 |
| YNL142W | 0.7 | 2.4 | 2.5 | 0.8 | 1.3 | 0.9 | 1.1 | 0.8 | 0.9 | 0.6 | 0.9 | 1.2 | 0.9 | 0.8 | 0.3 | 1.0 | 1.0 | 0.9 | 0.44 |
| YDL210W | 1.2 | 2.2 | 2.7 | 1.1 | 0.9 | 1.3 | 0.8 | 1.1 | 1.2 | 1.4 | 0.6 | 0.8 | 1.2 | 1.1 | 1.9 | 0.9 | 1.1 | 0.9 | 0.20 |
| YGL055W | 1.1 | 1.8 | 0.3 | 2.5 | 1.5 | 1.6 | 0.6 | 1.4 | 0.2 | 0.7 | 0.9 | 1.3 | 0.2 | 0.7 | 0.5 | 3.0 | 1.0 | 1.5 | 5.60 |
| YCL025C | 1.1 | 4.6 | 2.5 | 1.7 | 0.9 | 0.9 | 0.7 | 0.7 | 0.3 | 0.3 | 1.0 | 1.1 | 0.5 | 0.7 | 0.8 | 0.6 | 0.6 | 0.6 | 1.98 |
| YBR132C | 0.9 | 1.9 | 2.4 | 1.8 | 0.8 | 1.2 | 1.1 | 0.8 | 0.8 | 1.1 | 1.9 | 1.0 | 1.4 | 1.1 | 1.5 | 1.3 | 1.1 | 1.3 | 0.43 |
| YHL018W | 0.8 | 1.5 | 0.5 | 0.5 | 0.9 | 0.4 | 1.2 | 0.8 | 0.8 | 0.6 | 1.2 | 0.7 | 0.8 | 1.0 | 0.9 | 0.9 | 1.0 | 0.9 | 0.37 |
| YPL038W | 0.8 | 1.5 | 0.2 | 1.1 | 1.4 | 0.5 | 1.0 | 1.2 | 0.9 | 0.7 | 1.1 | 0.6 | 0.6 | 0.9 | 0.6 | 0.8 | 0.9 | 0.9 | 0.64 |
| YKR053C | 0.9 | 2.0 | 1.0 | 1.2 | 1.0 | 1.1 | 1.7 | 0.9 | 0.8 | 1.8 | 0.5 | 0.7 | 0.7 | 1.3 | 0.3 | 0.9 | 2.8 | 2.4 | 0.35 |
| YNL256W | 0.7 | 1.6 | 0.6 | 0.6 | 1.0 | 0.5 | 0.6 | 0.9 | 0.8 | 0.6 | 0.6 | 0.6 | 1.3 | 0.7 | 0.9 | 0.4 | 0.6 | 0.6 | 1.09 |
| YLR377C | 1.1 | 2.6 | 1.8 | 1.2 | 1.0 | 0.7 | 0.9 | 0.7 | 1.8 | 1.6 | 0.6 | 0.6 | 1.2 | 2.9 | 0.8 | 2.7 | 0.9 | 1.4 | 0.14 |
| YBR253W | 1.3 | 4.4 | 0.4 | 1.1 | 1.4 | 1.3 | 1.4 | 1.8 | 1.8 | 1.5 | 1.1 | 1.0 | 0.8 | 1.2 | 0.9 | 1.3 | 1.3 | 1.1 | 0.96 |
| YBL030C | 1.0 | 0.9 | 4.5 | 0.9 | 0.7 | 1.1 | 0.9 | 0.8 | 0.4 | 0.9 | 1.1 | 0.9 | 0.5 | 0.8 | 1.8 | 1.0 | 1.0 | 1.1 | 3.12 |
| YBR221C | 0.8 | 0.7 | 3.2 | 1.5 | 1.3 | 1.7 | 1.5 | 0.8 | 1.0 | 1.3 | 1.2 | 1.3 | 1.7 | 0.8 | 1.1 | 1.5 | 0.9 | 1.0 | 3.62 |
| YDR342C | 2.8 | 1.1 | 12.2 | 5.7 | 1.6 | 1.1 | 0.8 | 1.2 | 0.2 | 2.2 | 2.9 | 1.0 | 0.6 | 0.9 | 0.5 | 2.4 | 1.0 | 2.2 | 5.23 |
| YDR343C | 1.2 | 1.0 | 20.6 | 4.6 | 1.3 | 1.3 | 0.7 | 1.2 | 0.3 | 2.1 | 2.3 | 1.0 | 0.8 | 0.8 | 0.5 | 2.8 | 1.1 | 2.3 | 5.81 |
| YEL034W | 0.9 | 0.6 | 3.3 | 0.9 | 1.4 | 0.9 | 0.4 | 0.9 | 0.8 | 1.0 | 1.1 | 0.9 | 0.8 | 0.9 | 0.8 | 0.9 | 0.9 | 0.9 | 5.44 |
| YHR094C | 0.7 | 1.2 | 5.3 | 1.6 | 1.1 | 1.6 | 0.8 | 1.2 | 0.3 | 1.2 | 0.6 | 0.9 | 0.7 | 0.6 | 2.7 | 0.9 | 1.4 | 1.4 | 4.82 |
| YIL162W | 1.3 | 1.5 | 6.8 | 2.4 | 1.2 | 1.0 | 1.2 | 1.6 | 0.8 | 3.7 | 2.8 | 2.0 | 1.0 | 1.0 | 1.5 | 1.7 | 0.9 | 1.4 | 1.22 |
| YJR105W | 0.6 | 0.7 | 3.0 | 0.8 | 0.7 | 0.3 | 0.5 | 0.8 | 0.9 | 0.5 | 1.0 | 0.8 | 0.8 | 0.5 | 1.2 | 0.7 | 1.1 | 0.9 | 3.75 |
| YLR134W | 0.8 | 0.6 | 2.3 | 0.8 | 1.3 | 2.5 | 1.1 | 0.8 | 0.1 | 0.7 | 1.1 | 1.2 | 1.5 | 0.6 | 1.7 | 0.5 | 0.6 | 0.8 | 3.47 |
| YLR258W | 1.5 | 1.0 | 4.2 | 3.5 | 0.9 | 1.8 | 1.8 | 0.9 | 0.8 | 1.3 | 1.2 | 0.9 | 1.4 | 1.2 | 0.6 | 1.7 | 1.0 | 2.0 | 1.36 |
| YML058W | 1.9 | 1.2 | 5.8 | 1.9 | 0.6 | 0.6 | 0.6 | 0.7 | 1.1 | 1.2 | 3.3 | 0.9 | 1.4 | 1.1 | 2.5 | 1.3 | 1.3 | 1.5 | 2.14 |
| YMR083W | 1.4 | 1.7 | 2.6 | 1.1 | 1.5 | 1.8 | 1.7 | 1.1 | 0.4 | 1.1 | 1.6 | 0.7 | 0.9 | 0.8 | 1.3 | 1.3 | 0.9 | 1.1 | 2.52 |
| YOR178C | 1.4 | 1.3 | 4.8 | 2.3 | 1.0 | 2.7 | 0.9 | 0.9 | 0.2 | 1.7 | 4.1 | 1.7 | 1.0 | 1.0 | 1.1 | 1.7 | 1.0 | 1.5 | 0.56 |
| YPL028W | 0.7 | 0.9 | 2.6 | 0.9 | 1.0 | 1.2 | 1.3 | 0.9 | 1.2 | 0.8 | 1.2 | 1.4 | 0.9 | 0.8 | 0.6 | 2.2 | 1.4 | 1.8 | 4.35 |
| YPR113W | 1.1 | 0.8 | 3.7 | 1.3 | 2.0 | 1.1 | 1.2 | 0.9 | 0.4 | 0.8 | 2.0 | 0.7 | 0.7 | 1.3 | 1.0 | 2.3 | 1.2 | 1.9 | 2.85 |
| YPR183W | 0.9 | 1.5 | 3.5 | 0.8 | 0.9 | 1.2 | 1.2 | 1.0 | 0.9 | 0.6 | 2.5 | 1.2 | 1.6 | 0.7 | 0.9 | 0.8 | 1.2 | 1.0 | 1.17 |
| YBR011C | 1.3 | 1.8 | 2.5 | 1.1 | 0.9 | 2.0 | 1.1 | 1.3 | 2.2 | 1.0 | 1.7 | 1.1 | 1.5 | 0.9 | 2.1 | 1.7 | 0.8 | 1.1 | 3.68 |
| YCR034W | 0.8 | 0.4 | 2.5 | 0.9 | 0.9 | 0.3 | 0.3 | 0.9 | 0.1 | 0.2 | 0.8 | 0.5 | 0.2 | 0.6 | 0.9 | 0.3 | 0.9 | 0.7 | 3.77 |
| YDR050C | 1.6 | 1.3 | 2.3 | 1.8 | 0.9 | 1.7 | 1.5 | 1.3 | 0.4 | 1.3 | 2.0 | 1.4 | 2.0 | 1.2 | 1.9 | 1.3 | 1.1 | 2.3 | 6.26 |
| YDR178W | 2.0 | 2.0 | 3.4 | 2.2 | 0.9 | 2.1 | 0.6 | 0.9 | 0.9 | 0.8 | 2.0 | 1.3 | 1.5 | 1.5 | 0.7 | 3.0 | 1.2 | 2.3 | 2.27 |
| YDR284C | 0.8 | 1.5 | 3.0 | 2.9 | 0.8 | 1.8 | 1.4 | 0.9 | 0.9 | 1.0 | 1.3 | 0.9 | 1.2 | 1.3 | 0.9 | 1.7 | 1.5 | 1.5 | 1.44 |
| YDR345C | 0.8 | 0.9 | 5.6 | 2.6 | 1.3 | 1.2 | 1.1 | 1.2 | 0.2 | 1.1 | 1.5 | 1.4 | 1.2 | 0.8 | 0.8 | 1.0 | 1.3 | 1.8 | 5.65 |
| YDR400W | 1.0 | 2.0 | 2.2 | 0.7 | 1.0 | 1.1 | 0.6 | 1.4 | 1.1 | 1.4 | 1.3 | 0.4 | 0.7 | 0.9 | 0.8 | 1.1 | 1.1 | 0.8 | 0.56 |
| YEL063C | 0.7 | 0.8 | 2.4 | 1.3 | 1.2 | 1.3 | 0.7 | 0.6 | 0.8 | 1.7 | 0.8 | 1.1 | 1.1 | 0.8 | 1.3 | 1.0 | 0.7 | 0.7 | 1.12 |
| YER081W | 1.6 | 1.9 | 2.7 | 1.4 | 1.1 | 1.1 | 0.5 | 0.6 | 1.8 | 1.1 | 1.1 | 1.0 | 0.6 | 1.6 | 0.5 | 0.8 | 1.1 | 1.7 | 2.31 |
| YER120W | 0.9 | 1.1 | 2.5 | 0.8 | 0.8 | 1.5 | 0.9 | 0.6 | 0.8 | 0.9 | 1.1 | 1.3 | 1.0 | 0.9 | 1.5 | 1.0 | 1.2 | 1.0 | 1.52 |
| YFL011W | 1.2 | 0.7 | 3.7 | 3.3 | 1.3 | 1.0 | 0.8 | 0.8 | 0.3 | 1.6 | 2.1 | 0.9 | 0.8 | 0.8 | 1.7 | 1.2 | 0.8 | 1.0 | 1.25 |
| YGL012W | 1.0 | 0.8 | 3.0 | 1.2 | 1.5 | 1.6 | 0.9 | 0.8 | 0.3 | 0.9 | 0.6 | 0.6 | 0.3 | 0.8 | 0.6 | 1.2 | 0.9 | 1.1 | 4.88 |
| YGR191W | 0.8 | 0.7 | 1.6 | 1.5 | 0.8 | 1.1 | 0.7 | 0.7 | 0.1 | 0.7 | 1.6 | 0.7 | 1.0 | 0.7 | 1.5 | 0.7 | 0.8 | 0.8 | 1.58 |
| YGR204W | 0.5 | 1.1 | 2.7 | 1.3 | 0.6 | 0.6 | 0.8 | 0.4 | 0.9 | 2.3 | 2.1 | 1.0 | 1.3 | 0.5 | 1.6 | 0.6 | 1.0 | 0.7 | 1.49 |
| YHR025W | 0.6 | 0.8 | 2.4 | 0.6 | 0.9 | 0.8 | 0.8 | 0.8 | 0.2 | 0.4 | 2.1 | 0.9 | 0.7 | 0.6 | 2.1 | 0.5 | 1.0 | 0.6 | 0.68 |
| YHR123W | 0.7 | 1.2 | 1.8 | 1.0 | | 1.0 | 0.9 | 0.8 | 0.8 | 1.1 | 1.2 | 0.7 | 1.5 | 0.8 | 2.3 | 0.5 | 1.1 | 1.0 | 0.85 |
| YJL121C | 1.0 | 0.5 | 1.9 | 0.8 | 1.0 | 1.1 | 0.7 | 1.0 | 0.1 | 0.4 | 1.1 | 0.6 | 0.7 | 0.9 | 1.0 | 0.6 | 1.1 | 1.0 | 1.00 |
| YJR077C | 1.1 | 1.1 | 2.0 | 1.6 | 0.9 | 0.9 | 0.7 | 0.8 | 0.4 | 0.7 | 1.6 | 1.0 | 0.9 | 0.7 | 1.5 | 0.7 | 1.0 | 0.8 | 1.79 |
| YJR143C | 0.6 | 0.6 | 2.1 | 0.8 | 1.6 | 0.6 | 0.5 | 0.7 | 0.1 | 0.3 | 1.3 | 0.6 | 0.3 | 0.6 | 1.1 | 0.5 | 0.7 | 0.8 | 3.24 |
| YKL060C | 1.6 | 0.8 | 2.5 | 1.0 | 1.6 | 1.3 | 1.2 | 1.1 | 0.3 | 0.8 | 2.1 | 1.3 | 1.2 | 0.8 | 1.6 | 1.5 | 1.2 | 1.7 | 6.01 |
| YKL148C | 0.9 | 0.8 | 3.7 | 0.7 | 0.8 | 0.5 | 0.6 | 0.8 | 1.7 | 1.7 | 1.5 | 0.8 | 0.8 | 0.8 | 0.6 | 1.1 | 0.8 | 1.0 | 0.54 |
| YKL157W | 0.7 | 1.0 | 2.3 | 1.3 | 1.5 | 1.5 | 1.9 | 1.1 | 1.7 | 1.9 | 1.5 | 1.0 | 2.3 | 0.8 | 1.2 | 1.7 | 1.2 | 1.5 | 1.22 |
| YLR044C | 0.8 | 0.6 | 2.2 | 0.9 | 1.7 | 1.5 | 1.3 | 0.7 | 0.0 | 1.2 | 1.1 | 1.4 | 1.7 | 0.5 | 2.2 | 0.6 | 0.9 | 0.9 | 5.16 |
| YLR056W | 1.0 | 0.6 | 2.5 | 0.8 | 1.6 | 1.2 | 0.8 | 0.9 | 0.0 | 0.5 | 0.8 | 0.9 | 0.2 | 0.7 | 0.9 | 2.1 | 1.1 | 1.1 | 3.61 |
| YLR058C | 0.9 | 0.8 | 5.5 | 2.6 | 0.7 | 0.9 | 0.5 | 0.3 | 0.2 | 0.1 | 2.6 | 0.4 | 0.3 | 0.7 | 1.5 | 0.4 | 0.8 | 0.8 | 2.71 |
| YLR081W | 1.3 | 0.8 | 2.8 | 3.6 | 1.0 | 0.9 | 0.7 | 0.9 | 0.1 | 2.2 | 2.6 | 1.0 | 0.6 | 1.0 | 0.7 | 1.2 | 0.7 | 0.9 | 1.46 |
| YLR089C | 0.9 | 1.2 | 2.1 | 0.9 | 0.8 | 1.5 | 1.6 | 0.7 | 0.5 | 0.8 | 1.7 | 0.6 | 1.0 | 0.7 | 1.7 | 1.4 | 1.2 | 1.3 | 1.27 |
| YLR284C | 0.9 | 1.0 | 3.8 | 1.9 | 1.6 | 0.9 | 0.8 | 1.2 | 0.9 | 0.9 | 1.5 | 0.6 | 0.8 | 1.3 | 1.0 | 4.5 | 2.9 | 8.1 | 0.84 |
| YLR304C | 0.7 | 0.6 | 5.0 | 0.7 | 0.6 | 1.9 | 0.6 | 0.6 | 0.1 | 2.2 | 1.6 | 1.0 | 0.5 | 0.7 | 1.6 | 1.8 | 0.5 | 0.7 | 2.39 |
| YLR354C | 1.1 | 2.5 | 2.3 | 1.5 | 1.6 | 1.5 | 1.1 | 1.4 | 0.4 | 0.9 | 1.7 | 1.2 | 1.0 | 0.9 | 2.4 | 1.3 | 1.0 | 1.5 | 4.53 |
| YLR372W | 0.7 | 0.3 | 1.7 | 0.8 | 1.4 | 0.5 | 0.2 | 0.9 | 0.0 | 0.1 | 0.9 | 0.6 | 0.1 | 0.6 | 0.7 | 0.2 | 0.5 | 0.6 | 4.46 |
| YML022W | 0.9 | 0.6 | 2.0 | 1.1 | 1.3 | 0.8 | 0.6 | 1.1 | 0.3 | 0.6 | 1.2 | 0.6 | 0.2 | 0.9 | 0.6 | 0.6 | 0.5 | 0.7 | 4.93 |
| YMR011W | 1.3 | 1.0 | 9.4 | 5.5 | 0.9 | 0.6 | 0.8 | 1.1 | 0.0 | 0.7 | 1.5 | 0.8 | 0.4 | 0.6 | 0.6 | 1.2 | 1.3 | 1.6 | 4.95 |
| YMR015C | 0.7 | 0.7 | 1.9 | 0.9 | 1.7 | 1.3 | 0.4 | 0.7 | 0.1 | 0.3 | 1.0 | 0.9 | 0.4 | 0.8 | 1.2 | 1.5 | 1.0 | 1.0 | 2.51 |
| YMR205C | 0.5 | 0.7 | 2.3 | 0.8 | 1.2 | 1.2 | 0.9 | 0.7 | 0.3 | 0.8 | 1.0 | 1.1 | 1.3 | 0.5 | 0.9 | 0.9 | 0.6 | 0.5 | 4.75 |
| YMR261C | 0.7 | 1.6 | 3.6 | 0.8 | 0.9 | 0.6 | 1.3 | 0.7 | 1.6 | 1.3 | 0.8 | 0.9 | 1.6 | 0.6 | 0.8 | 1.6 | 0.7 | 1.1 | 0.78 |
| YMR323W | 0.8 | 1.1 | 2.9 | 1.1 | 0.7 | 0.4 | 1.1 | 0.8 | 0.5 | 1.3 | 2.5 | 1.2 | 1.8 | 1.2 | 37.3 | 0.6 | 1.1 | 0.6 | 1.04 |
| YOL086C | 1.1 | 0.5 | 2.2 | 1.1 | 1.9 | 1.7 | 1.9 | 0.8 | 0.1 | 1.2 | 2.6 | 1.3 | 1.7 | 0.6 | 1.6 | 1.4 | 1.1 | 1.3 | 4.19 |
| YOL156W | 1.1 | 0.7 | 2.5 | 1.1 | 1.2 | 1.2 | 1.0 | 1.0 | 2.3 | 2.1 | 1.1 | 0.9 | 1.7 | 0.9 | 1.9 | 0.9 | 0.9 | 0.9 | 0.53 |
| YOR002W | 0.8 | 1.1 | 1.9 | 0.9 | 1.1 | 1.1 | 1.0 | 0.9 | 0.8 | 0.9 | 1.2 | 0.5 | 0.6 | 0.9 | 1.5 | 0.8 | 0.9 | 1.1 | 1.49 |
| YOR085W | 0.7 | 0.8 | 1.9 | 1.0 | 1.0 | 0.6 | 0.6 | 0.7 | 0.5 | 0.6 | 1.1 | 0.8 | 1.0 | 0.7 | 1.8 | 0.8 | 0.9 | 0.9 | 1.86 |
| YOR108W | 0.8 | 0.9 | 2.3 | 0.6 | 1.1 | 1.6 | 1.2 | 0.9 | 0.4 | 1.1 | 1.4 | 0.7 | 1.3 | 0.9 | 0.7 | 1.0 | 1.1 | 1.1 | 1.07 |
| YOR128C | 0.9 | 0.7 | 2.1 | 2.1 | 1.1 | 1.0 | 0.7 | 0.6 | 0.3 | 0.4 | 0.8 | 0.5 | 0.4 | 0.7 | 1.6 | 0.5 | 1.2 | 1.2 | 2.14 |
| YOR142W | 1.0 | 1.2 | 3.4 | 1.0 | 1.4 | 1.6 | 1.0 | 0.8 | 1.1 | 1.5 | 1.5 | 0.8 | 1.4 | 0.8 | 1.1 | 0.8 | 0.9 | 1.0 | 1.31 |
| YOR176W | 0.7 | 2.6 | 2.7 | 0.9 | 1.0 | 0.9 | 0.5 | 1.0 | 1.0 | 0.5 | 1.4 | 1.3 | 1.5 | 0.8 | 0.8 | 1.4 | 1.2 | 2.1 | 1.23 |
| YPL057C | 1.9 | 1.1 | 3.2 | 3.1 | 1.9 | 0.6 | 0.6 | 1.0 | 0.4 | 1.4 | 1.6 | 1.0 | 1.3 | 0.7 | 1.1 | 2.5 | 2.2 | 2.0 | 2.08 |
| YPL135W | 0.9 | 1.2 | 2.5 | 1.2 | 1.6 | 1.3 | 1.2 | 1.0 | 0.5 | 2.8 | 1.3 | 1.1 | 1.2 | 1.1 | 1.5 | 1.2 | 1.5 | 1.7 | 1.50 |
| YCR010C | 1.6 | 1.6 | 1.6 | 4.2 | 1.7 | 1.3 | 1.3 | 0.8 | 0.9 | 4.7 | 1.1 | 0.8 | 1.0 | 1.2 | 1.2 | 2.8 | 1.9 | 1.5 | 0.26 |
| YBR003W | 0.9 | 0.8 | 1.0 | 1.9 | 0.7 | 1.2 | 1.1 | 0.9 | 1.0 | 1.3 | 1.3 | 0.8 | 1.2 | 0.8 | 0.7 | 2.0 | 1.1 | 1.3 | 1.06 |
| YBR020W | 1.0 | 0.5 | 1.9 | 1.9 | 1.0 | 0.8 | 0.7 | 1.0 | −0.4 | 1.1 | 1.0 | 0.8 | 1.1 | 1.0 | 1.3 | 0.7 | 0.8 | 0.9 | 0.27 |
| YDR123C | 1.2 | 0.8 | 0.4 | 2.1 | 1.6 | 0.7 | 0.9 | 1.3 | 0.5 | 0.6 | 0.6 | 0.5 | 0.9 | 0.7 | 1.2 | 0.8 | 1.3 | 1.0 | 0.30 |
| YDR277C | 1.6 | 1.2 | 1.0 | 3.0 | 0.8 | 1.1 | 1.2 | 1.7 | 0.2 | 0.9 | 1.2 | 0.9 | 1.2 | 1.3 | 0.4 | 2.3 | 1.2 | 1.7 | 0.89 |
| YDR408C | 1.0 | 1.0 | 1.4 | 4.1 | 0.9 | 0.9 | 0.8 | 0.7 | 0.3 | 0.5 | 1.7 | 0.6 | 0.5 | 0.9 | 1.0 | 1.0 | 1.1 | 1.1 | 2.16 |
| YDR483W | 1.1 | 1.0 | 1.3 | 2.0 | 0.7 | 1.5 | 0.8 | 1.0 | 0.4 | 1.1 | 1.4 | 0.8 | 1.3 | 0.8 | 1.5 | 1.2 | 1.2 | 1.4 | 3.24 |
| YGL115W | 0.9 | 0.6 | 1.2 | 2.0 | 1.2 | 1.2 | 1.4 | 1.0 | 1.2 | 1.2 | 1.0 | 0.9 | 0.7 | 1.2 | 0.7 | 1.7 | 0.8 | 1.2 | 2.46 |
| YGR096W | 1.5 | 1.0 | 1.1 | 4.6 | 1.2 | 1.7 | 0.9 | 1.0 | 0.8 | 0.8 | 0.8 | 0.8 | 1.1 | 1.2 | 0.9 | 0.8 | 0.8 | 1.0 | 0.49 |
| YGR288W | 0.9 | 0.5 | 1.0 | 2.6 | 0.8 | | 1.4 | 1.0 | 1.6 | 2.6 | 1.3 | 0.9 | 1.3 | 1.1 | 0.8 | 1.7 | 1.2 | 1.3 | 0.41 |
| YHR210C | 1.3 | 1.0 | 0.6 | 5.2 | 1.8 | 0.7 | 1.9 | 1.3 | −0.1 | 0.9 | 1.5 | 0.5 | 0.4 | 1.0 | 0.5 | 1.5 | 1.4 | 1.2 | 0.33 |
| YIL006W | 1.0 | 1.1 | 1.4 | 2.9 | 0.8 | 0.3 | 0.8 | 0.6 | 0.9 | 1.1 | 1.0 | 0.9 | 0.8 | 1.0 | 1.5 | 1.7 | 0.8 | 0.9 | 0.28 |
| YKR034W | 0.9 | 1.5 | 1.1 | 4.7 | 0.9 | 0.9 | 0.7 | 1.5 | 0.5 | 0.6 | 0.0 | 0.7 | 1.2 | 1.1 | 1.7 | 0.6 | 0.8 | 1.0 | 0.26 |
| YLR006C | 0.7 | 0.7 | 0.9 | 6.2 | 1.6 | 0.9 | 1.1 | 0.9 | 1.1 | 1.1 | 1.5 | 0.7 | 0.8 | 1.0 | 0.9 | 0.8 | 1.6 | 1.0 | 0.40 |
| YNL025C | 0.9 | 1.4 | 1.4 | 12.8 | 0.7 | 0.5 | 1.2 | 1.0 | 1.1 | 1.8 | 1.1 | 0.8 | 1.3 | 1.2 | 1.4 | 2.9 | 0.7 | 1.3 | 0.37 |
| YOL116W | 1.1 | 1.0 | 0.9 | 2.8 | 1.7 | 0.7 | 1.5 | 1.3 | 0.6 | 1.8 | 1.0 | 0.6 | 0.8 | 0.9 | 0.7 | 1.9 | 1.1 | 1.4 | 0.47 |
| YOR103C | 1.1 | 0.8 | 1.3 | 1.8 | 1.5 | 1.0 | 1.2 | 1.0 | 0.8 | 0.9 | 1.0 | 1.2 | 0.8 | 1.2 | 1.4 | 1.3 | 1.1 | 1.5 | 2.84 |
| YOR251C | 1.1 | 1.0 | 1.3 | 1.8 | 0.8 | 0.9 | 1.0 | 1.1 | 0.8 | 0.8 | 1.0 | 0.9 | 1.0 | 1.1 | 0.6 | 1.2 | 0.9 | 1.3 | 1.34 |
| YOR348C | 1.1 | 0.7 | 1.2 | 2.0 | 0.9 | 0.7 | 0.8 | 0.8 | 0.5 | 0.4 | 1.1 | 0.7 | 1.1 | 1.2 | 1.6 | 1.7 | 0.9 | 0.9 | 0.18 |
| YPL148C | 1.2 | 0.8 | 0.9 | 2.3 | 1.1 | 0.6 | 1.0 | 0.9 | 0.7 | 1.5 | 3.5 | 0.7 | 0.6 | 1.6 | 0.6 | 1.6 | 1.0 | 1.1 | 0.48 |
| YGL205W | 0.9 | 0.9 | 0.7 | 0.8 | 1.1 | 1.6 | 0.8 | 1.2 | 0.5 | 0.6 | 0.4 | 1.1 | 1.0 | 1.0 | 2.0 | 4.1 | 3.9 | 9.1 | 0.24 |
| YNL192W | 1.1 | 1.2 | 1.5 | 1.6 | 0.9 | 0.6 | 1.0 | 0.8 | 0.6 | 1.8 | 1.1 | 1.1 | 2.7 | 0.7 | 2.1 | 0.8 | 2.3 | 2.8 | 1.31 |
| YOL108C | 1.4 | 1.5 | 0.4 | 1.9 | 2.1 | 0.9 | 1.1 | 1.3 | 1.6 | 0.9 | 1.1 | 1.0 | 1.3 | 0.9 | 1.5 | 1.8 | 1.5 | 2.7 | 0.87 |
| YPR165W | 1.3 | 0.7 | 1.6 | 1.0 | 1.5 | 1.8 | 1.0 | 1.0 | 0.9 | 0.7 | 1.0 | 0.8 | 1.0 | 1.0 | 0.9 | 1.0 | 2.3 | 2.7 | 3.64 |
| YDR073W | 1.7 | 0.9 | 0.7 | 1.4 | 1.2 | 1.3 | 1.2 | 1.3 | 0.5 | 1.2 | 1.0 | 0.8 | 0.6 | 1.2 | 0.8 | 1.6 | 1.8 | 2.3 | 1.27 |
| YER015W | 0.7 | 0.9 | 1.1 | 1.5 | 1.3 | 0.9 | 1.2 | 1.1 | 0.4 | 0.9 | 1.0 | 0.9 | 0.8 | 1.1 | 0.8 | 3.0 | 1.6 | 2.6 | 0.41 |
| YJL167W | 0.9 | 1.4 | 1.4 | 1.1 | 0.7 | 1.3 | 1.5 | 1.5 | 0.9 | 0.9 | 1.2 | 1.3 | 0.9 | 0.9 | 1.6 | 1.4 | 1.4 | 2.1 | 2.94 |
| YJL216C | 0.8 | 4.7 | 2.2 | 0.9 | 0.8 | | 1.5 | 1.9 | 1.5 | 1.3 | 1.1 | 1.0 | 1.1 | 1.1 | 1.2 | 1.5 | 1.2 | 2.0 | 0.25 |
| YKR009C | 1.0 | 1.2 | 1.5 | 0.9 | 1.0 | 1.8 | 1.0 | 0.9 | 2.4 | 2.1 | 1.2 | 1.0 | 1.8 | 1.0 | 1.0 | 4.6 | 1.9 | 2.5 | 0.27 |
| YOR180C | 0.8 | 1.1 | 1.0 | 1.0 | 1.0 | 0.7 | 0.8 | 0.9 | 0.3 | 0.4 | 0.6 | 0.8 | 0.9 | 1.0 | 1.1 | 2.4 | 0.9 | 2.3 | 0.55 |
| YBR036C | 0.8 | 1.7 | 1.8 | 1.2 | 1.2 | 1.3 | 1.3 | 0.7 | 1.0 | 1.0 | 1.5 | 1.0 | 1.9 | 0.8 | 1.5 | 1.6 | 2.1 | 1.8 | 2.05 |
| YDR297W | 1.5 | 0.8 | 0.8 | 1.1 | 0.9 | 1.4 | 0.6 | 1.5 | 0.3 | 0.7 | 1.4 | 1.0 | 0.8 | 0.9 | 1.4 | 1.4 | 1.9 | 1.9 | 1.73 |
| YDR387C | 0.8 | 0.9 | 1.2 | 2.2 | 0.9 | 1.2 | 1.3 | 0.7 | 1.4 | 2.0 | 2.0 | 0.9 | 1.3 | 0.9 | 1.4 | 2.7 | 1.2 | 1.1 | 0.86 |
| YOL096C | 1.1 | 1.0 | 1.2 | 1.6 | 1.2 | 1.3 | 1.7 | 1.0 | 1.3 | 1.4 | 1.2 | 1.2 | 1.4 | 1.4 | 1.0 | 2.3 | 1.2 | 1.2 | 0.64 |
| YPR184W | 1.2 | 1.4 | 5.7 | 2.2 | 0.9 | 1.5 | 1.3 | 0.7 | 1.9 | 3.2 | 3.6 | 1.4 | 2.5 | 1.3 | 1.5 | 3.1 | 1.0 | 1.7 | 0.37 |
| YBR298C | 1.1 | 1.0 | 1.6 | 2.3 | 1.4 | 0.4 | 1.7 | 0.5 | 0.3 | 1.1 | 1.5 | 1.1 | 0.8 | 0.9 | 0.2 | 2.1 | 0.9 | 0.8 | 0.88 |
| YDL078C | 0.7 | 1.1 | 1.2 | 0.8 | 1.3 | 1.1 | 1.7 | 1.0 | 1.1 | 1.0 | 1.7 | 0.8 | 1.0 | 0.8 | 0.5 | 2.2 | 1.1 | 2.0 | 1.65 |
| YDL215C | 1.0 | 1.5 | 1.0 | 1.6 | 1.5 | 0.9 | 1.9 | 1.1 | 1.7 | 2.4 | 0.8 | 0.8 | 1.5 | 1.0 | 1.1 | 2.2 | 1.1 | 1.6 | 0.91 |
| YGL035C | 0.7 | 1.4 | 4.3 | 1.4 | 1.1 | 0.6 | 1.2 | 1.3 | 0.9 | 1.5 | 1.3 | 0.9 | 1.1 | 1.1 | 1.0 | 2.3 | 0.9 | 1.2 | 0.61 |
| YGR287C | 1.3 | 1.0 | 1.3 | 1.5 | 0.8 | 0.8 | 2.2 | 1.3 | 2.6 | 4.2 | 1.5 | 1.1 | 1.7 | 1.1 | 2.3 | 3.0 | 1.0 | 1.1 | 0.38 |
| YJL070C | 1.3 | 0.9 | 1.1 | 1.2 | 1.3 | 2.0 | 1.4 | 1.1 | 1.0 | 1.1 | 1.3 | 1.0 | 1.8 | 1.3 | 3.5 | 2.2 | 1.1 | 1.2 | 0.34 |
| YLR351C | 1.0 | 1.4 | 1.3 | 1.5 | 1.3 | 1.4 | 1.4 | 1.2 | 0.8 | 1.2 | 1.1 | 0.9 | 0.9 | 1.4 | 1.2 | 2.0 | 1.3 | 1.8 | 1.75 |
| YLR375W | 0.9 | 1.5 | 0.9 | 0.9 | 1.0 | 1.3 | 0.9 | 1.0 | 1.6 | 1.2 | 1.6 | 0.9 | 1.6 | 0.9 | 1.3 | 2.2 | 1.0 | 1.0 | 1.05 |
| YMR267W | 0.8 | 1.8 | 0.7 | 0.9 | 1.0 | 1.4 | 1.2 | 1.5 | 0.4 | 0.8 | 1.0 | 0.8 | 0.7 | 1.0 | 0.9 | 2.6 | 0.9 | 1.3 | 0.94 |
| YMR278W | 0.7 | 1.7 | 1.4 | 1.0 | 1.2 | 1.3 | 1.8 | 1.1 | 1.4 | 1.7 | 1.1 | 1.0 | 2.5 | 0.8 | 1.8 | 2.5 | 1.0 | 1.5 | 0.62 |
| YMR293C | 0.9 | 1.5 | 1.6 | 2.2 | 0.6 | 0.5 | 1.2 | 1.0 | 0.7 | 1.3 | 0.4 | 0.9 | 0.8 | 1.3 | 0.7 | 2.2 | 0.8 | 1.0 | 0.37 |
| YNR072W | 1.1 | 1.5 | 0.5 | 0.7 | 1.0 | 0.9 | 1.5 | 0.9 | 0.7 | 1.8 | 1.4 | 1.0 | 1.5 | 1.0 | 1.8 | 2.1 | 1.3 | 1.0 | 0.26 |
| YOR363C | 0.8 | 2.0 | 0.9 | 1.3 | 0.7 | 1.2 | 0.7 | 1.1 | 1.2 | 1.6 | 1.2 | 0.9 | 1.3 | 0.9 | 1.1 | 2.1 | 1.3 | 1.8 | 0.40 |

TABLE 8
Detoxification protein genes

| | The level of expressed mRNA in the presence of chemical substance/ | | | | | | | | | | | | | | | | | | |
|---|---|---|---|---|---|---|---|---|---|---|---|---|---|---|---|---|---|---|---|
| | The level of expressed mRNA in the absence of chemical substance | | | | | | | | | | | | | | | | | | |
| yeast genes | (1) | (2) | (3) | (4) | (5) | (6) | (7) | (8) | (9) | (10) | (11) | (12) | (13) | (14) | (15) | (16) | (17) | (18) | Intensity |
| YDR453C | 1.1 | 2.7 | 4.4 | 3.0 | 0.7 | 1.7 | 1.0 | 1.3 | 4.5 | 6.1 | 2.3 | 0.9 | 1.4 | 3.8 | 2.3 | 5.6 | 1.0 | 1.5 | 1.53 |
| YLL060C | 12.5 | 4.2 | 2.3 | 2.6 | 0.9 | 1.4 | 2.0 | 5.8 | 13.0 | 23.2 | 14.1 | 1.1 | 1.8 | 10.6 | 1.5 | 3.6 | 1.9 | 2.3 | 0.57 |
| YBL064C | 2.6 | 2.8 | 3.9 | 1.7 | 0.8 | 5.2 | 2.5 | 2.4 | 4.5 | 3.8 | 2.9 | 1.7 | 4.1 | 5.0 | 1.3 | 5.0 | 1.2 | 2.3 | 1.67 |
| YBR008C | 3.0 | 2.4 | 4.9 | 0.7 | 0.9 | 1.0 | 0.9 | 2.8 | 54.6 | 21.1 | 9.4 | 0.8 | 2.0 | 4.1 | 0.8 | 3.1 | 2.0 | 1.2 | 0.37 |
| YOR153W | 1.6 | 0.9 | 5.1 | 1.1 | 1.0 | 7.4 | 2.6 | 0.5 | 3.2 | 1.2 | 1.0 | 4.0 | 11.8 | 0.3 | 3.6 | 1.6 | 2.5 | 3.1 | 1.91 |
| YHL047C | 0.6 | 4.4 | 1.0 | 1.0 | 1.2 | 8.4 | 16.8 | 1.5 | 1.2 | 11.9 | 1.0 | 2.6 | 3.8 | 0.5 | 1.1 | 2.3 | 1.2 | 1.2 | 0.74 |
| YCL035C | 2.0 | 2.3 | 1.5 | 1.7 | 1.5 | 5.1 | 2.7 | 1.4 | 1.9 | 2.3 | 2.1 | 1.5 | 2.8 | 2.5 | 1.9 | 5.5 | 2.6 | 4.2 | 1.74 |
| YGR197C | 1.1 | 2.4 | 2.5 | 1.0 | 0.7 | 0.5 | 1.3 | 0.8 | 16.7 | 3.2 | 1.1 | 1.2 | 2.4 | 1.5 | 1.0 | 1.5 | 1.7 | 1.9 | 0.37 |
| YHR055C | 4.4 | 1.8 | 0.8 | 1.2 | 2.7 | 1.6 | 1.1 | 2.1 | 3.9 | 3.3 | 1.8 | 1.2 | 3.0 | 1.5 | 0.5 | 0.8 | 0.9 | 0.6 | 3.96 |
| YNL239W | 1.0 | 2.4 | 2.4 | 1.2 | 1.2 | 1.8 | 2.1 | 1.3 | 2.5 | 6.0 | 1.9 | 1.5 | 3.6 | 1.3 | 1.7 | 1.5 | 1.1 | 1.4 | 0.68 |
| YNL241C | 1.3 | 2.5 | 4.3 | 1.0 | 0.8 | 0.9 | 3.2 | 0.9 | 3.4 | 7.4 | 3.0 | 2.0 | 4.9 | 1.1 | 7.0 | 2.8 | 1.0 | 1.0 | 0.68 |
| YBR293W | 1.9 | 3.1 | 1.0 | 1.8 | 0.8 | 0.9 | 0.9 | 0.9 | 5.5 | 2.4 | 1.1 | 1.0 | 3.0 | 1.4 | 3.1 | 1.6 | 1.0 | 0.9 | 0.94 |
| YDL100C | 1.0 | 1.2 | 1.1 | 0.8 | 1.0 | 2.1 | 1.6 | 1.7 | 5.4 | 3.2 | 2.8 | 1.5 | 2.5 | 1.4 | 1.0 | 1.3 | 1.2 | 1.4 | 2.60 |
| YER185W | 2.0 | 3.1 | 2.1 | 7.0 | 1.6 | 1.2 | 1.1 | 1.0 | 2.9 | 1.2 | 1.6 | 1.1 | 2.1 | 1.6 | 1.7 | 0.6 | 2.6 | 1.1 | 0.26 |
| YGL013C | 0.9 | 1.2 | 0.8 | 0.7 | 1.1 | 2.3 | 0.9 | 1.4 | 3.0 | 1.5 | 0.9 | 1.0 | 2.3 | 0.8 | 1.0 | 0.9 | 1.1 | 1.0 | 0.43 |
| YHR053C | 3.5 | 2.1 | 0.7 | 1.1 | 2.9 | 1.3 | 1.2 | 2.2 | 4.5 | 2.9 | 1.4 | 1.1 | 1.9 | 1.5 | 0.3 | 0.7 | 1.0 | 0.5 | 3.99 |
| YIR038C | 1.3 | 3.5 | 4.6 | 2.0 | 0.7 | 2.5 | 2.0 | 1.3 | 4.6 | 4.5 | 3.4 | 1.1 | 2.5 | 2.5 | 1.2 | 6.0 | 2.8 | 2.0 | 1.11 |
| YKL026C | 2.7 | 2.0 | 1.2 | 4.9 | 1.0 | 1.8 | 2.5 | 1.8 | 7.5 | 3.0 | 3.0 | 1.0 | 2.3 | 1.8 | 0.8 | 6.6 | 1.6 | 3.3 | 0.61 |
| YLL028W | 0.6 | 0.9 | 4.6 | 0.5 | 0.9 | 1.7 | 1.0 | 0.7 | 1.2 | 2.3 | 0.7 | 1.3 | 4.1 | 0.7 | 3.1 | 0.6 | 1.2 | 1.2 | 1.02 |
| YOR273C | 0.7 | 1.0 | 1.7 | 1.0 | 1.1 | 0.4 | 0.4 | 0.6 | 0.6 | 0.5 | 1.5 | 0.8 | 2.6 | 0.5 | 0.8 | 0.6 | 3.2 | 3.1 | 1.20 |
| YGR138C | 1.1 | 1.6 | 1.0 | 1.2 | 0.8 | 0.8 | 0.7 | 1.0 | 0.5 | 1.0 | 0.7 | 0.6 | 0.6 | 0.9 | 4.5 | 0.7 | 0.7 | 0.7 | 1.24 |
| YOR247W | 1.7 | 0.6 | 3.6 | 1.4 | 0.6 | 0.2 | 0.5 | 0.7 | 0.1 | 0.4 | 2.9 | 0.6 | 0.3 | 0.4 | 7.3 | 0.2 | 1.2 | 0.9 | 2.23 |
| YPL163C | 0.6 | 0.7 | 1.4 | 0.9 | 0.6 | 0.6 | 0.4 | 0.5 | 0.1 | 0.4 | 0.5 | 0.9 | 0.4 | 0.6 | 5.3 | 0.6 | 1.4 | 1.4 | 1.05 |
| YHL040C | 1.6 | 4.4 | 1.5 | 0.4 | 1.1 | 7.3 | 4.0 | 1.5 | 1.1 | 11.3 | 1.7 | 2.0 | 2.0 | 1.1 | 3.4 | 1.0 | 1.4 | 1.0 | 0.69 |
| YEL065W | 0.3 | 3.8 | 1.2 | 0.4 | 1.4 | 2.3 | 4.3 | 0.9 | 0.1 | 4.9 | 0.6 | 1.7 | 0.8 | 0.4 | 2.2 | 2.6 | 0.7 | 0.6 | 2.10 |
| YIR002C | 0.8 | 1.1 | 0.5 | 0.6 | 1.2 | 2.2 | 1.3 | 1.2 | 1.7 | 1.4 | 0.8 | 1.3 | 1.2 | 1.1 | 1.1 | 1.4 | 1.1 | 1.2 | 0.58 |
| YNL259C | 1.6 | 4.5 | 1.1 | 1.2 | 1.1 | 3.9 | 3.8 | 1.5 | 1.6 | 2.1 | 1.6 | 0.7 | 1.1 | 1.4 | 0.7 | 1.7 | 2.6 | 2.4 | 1.22 |
| YBR145W | 1.5 | 0.7 | 2.8 | 0.9 | 1.1 | 11.5 | 58.8 | 1.0 | 0.1 | 1.1 | 1.1 | 1.0 | 2.0 | 2.2 | 1.2 | 3.6 | 1.7 | 2.0 | 2.17 |
| YLR043C | 1.4 | 1.4 | 2.0 | 1.8 | 2.0 | 1.4 | 2.6 | 1.1 | 2.8 | 2.5 | 1.4 | 0.8 | 2.0 | 2.1 | 0.5 | 1.8 | 1.5 | 2.2 | 2.12 |
| YGR209C | 1.6 | 3.1 | 3.5 | 1.5 | 1.9 | 1.9 | 1.7 | 1.6 | 2.4 | 6.3 | 3.1 | 0.9 | 1.4 | 5.0 | 1.3 | 1.9 | 1.3 | 2.4 | 3.17 |
| YDL168W | 2.3 | 2.0 | 2.1 | 0.9 | 1.2 | 1.4 | 1.1 | 1.7 | 8.2 | 4.7 | 1.9 | 0.6 | 1.3 | 1.6 | 1.2 | 1.1 | 0.9 | 0.8 | 1.08 |
| YDR513W | 2.2 | 2.5 | 2.3 | 2.6 | 0.9 | 2.1 | 1.6 | 1.6 | 4.6 | 3.1 | 2.0 | 0.9 | 1.8 | 2.0 | 1.3 | 3.8 | 1.3 | 3.2 | 3.10 |
| YGR088W | 1.3 | 1.2 | 7.7 | 2.4 | 1.0 | 1.3 | 0.8 | 0.8 | 1.5 | 3.2 | 3.9 | 1.1 | 1.2 | 1.3 | 0.7 | 5.6 | 0.9 | 2.0 | 0.75 |
| YHR048W | 2.5 | 1.4 | 1.4 | 1.7 | 1.0 | 0.8 | 0.8 | 1.9 | 4.5 | 2.7 | 2.0 | 0.9 | 1.1 | 1.7 | 0.9 | 1.4 | 0.7 | 0.9 | 0.26 |
| YJL101C | 2.1 | 1.9 | 2.1 | 1.2 | 1.4 | 1.0 | 1.0 | 1.3 | 3.1 | 3.6 | 2.7 | 0.7 | 1.0 | 1.0 | 0.5 | 1.3 | 0.9 | 1.0 | 1.13 |
| YML116W | 4.1 | 1.3 | 1.5 | 1.4 | 1.2 | 0.9 | 1.4 | 2.2 | 1.4 | 3.1 | 4.3 | 0.5 | 0.8 | 2.0 | 1.9 | 1.0 | 1.0 | 1.0 | 0.94 |
| YMR038C | 1.9 | 1.7 | 1.8 | 0.8 | 1.5 | 1.0 | 1.1 | 1.9 | 2.4 | 2.6 | 2.7 | 0.9 | 1.7 | 1.1 | 0.6 | 1.5 | 1.2 | 1.3 | 1.76 |
| YBR244W | 0.6 | 1.4 | 2.0 | 0.9 | 1.7 | 1.0 | 0.6 | 1.0 | 1.4 | 3.0 | 1.5 | 0.5 | 0.4 | 3.7 | 0.4 | 0.7 | 1.2 | 1.2 | 3.42 |
| YCL069W | 0.9 | 15.7 | 0.9 | 0.8 | 0.9 | | 1.4 | 1.3 | 1.2 | 6.9 | 1.0 | 0.9 | 0.8 | 1.3 | 1.4 | 0.9 | 1.0 | 1.0 | 0.25 |
| YKR105C | 0.8 | 0.9 | 0.9 | 1.5 | 1.0 | 1.2 | 1.0 | 1.3 | 1.0 | 5.2 | 0.0 | 0.8 | 2.5 | 1.2 | 1.5 | 0.7 | 0.8 | 1.0 | 0.26 |
| YOL158C | 0.7 | 4.0 | 2.4 | 0.9 | 1.2 | 2.0 | 1.6 | 0.7 | 1.7 | 6.1 | 0.7 | 0.9 | 1.4 | 1.0 | 0.9 | 1.2 | 1.4 | 1.7 | 1.30 |
| YDR256C | 0.8 | 1.4 | 0.7 | 1.3 | 1.2 | 0.5 | 0.6 | 0.8 | 5.2 | 2.0 | 1.4 | 0.8 | 1.1 | 1.6 | 1.0 | 4.3 | 1.0 | 2.0 | 0.30 |
| YGL254W | 1.2 | 1.1 | 0.8 | 0.6 | 1.1 | 0.7 | 1.3 | 1.4 | 3.1 | 1.8 | 1.1 | 1.2 | 2.4 | 1.3 | 1.1 | 1.4 | 0.8 | 1.2 | 0.53 |
| YKL064W | 0.6 | 0.9 | 0.4 | 0.8 | 1.2 | 1.5 | 1.1 | 0.9 | 2.4 | 1.6 | 0.7 | 0.7 | 1.6 | 1.1 | 0.7 | 1.4 | 1.3 | 1.3 | 0.60 |
| YLL057C | 2.2 | 59.9 | 1.8 | 0.9 | 1.1 | 1.0 | 1.6 | 1.4 | 121.8 | 2.9 | 1.3 | 0.7 | 1.4 | 1.1 | 1.4 | 0.9 | 3.0 | 1.3 | 0.19 |
| YPR200C | 47.7 | 1.7 | 0.8 | 5.0 | 1.6 | 0.8 | 1.3 | 1.4 | 4.7 | 3.8 | 2.0 | 0.4 | 0.6 | 2.0 | 0.7 | 1.2 | 1.1 | 1.0 | 0.27 |
| YJR104C | 1.4 | 1.8 | 2.9 | 1.2 | 1.5 | 1.4 | 1.1 | 1.5 | 1.9 | 2.8 | 2.6 | 0.9 | 0.9 | 2.0 | 1.3 | 2.9 | 0.9 | 1.1 | 3.50 |
| YKR106W | 1.3 | 0.8 | 2.1 | 1.4 | 1.2 | 1.6 | 0.9 | 1.3 | 10.5 | 7.4 | 1.8 | 0.8 | 1.4 | 1.7 | | 2.8 | 0.9 | 0.8 | 0.16 |
| YOR031W | 2.6 | 1.7 | 1.3 | 3.4 | 1.4 | 0.9 | 1.3 | 0.9 | 0.7 | 1.3 | 1.2 | 0.8 | 1.8 | 1.5 | 1.4 | 6.2 | 1.7 | 2.4 | 0.52 |
| YHR008C | 1.3 | 6.9 | 4.8 | 1.8 | 0.6 | 1.0 | 0.7 | 0.9 | 1.7 | 2.4 | 2.1 | 0.7 | 1.2 | 1.6 | 1.7 | 2.2 | 0.9 | 1.0 | 1.04 |
| YML028W | 1.0 | 2.2 | 4.4 | 1.3 | 1.6 | 1.2 | 0.8 | 0.8 | 0.8 | 1.8 | 1.0 | 1.6 | 1.6 | 2.0 | 2.2 | 1.3 | 1.1 | 1.5 | 4.85 |
| YEL027W | 1.2 | 0.6 | 3.4 | 1.1 | 1.0 | 1.1 | 0.8 | 0.9 | 0.9 | 1.1 | 0.9 | 0.9 | 0.7 | 1.4 | 1.5 | 1.6 | 1.0 | 1.4 | 4.75 |
| YKR066C | 0.9 | 1.4 | 5.5 | 0.7 | 1.0 | 0.9 | 0.5 | 0.7 | 0.7 | 2.4 | 1.3 | 0.9 | 0.6 | 1.5 | 0.6 | 1.3 | 0.9 | 1.1 | 1.25 |
| YDR538W | 0.9 | 1.1 | 2.4 | 1.2 | 1.0 | 1.0 | 1.2 | 1.0 | 0.3 | 0.8 | 0.9 | 0.7 | 0.7 | 0.9 | 1.0 | 0.9 | 1.0 | 0.9 | 0.53 |
| YMR015C | 0.7 | 0.7 | 1.9 | 0.9 | 1.7 | 1.3 | 0.4 | 0.7 | 0.1 | 0.3 | 1.0 | 0.9 | 0.4 | 0.8 | 1.2 | 1.5 | 1.0 | 1.0 | 2.51 |
| YOR251C | 1.1 | 1.0 | 1.3 | 1.8 | 0.8 | 0.9 | 1.0 | 1.1 | 0.8 | 0.8 | 1.0 | 0.9 | 1.0 | 1.1 | 0.6 | 1.2 | 0.9 | 1.3 | 1.34 |
| YLR046C | 0.7 | 1.7 | 0.9 | 0.8 | 1.0 | 3.8 | 1.5 | 0.5 | 1.0 | 1.4 | 0.9 | 0.8 | 1.2 | 1.3 | 0.8 | 1.3 | 5.2 | 5.4 | 0.86 |
| YGR224W | 0.9 | 1.2 | 1.2 | 1.2 | 1.4 | 0.8 | 0.4 | 0.6 | 0.9 | 0.9 | 0.8 | 0.6 | 0.8 | 0.9 | 1.1 | 1.0 | 2.8 | 1.3 | 0.27 |
| YFL050C | 0.7 | 1.0 | 1.1 | 2.2 | 1.0 | 0.9 | 0.8 | 0.9 | 0.5 | 1.6 | 1.2 | 0.9 | 1.2 | 1.0 | 1.4 | 2.3 | 0.8 | 1.0 | 0.36 |
| YCR083W | 1.1 | 2.8 | 1.5 | 1.8 | 1.6 | 2.4 | 1.8 | 1.5 | 1.8 | 1.8 | 1.8 | 1.1 | 1.8 | 2.4 | 1.0 | 3.1 | 1.3 | 1.3 | 0.98 |

TABLE 9
Genes belonging to other category

The level of expressed mRNA in the presence of chemical substance/
The level of expressed mRNA in the absence of chemical substance

| yeast genes | (1) | (2) | (3) | (4) | (5) | (6) | (7) | (8) | (9) | (10) | (11) | (12) | (13) | (14) | (15) | (16) | (17) | (18) | Intensity |
|---|---|---|---|---|---|---|---|---|---|---|---|---|---|---|---|---|---|---|---|
| YKR076W | 5.0 | 2.6 | 3.5 | 2.2 | 1.0 | 4.7 | 4.9 | 6.2 | 43.4 | 43.1 | 18.6 | 1.5 | 5.1 | 14.7 | 3.2 | 9.6 | 1.8 | 2.7 | 0.27 |
| YNL335W | 1.2 | 0.7 | 1.5 | 2.3 | 1.5 | 1.6 | 1.3 | 1.7 | 1.6 | 5.3 | 1.1 | 0.8 | 0.9 | 4.6 | 1.7 | 1.0 | 1.2 | 1.1 | 0.22 |
| YBR173C | 1.1 | 2.4 | 3.4 | 1.6 | 0.7 | 1.3 | 1.6 | 1.2 | 6.0 | 5.1 | 2.5 | 1.7 | 3.8 | 3.0 | 15.4 | 2.1 | 0.9 | 1.6 | 1.15 |
| YFL022C | 0.8 | 0.9 | 1.5 | 0.6 | 0.7 | 0.9 | 0.3 | 0.8 | 0.5 | 0.5 | 1.2 | 0.6 | 0.6 | 3.1 | 1.1 | 1.3 | 0.9 | 0.8 | 1.07 |
| YGL158W | 1.1 | 2.8 | 1.9 | 12.9 | 1.0 | 0.4 | 1.3 | 1.3 | −0.4 | 0.9 | 0.2 | 1.3 | 1.0 | 2.3 | 2.3 | 8.2 | 0.8 | 1.1 | 0.18 |
| YHR139C | 3.0 | 4.6 | 6.8 | 9.3 | 1.3 | 1.9 | 1.5 | 1.3 | 107.2 | 46.6 | 3.5 | 1.6 | 7.0 | 2.5 | 1.9 | 17.1 | 1.4 | 1.3 | 0.34 |
| YGR213C | 5.4 | 2.9 | 1.0 | 6.8 | 1.2 | 4.1 | 7.0 | 2.0 | 7.5 | 6.9 | 1.6 | 10.6 | 30.4 | 1.0 | 6.5 | 5.2 | 12.8 | 8.5 | 0.22 |
| YKL165C | 0.5 | 2.0 | 1.1 | 0.6 | 0.9 | 0.6 | 2.1 | 1.5 | 0.4 | 0.4 | 0.3 | 2.4 | 3.2 | 0.8 | 2.1 | 1.2 | 0.9 | 1.0 | 1.08 |
| YBL005W-A | 1.0 | 0.9 | 0.5 | 0.9 | 1.3 | 1.2 | 1.4 | 1.0 | 0.8 | 1.5 | 0.8 | 1.6 | 2.9 | 1.4 | 0.9 | 1.1 | 1.3 | 1.0 | 0.48 |
| YBL041W | 1.2 | 1.4 | 0.9 | 1.0 | 1.6 | 1.7 | 1.6 | 1.7 | 2.8 | 2.6 | 1.6 | 1.6 | 2.6 | 2.0 | 1.3 | 2.3 | 1.3 | 2.0 | 3.03 |
| YBL078C | 2.3 | 2.7 | 1.3 | 2.6 | 0.7 | 2.4 | 3.1 | 1.6 | 12.3 | 15.2 | 4.6 | 3.1 | 5.0 | 3.6 | 2.5 | 4.4 | 1.8 | 2.8 | 0.65 |
| YCL020W | 1.1 | 2.5 | 0.5 | 1.1 | 0.5 | 1.0 | 1.5 | 1.7 | 0.9 | 2.4 | 1.3 | 1.5 | 3.0 | 1.7 | 2.5 | 1.3 | 0.9 | 1.3 | 1.46 |
| YDL007W | 0.8 | 1.4 | 1.3 | 0.9 | 1.0 | 1.1 | 1.2 | 1.4 | 3.8 | 2.4 | 1.3 | 1.1 | 2.4 | 1.4 | 1.4 | 0.8 | 0.8 | 1.5 | 2.43 |
| YDL097C | 0.9 | 1.7 | 0.9 | 1.2 | 0.8 | 1.3 | 1.6 | 1.5 | 4.5 | 2.3 | 1.5 | 1.3 | 4.0 | 1.4 | 1.1 | 1.1 | 1.0 | 1.3 | 2.06 |
| YDL126C | 0.5 | 1.4 | 2.3 | 0.7 | 0.9 | 1.2 | 1.5 | 0.8 | 2.7 | 2.0 | 1.4 | 1.3 | 4.3 | 0.6 | 1.7 | 0.7 | 0.9 | 0.9 | 3.24 |
| YER012W | 1.4 | 1.2 | 1.2 | 1.0 | 1.9 | 2.4 | 2.4 | 1.8 | 6.7 | 2.7 | 1.9 | 1.5 | 2.9 | 4.6 | 1.7 | 2.2 | 1.5 | 1.3 | 1.84 |
| YFR010W | 1.1 | 1.1 | 0.8 | 1.2 | 0.9 | 1.3 | 1.4 | 1.6 | 4.3 | 3.4 | 1.5 | 1.2 | 3.7 | 1.1 | 1.5 | 1.3 | 0.9 | 1.4 | 1.89 |
| YFR024C | 0.8 | 25.0 | 2.3 | 1.1 | 0.7 | 1.4 | 3.0 | 0.9 | 5.3 | 4.1 | 1.9 | 1.5 | 2.8 | 1.2 | 0.9 | 1.9 | 1.0 | 1.4 | 1.06 |
| YGL048C | 1.0 | 1.3 | 0.7 | 1.3 | 1.8 | 1.5 | 2.0 | 1.5 | 2.4 | 2.1 | 1.5 | 1.3 | 2.5 | 1.2 | 1.0 | 1.6 | 1.4 | 2.0 | 3.07 |
| YGL141W | 0.8 | 1.2 | 1.1 | 0.7 | 1.4 | 3.0 | 1.7 | 0.8 | 4.5 | 1.9 | 0.6 | 1.4 | 3.6 | 1.2 | 1.5 | 1.2 | 1.4 | 1.2 | 0.44 |
| YGL180W | 1.3 | 1.3 | 1.7 | 0.6 | 1.2 | 1.1 | 2.4 | 1.2 | 7.2 | 3.7 | 1.3 | 1.4 | 2.6 | 1.0 | 1.3 | 1.7 | 1.0 | 1.1 | 0.28 |
| YGR048W | 0.7 | 2.7 | 1.4 | 1.3 | 1.0 | 1.0 | 1.9 | 1.4 | 4.4 | 3.1 | 1.1 | 1.1 | 2.5 | 1.3 | 0.8 | 1.1 | 0.9 | 1.2 | 0.93 |
| YGR135W | 1.0 | 1.0 | 1.0 | 1.1 | 1.6 | 1.5 | 2.2 | 1.8 | 1.5 | 2.3 | 1.6 | 1.2 | 2.8 | 2.6 | 1.5 | 1.5 | 1.0 | 1.9 | 3.10 |
| YGR201C | 1.7 | 11.9 | 2.4 | 2.0 | 0.8 | 3.1 | 2.6 | 1.6 | 5.4 | 3.5 | 1.6 | 1.9 | 8.6 | 1.6 | 0.5 | 14.4 | 1.4 | 2.1 | 0.60 |
| YHL030W | 0.5 | 0.8 | 1.0 | 0.7 | 0.7 | 1.9 | 0.9 | 0.8 | 2.7 | 2.5 | 1.1 | 1.1 | 3.8 | 1.0 | 2.0 | 0.5 | 0.8 | 0.9 | 0.66 |
| YHR166C | 0.9 | 1.1 | 1.0 | 0.7 | 1.0 | 1.8 | 1.5 | 1.0 | 2.6 | 1.2 | 0.6 | 1.7 | 6.9 | 1.2 | 0.9 | 1.1 | 1.4 | 1.5 | 1.35 |
| YJR069C | 0.8 | 0.8 | 0.9 | 0.6 | 0.9 | 1.2 | 1.3 | 0.9 | 0.7 | 0.6 | 0.9 | 1.1 | 3.9 | 0.6 | 0.4 | 0.8 | 1.0 | 0.9 | 1.73 |
| YKL073W | 0.6 | 1.4 | 0.5 | 0.6 | 1.2 | 0.5 | 2.1 | 1.6 | 1.3 | 1.1 | 0.5 | 1.5 | 3.3 | 0.7 | 1.6 | 1.0 | 1.2 | 1.5 | 1.29 |
| YKL103C | 1.6 | 4.1 | 2.5 | 2.1 | 0.9 | 2.7 | 2.8 | 1.6 | 8.3 | 9.9 | 2.8 | 1.3 | 3.8 | 2.0 | 2.1 | 1.2 | 1.9 | 2.2 | 0.56 |
| YLR080W | 1.0 | 1.4 | 1.2 | 1.7 | 1.1 | 2.5 | 2.8 | 1.3 | 2.3 | 1.5 | 1.9 | 0.8 | 3.6 | 1.2 | 0.7 | 3.0 | 1.6 | 2.2 | 0.41 |
| YLR107W | 1.2 | 0.9 | 0.5 | 1.2 | 1.7 | 2.3 | 1.6 | 1.1 | 1.8 | 1.2 | 0.8 | 1.0 | 2.9 | 1.6 | 1.1 | 1.6 | 1.8 | 2.4 | 0.94 |
| YLR121C | 1.7 | 1.7 | 1.1 | 1.2 | 1.0 | 1.6 | | 1.3 | 2.2 | 2.0 | 2.2 | 1.1 | 4.0 | 1.2 | 10.7 | 1.5 | 2.7 | 1.4 | 0.30 |
| YLR336C | 0.6 | 0.8 | 0.7 | 0.4 | 0.9 | 0.8 | 1.8 | 1.0 | −0.3 | 0.5 | 0.5 | 1.9 | 4.6 | 0.6 | 0.8 | 1.3 | 1.5 | 1.0 | 0.71 |
| YLR370C | 1.1 | 1.0 | 1.1 | 1.4 | 1.4 | 4.1 | 2.1 | 0.8 | 1.5 | 1.9 | 1.0 | 1.2 | 3.1 | 1.9 | 1.5 | 1.6 | 1.6 | 2.0 | 1.41 |
| YLR423C | 1.2 | 1.2 | 0.7 | 1.9 | 1.5 | 1.2 | 3.8 | 2.0 | 1.2 | 1.0 | 1.0 | 1.7 | 3.9 | 1.2 | 1.3 | 1.9 | 1.2 | 1.6 | 0.34 |
| YML092C | 1.1 | 3.1 | 2.4 | 1.1 | 0.9 | 1.9 | 1.2 | 1.2 | 6.4 | 4.0 | 2.5 | 1.2 | 2.3 | 1.6 | 1.4 | 2.2 | 1.3 | 1.5 | 1.72 |
| YML130C | 1.5 | 2.7 | 5.3 | 1.2 | 0.9 | 0.6 | 2.9 | 2.7 | 7.8 | 4.2 | 1.9 | 2.3 | 5.8 | 1.3 | 2.4 | 0.8 | 1.2 | 1.2 | 1.59 |
| YMR214W | 0.8 | 3.8 | 1.0 | 1.4 | 0.8 | 0.9 | 1.0 | 1.1 | 2.1 | 1.0 | 0.7 | 1.0 | 2.4 | 1.1 | 4.8 | 0.7 | 1.1 | 1.0 | 0.88 |
| YMR297W | 0.7 | 2.0 | 3.6 | 1.9 | 0.6 | 1.8 | 1.2 | 0.8 | 1.9 | 1.0 | 2.5 | 1.2 | 4.2 | 0.5 | 2.4 | 1.9 | 1.1 | 1.4 | 4.10 |
| YNL036W | 2.0 | 1.9 | 3.2 | 1.0 | 1.6 | 1.1 | 2.0 | 2.1 | 6.6 | 5.0 | 1.7 | 1.4 | 3.3 | 5.1 | 1.6 | 1.3 | 1.4 | 1.9 | 2.04 |
| YOL005C | 1.4 | 1.4 | 1.1 | 0.8 | 1.6 | 1.1 | 1.1 | 1.0 | 0.9 | 1.0 | 1.6 | 1.2 | 2.9 | 1.2 | 0.9 | 1.0 | 1.2 | 1.2 | 1.71 |
| YOR134W | 2.0 | 3.6 | 1.0 | 9.5 | 0.9 | 1.4 | 1.1 | 1.5 | 1.5 | 8.2 | 1.9 | 2.8 | 4.4 | 1.3 | 1.6 | 7.7 | 2.2 | 4.6 | 0.28 |
| YOR362C | 1.0 | 1.7 | 1.0 | 0.8 | 1.7 | 1.4 | 1.6 | 1.4 | 4.3 | 3.0 | 1.3 | 1.3 | 2.5 | 1.4 | 1.0 | 1.5 | 1.1 | 1.8 | 2.55 |
| YAR009C | 0.8 | 1.3 | 0.6 | 1.0 | 1.4 | 1.6 | 2.7 | 1.4 | 0.9 | 1.4 | 0.9 | 1.3 | 2.0 | 1.0 | 2.1 | 0.6 | 0.8 | 1.2 | 7.11 |
| YBL101C | 1.0 | 1.1 | 1.9 | 0.9 | 1.0 | 0.9 | 0.8 | 0.8 | 3.0 | 1.4 | 0.8 | 1.1 | 3.5 | 0.9 | 2.7 | 1.4 | 1.0 | 1.1 | 0.39 |
| YBR046C | 1.4 | 1.4 | 1.7 | 1.8 | 1.3 | 2.6 | 3.9 | 1.6 | 4.6 | 6.2 | 2.9 | 1.2 | 2.0 | 1.3 | 2.1 | 3.0 | 1.2 | 2.0 | 0.63 |
| YBR139W | 1.2 | 1.4 | 3.1 | 1.4 | 0.8 | 2.4 | 1.9 | 0.7 | 2.3 | 1.6 | 2.3 | 1.2 | 2.7 | 1.1 | 5.6 | 2.3 | 1.2 | 2.0 | 1.21 |
| YBR170C | 0.8 | 1.2 | 0.6 | 1.4 | 1.5 | 1.4 | 1.9 | 1.3 | 6.1 | 4.2 | 1.5 | 1.2 | 2.7 | 1.3 | 1.9 | 1.3 | 1.1 | 1.4 | 0.72 |
| YBR177C | 0.8 | 1.1 | 4.0 | 1.2 | 1.2 | 2.3 | 0.9 | 0.9 | 2.1 | 1.7 | 0.6 | 1.2 | 3.1 | 1.3 | 1.4 | 0.4 | 0.8 | 0.9 | 0.76 |
| YBR212W | 0.7 | 1.2 | 2.6 | 1.2 | 1.0 | 1.4 | 0.9 | 0.6 | 2.7 | 2.3 | 1.1 | 0.9 | 2.2 | 0.7 | 1.0 | 1.5 | 1.1 | 1.0 | 0.95 |
| YBR239C | 1.0 | 1.3 | 0.9 | 1.5 | 0.8 | 1.5 | 1.0 | 1.3 | 0.4 | 1.2 | 0.7 | 1.1 | 1.9 | 1.1 | 1.0 | 2.7 | 1.0 | 0.9 | 0.31 |
| YCL033C | 1.1 | 2.0 | 1.7 | 1.7 | 1.2 | 4.6 | 2.4 | 1.1 | 2.3 | 1.4 | 1.9 | 1.6 | 2.0 | 1.8 | 0.8 | 3.6 | 2.0 | 2.0 | 1.14 |
| YCR062W | 0.8 | 1.9 | 2.7 | 2.8 | 1.4 | 1.9 | 0.9 | 0.7 | 0.7 | 1.3 | 1.2 | 1.3 | 2.2 | 1.0 | 2.3 | 1.1 | 1.1 | 1.0 | 0.41 |
| YCR067C | 0.9 | 0.7 | 1.2 | 1.3 | 0.7 | 0.8 | 0.8 | 0.7 | 1.8 | 1.6 | 1.2 | 0.9 | 2.0 | 0.9 | 2.1 | 1.4 | 1.1 | 0.9 | 0.62 |
| YDL020C | 2.3 | 1.2 | 3.4 | 1.1 | 1.2 | 2.5 | 1.0 | 2.2 | 5.6 | 2.9 | 1.9 | 1.1 | 3.4 | 1.2 | 1.5 | 1.8 | 1.5 | 1.7 | 1.36 |
| YDR168W | 1.2 | 1.4 | 1.0 | 0.8 | 1.5 | 1.3 | 2.4 | 2.1 | 3.7 | 2.3 | 1.5 | 1.2 | 2.4 | 1.4 | 1.9 | 1.4 | 1.2 | 1.4 | 1.28 |
| YDR169C | 1.0 | 1.4 | 1.1 | 1.3 | 1.3 | 1.2 | 1.1 | 1.0 | 1.6 | 1.4 | 0.9 | 0.9 | 1.9 | 0.9 | 1.4 | 0.9 | 1.4 | 1.1 | 0.40 |
| YDR188W | 0.8 | 1.2 | 1.0 | 0.8 | 0.8 | 0.8 | 0.9 | 1.0 | 2.9 | 1.6 | 0.9 | 0.9 | 1.7 | 0.7 | 1.3 | 0.9 | 0.7 | 0.8 | 1.78 |
| YDR264C | 0.5 | 2.6 | 0.9 | 1.4 | 0.9 | 1.8 | 3.2 | 0.8 | 1.1 | 3.4 | 0.9 | 1.1 | 2.1 | 0.7 | 2.3 | 1.5 | 1.2 | 1.4 | 1.63 |
| YDR304C | 1.0 | 1.8 | 2.7 | 1.3 | 1.5 | 2.1 | 2.0 | 1.3 | 3.9 | 1.9 | 1.2 | 1.3 | 2.3 | 1.7 | 1.0 | 4.0 | 1.6 | 1.8 | 2.23 |
| YDR403W | 1.0 | 1.2 | 1.6 | 1.0 | 1.2 | 1.1 | 1.2 | 1.2 | 4.1 | 3.6 | 1.0 | 1.0 | 2.0 | 1.1 | 0.9 | 0.9 | 0.9 | 1.0 | 0.35 |
| YDR427W | 0.8 | 1.1 | 0.8 | 0.8 | 1.5 | 1.9 | 1.4 | 1.2 | 2.3 | 2.0 | 1.2 | 1.1 | 2.1 | 1.0 | 1.3 | 1.1 | 1.0 | 1.5 | 2.37 |
| YEL012W | 1.3 | 1.8 | 1.0 | 1.8 | 1.7 | 1.6 | 4.7 | 1.4 | 4.6 | 4.2 | 2.2 | 1.2 | 2.2 | 1.9 | 1.1 | 2.5 | 1.5 | 2.2 | 0.74 |
| YER009W | 1.3 | 0.6 | 2.4 | 1.2 | 1.2 | 2.0 | 2.8 | 1.0 | 0.7 | 1.0 | 1.4 | 1.1 | 2.0 | 1.6 | 0.9 | 1.5 | 2.0 | 2.1 | 3.35 |
| YER021W | 0.8 | 1.0 | 0.9 | 0.9 | 1.4 | 1.1 | 1.5 | 1.2 | 3.0 | 2.2 | 1.3 | 1.0 | 1.8 | 1.3 | 0.8 | 1.4 | 0.8 | 1.5 | 2.02 |
| YER094C | 1.0 | 1.3 | 0.9 | 1.1 | 1.9 | 1.1 | 1.8 | 1.3 | 2.3 | 1.7 | 1.3 | 1.1 | 2.1 | 1.5 | 0.8 | 1.5 | 1.3 | 1.8 | 3.01 |
| YER177W | 1.4 | 1.5 | 2.1 | 1.5 | 0.9 | 1.6 | 1.2 | 1.1 | 1.8 | 1.7 | 1.9 | 1.1 | 2.2 | 0.9 | 1.4 | 1.0 | 0.9 | 2.0 | 5.81 |
| YFL029C | 1.0 | 1.2 | 1.2 | 1.8 | 1.7 | 1.0 | 1.6 | 1.5 | 4.3 | 2.9 | 2.1 | 1.1 | 1.8 | 1.9 | 1.3 | 1.2 | 1.2 | 1.4 | 0.49 |
| YFL038C | 1.0 | 1.2 | 0.7 | 1.3 | 1.5 | 1.3 | 1.7 | 1.4 | 2.1 | 1.9 | 1.7 | 1.1 | 2.1 | 1.5 | 1.5 | 2.1 | 1.2 | 1.6 | 2.31 |
| YFR004W | 1.0 | 1.5 | 0.8 | 1.4 | 1.0 | 1.5 | 1.3 | 1.4 | 3.6 | 2.6 | 1.3 | 1.2 | 2.0 | 2.1 | 1.1 | 1.3 | 1.0 | 1.4 | 2.44 |
| YFR050C | 0.9 | 1.2 | 1.1 | 1.3 | 1.7 | 1.5 | 1.4 | 1.4 | 3.1 | 2.1 | 1.4 | 1.5 | 2.1 | 1.6 | 1.5 | 1.5 | 1.1 | 1.7 | 1.70 |
| YGL011C | 1.2 | 1.5 | 1.1 | 1.1 | 1.8 | 1.6 | 1.5 | 1.3 | 2.0 | 2.2 | 1.5 | 1.0 | 2.1 | 3.0 | 1.3 | 1.7 | 1.3 | 1.5 | 1.78 |
| YGL094C | 0.8 | 1.3 | 0.3 | 0.9 | 1.3 | 1.0 | 0.8 | 1.0 | 1.4 | 1.1 | 0.6 | 0.8 | 2.3 | 0.9 | 1.9 | 1.0 | 1.0 | 0.9 | 0.51 |
| YGL150C | 0.7 | 0.8 | 1.2 | 1.0 | 0.8 | 1.1 | 0.8 | 0.8 | 1.4 | 0.9 | 0.4 | 0.8 | 2.4 | 1.0 | 1.3 | 0.9 | 0.9 | 0.8 | 0.62 |
| YGL207W | 0.5 | 0.6 | 0.9 | 0.6 | 1.3 | 0.9 | 0.6 | 1.0 | 1.4 | 1.1 | 0.7 | 0.7 | 2.5 | 0.8 | 1.2 | 0.7 | 0.9 | 0.8 | 0.88 |
| YGR232W | 1.2 | 1.2 | 1.2 | 1.7 | 1.5 | 1.9 | 2.0 | 1.1 | 3.4 | 2.2 | 1.3 | 1.1 | 2.4 | 2.1 | 0.9 | 1.8 | 1.3 | 1.2 | 0.96 |
| YGR248W | 1.3 | 1.7 | 3.2 | 4.0 | 0.5 | 2.3 | 1.1 | 1.2 | 2.8 | 3.2 | 2.3 | 1.1 | 2.0 | 1.5 | 1.5 | 7.9 | 1.0 | 1.5 | 0.52 |
| YGR253C | 1.3 | 1.4 | 0.7 | 1.3 | 2.0 | 1.4 | 1.6 | 2.0 | 4.0 | 3.1 | 2.4 | 1.1 | 3.4 | 1.8 | 1.1 | 1.8 | 1.5 | 2.1 | 2.01 |
| YHR027C | 0.5 | 0.8 | 1.2 | 0.6 | 1.1 | 0.4 | 0.9 | 0.6 | 3.6 | 2.2 | 1.1 | 1.0 | 2.1 | 0.7 | 1.5 | 0.9 | 0.8 | 1.0 | 2.06 |
| YHR161C | 0.6 | 1.1 | 1.3 | 2.1 | 1.5 | 1.2 | 1.8 | 0.7 | 2.5 | 2.0 | 1.2 | 1.0 | 1.8 | 1.0 | 1.4 | 1.7 | 1.4 | 1.5 | 0.86 |
| YHR169W | 0.6 | 0.5 | 0.4 | 0.4 | 1.1 | 1.2 | 0.9 | 0.9 | 0.0 | 0.2 | 0.4 | 1.1 | 3.3 | 1.1 | 0.6 | 1.0 | 1.6 | 1.3 | 0.81 |
| YIL010W | 1.0 | 1.2 | 1.4 | 0.8 | 1.2 | 4.4 | 1.5 | 0.9 | 1.4 | 2.2 | 1.4 | 1.4 | 2.3 | 1.7 | 1.1 | 1.7 | 1.5 | 1.5 | 0.70 |
| YIL034C | 1.0 | 0.8 | 1.5 | 3.0 | 0.7 | 1.7 | 1.2 | 0.9 | 2.1 | 1.3 | 1.6 | 1.0 | 2.6 | 1.1 | 1.0 | 1.7 | 1.2 | 1.5 | 0.99 |
| YIL142W | 0.7 | 1.1 | 0.9 | 1.1 | 0.8 | 1.1 | 0.8 | 1.1 | 4.4 | 3.0 | 1.7 | 1.1 | 1.8 | 1.0 | 1.3 | 0.7 | 0.9 | 0.8 | 1.84 |
| YIR039C | 1.4 | 1.8 | 4.3 | 4.2 | 1.5 | 3.2 | 4.1 | 1.1 | 4.4 | 4.1 | 3.8 | 1.4 | 2.9 | 1.9 | 2.0 | 4.2 | 1.2 | 1.1 | 0.45 |
| YJL001W | 0.9 | 1.7 | 1.2 | 2.0 | | 1.7 | 1.6 | 1.3 | 3.1 | 2.8 | 1.5 | 1.6 | 2.9 | 2.5 | 1.2 | 1.8 | 1.0 | 1.6 | 3.00 |
| YJL035C | 0.9 | 1.3 | 0.8 | 0.6 | 1.4 | 0.7 | 1.3 | 1.5 | 1.7 | 1.4 | 0.8 | 1.1 | 2.4 | 1.6 | 0.7 | 0.6 | 1.0 | 1.0 | 0.82 |
| YJL053W | 1.1 | 0.9 | 1.7 | 1.3 | 1.1 | 2.0 | 1.7 | 1.3 | 2.2 | 2.2 | 1.6 | 1.1 | 1.9 | 1.3 | 1.1 | 1.7 | 1.3 | 1.6 | 0.80 |
| YJL164C | 1.1 | 1.1 | 1.0 | 1.9 | 1.1 | 2.7 | 3.1 | 0.9 | 2.1 | 0.8 | 1.5 | 1.0 | 2.1 | 0.9 | 0.8 | 2.1 | 1.3 | 1.7 | 1.04 |
| YJL210W | 1.2 | 1.4 | 1.6 | 2.0 | 0.6 | 0.6 | 1.0 | 1.0 | 2.0 | 0.9 | 2.4 | 1.0 | 2.2 | 0.9 | 0.8 | 2.3 | 0.9 | 1.0 | 0.70 |
| YJR117W | 1.0 | 2.1 | 2.3 | 0.7 | 1.2 | 1.3 | 1.0 | 0.8 | 4.7 | 2.3 | 1.0 | 1.2 | 2.8 | 0.8 | 2.2 | 0.7 | 0.8 | 0.9 | 1.34 |
| YKL007W | 1.2 | 1.6 | 1.2 | 0.9 | 1.0 | 0.9 | 1.5 | 1.2 | 2.3 | 2.4 | 1.8 | 1.3 | 2.5 | 0.7 | 1.0 | 1.0 | 1.4 | 1.3 | 1.13 |
| YKL117W | 1.0 | 1.3 | 0.8 | 1.0 | 1.8 | 1.0 | 1.3 | 1.5 | 2.9 | 2.1 | 1.6 | 1.0 | 2.4 | 1.6 | 1.6 | 1.8 | 1.2 | 1.8 | 3.73 |
| YKL193C | 1.3 | 0.8 | 1.1 | 1.4 | 1.1 | 0.9 | 2.1 | 1.4 | 1.0 | 1.6 | 1.7 | 0.9 | 2.1 | 1.2 | 1.2 | 2.4 | 1.2 | 2.3 | 0.71 |
| YLR120C | 1.2 | 1.9 | 2.4 | 1.6 | 1.1 | 1.9 | 1.5 | 0.9 | 1.8 | 1.6 | 1.2 | 1.6 | 4.8 | 0.8 | 2.7 | 1.7 | 3.5 | 2.9 | 1.53 |
| YLR136C | 1.3 | 9.0 | 0.6 | 3.3 | 1.6 | 1.7 | 3.9 | 1.1 | 0.9 | 6.0 | 1.2 | 1.3 | 2.9 | 0.7 | 2.1 | 1.2 | 1.8 | 1.4 | 0.41 |
| YLR178C | 1.7 | 6.9 | 8.8 | 4.6 | 1.0 | 4.2 | 3.3 | 0.9 | 4.3 | 4.1 | 3.7 | 2.2 | 7.5 | 2.8 | 2.3 | 10.6 | 2.0 | 3.6 | 1.03 |
| YLR327C | 5.5 | 3.3 | 14.4 | 8.2 | 1.3 | 5.1 | 5.7 | 2.2 | 3.0 | 8.3 | 5.0 | 1.4 | 2.8 | 1.5 | 0.8 | 2.8 | 3.3 | 5.3 | 2.23 |
| YLR356W | 1.0 | 2.9 | 4.9 | 1.2 | 1.1 | 3.4 | 1.1 | 0.7 | 2.3 | 1.6 | 3.1 | 1.5 | 2.5 | 2.2 | 2.8 | 0.9 | 1.1 | 1.0 | 0.41 |
| YLR362W | 1.1 | 1.2 | 1.0 | 3.0 | 1.5 | 0.8 | 1.9 | 1.4 | 3.5 | 3.3 | 1.5 | 0.8 | 2.4 | 1.2 | 1.3 | 1.9 | 1.2 | 1.3 | 0.35 |
| YLR429W | 0.7 | 1.0 | 0.6 | 0.9 | 1.4 | 0.8 | 1.3 | 1.0 | 0.9 | 1.3 | 0.9 | 1.1 | 2.3 | 0.7 | 1.5 | 1.0 | 1.0 | 0.9 | 0.86 |
| YMR004W | 0.9 | 0.9 | 1.3 | 1.3 | 1.0 | 0.5 | 1.2 | 1.0 | 94.2 | 3.4 | 2.5 | 1.0 | 2.2 | 2.2 | 1.3 | 1.3 | 0.9 | 1.3 | 0.52 |
| YMR219W | 0.7 | 1.1 | 1.0 | 0.7 | 1.6 | 0.8 | | 1.1 | 2.3 | 0.9 | 1.5 | 1.5 | 2.1 | 0.5 | 1.7 | 0.8 | 1.1 | 0.9 | 0.23 |
| YMR275C | 0.7 | 0.7 | 1.2 | 0.6 | 0.7 | 1.4 | 0.8 | 0.8 | 2.4 | 2.1 | 1.1 | 0.7 | 2.1 | 0.8 | 1.7 | 1.0 | 0.9 | 0.7 | 0.79 |
| YMR314W | | 1.2 | 0.9 | 1.3 | | 1.3 | 1.1 | 1.2 | 5.9 | 2.7 | 1.8 | 1.0 | 2.6 | 1.0 | 1.2 | 1.6 | 1.3 | 1.6 | 1.67 |
| YNL006W | 1.1 | 2.1 | 1.3 | 1.5 | 0.9 | 1.0 | 1.6 | 1.1 | 4.0 | 4.0 | 1.1 | 0.7 | 2.3 | 0.8 | 0.7 | 0.9 | 1.0 | 1.3 | 1.20 |
| YNL007C | 0.7 | 2.4 | 5.1 | 0.8 | 1.2 | 0.6 | 1.2 | 0.8 | 3.0 | 1.7 | 1.3 | 0.7 | 2.1 | 0.4 | 0.8 | 0.5 | 1.1 | 1.0 | 3.62 |
| YNL093W | 2.1 | 6.4 | 0.4 | 4.5 | 1.2 | 1.6 | 1.4 | 0.8 | 1.7 | 2.0 | 1.6 | 1.1 | 2.2 | 1.1 | 0.6 | 4.9 | 3.0 | 3.1 | 0.32 |
| YNL333W | 1.0 | 1.4 | 1.7 | 2.3 | 1.5 | 1.5 | 2.9 | 2.1 | 2.3 | 2.1 | 2.0 | 1.0 | 2.2 | 1.9 | 1.0 | 1.4 | 1.4 | 1.1 | 0.49 |
| YNR010W | 1.3 | 1.2 | 0.5 | 2.8 | 1.8 | 1.3 | 1.5 | 1.1 | 1.7 | 0.9 | 1.4 | 0.8 | 2.8 | 1.3 | 0.8 | 1.2 | 1.7 | 2.3 | 0.37 |
| YNR069C | 1.5 | 1.3 | 0.7 | 0.4 | 1.4 | 1.7 | 1.4 | 1.4 | 5.5 | 5.8 | 2.5 | 1.2 | 3.4 | 2.1 | 1.0 | 0.8 | 1.1 | 0.9 | 0.18 |
| YOL164W | 0.9 | 4.4 | 1.0 | 1.5 | 1.6 | 1.2 | 2.2 | 1.0 | 4.9 | 3.6 | 1.0 | 1.1 | 3.4 | 1.3 | 1.0 | 1.1 | 1.1 | 1.3 | 0.45 |
| YOR036W | 1.8 | 0.9 | 1.4 | 1.6 | 1.1 | 1.7 | 1.8 | 1.4 | 1.2 | 1.9 | 1.3 | 1.2 | 2.1 | 1.2 | 1.2 | 1.3 | 1.6 | 1.7 | 0.98 |
| YOR117W | 0.8 | 1.3 | 1.7 | 0.8 | 1.2 | 1.1 | 3.3 | 1.0 | 3.5 | 1.8 | 1.2 | 2.5 | 2.3 | 1.0 | 1.4 | 1.2 | 1.4 | 0.9 | 1.42 |
| YOR124C | 0.6 | 1.1 | 1.3 | 0.8 | 1.1 | 0.9 | 1.1 | 1.3 | 2.0 | 1.9 | 1.2 | 0.9 | 2.1 | 0.8 | 1.3 | 0.9 | 1.0 | 1.0 | 0.65 |
| YOR132W | 0.9 | 1.6 | 0.8 | 2.8 | 1.1 | 1.6 | 1.4 | 1.2 | 2.8 | 1.6 | 1.3 | 0.7 | 2.5 | 0.7 | 1.5 | 1.6 | 1.2 | 1.6 | 0.46 |
| YOR157C | 1.0 | 1.3 | 1.2 | 1.3 | 0.7 | 1.2 | 1.2 | 1.2 | 5.7 | 2.6 | 1.6 | 1.3 | 2.4 | 2.2 | 1.9 | 0.9 | 1.2 | 1.2 | 1.23 |
| YOR185C | 1.1 | 1.3 | 1.9 | 1.1 | 1.2 | 3.0 | 1.3 | 1.4 | 1.9 | 2.9 | 1.2 | 1.5 | 2.2 | 2.1 | 1.0 | 2.2 | 0.9 | 1.1 | 1.67 |
| YOR259C | 0.8 | 1.1 | 0.8 | 0.9 | 2.0 | 0.8 | 1.3 | 1.1 | 2.7 | 1.5 | 1.0 | 0.8 | 1.8 | 1.2 | 1.3 | 1.4 | 0.9 | 1.5 | 2.33 |
| YOR261C | 1.0 | 1.4 | 0.6 | 0.9 | 1.5 | 1.4 | 1.9 | 2.6 | 5.5 | 2.5 | 2.1 | 0.9 | 2.5 | 1.8 | 1.3 | 2.0 | 1.0 | 1.9 | 2.80 |
| YOR288C | 0.9 | 1.3 | 1.1 | 1.1 | 1.1 | 1.0 | 2.0 | 1.9 | 0.9 | 1.0 | 1.4 | 1.4 | 2.4 | 0.8 | 1.8 | 1.4 | 1.5 | 1.6 | 0.79 |
| YPL109C | 1.1 | 2.5 | 0.5 | 1.2 | 1.2 | 1.2 | 1.8 | 1.1 | 1.2 | 1.5 | −0.2 | 0.9 | 1.8 | 1.3 | 1.3 | 1.9 | 1.2 | 1.2 | 0.21 |
| YPL149W | 1.5 | 1.5 | 0.6 | 2.8 | 0.8 | 0.9 | 1.2 | 1.1 | 5.6 | 3.7 | 1.2 | 0.9 | 2.3 | 1.0 | 0.8 | 1.2 | 2.2 | 2.9 | 1.39 |
| YPL154C | 0.7 | 3.3 | 4.2 | 1.2 | 1.2 | 3.5 | 1.5 | 0.8 | 2.8 | 1.7 | 1.8 | 1.5 | 3.1 | 0.8 | 4.0 | 1.8 | 1.6 | 2.0 | 3.78 |
| YPR103W | 0.7 | 1.1 | 1.3 | 0.7 | 1.7 | 1.2 | 1.6 | 1.0 | 5.5 | 2.0 | 0.9 | 1.2 | 2.8 | 1.8 | 1.0 | 1.1 | 1.2 | 1.5 | 1.93 |
| YPR108W | 0.8 | 1.0 | 1.3 | 0.6 | 1.3 | 1.5 | 1.6 | 1.6 | 3.7 | 2.8 | 1.8 | 1.0 | 2.6 | 1.0 | 1.3 | 1.2 | 1.0 | 1.3 | 2.25 |
| YCL027W | 0.7 | 2.0 | 1.0 | 1.0 | 1.2 | 1.3 | 1.0 | 0.6 | 0.7 | 16.3 | 0.9 | 0.8 | 1.1 | 1.2 | 3.6 | 1.1 | 0.7 | 0.8 | 0.30 |
| YDR055W | 1.7 | 2.1 | 2.2 | 2.8 | 1.7 | 3.9 | 1.5 | 1.6 | 0.8 | 2.4 | 2.2 | 1.4 | 3.1 | 0.9 | 5.7 | 3.4 | 2.0 | 1.9 | 1.55 |
| YEL042W | 0.6 | 0.9 | 1.7 | 0.9 | 0.7 | 0.5 | 0.5 | 0.7 | 0.2 | 0.3 | 1.6 | 1.0 | 0.4 | 0.6 | 4.8 | 0.6 | 0.7 | 0.8 | 1.21 |
| YGR136W | 0.9 | 1.2 | 1.3 | 0.7 | 1.2 | 1.1 | 1.5 | 1.3 | 2.1 | 2.2 | 1.3 | 1.2 | 1.7 | 1.1 | 2.6 | 2.2 | 1.3 | 1.8 | 1.42 |
| YHR142W | 1.5 | 0.8 | 2.1 | 0.8 | 1.3 | 1.2 | 0.9 | 1.3 | 0.4 | 0.9 | 1.1 | 1.3 | 1.2 | 1.2 | 4.8 | 1.0 | 1.3 | 1.3 | 1.17 |
| YJL073W | 0.8 | 0.8 | 2.5 | 0.7 | 1.2 | 2.0 | | 1.0 | 1.7 | 0.7 | 0.5 | 1.0 | 1.6 | 1.1 | 2.6 | 0.8 | 1.1 | 1.0 | 0.43 |
| YJR004C | 0.2 | 0.8 | 3.4 | 0.6 | 1.1 | 1.2 | 0.4 | 0.4 | 0.3 | 0.7 | 0.7 | 0.8 | 0.3 | 0.7 | 3.2 | 0.3 | 0.4 | 0.4 | 2.12 |
| YKL039W | 0.8 | 1.1 | 2.4 | 0.8 | 0.7 | 4.9 | 2.0 | 0.8 | 1.3 | 3.3 | 1.0 | 0.9 | 1.9 | 0.8 | 3.1 | 1.5 | 1.2 | 1.3 | 1.13 |
| YLR250W | 1.5 | 1.0 | 1.0 | 1.4 | 1.4 | 1.1 | 1.9 | 1.3 | 2.7 | 2.4 | 1.4 | 1.0 | 1.2 | 2.5 | 2.6 | 2.1 | 1.4 | 2.2 | 2.90 |
| YOR181W | 1.0 | 0.9 | 1.1 | 1.4 | 0.7 | 0.9 | 0.7 | 0.7 | 0.6 | 0.5 | 1.0 | 0.8 | 1.2 | 0.9 | 3.4 | 1.0 | 1.1 | 0.6 | 0.39 |
| YOR198C | 1.0 | 0.7 | 1.4 | 1.0 | 0.8 | 0.9 | 1.6 | 1.3 | 2.4 | 1.4 | 1.4 | 1.5 | 1.8 | 1.1 | 3.8 | 0.8 | 1.1 | 1.5 | 1.76 |
| YPL089C | 1.2 | 1.0 | 0.5 | 1.8 | 1.4 | 0.8 | 0.9 | 0.8 | 0.4 | 2.5 | 0.9 | 0.7 | 1.3 | 0.8 | 2.6 | 0.9 | 1.5 | 1.1 | 0.37 |
| YBR214W | 0.9 | 2.1 | 5.7 | 2.0 | 1.1 | 0.8 | 1.1 | 0.7 | 2.2 | 3.5 | 3.2 | 1.2 | 2.1 | 0.9 | 5.1 | 1.3 | 1.1 | 1.2 | 0.51 |
| YDR085C | 1.9 | 1.8 | 1.6 | 3.4 | 0.9 | 2.1 | 4.1 | 2.0 | 0.4 | 2.3 | 1.7 | 1.6 | 1.1 | 1.6 | 3.1 | 2.3 | 1.3 | 1.8 | 0.37 |
| YDR259C | 1.4 | 1.0 | 0.4 | 1.4 | 1.0 | 0.5 | 2.8 | 1.4 | 1.0 | 1.8 | 1.2 | 1.0 | 2.0 | 1.5 | 2.9 | 0.9 | 1.0 | 0.8 | 0.28 |
| YDR388W | 0.7 | 0.7 | 1.2 | 1.0 | 0.7 | 1.0 | 1.1 | 0.8 | 1.4 | 0.9 | 1.6 | 0.8 | 1.7 | 0.6 | 2.6 | 1.0 | 1.2 | 1.3 | 0.99 |
| YDR432W | 0.5 | 0.8 | 1.0 | 1.4 | 1.6 | 0.6 | 0.5 | 0.5 | 0.7 | 0.8 | 0.6 | 0.7 | 1.4 | 0.5 | 2.6 | 0.6 | 0.7 | 0.7 | 2.38 |
| YDR481C | 0.7 | 2.3 | 1.3 | 0.7 | 1.0 | 0.9 | 0.7 | 1.0 | 1.9 | 1.6 | 1.2 | 0.9 | 1.5 | 0.9 | 3.5 | 1.3 | 1.2 | 1.4 | 1.43 |
| YDR510W | 1.0 | 1.3 | 1.5 | 1.3 | 0.7 | 1.0 | 1.6 | 1.5 | 2.7 | 2.5 | 1.6 | 1.4 | 2.0 | 1.9 | 2.2 | 1.5 | 1.1 | 1.5 | 1.57 |
| YGR189C | 1.1 | 0.9 | 1.5 | 3.5 | 0.7 | 0.5 | 0.6 | 1.0 | 0.3 | 0.9 | 1.5 | 1.3 | 1.2 | 0.9 | 9.3 | 0.6 | 2.3 | 1.7 | 1.51 |
| YIL123W | 1.0 | 0.5 | 1.2 | 1.5 | 0.6 | 0.5 | 0.5 | 0.6 | 0.1 | 0.2 | 1.2 | 0.8 | 0.4 | 0.3 | 4.8 | 0.7 | 1.3 | 1.0 | 1.67 |
| YIL140W | 0.7 | 1.0 | 1.5 | 1.4 | 0.8 | 0.5 | 0.5 | 0.7 | 0.7 | 0.6 | 0.9 | 1.0 | 0.5 | 0.9 | 3.3 | 0.6 | 0.7 | 1.0 | 0.62 |
| YKL096W | 1.4 | 0.6 | 8.1 | 1.6 | 0.6 | 0.6 | 0.2 | 0.5 | 0.0 | 0.5 | 3.5 | 0.3 | 0.1 | 0.5 | 2.3 | 0.4 | 0.5 | 0.7 | 2.04 |
| YLR391W | | 0.7 | 2.9 | 0.8 | 1.2 | 1.5 | 0.7 | 0.7 | 0.4 | 0.7 | 1.4 | 1.0 | 0.9 | 0.5 | 3.9 | 1.3 | 1.4 | 0.9 | 1.72 |
| YMR094W | 1.0 | 0.5 | 1.3 | 1.7 | 0.9 | 0.5 | 0.8 | 1.0 | −0.1 | 0.9 | 1.0 | 0.9 | 1.4 | 2.8 | 6.6 | 1.0 | 1.0 | 1.0 | 0.23 |
| YMR104C | 1.9 | 14.3 | 2.2 | 2.6 | 0.7 | 1.9 | 1.5 | 0.9 | 0.6 | 3.2 | 1.1 | 1.0 | 1.5 | 1.1 | 2.5 | 2.3 | 2.8 | 1.9 | 0.51 |
| YMR276W | 0.6 | 1.4 | 1.5 | 0.7 | 1.3 | 0.8 | 1.1 | 0.8 | 1.8 | 1.7 | 1.9 | 1.2 | 1.9 | 1.2 | 3.3 | 0.9 | 1.2 | 0.7 | 0.47 |
| YOL013C | 0.9 | 1.0 | 1.0 | 1.5 | 0.7 | 1.1 | 1.7 | 1.0 | 0.8 | 1.0 | 1.4 | 1.1 | 1.8 | 0.9 | 2.7 | 1.3 | 1.1 | 1.4 | 0.65 |
| YOR355W | 0.7 | 0.6 | 1.1 | 0.8 | 1.0 | 1.6 | 1.1 | 0.8 | 0.1 | 0.8 | 1.2 | 0.9 | 1.1 | 0.6 | 3.2 | 0.8 | 0.8 | 0.9 | 0.98 |
| YCR071C | 1.1 | 0.7 | 0.9 | 0.8 | 1.2 | 2.5 | 1.4 | 0.9 | 0.6 | 1.2 | 1.1 | 1.1 | 1.3 | 1.7 | 1.1 | 1.5 | 1.2 | 1.3 | 0.96 |
| YDL008W | 1.1 | 1.0 | 1.0 | 1.1 | 1.8 | 2.7 | 2.0 | 1.2 | 2.3 | 3.0 | 0.9 | 1.2 | 2.0 | 2.2 | 0.7 | 1.3 | 1.5 | 1.6 | 0.95 |
| YDR115W | 1.3 | 0.9 | 0.6 | 1.5 | 1.4 | 2.3 | 1.6 | 1.3 | 1.1 | 1.0 | 1.2 | 0.9 | 1.1 | 1.5 | 0.8 | 2.2 | 1.2 | 1.4 | 1.50 |
| YER130C | 0.9 | 1.6 | 0.5 | 0.8 | 1.7 | 2.7 | 1.5 | 1.2 | 0.5 | 1.3 | 0.9 | 1.0 | 1.6 | 0.8 | 1.1 | 0.7 | 3.0 | 2.5 | 1.36 |
| YMR226C | 1.1 | 1.8 | 1.4 | 1.6 | 0.9 | 2.6 | 1.8 | 1.6 | 2.6 | 2.2 | 1.4 | 1.1 | 1.6 | 1.6 | 2.0 | 1.7 | 1.6 | 2.0 | 2.03 |
| YOR383C | 0.9 | 4.3 | 2.6 | 0.9 | 0.9 | 6.8 | 8.0 | 1.0 | 1.2 | 4.5 | 1.4 | 1.1 | 1.2 | 0.6 | 1.1 | 3.0 | 1.4 | 1.1 | 0.68 |
| YAR010C | 0.8 | 1.2 | 1.2 | 0.8 | 1.0 | 2.3 | 1.9 | 1.3 | 1.2 | 1.1 | 0.9 | 1.0 | 1.4 | 1.0 | 1.8 | 0.7 | 1.5 | 1.0 | 4.69 |
| YBL043W | 1.2 | 1.2 | 1.1 | 1.3 | 1.1 | 26.9 | 1.5 | 2.1 | 0.5 | 14.7 | 1.5 | 0.9 | 1.8 | 1.9 | 1.8 | 2.3 | 2.3 | 1.8 | 0.47 |
| YCR004C | 1.7 | 1.0 | 12.0 | | 1.2 | 1.8 | 1.4 | 1.2 | 4.7 | 2.1 | 1.9 | 0.8 | 1.3 | 3.7 | 0.8 | 4.9 | 1.4 | 3.5 | 3.02 |
| YCR088W | 0.9 | 1.1 | 1.2 | 0.9 | 1.1 | 2.0 | 1.2 | 0.5 | 0.8 | 0.9 | 1.0 | 1.3 | 1.9 | 0.9 | 2.8 | 0.7 | 1.4 | 0.9 | 0.47 |
| YDL238C | 0.7 | 1.0 | 1.1 | 1.7 | 0.9 | 2.1 | 1.4 | 5.8 | 1.7 | 2.7 | 1.1 | 0.9 | 2.0 | 1.2 | 1.3 | 1.7 | 1.1 | 1.3 | 0.35 |
| YDR084C | 0.9 | 0.9 | 1.5 | 1.1 | 1.2 | 2.1 | 0.9 | 0.7 | 0.6 | 0.6 | 0.7 | 0.7 | 1.0 | 1.0 | 3.9 | 1.0 | 2.0 | 1.3 | 1.12 |
| YDR104C | 0.8 | 1.1 | 1.2 | 1.2 | 1.3 | 1.7 | 1.0 | 1.3 | 2.8 | 2.7 | 1.0 | 1.1 | 1.6 | 1.4 | 1.4 | 0.9 | 0.9 | 1.0 | 0.34 |
| YDR315C | 1.0 | 1.4 | 1.0 | 1.3 | 0.9 | 9.4 | 1.4 | 1.4 | 1.1 | 1.6 | 1.1 | 0.6 | 1.6 | 1.4 | 1.5 | 1.5 | 1.2 | 1.2 | 0.48 |
| YDR358W | 0.7 | 1.1 | 0.7 | 1.1 | 1.4 | 2.5 | 2.8 | 1.2 | 2.9 | 3.2 | 1.0 | 1.1 | 3.2 | 1.2 | 0.9 | 2.2 | 1.9 | 1.9 | 0.50 |
| YEL066W | 2.0 | 2.9 | 1.5 | 2.2 | 0.8 | 2.2 | 1.8 | 1.4 | 1.3 | 2.4 | 1.9 | 0.8 | 1.6 | 1.9 | 1.0 | 1.9 | 1.0 | 1.1 | 0.64 |
| YER039C | 1.0 | 1.2 | 1.0 | 4.1 | 1.5 | 1.6 | 1.3 | 0.7 | 1.2 | 0.8 | 1.2 | 0.9 | 2.1 | 1.2 | 1.2 | 1.8 | 1.7 | 1.1 | 0.42 |
| YER107C | 0.9 | 0.5 | 1.0 | 0.6 | 1.3 | 2.5 | 1.0 | 0.8 | 0.4 | 0.5 | 0.7 | 0.7 | 0.9 | 1.1 | 0.7 | 0.9 | 1.1 | 1.2 | 0.92 |
| YFL028C | 1.2 | 1.1 | 0.7 | 1.5 | 1.7 | 1.7 | 2.0 | 1.5 | 2.7 | 1.8 | 1.3 | 1.1 | 1.4 | 1.6 | 0.8 | 1.9 | 1.2 | 1.5 | 1.28 |
| YFL043C | 1.0 | 1.2 | 0.6 | 1.2 | 1.2 | 1.8 | 1.8 | 1.0 | 2.2 | 3.8 | 1.1 | 0.9 | 1.5 | 1.5 | 1.1 | 2.0 | 1.5 | 1.2 | 0.38 |
| YGL229C | 0.6 | 0.7 | 0.9 | 0.8 | 1.0 | 2.2 | 2.2 | 0.7 | 1.8 | 1.7 | 1.1 | 1.1 | 1.7 | 1.0 | 0.2 | 1.8 | 1.1 | 0.9 | 0.44 |
| YGR257C | 0.7 | 0.8 | 0.9 | 1.1 | 1.3 | 2.1 | 2.4 | 1.0 | 1.5 | 1.9 | 1.2 | 0.9 | 2.0 | 0.9 | 1.3 | 1.3 | 1.1 | 1.3 | 0.84 |
| YHR004C | 0.9 | 1.1 | 0.8 | 1.1 | 1.5 | 2.0 | 1.5 | 0.9 | 1.1 | 0.6 | 0.8 | 0.9 | 1.1 | 0.8 | 1.2 | 2.4 | 1.4 | 1.6 | 0.84 |
| YHR071W | 1.5 | 1.1 | 0.6 | 1.6 | 1.5 | 2.0 | 1.0 | 1.7 | 1.0 | 7.2 | 1.8 | 0.8 | 2.3 | 1.6 | 3.0 | 1.7 | 2.6 | 1.0 | 0.79 |
| YJL089W | 1.1 | 1.5 | 1.0 | 0.8 | 1.1 | 1.6 | 1.1 | 1.1 | 1.6 | 3.9 | 0.6 | 1.2 | 1.8 | 1.8 | 6.4 | 1.2 | 1.2 | 1.1 | 0.21 |
| YJL116C | 0.9 | 1.8 | 1.3 | 1.9 | 1.3 | 3.4 | 0.8 | 1.8 | 2.4 | 2.6 | 2.9 | 1.1 | 4.3 | 2.6 | 1.0 | 1.3 | 1.2 | 0.7 | 1.01 |
| YJR086W | 1.2 | 1.1 | 0.8 | 1.4 | 1.3 | 1.9 | 1.7 | 1.1 | 1.4 | 1.1 | 1.0 | 0.8 | 0.9 | 2.2 | 2.0 | 1.4 | 1.4 | 1.6 | 1.24 |
| YKL008C | 1.2 | 0.6 | 2.0 | 0.8 | 0.9 | 3.8 | 1.4 | 1.2 | 0.3 | 0.4 | 1.5 | 1.5 | 1.7 | 0.8 | 1.3 | 2.1 | 1.4 | 1.9 | 2.11 |
| YKL013C | 1.4 | 0.9 | 1.4 | 1.1 | 1.9 | 1.9 | 1.8 | 0.9 | 1.5 | 1.4 | 1.1 | 0.7 | 1.3 | 1.4 | 1.2 | 1.5 | 1.6 | 1.7 | 1.55 |
| YKL041W | 1.2 | 0.8 | 1.1 | 1.0 | 1.3 | 2.2 | 1.0 | 1.6 | 1.6 | 1.5 | 1.2 | 0.8 | 1.3 | 1.3 | 1.0 | 2.5 | 1.1 | 1.3 | 0.91 |
| YKL139W | 0.9 | 0.7 | 1.4 | 0.8 | 2.8 | 3.5 | 2.6 | 1.2 | 1.6 | 1.5 | 1.4 | 0.8 | 1.6 | 1.0 | 1.0 | 1.3 | 1.9 | 0.8 | 0.62 |
| YKR014C | 1.1 | 1.3 | 0.7 | 1.1 | 1.5 | 2.1 | 1.7 | 1.6 | 2.4 | 2.0 | 1.1 | 1.1 | 1.5 | 1.8 | 1.4 | 2.4 | 1.0 | 1.7 | 2.05 |
| YLR093C | 1.3 | 0.8 | 0.7 | 1.2 | 1.4 | 2.9 | 1.9 | 1.2 | 1.5 | 1.3 | 1.0 | 1.0 | 1.3 | 1.3 | 0.9 | 2.4 | 1.7 | 3.2 | 1.90 |
| YLR118C | 1.0 | 1.1 | 1.6 | 1.0 | 1.0 | 2.5 | 1.5 | 1.0 | 1.1 | 1.3 | 1.2 | 1.0 | 1.4 | 1.5 | 0.8 | 3.5 | 1.3 | 1.9 | 1.17 |
| YLR251W | 1.8 | 0.8 | 4.2 | 4.6 | 1.6 | 2.1 | 2.6 | 1.2 | 2.1 | 1.3 | 4.1 | 0.8 | 1.7 | 1.3 | 0.8 | 2.5 | 2.1 | 3.8 | 0.78 |
| YMR027W | 0.8 | 2.6 | 0.7 | 0.8 | 1.1 | 2.1 | 2.0 | 0.9 | 1.1 | 1.4 | 1.1 | 0.9 | 2.4 | 0.9 | 4.0 | 0.8 | 1.3 | 1.2 | 4.05 |
| YMR262W | 1.0 | 1.3 | 1.9 | 1.1 | 1.2 | 2.0 | 1.9 | 1.0 | 3.0 | 1.2 | 1.3 | 0.8 | 1.7 | 0.9 | 0.3 | 1.0 | 1.0 | 1.2 | 0.65 |
| YNL214W | 1.2 | 0.9 | 1.1 | 1.1 | 1.4 | 2.3 | 1.3 | 1.1 | 1.5 | 1.5 | 0.6 | 0.8 | 1.2 | 1.7 | 0.9 | 2.0 | 1.1 | 1.2 | 0.47 |
| YOR149C | 0.7 | 0.9 | 1.0 | 0.8 | 1.1 | 2.3 | 1.0 | 0.7 | 0.4 | 1.0 | 1.0 | 0.9 | 1.3 | 0.9 | 1.3 | 1.1 | 1.0 | 1.2 | 0.76 |
| YOR165W | 0.9 | 1.5 | 0.7 | 0.8 | 0.8 | 2.2 | 0.9 | 1.1 | 0.8 | 1.3 | 1.3 | 0.7 | 0.9 | 0.8 | 0.8 | 1.0 | 0.9 | 1.0 | 1.03 |
| YOR285W | 1.4 | 6.0 | 3.5 | 1.9 | 1.7 | 2.1 | 4.0 | 1.2 | 4.0 | 2.9 | 2.3 | 0.9 | 2.1 | 1.6 | 2.0 | 2.9 | 2.3 | 3.3 | 2.62 |
| YOR367W | 1.0 | 0.9 | 1.7 | 0.9 | 1.1 | 3.3 | 1.9 | 1.1 | 1.1 | 1.1 | 1.4 | 0.5 | 0.8 | 1.5 | 1.1 | 1.3 | 1.4 | 1.4 | 0.60 |
| YPL018W | 1.0 | 1.4 | 1.0 | 0.5 | 1.8 | 2.1 | 0.9 | 1.1 | 1.4 | 1.2 | 1.3 | 1.1 | 2.2 | 1.3 | 0.9 | 0.8 | 1.3 | 0.8 | 0.22 |
| YPL203W | 1.3 | 1.0 | 2.2 | 1.0 | 2.1 | 1.8 | 2.1 | 1.1 | 1.8 | 2.1 | 1.5 | 0.9 | 1.5 | 1.0 | 0.6 | 2.3 | 1.3 | 1.7 | 0.79 |
| YPL255W | 0.8 | 0.3 | 1.1 | 0.6 | 0.9 | 2.0 | 0.6 | 0.9 | 0.1 | 0.1 | 0.7 | 0.6 | 0.6 | 0.7 | 0.6 | 0.7 | 0.8 | 0.7 | 0.55 |
| YPR073C | 1.2 | 1.9 | 0.5 | 1.4 | 1.8 | 2.0 | 2.5 | 1.5 | 1.3 | 2.6 | 0.9 | 0.8 | 1.4 | 1.5 | 0.8 | 1.8 | 1.4 | 1.8 | 1.55 |
| YDR518W | 0.8 | 1.7 | 1.1 | 1.2 | 1.6 | 1.2 | 2.7 | 1.1 | 0.2 | 1.2 | 1.0 | 1.5 | 1.8 | 0.8 | 2.5 | 1.7 | 1.4 | 1.6 | 0.93 |
| YOR381W | 0.8 | 2.0 | 0.8 | 1.8 | 1.0 | 1.0 | 3.6 | 0.8 | 1.2 | 3.5 | 2.6 | 1.1 | 1.7 | 0.8 | 0.6 | 2.5 | 0.8 | 0.9 | 0.38 |
| YBR109C | 1.6 | 1.3 | 2.4 | 1.1 | 1.0 | 1.4 | 2.4 | 1.7 | 3.8 | 3.3 | 1.5 | 1.4 | 2.0 | 2.0 | 1.4 | 1.9 | 1.4 | 1.9 | 2.15 |
| YBR201W | 1.2 | 4.3 | 1.2 | 2.4 | 1.2 | 1.0 | 3.4 | 2.2 | 0.8 | 1.0 | 1.1 | 1.7 | 1.9 | 1.1 | 1.6 | 1.5 | 1.8 | 1.6 | 0.75 |
| YDR041W | 1.2 | 1.1 | 0.6 | 1.8 | 2.0 | 1.6 | 2.4 | 1.2 | 1.3 | 1.3 | 1.2 | 0.8 | 0.9 | 1.4 | 1.0 | 2.4 | 1.5 | 2.5 | 1.85 |
| YER136W | 0.9 | 1.1 | 1.1 | 1.4 | 1.6 | 1.3 | 2.1 | 1.3 | 1.4 | 1.6 | 1.7 | 0.9 | 1.3 | 0.8 | 1.2 | 1.6 | 1.3 | 1.7 | 2.31 |
| YER159C | 1.1 | 0.9 | 0.2 | 1.6 | 2.1 | 1.3 | 2.0 | 1.4 | 0.2 | 0.9 | 1.6 | 0.9 | 0.9 | 1.1 | 2.2 | 1.1 | 1.6 | 1.9 | 0.99 |
| YJL030W | 1.6 | 1.4 | 0.5 | 1.0 | 1.3 | 1.1 | 1.9 | 1.6 | 2.3 | 1.9 | 1.4 | 1.0 | 1.4 | 2.1 | 1.5 | 1.9 | 1.3 | 1.5 | 0.87 |
| YJR029W | 0.8 | 1.3 | 0.6 | 0.8 | | 1.0 | 2.4 | 0.9 | 1.1 | 1.5 | 0.6 | 1.0 | 2.1 | 1.1 | 1.8 | 0.7 | 0.8 | 1.1 | 5.84 |
| YJR099W | 1.4 | 0.9 | 1.1 | 1.3 | 1.5 | 2.1 | 2.0 | 1.4 | 1.4 | 2.4 | 1.3 | 0.7 | 1.5 | 2.1 | 0.5 | 2.0 | 1.1 | 1.7 | 0.94 |
| YJR122W | 2.0 | 1.4 | 2.5 | 1.1 | 1.3 | 1.4 | 1.8 | 1.4 | 5.3 | 6.1 | 2.4 | 0.9 | 1.3 | 1.4 | 0.8 | 1.9 | 1.0 | 1.1 | 0.43 |
| YJR125C | 1.0 | 1.0 | 1.2 | 1.0 | 1.2 | 1.3 | 1.9 | 0.9 | 1.8 | 1.3 | 1.1 | 0.9 | 1.5 | 1.1 | 1.3 | 2.0 | 1.2 | 1.4 | 0.90 |
| YKL190W | 1.1 | 1.3 | 0.4 | 0.9 | | 1.5 | 2.0 | 1.1 | 1.8 | 1.9 | 1.5 | 0.9 | 1.3 | 1.7 | 1.4 | 1.8 | 1.3 | 1.3 | 1.14 |
| YLL051C | 0.8 | 1.1 | 1.0 | 1.5 | 1.4 | 1.2 | 2.0 | 0.9 | 0.9 | 1.8 | 0.7 | 1.1 | 1.3 | 0.8 | 1.2 | 0.8 | 1.0 | 1.0 | 0.81 |
| YLR090W | 0.7 | 1.1 | 0.8 | 1.3 | 1.2 | 1.3 | 2.2 | 0.9 | 1.8 | 1.4 | 1.0 | 0.8 | 1.7 | 1.0 | 0.8 | 1.9 | 0.8 | 1.0 | 0.67 |
| YMR051C | 0.9 | 2.8 | 0.8 | 1.3 | 1.7 | 1.2 | 2.6 | 0.9 | 1.0 | 1.4 | 1.1 | 0.9 | 1.3 | 1.2 | 1.7 | 1.0 | 1.8 | 1.2 | 3.91 |
| YMR139W | 1.0 | 3.4 | 2.5 | 1.5 | 1.2 | 1.5 | 2.2 | 0.9 | 2.8 | 2.4 | 1.3 | 0.9 | 1.6 | 1.3 | 0.9 | 2.3 | 1.1 | 2.0 | 0.89 |
| YNL015W | 2.3 | 6.7 | 1.8 | 2.4 | 1.8 | 1.7 | 3.5 | 2.2 | 2.1 | 4.3 | 2.1 | 2.8 | 3.9 | 1.6 | 1.4 | 5.3 | 3.0 | 5.2 | 1.03 |
| YNL079C | 1.1 | 1.0 | 0.4 | 1.0 | 1.7 | 1.1 | 1.9 | 1.6 | 1.5 | 1.2 | 1.3 | 1.2 | 1.8 | 1.0 | 1.5 | 1.4 | 1.0 | 1.9 | 3.26 |
| YNL223W | 1.1 | 1.2 | 0.6 | 1.7 | 1.0 | 1.8 | 2.2 | 1.4 | 3.3 | 1.9 | 1.3 | 1.3 | 2.0 | 1.2 | 1.0 | 2.1 | 1.0 | 1.5 | 0.34 |
| YNR007C | 1.2 | 1.8 | 0.7 | 1.0 | 1.5 | 1.3 | 1.9 | 1.2 | 2.5 | 2.0 | 0.7 | 0.9 | 1.5 | 1.5 | 0.6 | 2.0 | 1.4 | 1.6 | 0.49 |
| YNR035C | 0.8 | 1.2 | 1.7 | 1.0 | 0.8 | 1.1 | 2.6 | 1.1 | 2.9 | 1.5 | 1.5 | 1.1 | 1.7 | 1.0 | 1.3 | 1.6 | 1.4 | 1.8 | 1.60 |
| YOL016C | 1.3 | 2.0 | 0.4 | 2.0 | 1.6 | 0.9 | 3.0 | 1.3 | 1.9 | 1.6 | 1.3 | 1.4 | 3.0 | 0.7 | 0.5 | 1.6 | 4.3 | 3.8 | 2.10 |
| YOL104C | 1.0 | 1.7 | 0.2 | 1.8 | 1.9 | 1.4 | 2.6 | 1.4 | 0.2 | 1.7 | 1.2 | 0.8 | 0.6 | 2.2 | 1.3 | 0.5 | 1.1 | 0.9 | 0.32 |
| YPR107C | 1.4 | 1.0 | 1.2 | 1.9 | 1.5 | 1.3 | 2.0 | 1.3 | 2.4 | 2.4 | 1.2 | 0.7 | 1.4 | 1.7 | 1.0 | 1.4 | 1.2 | 1.5 | 0.81 |
| YOL152W | 0.2 | 0.6 | 1.0 | 0.8 | 0.7 | 1.8 | 2.4 | 4.1 | 0.9 | 1.2 | 5.9 | 0.8 | 0.5 | 0.4 | 0.8 | 1.2 | 1.1 | 1.2 | 0.91 |
| YAL007C | 1.3 | 0.9 | 0.9 | 1.5 | 0.6 | 0.7 | 1.5 | 1.7 | 1.1 | 0.9 | 2.0 | 1.3 | 2.0 | 1.3 | 2.5 | 0.8 | 1.2 | 1.5 | 1.74 |
| YDL043C | 1.0 | 0.8 | 0.8 | 1.2 | 1.1 | 0.5 | 0.9 | 1.3 | 0.8 | 0.8 | 3.9 | 0.9 | 0.8 | 1.2 | 0.8 | 1.2 | 0.8 | 0.8 | 0.78 |
| YDL212W | 1.0 | 1.2 | 1.6 | 1.2 | 1.1 | 0.4 | 0.7 | 0.9 | 0.4 | 0.6 | 2.0 | 0.8 | 0.5 | 1.0 | 1.1 | 0.9 | 1.1 | 1.7 | 4.27 |
| YDR183W | 1.2 | 0.7 | 0.9 | 1.4 | 1.5 | 1.4 | 1.8 | 1.8 | 2.5 | 2.9 | 1.8 | 1.1 | 1.4 | 1.9 | 1.4 | 1.3 | 0.8 | 1.0 | 0.61 |
| YGL089C | 0.4 | 0.7 | 2.0 | 1.0 | 1.5 | 0.5 | 0.4 | 1.0 | 0.0 | 1.9 | 2.3 | 0.4 | 0.2 | 1.4 | 0.9 | 0.2 | 0.3 | 0.6 | 3.00 |
| YGL096W | 1.9 | 2.9 | 1.2 | 2.5 | 0.9 | 0.5 | 1.8 | 1.7 | 2.1 | 3.6 | 1.7 | 1.1 | 1.2 | 1.4 | 0.8 | 1.7 | 1.1 | 1.1 | 0.54 |
| YGR006W | 1.2 | 1.1 | 0.9 | 2.9 | 0.5 | 0.5 | 1.0 | 1.2 | 0.7 | 0.9 | 1.6 | 0.8 | 1.4 | 1.1 | 1.2 | 1.5 | 0.9 | 1.0 | 0.40 |
| YHL034C | 0.7 | 1.0 | 0.7 | 0.8 | 1.9 | 1.1 | 1.4 | 1.4 | 1.9 | 1.9 | 2.2 | 0.8 | 1.6 | 1.0 | 1.4 | 1.6 | 1.2 | 1.5 | 2.11 |
| YHR163W | 0.9 | 1.2 | 1.3 | 1.2 | 0.8 | 1.9 | 0.9 | 1.3 | 1.4 | 1.1 | 2.9 | 0.7 | 1.1 | 1.5 | 0.7 | 1.2 | 1.1 | 1.2 | 1.71 |
| YIR024C | 1.5 | 0.9 | 0.9 | 2.0 | 1.1 | 0.8 | 1.5 | 1.4 | 5.6 | 2.9 | 6.3 | 1.1 | 1.6 | 2.6 | 0.9 | 2.2 | 0.9 | 1.4 | 0.75 |
| YKL070W | 1.0 | 0.8 | 1.7 | 0.9 | 1.3 | 1.1 | 1.1 | 1.5 | 15.0 | 11.0 | 10.4 | 0.6 | 1.0 | 2.2 | 0.8 | 0.9 | 1.0 | 0.9 | 0.29 |
| YLL050C | 1.4 | 1.0 | 1.2 | 1.7 | | 1.9 | 1.4 | 1.3 | 1.0 | 1.0 | 2.3 | 0.9 | 1.8 | 1.2 | 1.6 | 1.3 | 1.2 | 2.1 | 3.43 |
| YLR220W | 0.9 | 0.6 | 3.0 | 1.0 | 0.9 | 0.3 | 0.4 | 1.0 | 0.4 | 0.4 | 2.4 | 0.7 | 0.5 | 1.4 | 0.7 | 1.1 | 0.8 | 0.8 | 2.28 |
| YLR390W | 0.9 | 0.9 | 1.2 | 1.7 | 1.2 | 0.7 | 1.5 | 0.8 | 1.7 | 1.7 | 1.9 | 1.0 | 1.1 | 1.1 | 0.5 | 2.0 | 1.0 | 0.9 | 0.51 |
| YOL044W | 1.0 | 1.4 | 1.0 | 1.3 | 1.0 | 1.2 | 1.2 | 1.1 | 1.6 | 1.4 | 2.1 | 0.7 | 1.3 | 1.1 | 0.6 | 1.3 | 1.6 | 1.9 | 0.65 |
| YOL147C | 1.6 | 1.1 | 2.4 | 1.5 | 0.7 | 0.9 | 0.6 | 1.5 | 0.5 | 0.7 | 2.8 | 0.7 | 0.8 | 0.9 | 0.8 | 2.4 | 2.0 | 1.7 | 1.09 |
| YDR069C | 1.4 | 0.8 | 3.0 | 1.5 | 1.6 | 0.7 | 1.0 | 1.8 | 1.8 | 4.5 | 0.8 | 0.7 | 1.3 | 1.2 | 1.0 | 1.3 | 1.0 | 1.3 | 0.33 |
| YER131W | 1.5 | 0.3 | 0.9 | 0.7 | 1.6 | 0.9 | 0.6 | 2.0 | 0.0 | 0.6 | 1.0 | 0.9 | 0.2 | 0.8 | 0.9 | 0.4 | 0.9 | 0.9 | 5.32 |
| YGR044C | 0.8 | 1.3 | 0.7 | 1.2 | 1.1 | 1.4 | 1.3 | 1.8 | 0.9 | 0.8 | 2.1 | 0.8 | 1.4 | 1.0 | 0.5 | 3.3 | 1.1 | 2.6 | 1.43 |
| YMR240C | 1.1 | 0.6 | 2.5 | 0.7 | 1.4 | 0.8 | 1.1 | 2.3 | 0.5 | 1.1 | 1.4 | 0.6 | 0.8 | 1.1 | 1.0 | 1.0 | 1.1 | 1.1 | 0.39 |
| YBR105C | 1.7 | 1.5 | 0.9 | 2.0 | 0.8 | 1.8 | 2.0 | 1.2 | 1.1 | 3.3 | 1.1 | 1.2 | 1.1 | 1.7 | 1.4 | 1.3 | 1.3 | 1.1 | 0.96 |
| YBR182C | 2.0 | 1.9 | 1.0 | 2.4 | 1.4 | 1.0 | 1.7 | 1.3 | 0.6 | 3.2 | 1.5 | 1.3 | 1.2 | 1.2 | 3.1 | 1.1 | 3.4 | 1.6 | 0.41 |
| YBR186W | 1.0 | 1.0 | 1.1 | 1.7 | 1.0 | 0.5 | 0.9 | 0.9 | 1.0 | 2.4 | 0.9 | 0.8 | 0.7 | 1.1 | 0.9 | 1.2 | 0.7 | 1.0 | 0.31 |
| YEL052W | 1.1 | 1.0 | 2.0 | 2.0 | 1.3 | 1.2 | 1.2 | 1.2 | 3.0 | 3.4 | 1.7 | 1.1 | 1.5 | 1.1 | 0.7 | 2.1 | 0.9 | 1.0 | 0.90 |
| YER098W | 0.9 | 0.9 | 0.5 | 1.1 | 1.4 | 1.6 | 1.3 | 1.7 | 2.8 | 3.3 | 1.4 | 1.0 | 1.1 | 1.3 | 0.9 | 1.7 | 1.2 | 1.5 | 0.35 |
| YGL240W | 0.9 | 1.2 | 0.8 | 1.8 | 1.2 | 1.1 | 1.2 | 0.8 | 2.0 | 3.4 | 0.9 | 0.8 | 1.7 | 1.3 | 1.1 | 1.0 | 1.2 | 0.8 | 0.48 |
| YGR067C | 2.2 | 1.4 | 1.2 | 4.6 | 1.6 | 1.3 | 1.5 | 1.5 | 0.7 | 6.7 | 1.0 | 0.7 | 1.0 | 0.9 | 1.2 | 1.7 | 1.2 | 1.0 | 0.11 |
| YGR133W | 1.0 | 1.1 | 0.7 | 0.6 | 1.3 | 1.2 | 1.2 | 1.2 | 1.3 | 3.6 | 1.6 | 0.9 | 1.2 | 1.4 | 1.0 | 1.5 | 1.0 | 1.0 | 0.35 |
| YHR124W | 1.1 | 1.1 | 1.5 | 2.0 | 0.9 | 0.8 | 1.2 | 1.1 | 3.0 | 6.8 | 1.1 | 1.2 | 1.7 | 2.7 | 0.8 | 1.8 | 1.3 | 1.1 | 0.24 |
| YJL103C | 1.3 | 1.8 | 3.8 | 1.3 | 1.1 | 1.0 | 0.7 | 0.8 | 2.3 | 2.4 | 1.5 | 0.8 | 1.4 | 1.0 | 1.3 | 0.9 | 1.4 | 1.2 | 0.44 |
| YJR036C | 1.2 | 1.2 | 1.0 | 4.3 | 1.5 | 0.9 | 1.0 | 1.0 | 1.4 | 3.0 | 1.1 | 0.9 | 1.1 | 1.3 | 1.8 | 2.0 | 0.9 | 1.2 | 0.31 |
| YLR216C | 1.2 | 2.3 | 3.3 | 1.1 | 1.2 | 1.2 | 5.2 | 1.3 | 14.1 | 4.1 | 1.8 | 0.7 | 1.2 | 1.1 | 1.2 | 1.5 | 1.2 | 1.3 | 1.44 |
| YLR389C | 0.8 | 6.4 | 2.3 | 0.6 | 0.7 | 0.2 | 1.0 | 1.0 | 2.4 | 2.8 | 0.9 | 0.8 | 1.9 | 0.8 | 1.0 | 1.4 | 1.0 | 1.0 | 0.60 |
| YNL128W | 1.3 | 4.6 | 1.1 | 0.8 | 1.2 | 1.1 | 1.0 | 1.3 | 2.9 | 2.4 | 0.9 | 0.9 | 1.4 | 1.4 | 1.1 | 0.6 | 0.9 | 0.9 | 0.21 |
| YOL133W | 1.0 | 1.6 | 1.4 | 1.2 | 0.9 | 1.6 | 1.8 | 1.0 | 4.7 | 3.8 | 1.5 | 0.6 | 1.0 | 1.4 | 1.3 | 2.0 | 1.3 | 1.3 | 0.87 |
| YOR133W | 1.1 | 1.3 | 1.6 | 1.3 | 1.3 | 0.9 | 1.1 | 0.8 | 3.1 | 4.9 | 1.3 | 0.8 | 1.3 | 1.1 | 1.8 | 1.3 | 1.2 | 1.2 | 0.26 |
| YOR227W | 1.1 | 2.4 | 3.2 | 0.9 | 0.5 | 1.0 | 1.0 | 0.7 | 2.9 | 3.0 | 1.2 | 1.0 | 1.7 | 1.1 | 3.7 | 2.2 | 0.8 | 1.3 | 0.35 |
| YPR015C | 1.3 | 1.3 | 1.5 | 1.7 | 1.1 | 0.7 | 1.3 | 1.2 | 5.6 | 5.3 | 1.0 | 0.7 | 1.5 | 1.5 | 1.6 | 2.0 | 1.0 | 1.0 | 0.32 |
| YPR086W | 1.0 | 1.4 | 0.7 | 0.7 | 1.2 | 1.1 | 1.6 | 1.3 | 3.0 | 2.9 | 1.3 | 0.8 | 1.0 | 1.0 | 1.3 | 1.4 | 1.2 | 1.2 | 1.37 |
| YBL056W | 0.7 | 0.7 | 1.1 | 1.2 | 1.8 | 1.6 | 1.4 | 1.3 | 3.6 | 2.2 | 0.7 | 0.9 | 2.7 | 0.9 | 1.0 | 1.7 | 1.1 | 1.5 | 2.49 |
| YBR026C | 1.1 | 1.7 | 2.8 | 0.9 | 1.2 | 0.1 | | 1.1 | 3.4 | 1.6 | 1.6 | 1.2 | 1.5 | 1.5 | 0.7 | 0.5 | 0.8 | 0.9 | 0.59 |
| YBR123C | 0.8 | 0.7 | 1.6 | 1.8 | 1.1 | 1.3 | 0.9 | 0.9 | 3.2 | 2.5 | 1.1 | 1.0 | 1.4 | 1.1 | 1.0 | 1.2 | 0.8 | 1.0 | 0.62 |
| YDR099W | 0.9 | 1.0 | 2.0 | 1.3 | 1.1 | 2.2 | 1.3 | 1.1 | 2.4 | 1.3 | 1.1 | 0.9 | 1.5 | 0.9 | 1.9 | 1.4 | 1.2 | 1.7 | 3.51 |
| YDR177W | 1.2 | 1.7 | 1.0 | 1.0 | 1.0 | 1.4 | 1.3 | 1.6 | 3.5 | 1.9 | 1.3 | 1.2 | 1.7 | 2.4 | 0.8 | 1.3 | 1.3 | 1.5 | 1.77 |
| YDR392W | 0.9 | 2.9 | 1.1 | 1.3 | 1.2 | 0.6 | 1.1 | 1.1 | 4.3 | 1.5 | 1.2 | 0.8 | 1.4 | 1.3 | 1.1 | 1.4 | 1.0 | 1.2 | 0.63 |
| YDR394W | 0.9 | 1.2 | 1.3 | 0.8 | 1.2 | 1.1 | 1.2 | 1.3 | 3.8 | 2.0 | 1.8 | 0.6 | 1.0 | 1.2 | 1.1 | 1.0 | 1.1 | 0.8 | 0.97 |
| YER184C | 2.3 | 1.6 | 1.1 | 2.3 | 1.0 | 0.6 | 0.9 | 1.1 | 4.6 | 3.2 | 1.3 | 0.8 | 1.6 | 1.4 | 1.4 | 1.2 | 1.6 | 2.3 | 0.29 |
| YFL059W | 1.2 | 1.4 | 1.2 | 2.2 | 1.1 | 1.0 | 1.3 | 1.5 | 4.1 | 4.2 | 1.5 | 1.2 | 2.3 | 2.4 | 0.9 | 1.6 | 1.3 | 1.1 | 0.57 |
| YGL185C | 1.3 | 1.9 | 1.9 | 1.9 | 0.8 | 0.8 | 1.9 | 1.5 | 4.1 | 2.3 | 1.5 | 1.5 | 1.7 | 1.5 | 0.9 | 15.8 | 1.3 | 1.6 | 0.23 |
| YHL019C | 1.1 | 0.8 | 1.2 | 0.6 | 1.0 | 1.2 | 1.1 | 1.0 | 2.1 | 1.4 | 1.3 | 0.8 | 0.8 | 0.8 | 1.2 | 1.3 | 1.0 | 1.0 | 0.55 |
| YHR012W | 1.1 | 1.0 | 1.2 | 1.0 | | 1.6 | 1.2 | 1.2 | 4.9 | 2.2 | 1.5 | 0.9 | 1.7 | 1.7 | 1.0 | 1.0 | 1.2 | 1.4 | 1.11 |
| YHR028C | 0.8 | 1.2 | 1.4 | 0.8 | 1.0 | 0.9 | 1.2 | 0.8 | 3.7 | 2.2 | 1.4 | 0.7 | 0.6 | 1.1 | 1.1 | 0.7 | 1.0 | 1.0 | 0.92 |
| YHR109W | 0.8 | 1.0 | 0.0 | 0.7 | 1.3 | 1.4 | 1.4 | 1.2 | 2.4 | 1.8 | 0.7 | 1.0 | 1.4 | 1.5 | 0.9 | 1.3 | 0.9 | 1.1 | 0.22 |
| YHR156C | 1.3 | 1.3 | 0.9 | 2.1 | 1.9 | 1.0 | 1.2 | 1.2 | 3.6 | 1.9 | 0.6 | 0.7 | 1.0 | 1.9 | 1.2 | 1.4 | 0.9 | 1.3 | 0.47 |
| YIL159W | 1.1 | 1.6 | 1.3 | 0.7 | 1.2 | 2.3 | 0.6 | 1.1 | 5.9 | 1.6 | 0.8 | 0.9 | 1.5 | 2.0 | 1.0 | 0.7 | 0.9 | 0.8 | 0.29 |
| YJL154C | 0.6 | 2.7 | 0.7 | 0.6 | 1.2 | 1.3 | 1.5 | 1.0 | 2.3 | 2.0 | 0.5 | 0.9 | 1.6 | 0.8 | 1.2 | 1.2 | 1.2 | 1.1 | 0.62 |
| YJR110W | 1.0 | 0.6 | 1.0 | 1.4 | 1.3 | 0.9 | 0.9 | 1.1 | 4.9 | 2.3 | 1.2 | 0.7 | 0.9 | 1.1 | 1.2 | 1.1 | 0.8 | 1.0 | 0.58 |
| YKL025C | 0.7 | 1.1 | 1.1 | 0.9 | 1.1 | 0.9 | 0.9 | 0.9 | 2.8 | 1.7 | 1.1 | 0.8 | 1.3 | 0.9 | 1.2 | 1.1 | 0.9 | 1.0 | 0.54 |
| YKL171W | 0.9 | 1.3 | 1.2 | 2.1 | 0.9 | 0.4 | 0.9 | 0.8 | 8.2 | 1.8 | 2.8 | 1.1 | 1.5 | 1.1 | 1.2 | 2.1 | 0.9 | 1.2 | 0.45 |
| YKL196C | 1.2 | 1.1 | 0.5 | 0.9 | 1.3 | 0.9 | 1.5 | 1.3 | 4.3 | 2.0 | 1.6 | 1.0 | 1.8 | 2.5 | 1.9 | 1.9 | 1.3 | 2.0 | 2.41 |
| YKR068C | 1.2 | 1.5 | 1.0 | 1.8 | 1.1 | 1.4 | 1.4 | 1.3 | 2.4 | 1.9 | 1.0 | 1.0 | 1.1 | 1.6 | 0.8 | 1.4 | 1.4 | 1.7 | 1.72 |
| YLR144C | 0.8 | 1.5 | 4.0 | 1.3 | 0.7 | 0.9 | 0.7 | 1.1 | 2.8 | 1.5 | 1.0 | 0.7 | 2.3 | 1.2 | 1.9 | 1.7 | 1.0 | 1.0 | 0.44 |
| YML112W | 1.0 | 1.0 | 1.1 | 2.0 | 1.0 | 1.4 | 0.9 | 1.3 | 3.2 | 2.4 | 1.0 | 1.0 | 1.3 | 1.8 | 0.8 | 0.9 | 0.8 | 0.9 | 0.49 |
| YOL038W | 0.9 | 1.0 | 0.4 | 1.6 | 1.8 | 1.2 | 1.4 | 1.4 | 3.5 | 2.7 | 2.0 | 0.9 | 1.8 | 2.1 | 1.7 | 1.6 | 1.1 | 1.4 | 1.29 |
| YOR257W | 1.1 | 2.5 | 0.8 | 1.1 | 1.1 | 0.8 | 1.3 | 1.5 | 3.4 | 1.4 | 1.0 | 0.7 | 0.8 | 1.5 | 0.7 | 1.6 | 1.1 | 1.6 | 0.68 |
| YOR265W | 1.4 | 1.0 | 0.8 | 1.0 | 1.2 | 1.4 | 1.3 | 1.4 | 3.5 | 2.2 | 1.3 | 0.8 | 1.8 | 2.5 | 1.2 | 1.1 | 1.0 | 1.2 | 0.93 |
| YPL124W | 1.3 | 1.3 | 0.7 | 0.7 | 1.6 | 0.8 | 0.9 | 1.8 | 2.2 | 1.0 | 0.9 | 0.7 | 0.9 | 1.6 | 0.6 | 0.8 | 1.3 | 1.2 | 0.41 |
| YPR125W | 0.6 | 1.3 | 0.9 | | 1.2 | 0.7 | 1.3 | 1.2 | 4.1 | 1.8 | 0.9 | 1.1 | 1.9 | 1.6 | 2.0 | 0.6 | 0.8 | 0.7 | 0.73 |
| YPR168W | 0.9 | 1.0 | 0.2 | 0.5 | 1.3 | 0.4 | 1.1 | 1.1 | 4.7 | 2.7 | 1.2 | 0.5 | 1.0 | 1.1 | 1.2 | 1.3 | 0.9 | 0.8 | 0.29 |
| YPR180W | 0.9 | 1.2 | 1.6 | 1.0 | 1.0 | 0.8 | 2.4 | 1.3 | 2.3 | 1.1 | 0.5 | 0.7 | 1.3 | 1.2 | 1.3 | 1.0 | 1.0 | 1.4 | 0.61 |
| YPR193C | 1.0 | 2.2 | 0.6 | 1.1 | 1.4 | 1.3 | 1.4 | 1.1 | 3.5 | 4.1 | 0.1 | 0.9 | 1.6 | 1.6 | 0.9 | 1.6 | 1.1 | 0.9 | 0.17 |
| YBR045C | 0.9 | 1.0 | 2.2 | 0.7 | 1.0 | 0.8 | 0.8 | 1.0 | 3.6 | 1.1 | 0.4 | 1.3 | 1.3 | 1.0 | 2.0 | 0.8 | 1.1 | 0.9 | 1.29 |
| YBR128C | 1.0 | 1.1 | 2.1 | 1.0 | 1.1 | 1.5 | 2.1 | 1.2 | 2.8 | 1.2 | 1.3 | 0.7 | 1.3 | 1.4 | 1.4 | 2.0 | 1.2 | 1.8 | 0.44 |
| YCL055W | 0.7 | 2.2 | 0.7 | 1.3 | 1.2 | 1.4 | 1.0 | 1.3 | 2.1 | 1.5 | 1.4 | 0.9 | 0.9 | 1.8 | 1.5 | 0.7 | 0.8 | 1.1 | 0.66 |
| YCR019W | 1.0 | 1.6 | 1.0 | 0.8 | 1.4 | 1.5 | 1.1 | 1.5 | 2.8 | 1.6 | 2.0 | 1.0 | 1.6 | 1.2 | 0.7 | 1.4 | 1.0 | 1.0 | 0.45 |
| YDL065C | 1.2 | 0.9 | 1.0 | 0.7 | 1.4 | 1.1 | 1.3 | 1.4 | 2.4 | 1.5 | 1.4 | 0.6 | 0.7 | 1.4 | 1.3 | 1.5 | 1.1 | 1.2 | 1.08 |
| YDL143W | 0.8 | 0.8 | 1.2 | 1.1 | 1.0 | 1.0 | 0.9 | 1.2 | 2.1 | 1.8 | 1.3 | 1.1 | 1.9 | 0.9 | 1.4 | 0.9 | 0.8 | 0.8 | 1.86 |
| YDL197C | 1.0 | 1.1 | 0.9 | 2.1 | 1.5 | 1.2 | 1.0 | 1.0 | 2.6 | 1.5 | 1.1 | 0.8 | 1.4 | 1.1 | 1.3 | 0.9 | 1.3 | 0.8 | 0.31 |
| YDL230W | 1.0 | 0.8 | 1.2 | 1.3 | 1.4 | 0.9 | 1.1 | 0.9 | 2.6 | 1.2 | 0.9 | 0.6 | 1.3 | 1.0 | 1.4 | 1.7 | 1.0 | 1.2 | 0.42 |
| YDR212W | 0.8 | 1.2 | 1.3 | 0.8 | 1.4 | 1.4 | 1.0 | 1.3 | 3.5 | 1.7 | 1.8 | 0.8 | 2.0 | 1.0 | 1.2 | 0.7 | 0.6 | 1.0 | 2.13 |
| YDR257C | 0.8 | 5.3 | 0.6 | 0.8 | 1.6 | 1.1 | 1.0 | 1.0 | 3.0 | 1.6 | 1.3 | 0.7 | 1.0 | 1.0 | 1.3 | 1.0 | 1.1 | 0.8 | 0.62 |
| YDR329C | 0.9 | 0.8 | 1.0 | 1.7 | 1.9 | 1.2 | 1.6 | 1.2 | 2.5 | 1.0 | 1.0 | 0.8 | 1.1 | 1.1 | 1.0 | 1.6 | 1.2 | 1.6 | 0.95 |
| YDR488C | 1.0 | 0.3 | 0.9 | 0.9 | 0.9 | 0.9 | 0.7 | 0.9 | 2.1 | 0.4 | 0.4 | 0.6 | 1.3 | 0.8 | 3.5 | 0.7 | 0.9 | 0.9 | 0.32 |
| YDR505C | 0.8 | 1.0 | 1.0 | 1.4 | 1.2 | 1.1 | 1.2 | 0.8 | 2.5 | 1.0 | 1.0 | 0.7 | 1.6 | 0.9 | 1.1 | 1.6 | 1.0 | 1.5 | 0.84 |
| YDR506C | 0.9 | 0.7 | 1.3 | 0.7 | 0.9 | 0.9 | 0.6 | 1.0 | 1.9 | 1.4 | 1.1 | 0.9 | 0.3 | 0.5 | 0.9 | 0.4 | 0.6 | 0.5 | 1.90 |
| YDR515W | 0.9 | 1.1 | 0.8 | 0.8 | 1.5 | 0.8 | 0.6 | 1.4 | 5.8 | 2.7 | 1.0 | 0.8 | 0.7 | 0.8 | 1.3 | 0.5 | 0.7 | 0.5 | 1.02 |
| YEL005C | 1.1 | 1.3 | 0.8 | 1.1 | 1.6 | 1.9 | 2.0 | 1.2 | 2.3 | 1.6 | 1.6 | 0.8 | 1.2 | 1.7 | 0.8 | 3.4 | 1.1 | 1.4 | 0.45 |
| YER048C | 0.8 | 1.2 | 0.4 | 1.4 | 1.8 | 1.9 | 1.9 | 1.6 | 2.3 | 1.1 | 1.6 | 0.8 | 1.1 | 1.0 | 0.6 | 1.8 | 1.3 | 1.6 | 1.08 |
| YER078C | 0.9 | 0.8 | 1.8 | 0.8 | 0.9 | 1.4 | 1.3 | 1.1 | 2.3 | 1.5 | 1.3 | 1.0 | 1.5 | 1.0 | 0.9 | 1.0 | 0.9 | 0.9 | 0.49 |
| YER089C | 0.5 | 0.5 | 1.4 | 1.1 | 0.8 | 0.6 | 0.9 | 0.5 | 2.0 | 2.3 | 1.2 | 0.7 | 1.3 | 0.6 | 1.0 | 0.8 | 0.7 | 0.6 | 0.75 |
| YER100W | 1.1 | 1.1 | 0.7 | 0.7 | 0.9 | 0.5 | 0.6 | 1.2 | 2.3 | 1.6 | 1.1 | 0.8 | 0.7 | 0.8 | 1.0 | 1.1 | 1.1 | 0.7 | 0.95 |
| YFR051C | 0.5 | 1.1 | 2.2 | 0.5 | 0.5 | 0.2 | 1.0 | 0.8 | 2.5 | 1.4 | 1.3 | 1.2 | 1.5 | 0.8 | 3.0 | 0.8 | 1.1 | 0.9 | 0.53 |
| YGL093W | 0.8 | 0.9 | 1.2 | 0.6 | 0.9 | 1.0 | 0.8 | 0.8 | 4.5 | 1.6 | 1.1 | 0.6 | 0.7 | 0.8 | 1.4 | 1.0 | 0.9 | 1.2 | 0.56 |
| YGL105W | 0.9 | 0.8 | 1.1 | 0.8 | 1.3 | 1.5 | 0.7 | 1.2 | 1.8 | 1.4 | 1.4 | 1.1 | 1.0 | 1.1 | 0.9 | 1.0 | 0.8 | 0.9 | 3.01 |
| YGL166W | 1.0 | 1.4 | 0.6 | 1.5 | 1.3 | 1.0 | 1.5 | 1.2 | 1.8 | 2.4 | 1.0 | 1.1 | 1.9 | 1.1 | 0.9 | 1.2 | 2.1 | 1.9 | 0.73 |
| YGL215W | 0.7 | 1.2 | 1.2 | 0.9 | 1.0 | 0.9 | 1.0 | 0.7 | 2.2 | 1.8 | 0.8 | 0.7 | 1.0 | 0.8 | 1.0 | 1.2 | 0.7 | 0.9 | 1.31 |
| YGL216W | 0.7 | 1.3 | 0.8 | 1.0 | 1.2 | 1.5 | 1.0 | 1.0 | 2.6 | 0.5 | 0.5 | 1.0 | 1.4 | 1.0 | 1.1 | 0.8 | 1.1 | 0.9 | 0.37 |
| YGL221C | 1.1 | 1.2 | 1.0 | 1.1 | 0.9 | 2.2 | 1.3 | 1.6 | 3.4 | 1.9 | 1.4 | 1.1 | 1.7 | 1.7 | 0.7 | 1.3 | 1.1 | 1.4 | 1.64 |
| YGR186W | 0.9 | 0.7 | 0.4 | 1.1 | 1.5 | 0.7 | 1.3 | 1.3 | 2.6 | 1.9 | 1.0 | 0.6 | 1.1 | 1.1 | 1.3 | 1.4 | 1.3 | 1.2 | 0.79 |
| YGR270W | 0.9 | 1.0 | 0.8 | 0.8 | 0.7 | 0.9 | 0.9 | 0.9 | 2.3 | 1.3 | 0.7 | 0.8 | 1.3 | 0.9 | 1.6 | 1.0 | 0.9 | 0.9 | 0.51 |
| YGR274C | 0.7 | 0.5 | 0.3 | 0.9 | 1.8 | 0.7 | 1.3 | 1.2 | 2.2 | 1.8 | 0.9 | 0.7 | 1.1 | 0.8 | 1.2 | 0.7 | 1.1 | 1.0 | 0.62 |
| YHR082C | 0.7 | 1.1 | 1.6 | 0.9 | 1.6 | 0.8 | 1.1 | 0.8 | 2.6 | 1.7 | 0.9 | 0.7 | 1.7 | 0.9 | 1.4 | 0.7 | 1.1 | 0.9 | 0.60 |
| YHR160C | 1.0 | 0.5 | 1.6 | 1.6 | 1.0 | 1.0 | 1.1 | 1.2 | 2.3 | 1.6 | −0.2 | 0.8 | 1.8 | 1.2 | 2.1 | 4.4 | 1.0 | 1.5 | 0.32 |
| YHR171W | 1.2 | 0.8 | 1.1 | 1.2 | 1.3 | 2.0 | 1.9 | 0.9 | 2.1 | 1.6 | 1.2 | 0.8 | 1.3 | 1.0 | 0.7 | 1.6 | 1.2 | 1.5 | 0.41 |
| YHR205W | 0.7 | 1.3 | 0.6 | 0.8 | 1.3 | 1.0 | 0.7 | 0.5 | 2.0 | 0.6 | 0.9 | 0.9 | 1.7 | 0.9 | 1.5 | 0.8 | 0.9 | 0.8 | 0.34 |
| YIL062C | 1.1 | 1.0 | 1.3 | 1.2 | 1.2 | 0.9 | 0.4 | 1.1 | 2.8 | 2.3 | 1.7 | 0.8 | 1.1 | 2.0 | 0.9 | 0.4 | 1.1 | 1.1 | 1.72 |
| YIL075C | 0.5 | 0.7 | 1.5 | 0.6 | 1.1 | 0.8 | 0.8 | 0.7 | 1.9 | 2.3 | 0.8 | 0.8 | 0.7 | 0.8 | 1.6 | 0.8 | 0.8 | 0.8 | 1.54 |
| YIR009W | 0.8 | 0.8 | 0.9 | 2.8 | 1.5 | 1.4 | 1.3 | 1.3 | 2.6 | 1.5 | 1.1 | 0.7 | 0.8 | 1.6 | 1.1 | 1.0 | 1.7 | 1.0 | 0.48 |
| YIR018W | 1.2 | 1.0 | 0.9 | 1.1 | 1.4 | 2.0 | 1.5 | 1.5 | 4.0 | 2.1 | 1.2 | 1.1 | 1.5 | 1.2 | 1.5 | 1.0 | 1.6 | 1.1 | 0.44 |
| YJR091C | 0.8 | 1.2 | 2.6 | 1.4 | 1.4 | 0.8 | 1.1 | 0.8 | 2.4 | 2.3 | 1.8 | 0.8 | 1.4 | 0.8 | 1.4 | 0.7 | 1.2 | 0.9 | 0.57 |
| YKL079W | 0.9 | 0.8 | 1.5 | 1.0 | 1.4 | 0.8 | 1.7 | 1.2 | 2.3 | 2.0 | 1.3 | 0.9 | 1.2 | 0.9 | 1.1 | 1.6 | 1.1 | 1.2 | 0.64 |
| YKR102W | 0.8 | 1.6 | 1.4 | 0.8 | 0.9 | | | 0.7 | 2.4 | 1.4 | 3.3 | 0.8 | 2.7 | 1.8 | 1.1 | 1.5 | 1.5 | 0.9 | 0.19 |
| YLL054C | 1.2 | 0.6 | 0.5 | 1.2 | 1.4 | 1.9 | 1.1 | 1.1 | 2.4 | 1.4 | 0.5 | 0.7 | 1.7 | 1.2 | 0.9 | 0.9 | 1.0 | 1.0 | 0.33 |
| YLR200W | 1.4 | 0.9 | 0.5 | 1.2 | 1.7 | 1.2 | 1.9 | 1.4 | 2.2 | 2.6 | 1.1 | 0.9 | 1.3 | 2.3 | 0.9 | 1.2 | 1.1 | 1.5 | 1.12 |
| YLR248W | 0.9 | 0.7 | 1.1 | 1.4 | 1.9 | 1.2 | 1.2 | 1.1 | 2.3 | 1.1 | 1.0 | 0.7 | 1.2 | 0.9 | 1.2 | 1.7 | 1.1 | 1.7 | 1.58 |
| YLR266C | 0.9 | 1.3 | 0.7 | 1.5 | 0.7 | 1.0 | 1.6 | 1.1 | 2.2 | 2.6 | 1.1 | 1.0 | 1.3 | 1.0 | 0.8 | 1.7 | 0.8 | 1.4 | 0.74 |
| YML088W | 0.9 | 1.6 | 0.3 | 0.5 | 1.5 | 0.7 | 1.1 | 1.1 | 4.0 | 2.2 | 0.5 | 1.0 | 1.3 | 1.1 | 1.4 | 0.8 | 0.8 | 0.8 | 0.58 |
| YMR091C | 1.0 | 0.7 | 0.8 | 1.0 | 0.7 | 0.8 | 0.8 | 1.3 | 2.1 | 1.3 | 0.9 | 0.8 | 0.9 | 1.5 | 0.9 | 1.3 | 0.9 | 1.2 | 1.03 |
| YMR110C | 1.0 | 1.4 | 1.8 | 1.9 | 1.3 | 2.1 | 1.9 | 0.9 | 2.3 | 1.6 | 1.6 | 0.9 | 1.8 | 0.8 | 0.9 | 2.7 | 1.5 | 2.5 | 1.63 |
| YMR255W | 1.2 | 1.4 | 0.7 | 1.8 | 1.3 | 0.9 | 1.5 | 1.4 | 2.8 | 2.4 | 1.2 | 0.9 | 1.1 | 1.6 | 1.0 | 1.3 | 1.5 | 2.0 | 0.97 |
| YNL039W | 0.7 | 0.8 | 0.3 | 0.4 | 2.1 | 0.8 | 1.1 | 1.2 | 2.1 | 1.8 | 0.4 | 0.9 | 0.8 | 0.8 | 1.3 | 1.3 | 0.9 | 1.1 | 0.61 |
| YNL077W | 1.5 | 1.1 | 2.3 | 1.2 | 1.5 | 0.9 | 1.2 | 1.0 | 4.4 | 5.1 | 1.0 | 0.7 | 1.5 | 0.5 | 0.3 | 0.6 | 1.3 | 1.0 | 0.78 |
| YNL083W | 0.8 | 1.4 | 1.3 | 1.2 | 0.8 | 0.7 | 0.9 | 1.1 | 3.1 | 1.6 | 0.9 | 0.9 | 1.2 | 0.9 | 0.8 | 1.8 | 0.8 | 1.1 | 0.37 |
| YNL147W | 1.3 | 2.4 | 0.6 | 0.7 | 2.0 | 1.5 | 1.4 | 1.8 | 2.1 | 2.9 | 1.2 | 0.8 | 1.0 | 2.0 | 1.2 | 1.7 | 1.0 | 1.6 | 1.32 |
| YNR006W | 1.1 | 1.8 | 0.5 | 0.9 | 1.5 | 0.9 | 1.5 | 0.9 | 2.2 | 2.3 | 0.8 | 0.9 | 1.6 | 0.9 | 1.2 | 1.1 | 1.8 | 1.2 | 0.40 |
| YNR034W | 0.9 | 1.4 | 5.3 | 1.6 | 1.0 | 0.9 | 1.3 | 1.0 | 3.0 | 2.1 | 1.3 | 0.9 | 1.4 | 0.9 | 1.1 | 2.3 | 0.7 | 1.1 | 0.64 |
| YNR047W | 0.7 | 1.0 | 1.7 | 0.5 | 1.1 | 1.0 | 1.0 | 0.8 | 2.0 | 1.2 | 0.6 | 0.9 | 1.5 | 0.9 | 1.2 | 1.5 | 0.9 | 1.0 | 0.44 |
| YOR023C | 0.7 | 1.2 | 1.3 | 0.8 | 1.2 | 1.8 | 1.1 | 0.6 | 3.5 | 1.3 | 0.9 | 1.1 | 1.9 | 0.9 | 1.6 | 0.8 | 1.1 | 0.8 | 0.37 |
| YOR058C | 2.0 | 1.4 | 2.0 | 0.6 | 0.9 | 0.9 | 0.8 | 1.7 | 6.4 | 4.5 | 1.4 | 0.7 | 1.7 | 1.7 | 1.1 | 1.6 | 0.9 | 1.0 | 0.31 |
| YOR069W | 0.8 | 1.6 | 0.7 | 1.3 | 1.0 | 0.5 | 1.2 | 1.1 | 1.9 | 1.4 | 2.8 | 0.9 | 1.1 | 1.3 | 1.4 | 1.5 | 0.8 | 1.1 | 0.69 |
| YOR229W | 0.9 | 0.9 | 1.0 | 0.8 | 1.5 | 1.3 | 1.1 | 0.8 | 2.5 | 0.8 | 0.7 | 0.8 | 1.3 | 1.0 | 1.2 | 0.9 | 0.9 | 1.0 | 0.58 |
| YOR256C | 1.0 | 0.8 | 0.9 | 1.5 | 1.7 | 0.9 | 1.4 | 1.1 | 2.7 | 1.8 | 1.7 | 0.7 | 1.6 | 1.1 | 1.1 | 1.5 | 1.2 | 1.5 | 0.53 |
| YPL020C | 1.1 | 0.7 | 0.7 | 1.3 | 0.8 | 0.8 | 1.1 | 1.1 | 2.3 | 1.6 | 1.1 | 0.9 | 1.1 | 1.1 | 1.6 | 1.2 | 1.3 | 1.4 | 0.91 |
| YPL105C | 0.6 | 0.8 | 0.6 | 0.7 | 1.1 | 1.2 | 0.7 | 1.0 | 3.4 | 1.4 | 0.6 | 0.7 | 1.0 | 0.6 | 1.1 | 0.5 | 0.8 | 0.6 | 0.80 |
| YPR066W | 1.2 | 2.2 | 1.1 | 2.2 | 1.3 | 0.7 | 1.3 | 0.8 | 2.3 | 1.9 | 0.9 | 0.8 | 1.4 | 1.2 | 0.7 | 1.6 | 1.1 | 1.2 | 0.41 |
| YPR081C | 0.9 | 1.0 | 1.0 | 1.1 | 0.8 | 1.1 | 1.1 | 1.0 | 4.2 | 2.6 | 0.9 | 1.0 | 1.4 | 1.1 | 1.3 | 1.8 | 0.9 | 1.3 | 0.34 |
| YPR140W | 0.9 | 1.2 | 0.8 | 1.8 | 0.9 | 3.0 | 1.9 | 1.1 | 2.1 | 1.3 | 0.8 | 0.8 | 1.3 | 1.4 | 1.7 | 2.4 | 0.8 | 1.3 | 0.47 |
| YPR155C | 0.9 | 2.5 | 1.5 | 1.4 | 0.9 | 0.9 | 1.4 | 1.0 | 2.0 | 1.3 | 0.9 | 0.9 | 1.3 | 1.2 | 0.5 | 3.4 | 0.8 | 1.6 | 0.40 |
| YPR185W | 0.7 | 0.9 | 0.3 | 0.4 | 1.1 | 0.9 | 1.2 | 1.0 | 2.5 | 1.7 | 0.9 | 0.8 | 1.7 | 1.0 | 1.1 | 1.3 | 1.3 | 1.1 | 0.68 |
| YBR076W | 2.3 | 1.1 | 1.4 | 5.0 | 0.9 | 0.8 | 1.0 | 0.7 | 0.4 | 1.8 | 0.8 | 0.9 | 1.9 | 1.2 | 1.1 | 0.5 | 1.4 | 1.2 | 0.36 |
| YDR373W | 1.7 | 0.8 | 0.5 | 1.3 | 1.8 | 0.9 | 1.4 | 1.9 | 2.5 | 2.8 | 1.7 | 0.5 | 1.0 | 2.1 | 1.6 | 1.4 | 1.2 | 1.6 | 1.08 |
| YFR014C | 2.1 | 2.4 | 0.8 | 2.6 | 0.7 | 0.8 | 1.4 | 1.3 | 1.0 | 1.6 | 1.8 | 0.9 | 1.3 | 1.0 | 0.6 | 2.3 | 1.2 | 2.9 | 0.94 |
| YHR136C | 2.4 | 13.6 | 1.4 | 2.1 | 2.4 | 1.4 | 0.5 | 1.3 | 0.1 | 2.0 | 2.0 | 1.2 | 0.3 | 1.7 | 0.5 | 1.3 | 1.6 | 2.5 | 1.33 |
| YIL129C | 0.8 | 5.8 | 1.0 | 1.2 | | 0.3 | | | 2.1 | 1.8 | 1.5 | | 1.9 | 0.9 | 1.0 | 1.0 | 1.3 | 0.8 | 0.25 |
| YMR077C | 1.1 | 3.9 | 1.1 | 1.8 | 1.0 | 0.5 | 1.4 | 1.7 | 1.9 | 2.5 | 1.5 | 0.9 | 0.8 | 1.6 | 1.7 | 2.3 | 0.9 | 1.3 | 0.59 |
| YBR264C | 1.2 | 3.7 | 1.5 | 1.5 | 0.7 | 0.9 | 1.3 | 1.2 | 0.9 | 1.1 | 1.0 | 1.0 | 0.9 | 1.6 | 1.1 | 1.8 | 0.9 | 1.3 | 0.61 |
| YPL177C | 1.2 | 3.7 | 0.9 | 1.3 | 0.7 | 0.8 | 0.8 | 1.3 | 0.5 | 1.0 | 1.4 | 0.8 | 0.8 | 1.3 | 0.6 | 1.9 | 1.2 | 1.1 | 0.75 |
| YGL056C | 0.7 | 3.5 | 1.3 | 1.0 | 0.6 | 0.4 | 1.0 | 0.9 | 0.4 | 0.6 | 0.0 | 1.1 | 0.8 | 1.1 | 1.2 | 1.5 | 0.9 | 1.1 | 0.47 |
| YBL101W-A | 1.0 | 5.7 | 0.7 | 0.6 | 1.0 | 1.4 | 1.3 | 1.5 | 1.2 | 2.1 | 1.2 | 1.1 | 2.0 | 1.5 | 2.1 | 1.1 | 1.3 | 1.2 | 1.24 |
| YER072W | 1.6 | 2.3 | 1.6 | 1.3 | 1.2 | 1.5 | 0.7 | 1.2 | 0.5 | 1.1 | 1.7 | 1.1 | 0.6 | 1.5 | 0.9 | 1.1 | 1.7 | 1.7 | 1.69 |
| YMR112C | 1.2 | 4.2 | 0.9 | 1.2 | 1.0 | 0.7 | 1.5 | 1.6 | 1.4 | 1.3 | 0.9 | 1.0 | 0.9 | 1.8 | 0.9 | 1.4 | 1.1 | 1.6 | 0.57 |
| YJR058C | 1.3 | 2.0 | 1.3 | 1.5 | 1.1 | 1.6 | 1.3 | 1.2 | 1.9 | 2.6 | 1.2 | 0.7 | 0.8 | 1.8 | 0.8 | 1.2 | 1.3 | 1.2 | 1.00 |
| YML055W | 1.7 | 2.3 | 0.6 | 2.0 | 0.9 | 1.2 | 1.7 | 1.7 | 1.0 | 1.4 | 1.4 | 1.0 | 1.7 | 1.9 | 0.6 | 1.4 | 1.2 | 1.5 | 1.08 |
| YDR080W | 0.9 | 1.8 | 0.5 | 0.6 | 1.2 | 1.5 | 1.6 | 1.2 | 1.7 | 2.6 | 1.2 | 1.0 | 2.0 | 1.1 | 1.2 | 1.7 | 1.2 | 1.3 | 0.68 |
| YNR030W | 0.6 | 2.1 | 2.2 | 0.7 | 0.8 | 1.2 | 1.0 | 0.6 | 1.0 | 1.4 | 1.2 | 0.8 | 1.5 | 0.6 | 1.1 | 0.4 | 0.9 | 0.8 | 1.19 |
| YNL196C | 1.1 | 1.8 | 1.4 | 1.3 | 0.9 | 1.2 | 1.0 | 0.7 | 0.5 | 2.2 | 0.7 | 1.0 | 2.2 | 1.2 | 0.8 | 2.1 | 1.1 | 1.2 | 0.50 |
| YOR148C | 1.4 | 1.4 | 0.5 | 1.8 | 0.9 | 0.9 | 1.2 | 1.5 | 0.8 | 0.8 | 1.3 | 0.9 | 1.4 | 1.4 | 1.0 | 1.3 | 1.0 | 1.3 | 1.08 |
| YER029C | 1.0 | 1.6 | 0.4 | 1.9 | 1.7 | 1.2 | 1.1 | 1.5 | 1.4 | 1.1 | 0.8 | 0.9 | 0.9 | 1.4 | 1.1 | 1.7 | 0.9 | 1.3 | 1.20 |
| YLR360W | 0.9 | 2.0 | 0.5 | 0.7 | 1.9 | 1.4 | 1.7 | 1.3 | 0.9 | 1.3 | 0.9 | 0.9 | 0.7 | 1.1 | 0.8 | 2.1 | 1.2 | 1.4 | 0.53 |
| YGR239C | 1.3 | 1.3 | 0.7 | 1.3 | 1.2 | 2.3 | 1.2 | 1.5 | 0.5 | 3.8 | 1.4 | 1.0 | 1.0 | 1.4 | 1.3 | 1.1 | 1.1 | 1.3 | 0.49 |
| YDL229W | 0.9 | 1.9 | 0.4 | 0.6 | 1.2 | 1.0 | 1.3 | 1.2 | 1.4 | 0.9 | 1.6 | 0.9 | 1.0 | 1.3 | 1.2 | 1.0 | 1.2 | 1.1 | 0.76 |
| YJR027W | 0.8 | 1.9 | 0.8 | 0.7 | | 1.5 | 2.0 | 1.0 | 1.2 | 1.3 | 1.1 | 0.8 | 1.3 | 1.1 | 2.3 | 0.7 | 0.7 | 1.1 | 4.88 |
| YKL198C | 1.2 | 1.4 | 0.8 | 0.2 | 0.9 | 1.0 | 1.2 | 0.6 | 0.0 | 0.8 | 1.4 | 0.7 | 1.9 | 6.6 | 2.6 | 0.7 | 1.0 | 0.8 | 0.17 |
| YBR031W | 1.1 | 0.8 | 4.1 | 1.4 | 1.4 | 0.7 | 0.5 | 1.0 | 0.1 | 1.0 | 0.9 | 1.0 | 0.3 | 0.4 | 0.9 | 0.7 | 1.0 | 0.8 | 7.58 |
| YBR118W | 1.3 | 0.8 | 2.9 | 1.2 | 1.0 | 1.3 | 1.0 | 1.3 | 0.5 | 0.9 | 1.0 | 0.8 | 0.8 | 0.7 | 1.6 | 0.6 | 1.2 | 0.9 | 8.91 |
| YCR106W | 0.9 | 1.1 | 2.8 | 1.1 | 0.8 | 1.4 | 0.9 | 0.8 | 2.0 | 1.2 | 1.2 | 0.8 | 1.2 | 0.8 | 1.3 | 1.2 | 1.1 | 1.2 | 0.76 |
| YDR012W | 0.9 | 0.8 | 3.9 | 1.2 | 1.1 | 0.9 | 0.5 | 1.1 | 0.3 | 0.7 | 0.9 | 1.2 | 0.2 | 0.4 | 0.8 | 0.7 | 1.0 | 0.7 | 7.07 |
| YDR134C | 1.0 | 0.7 | 3.9 | 1.9 | 0.8 | 1.1 | 0.4 | 0.6 | 0.1 | 0.5 | 1.9 | 0.6 | 0.4 | 0.5 | 0.8 | 0.9 | 0.7 | 1.1 | 5.84 |
| YDR276C | 1.5 | 1.6 | 6.1 | 1.4 | 0.7 | 1.1 | 1.0 | 1.1 | 1.1 | 2.7 | 1.4 | 0.9 | 1.1 | 1.3 | 2.2 | 3.6 | 1.3 | 2.4 | 2.48 |
| YGR279C | 0.9 | 0.7 | 3.6 | 1.0 | 1.2 | 1.6 | 0.8 | 0.6 | 0.3 | 0.7 | 0.9 | 0.7 | 0.3 | 0.4 | 1.3 | 0.8 | 1.0 | 0.8 | 3.77 |
| YJL059W | 1.0 | 1.7 | 3.1 | 0.8 | 0.9 | 1.6 | 1.1 | 0.8 | 1.7 | 1.6 | 1.8 | 1.0 | 1.6 | 1.3 | 0.9 | 0.8 | 1.1 | 1.0 | 0.39 |
| YKL056C | 1.3 | 0.7 | 2.6 | 1.4 | 1.7 | 0.9 | 0.5 | 1.0 | 0.1 | 0.7 | 1.1 | 0.8 | 0.1 | 0.7 | 0.9 | 0.6 | 0.8 | 1.2 | 6.68 |
| YKL097W-A | 1.5 | 1.3 | 4.2 | 1.3 | 1.2 | 0.9 | 0.2 | 0.7 | 0.0 | 0.4 | 1.6 | 0.7 | 0.2 | 0.6 | 2.0 | 0.8 | 1.1 | 1.4 | 4.01 |
| YNL209W | 0.9 | 0.9 | 2.7 | 0.9 | 1.3 | 0.9 | 0.5 | 1.1 | 0.2 | 0.8 | 0.9 | 0.9 | 0.5 | 0.6 | 0.9 | 0.3 | 0.6 | 0.6 | 8.06 |
| YNL307C | 0.8 | 0.6 | 3.1 | 1.3 | 1.0 | 1.9 | 0.9 | 0.9 | 1.0 | 0.9 | 1.0 | 1.0 | 1.0 | 0.7 | 0.6 | 1.0 | 1.0 | 1.3 | 2.79 |
| YPR028W | 1.0 | 1.1 | 4.0 | 1.7 | 1.0 | 1.4 | 0.8 | 0.7 | 0.5 | 0.8 | 1.4 | 0.6 | 1.7 | 0.8 | 0.9 | 1.3 | 1.2 | 1.7 | 4.06 |
| YPR149W | 1.0 | 1.1 | 5.9 | 1.7 | 1.7 | 1.6 | 2.1 | 0.6 | 1.2 | 1.8 | 1.1 | 0.7 | 1.4 | 1.3 | 1.0 | 2.2 | 1.1 | 1.1 | 2.65 |
| YAL016W | 0.8 | 1.0 | 2.8 | 0.9 | 0.9 | 0.9 | 1.1 | 1.0 | 1.2 | 1.9 | 0.9 | 0.8 | 1.4 | 0.9 | 1.1 | 1.1 | 0.8 | 1.3 | 1.00 |
| YBR283C | 0.6 | 0.7 | 1.9 | 1.2 | 1.4 | 2.2 | 0.8 | 0.8 | 1.5 | 1.2 | 2.0 | 0.8 | 0.8 | 0.6 | 1.4 | 0.9 | 0.9 | 0.9 | 2.62 |
| YBR286W | 0.7 | 1.4 | 3.8 | 1.2 | 1.3 | 1.5 | 1.3 | 0.9 | 1.2 | 1.6 | 1.0 | 1.1 | 1.8 | 1.5 | 1.3 | 2.6 | 0.8 | 1.6 | 2.98 |
| YCL008C | 0.8 | 1.1 | 1.8 | 1.2 | 1.2 | 0.9 | 0.9 | 0.7 | 0.9 | 1.4 | 0.9 | 0.9 | 1.0 | 0.9 | 1.4 | 0.6 | 0.9 | 0.9 | 0.47 |
| YCR069W | 1.0 | 1.2 | 2.0 | 0.9 | 1.1 | 1.4 | 1.1 | 0.7 | 0.8 | 0.7 | 1.3 | 1.4 | 1.6 | 1.0 | 2.4 | 1.2 | 1.2 | 1.3 | 0.97 |
| YDL061C | 1.4 | 0.5 | 2.9 | 0.9 | 1.3 | 1.1 | 0.5 | 1.0 | 0.0 | 0.5 | 1.4 | 1.0 | 0.1 | 0.7 | 1.0 | 0.6 | 1.1 | 1.0 | 4.40 |
| YDR151C | 1.3 | 1.0 | 2.3 | 1.8 | 0.8 | 0.7 | 0.8 | 1.5 | 2.6 | 2.8 | 2.1 | 0.9 | 2.8 | 1.1 | 0.6 | 1.2 | 0.8 | 1.1 | 0.96 |
| YDR382W | 1.3 | 0.5 | 2.4 | 1.2 | 1.1 | 0.6 | 0.4 | 1.2 | 0.0 | 0.4 | 1.2 | 0.7 | 0.1 | 0.7 | 1.1 | 0.4 | 1.0 | 1.0 | 6.40 |
| YDR385W | 0.6 | 0.4 | 3.5 | 0.9 | 1.1 | 0.5 | 0.4 | 0.7 | 0.2 | 0.5 | 0.9 | 0.9 | 0.3 | 0.5 | 0.8 | 0.3 | 0.6 | 0.4 | 7.27 |
| YDR407C | 0.7 | 1.0 | 2.0 | 0.7 | 0.8 | 1.2 | 1.4 | 0.9 | 1.5 | 1.7 | 1.2 | 1.0 | 1.3 | 0.8 | 1.3 | 1.0 | 0.9 | 0.9 | 0.52 |
| YGL206C | 0.4 | 3.5 | 2.0 | 0.5 | 0.6 | 0.8 | 0.8 | 0.5 | 0.7 | 0.7 | 0.9 | 0.7 | 1.1 | 0.8 | 1.3 | 0.6 | 0.8 | 1.0 | 1.04 |
| YGR172C | 0.6 | 1.3 | 2.5 | 1.3 | 0.6 | 0.9 | 1.0 | 0.8 | 0.4 | 1.1 | 0.8 | 0.8 | 0.7 | 1.1 | 1.9 | 1.0 | 1.1 | 1.1 | 0.94 |
| YIL015W | 1.1 | 1.7 | 2.0 | 2.5 | 0.9 | 0.4 | 0.9 | 0.9 | 0.3 | 1.1 | 0.8 | 0.9 | 0.8 | 5.9 | 1.1 | 0.8 | 0.8 | 1.0 | 0.17 |
| YIL018W | 1.0 | 0.4 | 2.3 | 1.4 | | 0.9 | 0.4 | 0.8 | 0.0 | 0.3 | 1.2 | 0.6 | 0.0 | 0.6 | 0.6 | 0.4 | 0.8 | 0.8 | 6.27 |
| YJL138C | 1.0 | 0.7 | 2.3 | 1.4 | 0.9 | 0.8 | 0.8 | 1.2 | 0.2 | 0.7 | 1.2 | 0.8 | 0.3 | 0.7 | 0.9 | 0.6 | 0.9 | 1.0 | 7.38 |
| YJL191W | 1.0 | 0.4 | 2.2 | 0.7 | | 1.3 | 1.0 | 0.8 | 0.0 | 0.4 | 1.1 | 1.0 | 0.9 | 1.1 | 0.7 | 2.2 | 1.0 | 1.0 | 1.86 |
| YJR047C | 1.1 | 0.8 | 2.6 | 1.1 | 1.1 | 1.1 | 0.4 | 0.9 | 0.7 | 0.5 | 1.1 | 0.9 | 0.9 | 0.9 | 0.6 | 0.8 | 0.8 | 0.9 | 5.29 |
| YJR119C | 0.9 | 1.0 | 2.0 | 1.0 | 0.9 | 1.4 | 1.1 | 1.6 | 0.5 | 1.3 | 1.1 | 0.9 | 1.2 | 1.3 | 1.5 | 1.0 | 0.7 | 0.9 | 0.30 |
| YJR123W | 1.2 | 0.6 | 2.2 | 1.0 | 1.3 | 1.0 | 0.4 | 1.2 | 0.0 | 0.5 | 0.9 | 0.8 | 0.1 | 0.6 | 1.1 | 0.4 | 0.8 | 0.9 | 7.84 |
| YJR145C | 0.8 | 0.7 | 2.3 | 1.3 | | 0.4 | 0.4 | 1.0 | 0.0 | 0.4 | 0.8 | 0.6 | 0.0 | 0.6 | 0.8 | 0.5 | 0.8 | 0.9 | 4.82 |
| YLR110C | 1.4 | 0.8 | 2.7 | 1.2 | 1.7 | 0.9 | 0.4 | 0.8 | 0.3 | 0.8 | 3.2 | 0.7 | 0.5 | 0.5 | 1.7 | 0.8 | 0.9 | 1.1 | 3.99 |
| YLR264W | 1.1 | 0.4 | 2.2 | 1.0 | 1.1 | 0.7 | 0.4 | 1.0 | 0.0 | 0.3 | 0.8 | 1.1 | 0.3 | 1.2 | 1.2 | 0.5 | 1.0 | 1.1 | 3.91 |
| YLR340W | 0.8 | 0.4 | 2.6 | 1.0 | 0.6 | 0.7 | 0.3 | 0.9 | 0.0 | 0.5 | 1.4 | 0.9 | 0.1 | 0.5 | 1.3 | 0.2 | 0.9 | 0.7 | 6.55 |
| YLR388W | 1.6 | 0.4 | 1.9 | 1.0 | 2.5 | 0.7 | 0.5 | 1.2 | 0.2 | 0.7 | 1.0 | 0.6 | 0.1 | 0.8 | 0.6 | 0.5 | 0.8 | 1.0 | 4.48 |
| YMR092C | 0.6 | 1.2 | 1.9 | 1.3 | 0.8 | 0.9 | 1.2 | 0.9 | 1.5 | 1.7 | 1.2 | 0.9 | 1.7 | 0.8 | 1.1 | 1.2 | 0.9 | 1.3 | 1.43 |
| YMR101C | 1.1 | 1.8 | 1.9 | 2.9 | 0.9 | 0.4 | 0.9 | 0.8 | −4.0 | 1.6 | −0.3 | 0.7 | 0.8 | 1.1 | 1.0 | 0.9 | 0.7 | 0.8 | 0.16 |
| YNL069C | 1.2 | 0.3 | 2.8 | 2.3 | 0.7 | 0.4 | 0.3 | 1.0 | 0.0 | 0.4 | 1.0 | 0.6 | 0.1 | 0.9 | 1.0 | 0.3 | 0.8 | 0.6 | 4.03 |
| YNL135C | 1.1 | 0.9 | 2.0 | 1.4 | 1.7 | 0.9 | 0.8 | 0.5 | 0.7 | 0.5 | 0.7 | 0.8 | 0.9 | 0.5 | 1.0 | 1.4 | 1.2 | 1.7 | 3.05 |
| YOL039W | 1.2 | 0.3 | 3.9 | 0.7 | 1.3 | 0.4 | 0.3 | 1.0 | 0.0 | 0.9 | 1.1 | 0.7 | 0.1 | 0.5 | 0.8 | 0.4 | 0.7 | 0.7 | 4.94 |
| YOL120C | 1.0 | 0.3 | 2.3 | 0.8 | 1.5 | 0.3 | 0.4 | 0.8 | 0.0 | 0.3 | 1.0 | 0.7 | 0.1 | 0.6 | 0.6 | 0.3 | 0.6 | 0.8 | 4.42 |
| YOR230W | 1.2 | 2.3 | 3.0 | 1.0 | 0.9 | 1.1 | 0.6 | 0.6 | 0.6 | 0.7 | 1.6 | 1.4 | 1.5 | 0.7 | 0.6 | 1.6 | 1.2 | 1.6 | 1.76 |
| YOR298W | 0.9 | 0.9 | 3.2 | 0.5 | 1.5 | 0.9 | 0.6 | 0.7 | 0.6 | 1.0 | 0.1 | 0.7 | 1.1 | 2.1 | 1.2 | 0.5 | 1.0 | 0.8 | 0.41 |
| YPL048W | 0.7 | 0.7 | 2.6 | 1.2 | 0.7 | 0.6 | 0.6 | 0.8 | 1.6 | 0.6 | 1.7 | 1.0 | 0.9 | 0.6 | 1.5 | 1.3 | 1.2 | 1.0 | 2.11 |
| YPL179W | 1.0 | 1.5 | 2.4 | 1.0 | 1.1 | 1.6 | 1.2 | 0.8 | 1.2 | 1.1 | 1.1 | 1.0 | 1.3 | 0.8 | 1.2 | 1.4 | 1.3 | 1.3 | 1.38 |
| YPL218W | 1.5 | 0.9 | 2.5 | 1.7 | 1.1 | 1.5 | 1.3 | 1.4 | 1.1 | 1.2 | 1.1 | 1.2 | 1.0 | 1.5 | 1.1 | 1.7 | 1.1 | 1.8 | 2.98 |
| YPL220W | 1.3 | 0.9 | 2.3 | 1.4 | 1.1 | 1.3 | 0.6 | 1.2 | 0.0 | 0.5 | 1.3 | 0.5 | 0.1 | 0.6 | 0.6 | 0.5 | 1.1 | 1.1 | 8.33 |
| YPR080W | 1.3 | 1.3 | 2.8 | 1.2 | 1.4 | 1.4 | 0.9 | 1.3 | 0.7 | 1.1 | 1.0 | 1.0 | 1.3 | 0.6 | 1.5 | 0.6 | 1.1 | 1.0 | 8.03 |
| YPR181C | 0.5 | 0.8 | 2.3 | 0.6 | 1.0 | 2.0 | 1.3 | 0.5 | 1.0 | 0.7 | 1.3 | 1.0 | 1.7 | 2.4 | 1.5 | 1.6 | 1.8 | 1.6 | 1.84 |
| YBR290W | 1.3 | 1.4 | 0.8 | 3.0 | 1.0 | 1.1 | 1.4 | 1.5 | 2.0 | 1.8 | 1.2 | 1.0 | 1.7 | 1.4 | 0.9 | 1.3 | 1.2 | 2.1 | 1.34 |
| YCR091W | 1.3 | 1.1 | 0.6 | 2.2 | 1.3 | 1.3 | 1.3 | 1.1 | 0.8 | 1.2 | 0.8 | 1.1 | 1.3 | 1.3 | 0.7 | 2.7 | 1.3 | 1.4 | 0.22 |
| YFL026W | 1.0 | 0.8 | 1.2 | 5.0 | 1.4 | 1.0 | 1.2 | 1.3 | 0.6 | 1.5 | 1.3 | 0.8 | 1.1 | 1.1 | 1.0 | 1.0 | 1.0 | 1.0 | 0.27 |
| YOR003W | 0.9 | 1.1 | 1.3 | 3.3 | 1.6 | 1.1 | 1.1 | 0.9 | 1.6 | 1.3 | 0.7 | 0.8 | 1.0 | 1.2 | 2.8 | 1.3 | 1.0 | 1.1 | 0.30 |
| YCR038C | 0.9 | 1.1 | 0.9 | 2.5 | 0.9 | 0.9 | 0.9 | 0.9 | 0.7 | 0.7 | 1.1 | 0.8 | 1.0 | 1.1 | 0.7 | 1.0 | 0.9 | 1.1 | 0.31 |
| YDL119C | 1.0 | 0.8 | 0.6 | 2.2 | 0.9 | 0.6 | 1.3 | 1.1 | 1.3 | 1.1 | 1.5 | 0.9 | 1.1 | 1.2 | 0.5 | 1.2 | 1.0 | 1.0 | 0.77 |
| YDL220C | 0.9 | 1.3 | 1.1 | 1.6 | 1.2 | 1.8 | 1.1 | 0.8 | 0.2 | 1.3 | 0.8 | 1.0 | 1.6 | 0.9 | 1.6 | 0.8 | 1.1 | 0.9 | 0.24 |
| YDR125C | 1.1 | 0.9 | 1.0 | 1.7 | 1.1 | 0.8 | 1.0 | 1.2 | 0.7 | 1.9 | 1.0 | 0.8 | 0.9 | 1.0 | 0.8 | 1.7 | 0.9 | 1.2 | 0.35 |
| YDR225W | 1.0 | 0.6 | 0.6 | 1.6 | 1.9 | 0.5 | 0.4 | 0.8 | 0.3 | 0.7 | 2.7 | 0.4 | 0.2 | 0.8 | 0.1 | 0.8 | 1.1 | 1.8 | 5.54 |
| YER066W | 0.9 | 0.7 | 0.9 | 2.1 | 1.5 | 1.3 | 1.3 | 0.7 | 1.7 | 1.4 | 1.5 | 0.7 | 0.9 | 1.1 | 0.5 | 3.3 | 1.1 | 1.1 | 0.66 |
| YER076C | 0.8 | 0.9 | 1.2 | 1.9 | 1.1 | 0.6 | 0.8 | 0.7 | 0.2 | 1.2 | 0.7 | 0.7 | 1.1 | 0.9 | 0.8 | 1.3 | 1.0 | 1.0 | 0.33 |
| YFR006W | 0.9 | 1.8 | 1.1 | 3.4 | 0.7 | 0.8 | 1.4 | 1.0 | −1.6 | 0.7 | 1.2 | 1.0 | 0.9 | 0.9 | 0.8 | 1.2 | 0.9 | 1.3 | 1.54 |
| YGL208W | 0.9 | 1.8 | 1.1 | 2.2 | 0.7 | 0.6 | 1.1 | 0.7 | 1.4 | 1.3 | 1.3 | 0.8 | 1.5 | 0.9 | 1.8 | 3.1 | 0.8 | 1.3 | 0.32 |
| YGR023W | 1.0 | 0.9 | 1.1 | 4.2 | 1.0 | 1.2 | 1.0 | 1.2 | 1.2 | 3.2 | 2.4 | 0.9 | 1.4 | 1.3 | 1.1 | 0.9 | 1.2 | 1.5 | 0.69 |
| YGR108W | 0.9 | 3.9 | 0.3 | 3.6 | 0.8 | 0.1 | 0.3 | 0.5 | 0.0 | 0.1 | 0.5 | 0.4 | 0.3 | 0.8 | 0.2 | 0.4 | 0.7 | 1.0 | 0.93 |
| YHR195W | 1.1 | 1.5 | 0.6 | 2.4 | 1.5 | 1.0 | 1.3 | 1.6 | 1.8 | 0.8 | 1.4 | 0.9 | 1.2 | 1.2 | 0.7 | 3.7 | 1.0 | 1.5 | 1.12 |
| YIL050W | 1.0 | 1.3 | 1.3 | 2.4 | 0.9 | 0.7 | 1.1 | 1.2 | 1.3 | 1.9 | 0.9 | 1.0 | 1.0 | 1.3 | 0.6 | 2.7 | 1.0 | 1.1 | 0.52 |
| YJR050W | 1.0 | 0.6 | 0.7 | 2.1 | 1.2 | 1.4 | 1.1 | 1.8 | 2.0 | 1.3 | 0.8 | 0.8 | 1.9 | 1.3 | 0.9 | 1.5 | 0.9 | 1.1 | 0.85 |
| YKL093W | 1.0 | 1.6 | 1.4 | 3.6 | 0.8 | 0.8 | 0.7 | 0.9 | 1.5 | 1.6 | 0.9 | 0.9 | 1.2 | 1.1 | 0.8 | 2.9 | 0.8 | 1.0 | 0.38 |
| YMR053C | 1.1 | 1.0 | 0.8 | 2.1 | 0.9 | 1.3 | 0.9 | 1.0 | 2.2 | 1.4 | 1.3 | 0.9 | 1.5 | 1.3 | 2.4 | 2.3 | 1.1 | 1.6 | 0.31 |
| YNL139C | 0.8 | 1.1 | 0.8 | 2.0 | 1.9 | 1.0 | 1.0 | 0.9 | 0.6 | 0.7 | 0.8 | 0.5 | 1.2 | 0.7 | 1.4 | 1.1 | 1.1 | 1.0 | 0.47 |
| YOR122C | 1.0 | 0.7 | 1.8 | 1.9 | 1.7 | 1.3 | 1.2 | 1.0 | 1.1 | 0.9 | 1.5 | 0.8 | 1.0 | 1.2 | 1.6 | 1.5 | 1.0 | 2.2 | 3.06 |
| YOR312C | 1.2 | 0.4 | 1.0 | 1.9 | 0.7 | 0.6 | 0.6 | 1.2 | 0.0 | 0.3 | 0.8 | 0.7 | 0.1 | 0.7 | 0.7 | 0.5 | 1.2 | 1.0 | 5.24 |
| YOR327C | 2.3 | 1.8 | 0.5 | 1.9 | 1.0 | 1.4 | 1.4 | 1.9 | 1.4 | 2.2 | 2.1 | 1.0 | 2.1 | 1.5 | 0.9 | 1.5 | 2.2 | 2.1 | 1.46 |
| YPL001W | 0.9 | 0.9 | 1.0 | 2.5 | 1.7 | 0.7 | 1.0 | 1.2 | 0.7 | 1.0 | 1.3 | 0.7 | 0.8 | 1.3 | 1.3 | 0.8 | 0.9 | 1.2 | 0.74 |
| YPL230W | 1.2 | 1.7 | 1.8 | 4.0 | 0.8 | 1.0 | 1.1 | 1.0 | 0.9 | 2.7 | 3.1 | 1.0 | 2.0 | 1.3 | 0.7 | 2.0 | 1.0 | 1.6 | 0.47 |
| YER025W | 0.6 | 0.7 | 0.6 | 0.6 | 1.5 | 1.0 | 1.5 | 1.1 | 0.4 | 0.6 | 1.2 | 0.7 | 0.6 | 2.1 | 0.8 | 2.6 | 1.8 | 2.3 | 1.96 |
| YHR185C | 0.9 | 1.1 | 1.4 | 0.7 | 1.4 | 1.0 | 1.3 | 0.8 | −0.6 | 0.9 | 0.7 | 0.8 | 0.4 | 1.7 | 1.3 | 3.2 | 1.4 | 3.0 | 1.64 |
| YIL076W | 1.3 | 0.6 | 1.1 | 1.0 | 1.8 | 0.6 | 0.5 | 1.3 | 1.0 | 1.4 | 1.1 | 0.8 | 0.8 | 1.4 | 1.0 | 0.8 | 1.2 | 2.5 | 3.73 |
| YMR238W | 1.9 | 1.3 | 1.1 | 1.3 | 1.5 | 1.3 | 1.2 | 0.9 | 1.3 | 1.4 | 1.2 | 1.0 | 1.5 | 1.2 | 2.3 | 1.4 | 2.2 | 2.4 | 1.36 |
| YBR009C | 1.6 | 0.8 | 0.4 | 1.7 | 1.4 | 1.0 | 0.5 | 1.2 | 0.6 | 0.6 | 0.8 | 0.5 | 0.4 | 1.1 | 0.2 | 0.4 | 1.0 | 2.4 | 7.00 |
| YBR010W | 1.0 | 0.8 | 0.5 | 2.0 | 0.8 | 1.1 | 0.8 | 1.3 | 0.5 | 0.7 | 1.0 | 0.7 | 0.4 | 1.0 | 0.3 | 0.8 | 1.1 | 2.3 | 7.25 |
| YCL067C | 1.2 | 1.1 | 0.3 | 1.7 | 1.1 | 1.7 | 1.3 | 1.7 | 1.4 | 1.9 | 1.6 | 1.0 | 1.4 | 2.1 | 2.2 | 1.7 | 0.8 | 2.2 | 3.15 |
| YCR096C | 1.1 | 0.9 | 0.4 | 1.6 | 2.1 | 1.2 | 1.7 | 1.5 | 1.2 | 1.6 | 1.4 | 1.0 | 1.0 | 2.1 | 2.1 | 1.8 | 1.0 | 2.1 | 2.34 |
| YDL137W | 1.4 | 1.1 | 1.2 | 1.2 | 1.8 | 1.0 | 2.0 | 1.1 | 1.1 | 1.3 | 1.5 | 1.6 | 1.7 | 1.4 | 1.3 | 2.3 | 1.3 | 2.2 | 4.95 |
| YDL192W | 1.4 | 1.0 | 0.6 | 1.2 | 1.4 | 1.0 | 1.7 | 1.5 | 0.8 | 0.6 | 1.3 | 1.1 | 0.9 | 1.2 | 1.3 | 1.3 | 1.1 | 2.3 | 5.57 |
| YDR224C | 1.1 | 0.7 | 0.8 | 0.9 | 1.2 | 1.0 | 0.8 | 1.4 | 1.1 | 1.3 | 1.1 | 0.7 | 0.5 | 1.7 | 0.3 | 1.1 | 1.2 | 2.2 | 5.62 |
| YDR378C | 1.3 | 1.3 | 1.4 | 1.3 | 1.0 | 1.0 | 1.4 | 1.5 | 0.5 | 1.3 | 0.8 | 0.9 | 0.8 | 1.4 | 0.8 | 1.0 | 1.7 | 2.2 | 2.40 |
| YMR197C | 1.3 | 1.6 | 1.4 | 1.9 | 1.1 | 1.6 | 1.5 | 1.4 | 1.9 | 2.5 | 1.0 | 1.1 | 1.5 | 4.1 | 1.7 | 1.9 | 1.2 | 2.2 | 1.20 |
| YOL109W | 2.0 | 2.0 | 1.5 | 1.8 | 1.0 | 1.3 | 1.2 | 1.5 | 0.1 | 1.1 | 1.0 | 0.8 | 0.5 | 1.4 | 1.0 | 0.8 | 1.6 | 2.2 | 4.13 |
| YPL010W | 1.0 | 1.1 | 0.9 | 1.0 | 1.4 | 1.0 | 1.9 | 1.5 | 1.7 | 1.5 | 1.3 | 1.1 | 1.1 | 1.3 | 1.2 | 1.3 | 1.3 | 2.0 | 3.25 |
| YHR132C | 0.8 | 0.7 | 1.7 | 1.4 | 1.0 | 0.7 | 0.8 | 0.8 | 0.9 | 1.1 | 1.1 | 0.8 | 1.2 | 0.9 | 1.4 | 2.3 | 0.8 | 1.3 | 1.52 |
| YJL141C | 1.0 | 0.8 | 1.0 | 1.6 | 1.5 | 1.1 | 1.1 | 0.9 | 1.2 | 1.3 | 1.2 | 0.8 | 1.4 | 0.8 | 0.8 | 2.2 | 1.4 | 1.6 | 0.94 |
| YKR098C | 1.0 | 1.1 | 1.2 | 1.7 | 1.1 | 1.8 | 1.7 | 1.3 | 1.4 | 2.7 | 1.9 | 1.1 | 1.4 | 1.4 | 0.9 | 3.9 | 1.1 | 1.6 | 0.55 |
| YLR206W | 0.7 | 1.6 | 1.4 | 1.1 | 1.1 | 0.5 | 0.7 | 0.8 | 1.5 | 1.0 | 1.3 | 0.8 | 1.4 | 0.8 | 2.4 | 2.3 | 1.1 | 1.2 | 0.72 |
| YAL055W | 1.2 | 1.0 | 0.9 | 1.0 | 1.4 | 1.3 | 1.5 | 1.9 | 1.0 | 1.4 | 1.3 | 1.2 | 1.5 | 1.6 | 1.1 | 2.6 | 1.3 | 1.3 | 0.62 |
| YAR062W | 1.2 | 1.8 | 1.4 | 0.7 | 1.0 | 0.6 | 1.3 | 0.9 | 0.4 | 0.9 | −1.1 | 0.8 | 1.2 | 1.1 | 1.3 | 2.4 | 0.9 | 1.0 | 0.32 |
| YBL102W | 0.8 | 1.0 | 1.4 | 1.2 | 0.9 | 2.5 | 1.6 | 0.8 | 2.3 | 1.1 | 1.2 | 0.9 | 1.7 | 1.1 | 1.1 | 2.0 | 2.4 | 1.5 | 1.26 |
| YBR161W | 1.2 | 0.8 | 2.3 | 0.7 | 1.1 | | 3.0 | 0.9 | 0.4 | 0.4 | 1.1 | 0.6 | 1.0 | 0.9 | 1.5 | 2.1 | 1.5 | 2.2 | 0.72 |
| YCR039C | 1.9 | 2.1 | 0.3 | 0.9 | 1.9 | 1.5 | 1.4 | 1.6 | 1.8 | 2.2 | 1.2 | 0.8 | 1.0 | 1.7 | 1.9 | 2.1 | 1.0 | 2.1 | 1.73 |
| YDL018C | 1.3 | 0.9 | 0.9 | 1.1 | 1.8 | 1.4 | 1.2 | 1.3 | 1.0 | 1.0 | 0.9 | 0.7 | 1.0 | 1.5 | 1.1 | 3.1 | 1.5 | 1.7 | 0.84 |
| YDR022C | 1.1 | 1.2 | 0.8 | 0.9 | 1.5 | 2.2 | 1.8 | 1.4 | 0.9 | 1.4 | 0.9 | 0.7 | 1.3 | 2.1 | 1.0 | 2.1 | 1.3 | 1.2 | 0.47 |
| YDR181C | 1.1 | 1.2 | 0.8 | 1.4 | 0.9 | 0.7 | 1.0 | 1.2 | 2.0 | 0.9 | 0.8 | 0.7 | 0.7 | 1.3 | 1.2 | 2.0 | 0.8 | 1.2 | 0.48 |
| YGR036C | 0.9 | 0.8 | 0.9 | 1.1 | 1.3 | 1.3 | 1.0 | 0.8 | 0.9 | 0.9 | 0.9 | 1.0 | 1.3 | 1.0 | 0.7 | 2.0 | 1.3 | 1.1 | 1.12 |
| YGR120C | 1.2 | 1.8 | 0.5 | 1.0 | 1.8 | 1.5 | 1.6 | 1.3 | 0.3 | 1.2 | 1.1 | 0.7 | 0.5 | 1.0 | 1.2 | 1.9 | 1.1 | 1.2 | 0.36 |
| YGR131W | 0.8 | 2.1 | 1.9 | 1.1 | 0.9 | 1.9 | 1.1 | 1.1 | 1.9 | 5.8 | 1.1 | 0.5 | 1.4 | 2.0 | 0.8 | 2.6 | 1.3 | 1.0 | 0.41 |
| YGR167W | 1.1 | 1.1 | 0.6 | 1.5 | 0.9 | 1.7 | 1.4 | 1.4 | 1.4 | 1.9 | 1.1 | 1.1 | 1.6 | 1.7 | 1.6 | 2.8 | 1.3 | 1.7 | 1.79 |
| YHL024W | 1.2 | 0.8 | 1.6 | 1.1 | 1.5 | 1.2 | 1.3 | 0.8 | 2.1 | 3.6 | 1.1 | 1.6 | 1.6 | 2.1 | 2.9 | 3.8 | 1.4 | 1.4 | 0.58 |
| YJL113W | 1.2 | 1.9 | 1.2 | 1.2 | | 0.7 | 1.2 | 1.3 | 1.3 | 2.1 | 1.2 | 0.7 | 0.9 | 1.3 | 1.7 | 2.1 | 0.9 | 1.5 | 0.41 |
| YJL146W | 1.3 | 1.9 | 1.2 | 2.0 | 0.8 | 0.7 | 1.3 | 1.0 | 1.0 | 1.7 | −1.1 | 1.1 | 0.8 | 1.5 | 1.2 | 1.8 | 1.0 | 1.3 | 0.30 |
| YJR019C | 0.8 | 1.8 | 1.9 | 1.6 | 1.1 | 0.8 | 0.9 | 0.7 | 1.1 | 0.6 | 2.4 | 1.1 | 1.4 | 0.8 | 1.1 | 2.6 | 1.8 | 1.4 | 0.33 |
| YJR049C | 1.0 | 1.2 | 1.0 | 1.0 | 0.7 | 0.6 | 1.0 | 1.2 | 1.4 | 1.4 | 1.6 | 1.0 | 2.1 | 1.2 | 1.2 | 1.7 | 0.9 | 1.2 | 0.44 |
| YLR078C | 0.9 | 1.1 | 0.7 | 1.4 | | 0.8 | 1.2 | 1.1 | 0.8 | 1.1 | 2.9 | 1.0 | 0.8 | 1.2 | 0.6 | 2.2 | 1.2 | 1.2 | 1.02 |
| YNR037C | 0.9 | 0.9 | 0.9 | 1.7 | 1.2 | 0.7 | 1.7 | 1.1 | 1.3 | 1.7 | 1.5 | 0.7 | 2.0 | 1.2 | 0.8 | 1.9 | 1.4 | 1.6 | 1.34 |
| YOR028C | 1.4 | 0.7 | 1.1 | 2.3 | 1.6 | 1.0 | 1.2 | 1.5 | 1.2 | 2.0 | 1.5 | 0.6 | 0.8 | 1.3 | 1.1 | 3.7 | 0.8 | 1.7 | 0.78 |

The tables show that the expressed mRNA of about 700 of 2400 unknown yeast genes was induced by any one of chemical substances such as heavy metals, agricultural chemicals, surfactants (Table 1), as well as the expressed mRNA of 167 mitochondria-located genes (Table 2), 52 DNA repair genes (Table 3), 161 energy genes (Table 4), 142 transport facilitation protein genes (Table 5), 90 stress protein genes (Table 6), 142 metabolism genes (Table 7), 60 detoxification genes (Table 8), and 507 genes belonging to other category (Table 9). Here, when a ratio of an amount of mRNA expressed in the presence of the chemical substance to an amount of mRNA expressed in the absence of the chemical substance is two or more, then it is considered significant.

The present inventors understood that the induction of certain yeast genes by toxic substances is caused by the activation of the promoters of the genes by the toxic substances. Thus, the inventors prepared a vector that comprised a polynucleotide sequence comprising the promoter of the yeast gene, which sequence is operably linked to a polynucleotide encoding a marker protein; and transformed yeast cells with the vector. The working examples hereinafter illustrate the preparation of such vectors, transformation of yeast cells by use of said vectors, and the detection of toxic substances by use of the transformed cells.

Promoter assay is a method for determining variations of the level of intracellular genes without destroying cells on the basis of the level of expressed marker genes instead of the expressed mRNA. The gene selected to detect a chemical substance is expressed in the absence of chemical substances, and thus a marker protein also occurs in the absence of chemical substances. In the method according to the present invention, the behavior of yeast genes on the addition of test materials is determined on the basis of the level of expressed marker protein so that the presence or the absence, and the kind of toxic chemical substances are predicted. Thus, it is desired that the production of marker proteins is lower in the absence of chemical substances, and the production of marker proteins is higher in the presence of chemical substances.

Under the circumstance, yeast gene as used in promoter assay is selected, of which intensity (level of expressed gene in control cells/average level of expressed whole genes) is preferably 1.5 or less, more preferably 1 or less, even more preferably 0.5 or less, and of which expression magnification (expressed mRNA in the presence of chemical substance/expressed mRNA in the absence of chemical substance) is preferably 3 or more, more preferably 10 or more, even more preferably 20 or more.

### Example 2

Primers for PCR to amplify the polynucleotide comprising the promoter of yeast gene YKL071w were prepared. Primers were designed using a primer design software, Oligo4.0-S, Sequencher I Mackintosh version. The base sequence of the upper primer was:

| | |
|---|---|
| cgcaataatactggaaacatcaa, | (SEQ ID No: 2) |

whereas the base sequence of the lower primer was:

| | |
|---|---|
| atcgactttgtttgcttagaat. | (SEQ ID No: 3) |

For PCR, yeast chromosome (Saccharomyces cerevisiae S288C, Cat.40802, Research Genetics, Inc.) was used as template, and the commercially available kit (KOD DNA Polymerase; Code KOD-101, Toyobo) was used as reagents.

Type YEp shuttle vector pYES2 (pYES2, Cat no: V825-20, Invirtogen Corporation, USA), which can be replicated both in E. coli. and yeast was used as vector (R. W. Old, S. B. Primrose Principles of Gene Manipulation 5th Ed., BAIFUKAN CO., LTD, pp 138-165, pp. 234-263, 2000). The portion of vector pQBI 63 (Cat no. 54-0082, Wako Pure Chemical Industries, Ltd.) corresponding to a marker protein GFP was used as the polynucleotide encoding GFP (SEQ ID No: 4).

First, oxidase was inserted into the multiple cloning sites of pYES2 to give a vector. Thereafter, the GAL1 promoter of pYES2 was replaced with the polynucleotide comprising the promoter sequence of the intended yeast gene YKL071w (SEQ ID No: 4) to give an intended plasmid vector. Suitable restriction enzymes were selected, and the insertion of the polynucleotide comprising oxidase and the promoter sequence was conducted.

Next, the yeast Saccharomyces cerevisiae W303 was transformed with the resultant plasmid vector according to the following procedures:
1) Incubating the yeast Saccharomyces cerevisiae W303 in 200 ml of SD medium under shaking until OD 660 reaches 0.5;
2) Suspending the collected cells in 5 ml of TE-buffer;
3) Adding 250 μl of 2.5 M lithium acetate;
4) Dispending each 300 μl, adding 10 μl of above plasmid vector thereto, then incubating the suspensions at 30° C. for 30 minutes;
5) Adding 700 μl of 50% PEG4000, and incubating the mixture Under shaking at 30° C. for 60 minutes;
6) Giving heat shock (42° C., 5 minutes), and then immediately cooling the mixture;
7) Washing the mixture twice with 1 M sorbitol; and
8) Seeding it on agar plates made of minimum essential medium.

The transformations were confirmed on selective medium (SD medium (Yeast nitrogen base without amino acids (Difco 0919-15)+glucose+amino acids (adenine, histidine, tryptophan)). Colonies that were grown on agar plate of selective medium were further checked for auxotrophy for amino acids.

### Example 3

The transformed yeast produced in Example 2 is allowed to grow to a steady state by shake culturing in a SD medium (adenine, histidine, tryptophan, leucine) at 25° C.

The yeast in a steady state is diluted 500 times with the SD medium, and it is shake cultured at 25° C. for 15 hours. After confirming the logarithmic growth phase at which absorbance at 600 nm is within 0.2 to 0.5, different concentrations of TPN is loaded. Solutions containing final concentration of 0.0053 ppm, 0.053 ppm, and 0.53 ppm are defined as solutions A, B, and C, respectively.

In addition, as a reference solution, a solution free from TPN is defined as a standard solution S.

The sample solution A, B or C is placed in the first cuvette of the chemical substance measurement system shown in the block diagram of FIG. 2, and then, measurement of a dissolved oxygen rate is started.

To the sample solution A, B or C and the standard solution S is added pyridoxamine phosphate and then measurement is started.

By comparing a temporal change in a measured current value for the sample solution A, B or C and a temporal change in a measured current value for the standard solution S, an amount of pyridoxamine phosphate oxidase produced by stimulation of the chemical substance (TPN) in respective solutions for measurement may be determined and, thereby, existence or a quantity of the chemical substance may be detected.

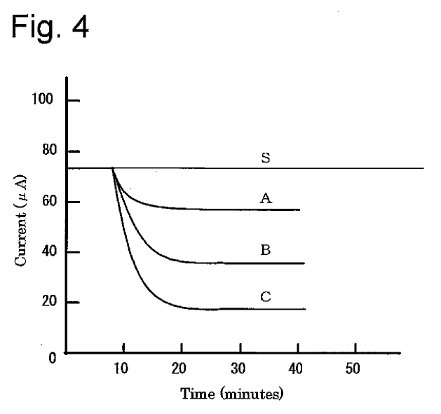

Temporal changes in current values for dissolved oxygen measurements of the sample solutions A, B and, C and the standard solution S are indicated in FIG. 4.

FIG. 4 demonstrates that the standard solution S does not show any change in a current value due to lack of oxidase expression, and that from the sample solutions A, B and C, as the concentration of the chemical substance (TPN) becomes larger, a decreasing rate of a current value at an initial stage becomes larger and a steady state value after reaching a plateau becomes lower.

1. A transformed cell which is transformed with a polynucleotide encoding an enzyme oxidizing a substrate by using oxygen as an electron acceptor in an enzymatic reaction or a vector comprising a polynucleotide in which a polynucleotide encoding oxidoreductase as a marker protein is operably linked at a downstream of a base sequence selected from a group consisting of a base sequence comprising a promoter responding to the chemical substance among promoters of yeast genes and a base sequence comprising a promoter from a gene that is homologous to the genes and is derived from other species.

2. The transformed cell according to claim 1, wherein the enzyme oxidizing a substrate by using oxygen as an electron acceptor in an enzymatic reaction is an enzyme selected from a group consisting of oxidase and oxygenase.

3. The transformed cell according to claim 1, wherein the polynucleotide encoding an enzyme oxidizing a substrate by using oxygen as an electron acceptor in an enzymatic reaction comprises a base sequence of an oxidase-related gene or a base sequence of an oxygenase-related gene among yeast genomic sequences.

4. The transformed cell according to claim 3, wherein the oxidase-related gene comprises a base sequence of a gene selected from Q0045, Q0250, Q0275, YBL064c, YBR035c, YBR244w, YDL067c, YDR044w, YDR079w, YDR231c, YDR453c, YDR506c, YEL045c, YER014w, YER021w, YER058w, YER141w, YER145c, YER153c, YER154w, YFL041w, YGL187c, YGL191w, YGL205w, YGR029w, YGR060w, YGR062c, YGR112w, YGR234w, YHR051, YHR116w, YIL111w, YIR037w, YJL003w, YJR034w, YKL026c, YKR066c, YLL009c, YLL018c-a, YLR038c, YLR142w, YLR205c, YLR395c, YML086c, YML129c. YMR020w, YMR058w, YMR256c, YNL052w, YOR350c, YPL132w, and YPR037c for a yeast gene and base sequences of a gene that is homologous to the genes and is derived from other species.

5. The transformed cell according to claim 3, wherein the oxygenase-related gene comprises a base sequence of a gene selected from YBL098w, YDR402c, YGL055w, YGR175c, YGR255c, YHR007c, YHR176w, YJR025c, YJR069c, YJR078w, YJR149w, YLL057c, YLR205c, and YMR015c for a yeast gene and base sequences of a gene that is homologous to the genes and is derived from other species.

6. The transformed cell according to claim 1, which is a transformed yeast.

7. A method for detecting a chemical substance which comprises:
detecting a dissolved oxygen rate with an oxygen electrode in a solution comprising a sample to be evaluated, a transformed cell which is transformed with a polynucleotide encoding an enzyme oxidizing a substrate by using oxygen as an electron acceptor in an enzymatic reaction or a vector comprising a polynucleotide in which a polynucleotide encoding an enzyme oxidizing a substrate by using oxygen as an electron acceptor in an enzymatic reaction is operably linked, and a substrate to the enzyme oxidizing a substrate by using oxygen as an electron acceptor in an enzymatic reaction;
collecting output signals from the oxygen electrode for a first time period at an interval of a second time period; and
calculating a change in the dissolved oxygen rate based on the change in the output signals to detect existence or a quantity of the chemical substance in the sample to be evaluated.

8. A device for measuring a chemical substance which comprises:
a measurement cuvette for containing one solution which has an oxygen electrode site at a predetermined location in the interior; and
a main structure capable of holding a plurality of measurement cuvettes, wherein the main structure having a signal output means to output a signal indicating existence or a quantity of the chemical substance in the solution based on the output signal from each of the oxygen electrodes.

9. The transformed cell according to claim 2, which is a transformed yeast.

10. The transformed cell according to claim 3, which is a transformed yeast.

11. The transformed cell according to claim 4, which is a transformed yeast.

12. The transformed cell according to claim 5, which is a transformed yeast.


SEQUENCE LISTING (4 sequences)

SEQ ID NO: 1 - DNA, 1653 bp, Photinus pyralis
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 2 - DNA, 23 bp, Saccharomyces cerevisiae
cgcaataatactggaaacatcaa

SEQ ID NO: 3 - DNA, 22 bp, Saccharomyces cerevisiae
atcgactttgtttgcttagaat

SEQ ID NO: 4 - DNA, 687 bp, Saccharomyces cerevisiae
atgactaaacaagctgaggagacccaaaagccaatcatatttgctcctgagacgtatcaatatgataaatttactttgaatgaaaaacaacttactgacgacccaatcgatcttttcaccaaatggttcaacgaagccaaggaagacccaagggaaacgttgccagaagcaattactttttcatccgcggaactacctagtgggagggtgtcgtccaggattcttctttttaaggagctcgaccatagaggttttactatttattctaactggggaacctctagaaaggctcatgatattgctaccaacccgaatgcggcaatcgtattcttttggaaggatctgcaaaggcaggtgagagttgaaggtatcacagagcatgttaacagagaaacttctgaaagatactttaagacgagacctcgtggatccaagatcggtgcatgggcttcccgccaatcggatgttatcaagaacagagaagaactagacgagttgacccaaaaaaacaccgaacgtttcaaggatgctgaagacatcccatgtccagattattggggtggcttgagaatcgttccactggaaattgagttctggcaaggtagaccctcgagattgcatgatagattcgtttacagaagaaaaacagaaaacgatccatggaaagtcgttagactagccccatga